(19) United States
(12) Patent Application Publication
(10) Pub. No.: US 2009/0298811 A1
(43) Pub. Date: Dec. 3, 2009

(54) BENZIMIDAZOLONE DERIVATIVES

(75) Inventors: Kazuo Ando, Aichi-ken (JP); Ingrid Price Buchler, Chesterfield, MO (US); Shridhar Gajanan Hedge, Chesterfield, MO (US); Makoto Kawai, Aichi-ken (JP); Tsutomu Masuda, Aichi-ken (JP); Hirofumi Omura, Aichi-ken (JP)

Correspondence Address: PFIZER INC.;PATENT DEPARTMENT, Bld 114 M/S 114, EASTERN POINT ROAD, GROTON, CT, 06340, US

(73) Assignee: Pfizer Inc

(21) Appl. No.: 12/440,646

(22) PCT Filed: Sep. 3, 2007
(86) PCT No.: PCT/IB2007/002583

§ 371 Date: Mar. 25, 2009

Related U.S. Application Data
(60) Provisional application No. 60/825,318, filed on Sep. 12, 2006.

(51) Int. Cl. A61K 31/55; A61K 31/5377; A61K 31/4965; A61K 31/437; A61K 31/4245; A61K 31/4184; C07D 405/14; C07D 413/06; C07D 403/08; C07D 471/04; C07D 413/12; C07D 235/24

(52) U.S. Cl. 514/217.1; 514/234.5; 514/255.05; 514/303; 514/364; 514/395; 540/603; 544/139; 544/405; 546/118; 548/143; 548/306.4

(57) ABSTRACT

This invention relates to compounds and methods for the treatment of a condition mediated by CB1 receptor activity in a mammalian subject including a human, which comprises administering to a mammal in need of such treatment a therapeutically effective amount of the compound of formula (I) or pharmaceutically acceptable salts thereof, wherein: A, B, R¹, R² and R³ are each as described herein. These compounds are useful in the treatment of a condition mediated by CB2 receptor binding activity such as, but not limited to, inflammatory pain, nociceptive pain, neuropathic pain, fibromyalgia, chronic low back pain, visceral pain, acute cerebral ischemia, pain, chronic pain, acute pain, post herpetic neuralgia, neuropathies, neuralgia, diabetic neuropathy, HIV-related neuropathy, nerve injury, rheumatoid arthritic pain, osteoarthritic pain, back pain, cancer pain, dental pain, fibromyalgia, neuritis, sciatica, inflammation, neurodegenerative disease, spasticity, epilepsy, Tourette's syndrome, Parkinson's disease, neuroprotection, anxiety, cough, broncho constriction, irritable bowel syndrome (IBS), inflammatory bowel disease (IBD), colitis, cerebrovascular ischemia, cachexia, nausea, emesis, chemotherapy-induced emesis, rheumatoid arthritis, asthma, Crohn's disease, ulcerative colitis, asthma, dermatitis, seasonal allergic rhinitis, gastroesophageal reflux disease (GERD), constipation, diarrhea, functional gastrointestinal disorder, cutaneous T cell lymphoma, multiple sclerosis, osteoarthritis, psoriasis, systemic lupus erythematosus, diabetes, glaucoma, osteoporosis, glomerulonephritis, renal ischemia, nephritis, hepatitis, cerebral stroke, vasodialation, hypertension, vasculitis, myocardial infarction, cerebral ischemia, reversible airway obstruction, adult respiratory disease syndrome, chronic obstructive pulmonary disease (COPD), cryptogenic fibrosing alveolitis and bronchitis.

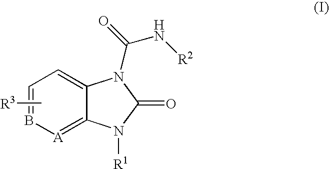

### BACKGROUND OF THE INVENTION

This invention relates to benzimidazolone derivatives. These compounds have cannabinoid CB1 receptor binding activity. The present invention relates to methods of treatment and use, comprising the above derivatives for the treatment of disease conditions mediated by CB1 receptor binding activity.

Cannabinoid receptors, endogenous cannabinoids and the enzymes that synthesize and degrade endocannabinoids make up the endocannabinoid system. CB1 and CB2 are two subtypes of cannabinoid receptors. CB1 and CB2 are both G protein coupled receptors. CB1 receptors primarily exist in the central nervous system, but are also found in some peripheral tissues including pituitary gland, immune cells, reproductive tissues, gastrointestinal tissues, sympathetic ganglia, heart, lung, urinary bladder and adrenal gland. CB2 receptors primarily exist in immune cells. Cannabinoid agonists are believed to be useful in the treatment of inflammatory pain, nociceptive pain, neuropathic pain, fibromyalgia, chronic low back pain, visceral pain, acute cerebral ischemia, pain, chronic pain, acute pain, post herpetic neuralgia, neuropathies, neuralgia, diabetic neuropathy, HIV-related neuropathy, nerve injury, rheumatoid arthritic pain, osteoarthritic pain, back pain, cancer pain, dental pain, fibromyalgia, neuritis, sciatica, inflammation, neurodegenerative disease, spasticity, epilepsy, Tourette's syndrome, Parkinson's disease, neuroprotection, anxiety, cough, broncho constriction, irritable bowel syndrome (IBS), inflammatory bowel disease (IBD), colitis, cerebrovascular ischemia, cachexia, nausea, emesis, chemotherapy-induced emesis, rheumatoid arthritis, asthma, Crohn's disease, ulcerative colitis, asthma, dermatitis, seasonal allergic rhinitis, gastroesophageal reflux disease (GERD), constipation, diarrhea, functional gastrointestinal disorder, cutaneous T cell lymphoma, multiple sclerosis, osteoarthritis, psoriasis, systemic lupus erythematosus, diabetes, glaucoma, osteoporosis, glomerulonephritis, renal ischemia, nephritis, hepatitis, cerebral stroke, vasodialation, hypertension, vasculitis, myocardial infarction, cerebral ischemia, reversible airway obstruction, adult respiratory disease syndrome, chronic obstructive pulmonary disease (COPD), cryptogenic fibrosing alveolitis and bronchitis (See Annu. Rev. Pharmacol. Toxicol. (2006) 46:101-22; Clinical Neuroscience Research (2005)5 185-199; Prostaglandins, Leukotrienes and Essential Fatty Acids (2002) 66(2&3), 101-121.)

Some cannabinoid agonists exhibit high affinity for both CB1 and CB2 receptors. Some CB agonists show a higher affinity for one of the CB1 or CB2 receptors. Compounds that have selective CB2 receptor binding activity may also have CB1 receptor binding activity and therefore may be useful in the treatment of CB1 mediated disorders. In the alternative, Compounds that have selective CB1 receptor binding activity may also have CB2 receptor binding activity and therefore may be useful in the treatment of CB2 mediated disorders.

Some of the compounds of this invention are described in PCT/IB06/000521, filed Mar. 2, 2006, which is herein incorporated by reference.

There is a need to provide new CB1 ligands that are good drug candidates. They should be well absorbed from the gastrointestinal tract, be metabolically stable and possess favorable pharmacokinetic properties. Furthermore, the Ideal drug candidate will exist in a physical form that is stable, non-hygroscopic and easily formulated.

### SUMMARY OF THE INVENTION

In this Invention, it has now been found out that the new class of benzimidazolone compounds show CB1 receptor binding activity and favorable properties as drug candidates, and thus are useful for the treatment of disease conditions mediated by CB1 binding activity such as inflammatory pain, nociceptive pain, neuropathic pain, fibromyalgia, chronic low back pain, visceral pain, acute cerebral ischemia, pain, chronic pain, acute pain, post herpetic neuralgia, neuropathies, neuralgia, diabetic neuropathy, HIV-related neuropathy, nerve injury, rheumatoid arthritic pain, osteoarthritic pain, back pain, cancer pain, dental pain, fibromyalgia, neuritis, sciatica, inflammation, neurodegenerative disease, spasticity, epilepsy, Tourette's syndrome, Parkinson's disease, neuroprotection, anxiety, cough, broncho constriction, irritable bowel syndrome (IBS), inflammatory bowel disease (IBD), colitis, cerebrovascular ischemia, cachexia, nausea, emesis, chemotherapy-induced emesis, rheumatoid arthritis, asthma, Crohn's disease, ulcerative colitis, asthma, dermatitis, seasonal allergic rhinitis, gastroesophageal reflux disease (GERD), constipation, diarrhea, functional gastrointestinal disorder, cutaneous T cell lymphoma, multiple sclerosis, osteoarthritis, psoriasis, systemic lupus erythematosus, diabetes, glaucoma, osteoporosis, glomerulonephritis, renal Ischemia, nephritis, hepatitis, cerebral stroke, vasodialation, hypertension, vasculitis, myocardial Infarction, cerebral ischemia, reversible airway obstruction, adult respiratory disease syndrome, chronic obstructive pulmonary disease (COPD), cryptogenic fibrosing alveolitis and bronchitis (hereinafter, referred as ‘CB1 Diseases’).

The present invention provides a method for the treatment of a condition mediated by CB1 receptor activity in a mammalian subject including a human, which comprises administering to a mammal in need of such treatment a therapeutically effective amount of the compound of formula (I):

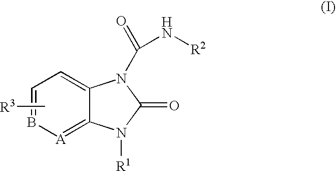

or a pharmaceutically acceptable salt thereof, wherein:

A is a carbon atom or a nitrogen atom;

B is a carbon atom or a nitrogen atom;

R¹ is a C₁-C₄ alkyl group substituted with 1 to 3 substituents independently selected from the group consisting of a halo group, a C₁-C₄ alkyl group; a hydroxy group; a C₁-C₄ alkoxy group; a mercapt group; a C₁-C₄ alkylthio group; a C₁-C₄ alkylsulfinyl group; a C₁-C₄ alkylsulfonyl group; an amino group; a C₁-C₄ alkylamino group; a di(C₁-C₄ alkyl)amino group; a (C₁-C₄ alkyl)(C₁-C₄ alkylsulfonyl)amino group; a cycloalkyl group; a cycloalkyl group substituted with 1 to 3 substituents selected from the group consisting of a hydroxy group, a C₁-C₄ alkoxy group and a C₁-C₄ alkyl group; a heterocyclyl group; a heterocyclyl group substituted with 1 to 3 substituents selected from the group consisting of a hydroxy group, a C₁-C₄ alkoxy group, a C₁-C₄ alkyl group and an oxo group; a cyano group; a heteroaryl group and a C₁-C₄ alkyl heteroaryl group;

R² is a cycloalkyl group; a cycloalkyl group substituted with 1 to 4 substituents selected from the group consisting of a hydroxy group, a C₁-C₄ hydroxyalkyl group, a C₁-C₄ alkoxy group, a C₆-C₁₀ aryloxy group, a mercapt group, a C₁-C₄ alkylthio group, a C₆-C₁₀ arylthio group, a carboxy group, a C₁-C₄ alkoxy-carbonyl group, a C₁-C₄ alkyl group, a C₂-C₄ alkenyl group, a C₂-C₄ alkynyl group and an amino-carbonyl group; a C₆-C₁₀ aryl group; a C₆-C₁₀ aryl group substituted with 1 to 3 substituents selected from the group consisting of a hydroxy group and a C₁-C₄ alkyl group; a heterocyclyl group; a heterocyclyl group substituted with 1 to 3 substituents selected from the group consisting of a hydroxy group and a C₁-C₄ alkyl group; a C₁-C₁₀ alkyl group; or a C₁-C₁₀ alkyl group substituted with 1 to 3 substituents independently selected from the group consisting of a cyano group, a hydroxy group, a trifluoromethyl group, a C₂-C₄ alkenyl group, a C₂-C₄ alkynyl group, a C₁-C₄ alkoxy group, a C₆-C₁₀ aryloxy group, a mercapt group, a C₁-C₄ alkylthio group, a C₁-C₄ alkylsulfinyl group, a C₁-C₄ alkylsulfonyl group, a C₁-C₄ alkylsulfonylamino group, a C₆-C₁₀ arylthio group, a carboxy group, a C₁-C₄alkyl-carbonyl group, a trifluoromethyl-carbonyl group, a C₁-C₄ alkoxy-carbonyl group, an amino carbonyl group, a C₁-C₄ alkylamino-carbonyl group, a C₁-C₄ hydroxyalkylamino-carbonyl group, a di(C₁-C₄ alkyl)amino-carbonyl group, a (C₁-C₄ hydroxyalkyl)(C₁-C₄ alkyl)amino-carbonyl group, a heterocyclyl-carbonyl group, a cycloalkyl group, a heterocyclyl group, a C₁-C₄ alkyl-substituted heterocyclyl group, a C₆-C₁₀ aryl group, a di(C₁-C₄ alkyl)amino group, a C₁-C₄alkoxy C₁-C₄ alkylamino-carbonyl group, an aryl C₁-C₄alkylamino-carbonyl group, and a heteroaryl C₁-C₄ alkylamino-carbonyl group and

R³ is a hydrogen atom, a halogen atom, a hydroxy group, a C₁-C₄ alkyl group, a C₁-C₄ haloalkyl group, a C₁-C₄ alkoxy group, a C₁-C₄ alkylthio group, a C₁-C₄ alkylsulfonyl group, a C₁-C₄ alkylsulfinyl group or an aminosulfonyl group.

The present invention is also directed to a compound or pharmaceutically acceptable salt thereof of formula (I) wherein:

A is a carbon atom or a nitrogen atom;

B is a carbon atom or a nitrogen atom;

R¹ is selected from the group consisting of H, CH₃—(CH₂)₄—, CH₃—(CH₂)₃—, cyano-(CH₂)₃—, cyano-(CH₂)₄—, CF₃—(CH₂)₂—, cyclobutyl-CH₂—, cyclobutyl-(CH₂)₂—, cyclopropyl-(CH₂)₃—, cyclopropyl-C(O)CH₂—, CH₃—CH₂—NH—C(O)CH₂—, (CH₃)₃—C—C(O)CH₂—, cyclohexyl-CH₂—, OH-cyclohexyl-CH₂—, F₂-cyclohexyl-CH₂—, F₂-cyclohexenyl-CH₂—, tetrahydrofuranyl-CH₂—, tetrahydropyranyl-CH₂—, fluoro-benzyl, CH₃—O-benzyl, cyano-benzyl, methyl-benzyl, chloro-benzyl, oxo-tetrahydrofuranyl-CH₂—, oxo-pyrrolidinyl-CH₂—, pyridinyl-CH₂—, pyrazinyl-CH₂—, pyrimidinyl-CH₂—, CH₃-pyrazolyl-CH₂—, CH₃-oxazolyl-CH₂—, CH₃-isoxazolyl-CH₂—, CH₃-oxadiazolyl-CH₂—, CH₃-thiazolyl-CH₂—, and CH₃-thiadiazolyl-CH₂—;

R² is selected from the group consisting of H, NR⁴R⁵—C(O)—CR⁶R⁷—, CR⁶R⁹R¹⁰—, (CH₃)₂—N—CH₂—C(CH₃)₂—CH₂—, tetrahydronaphthalenyl, OH-dihydroindenyl, OH-cyclohexyl, CH₃—CH₂-pyrrolidinyl-CH₂—, oxadiazolyl-CR¹¹R¹²— optionally substituted with CH₃, NH₂, (CH₃)₂—N—C(O)—, CH₃—NH—C(O), tetrahydronaphthalenyl-NH—C(O)—, azepanyl-C(O)—, oxopyrrolidinyl-(CH₂)₃—NH—C(O)—, CH₃O—CH₂)₂—NH—C(O)—, OH-cyclohexyl-NH—C(O)—, OH—CH₂-piperidinyl-C(O)—, CH₃—CH₂—, (CH₃)₂—CH—(CH₂)₂—NH—C(O)—, or (CH₃)₂—CH—; isoxazolyl-CR¹¹R¹²— optionally substituted with CH₃; furyl-CR¹¹R¹²— optionally substituted with CH₃ or CF₃; pyrazolyl-CR¹¹R¹²— optionally substituted with CH₃, (CH₃)₂—CH—, or CH₃—CH₂—; thiazolyl-CR¹¹R¹²— optionally substituted with CH₃, CH₃—CH₂—, or CF₃; and dihydroisochromenyl-CR¹¹R¹²—;

R³ is selected from the group consisting of H, F, Cl, bromo, difluoro, methyl, cyano, methoxy, trifluoromethyl, methylthio, methylsulfinyl, methylsulfonyl and aminosulfonyl;

R⁴ and R⁵ are H, OH—(CH₂)₂—, NH₂—C(O)—(CH₂)₂—, CH₃—O—(CH₂)₂—, benzyl, pyridinyl, cyclobutyl, (CH₃)₃—C—, cyclopropyl, CH₃, OH—(CH₂)₃—, or (OH)₂—CH₂—CH—CH₂—;

R⁶ and R⁷ are H, (CH₃)₃—C—, CH₃, benzyl, phenyl, tetrahydropyranyl, cyclohexyl, (CH₃)₂—CH—CH₂—, (CH₃)₂—CH—, or (OH)(CH₃)—CH—;

R⁸, R⁹ and R¹⁰ are H, (CH₃)₃—C—, NH₂—C(O)—, OH—CH₂—, (CH₃CH₂)₂—N—CH₂—, CH₃, (OH) (CH₃)₂—CH—, CH₃—NH—C(O)—CH₂—, cyclopropyl-NH—C(O)CH₂—, NH₂—C(O)—CH₂—NH—C(O)—CH₂—, or COOH—CH₂—; or two of R⁸, R⁹, or R¹⁰ form a cyclohexyl, and

R¹¹ and R¹² are H, CH₃, or (CH₃)₃C—.

Also, the present invention is directed to the method for the treatment of a condition mediated by CB1 receptor activity in a mammalian subject including a human, which comprises administering to a mammal in need of such treatment a therapeutically effective amount of a compound of formula (I) as described in the immediately preceeding paragraph.

Also, the present invention provides the use of a compound of formula (I) or a pharmaceutically acceptable salt thereof, each as described herein, for the manufacture of a medicament for the treatment of a condition mediated by CB1 receptor binding activity.

Also, the present invention also provides the use of a compound of formula (I) or a pharmaceutically acceptable salt thereof, each as described herein, for the manufacture of a medicament for the treatment of diseases selected from CB1 Diseases.

The compounds of the present invention may show less toxicity, good absorption, distribution, good solubility, less protein binding affinity other than CB1 receptor, less drug-drug interaction, and good metabolic stability.

### DETAILED DESCRIPTION OF THE INVENTION

The method of the present invention comprises administering to a mammal in need of such treatment a therapeutically effective amount of the compound or a pharmaceutically acceptable salt thereof wherein:

A is a carbon atom or a nitrogen atom;

B is a carbon atom or a nitrogen atom;

R¹ is a C₁-C₄ alkyl group substituted with 1 to 3 substituents independently selected from the group consisting of a C₁-C₄ alkyl group; a hydroxy group; a C₁-C₄ alkoxy group; a mercapt group; a C₁-C₄ alkylthio group; a C₁-C₄ alkylsulfinyl group; a C₁-C₄ alkylsulfonyl group; an amino group; a C₁-C₄ alkylamino group; a di(C₁-C₄ alkyl)amino group; a (C₁-C₄ alkyl)(C₁-C₄ alkylsulfonyl)amino group; a cycloalkyl group; a cycloalkyl group substituted with 1 to 3 substituents selected from the group consisting of a hydroxy group, a C₁-C₄ alkoxy group and a C₁-C₄ alkyl group; a heterocyclyl group; and a heterocyclyl group substituted with 1 to 3 substituents selected from the group consisting of a hydroxy group, a C₁-C₄ alkoxy group and a C₁-C₄ alkyl group;

R² is a cycloalkyl group; a cycloalkyl group substituted with 1 to 4 substituents selected from the group consisting of a hydroxy group, a C₁-C₄ hydroxyalkyl group, a C₁-C₄ alkoxy group, a C₆-C₁₀ aryloxy group, a mercapt group, a C₁-C₄ alkylthio group, a C₆-C₁₀ arylthio group, a carboxy group, a C₁-C₄ alkoxy-carbonyl group, a C₁-C₄ alkyl group, a C₂-C₄ alkenyl group and a C₂-C₄ alkynyl group; a C₆-C₁₀ aryl group; a C₆-C₁₀ aryl group substituted with 1 to 3 substituents selected from the group consisting of a hydroxy group and a C₁-C₄ alkyl group; a heterocyclyl group; a heterocyclyl group substituted with 1 to 3 substituents selected from the group consisting of a hydroxy group and a C₁-C₄ alkyl group; a C₁-C₁₀ alkyl group; or a C₁-C₁₀ alkyl group substituted with 1 to 3 substituents independently selected from the group consisting of a cyano group, a hydroxy group, a trifluoromethyl group, a C₂-C₄ alkenyl group, a C₂-C₄ alkynyl group, a C₁-C₄ alkoxy group, a C₆-C₁₀ aryloxy group, a mercapt group, a C₁-C₄ alkylthio group, a C₁-C₄ alkylsulfinyl group, a C₁-C₄ alkylsulfonyl group, a C₁-C₄ alkylsulfonylamino group, a C₆-C₁₀ arylthio group, a carboxy group, a C₁-C₄alkyl-carbonyl group, a trifluoromethyl-carbonyl group, a C₁-C₄ alkoxy-carbonyl group, an amino carbonyl group, a C₁-C₄ alkylamino-carbonyl group, a C₁-C₄ hydroxyalkylamino-carbonyl group, a di(C₁-C₄ alkyl)amino-carbonyl group, a (C₁-C₄ hydroxyalkyl)(C₁-C₄ alkyl)amino-carbonyl group, a heterocyclyl-carbonyl group, a cycloalkyl group, a heterocyclyl group, a C₁-C₄ alkyl-substituted heterocyclyl group and a C₆-C₁₀ aryl group; and

R³ is a hydrogen atom, a halogen atom, a hydroxy group, a C₁-C₄ alkyl group, C₁-C₄ haloalkyl group or a C₁-C₄ alkoxy group.

In another embodiment the method comprises compounds of formula (I) or a pharmaceutically acceptable salt thereof, wherein:

A is a carbon atom or a nitrogen atom;

B is a carbon atom or a nitrogen atom;

R¹ is a C₁-C₄ alkyl group substituted with 1 to 3 substituents independently selected from the group consisting of a halo group, a C₁-C₄ alkyl group; a C₁-C₄ alkylthio group; a cyano group; a heteroaryl group, a C₁-C₄ alkyl heteroaryl group; a cycloalkyl group; a cycloalkyl group substituted with hydroxy group, a heterocyclyl group; and a heterocyclyl group substituted with an oxo group;

R² is a cycloalkyl group substituted with a hydroxy group or an amino carbonyl group; a C₆-C₁₀ aryl group substituted with a hydroxy group; or a C₁-C₁₀ alkyl group substituted with 1 to 3 substituents independently selected from the group consisting of a hydroxy group, an amino carbonyl group, a di(C₁-C₄ alkyl)amino group, a cycloalkyl group, a heterocyclyl group, a C₁-C₄ alkyl-substituted heterocyclyl group, a C₆-C₁₀ aryl group, a C₁-C₄ alkoxy C₁-C₄ alkylamino-carbonyl group, an aryl C₁-C₄ alkylamino-carbonyl group, and a heteroaryl C₁-C₄ alkylamino-carbonyl group;

R³ is a hydrogen atom, a halogen atom, a hydroxy group, a C₁-C₄ alkyl group, a C₁-C₄ haloalkyl group, a C₁-C₄ alkoxy group, a C₁-C₄ alkylthio group, a C₁-C₄ alkylsulfonyl group, a C₁-C₄ alkylsulfinyl group or an aminosulfonyl group.

In another embodiment the method comprises the compounds of formula (I) or a pharmaceutically acceptable salt thereof, wherein A is a carbon atom and B is a carbon atom.

In another embodiment the CB1 mediated condition is selected from the group consisting of inflammatory pain, nociceptive pain, neuropathic pain, fibromyalgia, chronic low back pain, visceral pain, acute cerebral ischemia, pain, chronic pain, acute pain, post herpetic neuralgia, neuropathies, neuralgia, diabetic neuropathy, HIV-related neuropathy, nerve injury, rheumatoid arthritic pain, osteoarthritic pain, back pain, cancer pain, dental pain, fibromyalgia, neuritis, sciatica, inflammation, neurodegenerative disease, spasticity, epilepsy, Tourette's syndrome, Parkinson's disease, neuroprotection, anxiety, cough, broncho constriction, irritable bowel syndrome (IBS), inflammatory bowel disease (IBD), colitis, cerebrovascular ischemia, cachexia, nausea, emesis, chemotherapy-induced emesis, rheumatoid arthritis, asthma, Crohn's disease, ulcerative colitis, asthma, dermatitis, seasonal allergic rhinitis, gastroesophageal reflux disease (GERD), constipation, diarrhea, functional gastrointestinal disorder, cutaneous T cell lymphoma, multiple sclerosis, osteoarthritis, psoriasis, systemic lupus erythematosus, diabetes, glaucoma, osteoporosis, glomerulonephritis, renal, ischemia, nephritis, hepatitis, cerebral stroke, vasodialation, hypertension, vasculitis, myocardial infarction, cerebral ischemia, reversible airway obstruction, adult respiratory disease syndrome, chronic obstructive pulmonary disease (COPD), cryptogenic fibrosing alveolitis and bronchitis.

In another embodiment, the present invention provides a method of treatment of a condition mediated by CB1 receptor activity in a mammalian subject including a human, which comprises administering to a mammal in need of such treatment a therapeutically effective amount of a compound or pharmaceutically acceptable salt thereof selected from the group list above.

In one embodiment the CB1 Diseases are selected form the group consisting of neurodegenerative disease, spasticity, epilepsy, Tourette's syndrome, Parkinson's disease, neuroprotection, anxiety, cachexia, nausea, vasodialation and hypertension.

Another aspect of the invention provides for a compound of formula (I) or a pharmaceutically acceptable salt thereof, wherein:

A is a carbon atom or a nitrogen atom;

B is a carbon atom or a nitrogen atom;

R¹ is selected from the group consisting of H, CH₃—(CH₂)₄—, cyano-(CH₂)₃—, cyano-(CH₂)₄—, CF₃—(CH₂)₂—, cyclobutyl-CH₂—, cyclobutyl-(CH₂)₂—, cyclopropyl-(CH₂)—, cyclohexyl-CH₂—, OH-cyclohexyl-CH₂—, tetrahydrofuranyl-CH₂—, tetrahydropyranyl-CH₂—, oxo-tetrahydrofuranyl-CH₂—, oxo-pyrrolidinyl-CH₂—, pyridinyl-CH₂—, pyrazinyl-CH₂—, pyrimidinyl-CH₂—, CH₃-pyrazolyl-CH₂—, CH₃-oxazolyl-CH₂—, CH₃-isoxazolyl-CH₂—, CH₃-oxadiazolyl-CH₂— CH₃-thiazolyl-CH₂—, and CH₃-thiadiazolyl-CH₂—;

R² is selected from the group consisting of H, NR⁴R⁵—C(O)—CR⁶R⁷—, CR⁸R⁹R¹⁰—, (CH₃)₂—N—CH₂—C(CH₃)₂—CH₂—, tetrahydronaphthalenyl, OH-dihydroindenyl, OH-cyclohexyl, and CH₃—CH₂-pyrrolidinyl-CH₂—;

R³ is selected from the group consisting of H, F, Cl, methyl, cyano, methoxy, trifluoromethyl, methylthio, methylsulfinyl, methylsulfonyl and aminosulfonyl;

R⁴ and R⁵ are H, OH—(CH₂)₂—, NH₂—C(O)—(CH₂)₂—, CH₃—O—(CH₂)ᵣ, benzyl, or pyridinyl;

R⁶ and R⁷ are H, (CH₃)₃—C—, CH₃, benzyl, phenyl, tetrahydropyranyl, or cyclohexyl;

R⁸, R⁹ and R¹⁰ are H, (CH₃)₃—C—, NH₂—C(O)—, OH—CH₂—, (CH₃CH₂)₂—N—CH₂—, or CH₃; or two of R⁸, R⁹, or R¹⁰ form a cyclohexyl.

In another embodiment the compound a pharmaceutically acceptable salt thereof, wherein:

A is a carbon atom;

B is a carbon atom;

R¹ is selected from the group consisting of H, CH₃—(CH₂)₄—, cyano-(CH₂)₃—, cyano-(CH₂)₄—, CF₃—(CH₂)₂—, cyclobutyl-CH₂—, cyclobutyl-(CH₂)₂—, cyclopropyl-(CH₂)₃—, cyclohexyl-CH₂—, OH-cyclohexyl-CH₂—, tetrahydrofuranyl-CH₂—, tetrahydropyranyl-CH₂—, oxo-tetrahydrofuranyl-CH₂—, oxo-pyrrolidinyl-CH₂—, pyridinyl-CH₂—, pyrazinyl-CH₂—, pyrimidinyl-CH₂—, CH₃-pyrazolyl-CH₂—, CH₃-oxazolyl-CH₂—, CH₃-isoxazolyl-CH₂—, CH₃-oxadiazolyl-CH₂—, CH₃-thiazolyl-CH₂—, and CH₃-thiadiazolyl-CH₂—;

R² is selected from the group consisting of H, NR⁴R⁵—C(O)CR⁶R⁷—, CR⁸R⁹R¹⁰—, (CH₃)₂—N—CH₂—C(CH₃)₂—CH₂—, tetrahydronaphthalenyl, OH-dihydroindenyl, OH-cyclohexyl, and CH₃—CH₂-pyrrolidinyl-CH₂—;

R³ is selected from the group consisting of H, F, Cl, methyl, cyano, methoxy, trifluoromethyl, methylthio, methylsulfinyl, methylsulfonyl and aminosulfonyl;

R⁴ and R⁵ are H, OH—(CH₂)₂—, NH₂—C(O)—(CH₂)₂—, CH₃—O—(CH₂)₂—, benzyl, or pyridinyl;

R⁶ and R⁷ are H, (CH₃)₃—C—, CH₃, benzyl, phenyl, tetrahydropyranyl, or cyclohexyl;

R⁸, R⁹ and R¹⁰ are H, (CH₃)₃—C—, NH₂—C(O), OH—CH₂—, (CH₃CH₂)₂—N—CH₂—, or CH₃; or two of R⁸, R⁹, or R¹⁰ form a cyclohexyl.

In another embodiment the compound a pharmaceutically acceptable salt thereof, wherein R¹ is selected from the group consisting of

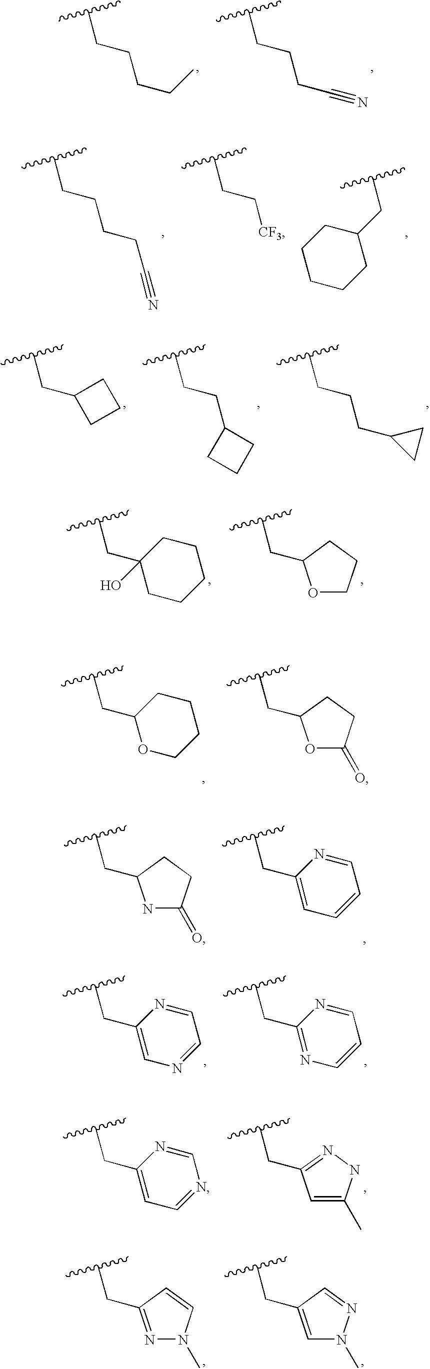

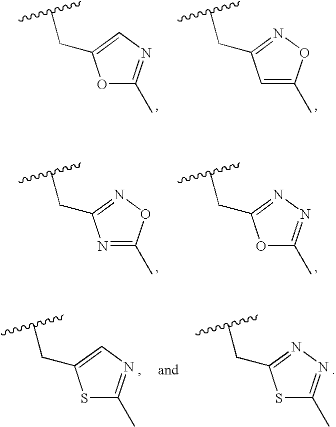

In another embodiment the compound or a pharmaceutically acceptable salt thereof, wherein R² is selected from the group consisting of

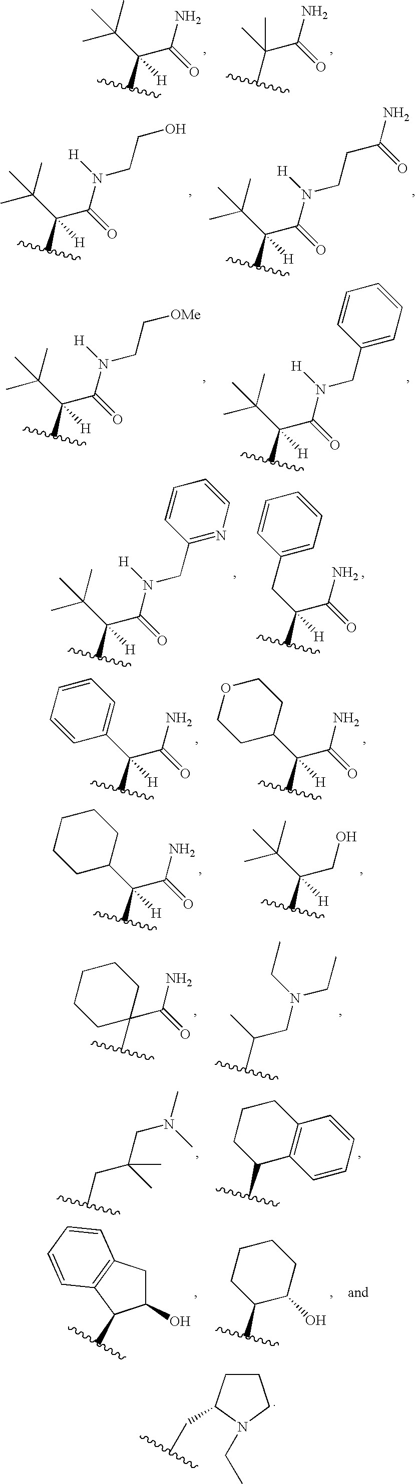

In another embodiment a method of treatment comprises administering to a mammal in need of such treatment a therapeutically effective amount of the compound N-[(1S)-1-(aminocarbonyl)-2,2-dimethylpropyl]-2-oxo-3-(tetrahydro-2H-pyran-4-ylmethyl)-2,3-dihydro-1H-benzimidazole-1-carboxamide or N-[(1S)-1-(aminocarbonyl)-2,2-dimethylpropyl]-4-methyl-3-[2-(methylthio)ethyl]-2-oxo-2,3-dihydro-1H-benzimidazole-1-carboxamide or a pharmaceutically acceptable salt thereof.

In another embodiment the compound selected from the group consisting of
N-[(1S)-1-(aminocarbonyl)-2,2-dimethylpropyl]-2-oxo-3-(tetrahydro-2H-pyran-4-lylmethyl)-2,3-dihydro-1H-benzimidazole-1-carboxamide;
N-[(1S)-1-(aminocarbonyl)-2,2-dimethylpropyl]-3-(cyclohexylmethyl)-6-fluoro-2-oxo-2,3-dihydro-1H-benzimidazole-1-carboxamide;
N-[(1S)-1-(aminocarbonyl)-2,2-dimethylpropyl]-6-chloro-3-(cyclohexylmethyl)-2-oxo-2,3-dihydro-1H-benzimidazole-1-carboxamide;
N-[(1S)-1-(aminocarbonyl)-2,2-dimethylpropyl]-3-(cyclohexylmethyl-6-methyl-2-oxo-2,3-dihydro-1H-benzimidazole-1-carboxamide;
N-[(1S)-1-(aminocarbonyl)-2,2-dimethylpropyl]-6-cyano-3-(cyclohexylmethyl)-2-oxo-2,3-dihydro-1H-benzimidazole-1-carboxamide;
N-[(1S)-1-(aminocarbonyl)-2,2-dimethylpropyl]-3-(cyclohexylmethyl)-5-fluoro-2-oxo-2,3-dihydro-1H-benzimidazole-1-carboxamide;
N-[(1S)-1-(aminocarbonyl)-2,2-dimethylpropyl]-5-chloro-3-(cyclohexylmethyl)-2-oxo-2,3-dihydro-1H-benzimidazole-1-carboxamide;
N-[(1S)-1-(aminocarbonyl)-2,2-dimethylpropyl]-5-cyano-3-(cyclohexylmethyl)-2-oxo-2,3-dihydro-1H-benzimidazole-1-carboxamide;
N-[(1S)-2,2-dimethylpropyl]-3-(cyclohexylmethyl)-4-fluoro-2-oxo-2,3-di hydro-1H-benzimidazole-1-carboxamide;
N-[(1)-1-(aminocarbonyl)-2,2-dimethylpropyl]-4-chloro-3-(cyclohexylmethyl)-2-oxo-2,3-dihydro-1H-benzimidazole-1-carboxamide;
N-[(1S)-1-(aminocarbonyl)-2,2-dimethylpropyl]-3-cyclohexylmethyl)-4-methyl-2-oxo-2,3-dihydro-1H-benzimidazole-1-carboxamide;
N-[(1S)-1-(aminocarbonyl)-2,2-dimethylpropyl]-4-cyano-3-(cyclohexylmethyl)-2-oxo-2,3-dihydro-1H-benzimidazole-1-carboxamide;
3-(cyclohexylmethyl)-N-[(1S)-1-(hydroxymethyl)-2,2-dimethylpropyl]-2-oxo-2,3-dihydro-1H-benzimidazole-1-carboxamide;
3-(cyclobutylmethyl)-N-[(1S)-1-(hydroxymethyl)-2,2-dimethylpropyl]-2-oxo-2,3-dihydro-1H-benzimidazole-1-carboxamide;
N-[(1S)-1-(hydroxymethyl)-2,2-dimethylpropyl]-2-oxo-3-pentyl-2,3-dihydro-1H-benzimidazole-1-carboxamide;
3-(cyclohexylmethyl)-N-{[(2S)-1-ethylpyrrolidin-2-yl]methyl}-2-oxo-2,3-dihydro-1H-benzimidazole-1-carboxamide;
3-(cyclobutylmethyl)-N-{[(2S)-1-ethylpyrrolidin-2-yl]methyl}-2-oxo-2,3-dihydro-1H-benzimidazole-1-carboxamide;
N-{[(2S)-1-ethylpyrrolidin-2-yl]methyl}-2-oxo-3-penty-2,3-dihydro-1H-benzimidazole-1-carboxamide;
3-(cyclohexylmethyl)-N-[3-(dimethylamino)-2,2-dimethylpropyl]-2-oxo-2,3-dihydro-1H-benzimidazole-1-carboxamide;
3-(cyclobutylmethyl)-N-[3-(dimethylamino 2,2-dimethylpropyl]-2-oxo-2,3-dihydro-1H-benzimidazole-1-carboxamide;
N-[3-(dimethylamino)-2,2-dimethylpropyl]-2-oxo-3-pentyl-2,3-dihydro-1H-benzimidazole-1-carboxamide;
3-(cyclohexylmethyl)-N-[2-(diethylamino)-1-methylethyl]-2-oxo-2,3-dihydro-1H-benzimidazole-1-carboxamide;
3-(cyclobutylmethyl)-N-[2-(diethylamino)-1-methylethyl]-2-oxo-2,3-dihydro-1H-benzimidazole-1-carboxamide;
N-[2-(diethylamino)-1-methylethyl]-2-oxo-3-pentyl-2,3-dihydro-1H-benzimidazole-1-carboxamide;
3-(cyclohexylmethyl)-N-[(1S,2R)-2-hydroxy-2,3-dihydro-1H-inden-1-yl]-2-oxo-2,3-dihydro-1H-benzimidazole-1-carboxamide;
3-(cyclobutylmethyl)-N-[(1S,2R)-2-hydroxy-2,3-dihydro-1H-inden-1-yl]-2-oxo-2,3-dihydro-1H-benzimidazole-1-carboxamide;
N-[(1S,2R)-2-hydroxy-2,3-dihydro-1H-inden-1-yl]-2-oxo-3-pentyl-2,3-dihydro-1H-benzimidazole-1-carboxamide;
3-(cyclohexylmethyl)-N-[(1S,2S)-2-hydroxycyclohexyl]-2-oxo-2,3-dihydro-1H-benzimidazole-1-carboxamide;
3-(cyclobutylmethyl)-N-[(1S,2S)-2-hydroxycyclohexyl]-2-oxo-2,3-dihydro-1H-benzimidazole-1-carboxamide;
N-[(1S,2S)-2-hydroxycyclohexyl]-2-oxo-3-pentyl-2,3-dihydro-1H-benzimidazole-1-carboxamide;
3-(cyclohexylmethyl)-N-(trans hydroxycyclohexyl)-2-oxo-2,3-dihydro-1H-benzimidazole-1-carboxamide;
3-(cyclobutylmethyl)-N-(trans-4-hydroxycyclohexyl)-2-oxo-2,3-dihydro-1H-benzimidazole-1-carboxamide;
N-(trans-4-hydroxycyclohexyl)-2-oxo-3-pentyl-2,3-dihydro-1H-benzimidazole-1-carboxamide;
3-(cyclohexylmethyl)-2-oxo-N-[(1S)-1,2,3,4-tetrahydronaphthalen-1-yl]-2,3-dihydro-1H-benzimidazole-1-carboxamide;
3-(cyclobutylmethyl)-2-oxo-N-[(1S)-1,2,3,4-tetrahydronaphthalen-1-yl]-2,3-dihydro-1H-benzimidazole-1-carboxamide;
2-oxo-3-pentyl-N-[(1S)-1,2,3,4-tetrahydronaphthalen-1-yl]-2,3-dihydro-1H-benzimidazole-1-carboxamide;
N-[(1S)-1-(aminocarbonyl)-2,2-dimethylpropyl]-3-(cyclohexylmethyl)-2-oxo-2,3-dihydro-1H-benzimidazole-1-carboxamide;
N-[(1S)-1-(aminocarbonyl)-2,2-dimethylpropyl]-3-(cyclobutylmethyl)-2-oxo-2,3-dihydro-1H-benzimidazole-1-carboxamide;
N-[(1S)-1-(aminocarbonyl)-2,2-dimethylpropyl]-2-oxo-3-pentyl-2,3-dihydro-1H-benzimidazole-1-carboxamide;
N-[(1S)-1-(aminocarbonyl)-2-methylpropyl]-3-(cyclohexylmethyl)-2-oxo-2,3-dihydro-1H-benzimidazole-1-carboxamide;
N-[(1S)-1-(aminocarbonyl)-2-methylpropyl]-3-(cyclobutylmethyl)-2-oxo-2,3-dihydro-1H-benzimidazole-1-carboxamide;
N-[(1S)-1-(aminocarbonyl)-2-methylpropyl]-2-oxo-3-pentyl-2,3-dihydro-1H-benzimidazole-1-carboxamide;
N-(2-amino-1,1-dimethyl-2-oxoethyl)-3-(cyclohexylmethyl)-2-oxo-2,3-dihydro-1H-benzimidazole-1-carboxamide;
N-(2-amino-1,1-dimethyl-2-oxoethyl)-3-(cyclobutylmethyl)-2-oxo-2,3-dihydro-1H-benzimidazole-1-carboxamide;
N-(2-amino-1,1-dimethyl-2-oxoethyl)-2-oxo-3-pentyl-2,3-dihydro-1H-benzimidazole-1-carboxamide;
N-[1-(aminocarbonyl)cyclohexyl]-2-(cyclohexylmethyl)-2-oxo-2,3-dihydro-1H-benzimidazole-1-carboxamide;
N-[1-(aminocarbonyl)cyclohexyl]-3-(cyclobutylmethyl)-2-oxo-2,3-dihydro-1H-benzimidazole-1-carboxamide;
N-[1-(aminocarbonyl)cyclohexyl]-2-oxo-3-pentyl-2,3-dihydro-1H-benzimidazole-1-carboxamide;
N-[(1S)-2-amino-2-oxo-1-phenylethyl]-3-(cyclohexylmethyl)-2-oxo-2,3-dihydro-1H-benzimidazole-1-carboxamide;
N-[(1S)-2-amino-2-oxo-1-phenylethyl]-3-(cyclobutylmethyl)-2-oxo-2,3-dihydro-1H-benzimidazole-1-carboxamide;
N-[(1S)-2-amino-2-oxo-1-phenylethyl]-2-oxo-3-pentyl-2,3-dihydro-1H-benzimidazole-1-carboxamide;
N-alpha-{[3(cyclohexylmethyl)-2-oxo-2,3-dihydro-1H-benzimidazol-1-yl]carbonyl}-L-phenylalaninamide;
N-alpha-{[3-(cyclobutylmethyl)-2-oxo-2,3-dihydro-1H-benzimidazol-1-yl]carbonyl}-L-phenylalaninamide;
N-alpha-[(2-oxo-3-pentyl-2,3-dihydro-1H-benzimidazol-1-yl)carbonyl]-L-phenylalaninamide;
N-[(1S)-1-(aminocarbonyl)-2,2-dimethylpropyl]-3-(2-cyclobutylethyl)-2-oxo-2,3-dihydro-1H-benzimidazole-1-carboxamide;
N-[(1S)-1-(aminocarbonyl)-2,2-dimethylpropyl]-1-3(3-cyclopropylpropyl)-2-oxo-2,3-dihydro-1H-benzimidazole-1-carboxamide;
N-[(1S)-1-(aminocarbonyl)-2,2-dimethylpropyl]-2-oxo-3-(pyridin-2-ylmethyl)-2,3-dihydro-1H-benzimidazole-1-carboxamide;
N-[(1S)-1-(aminocarbonyl)-2,2-dimethylpropyl]-2-oxo-3-(pyridin-2-ylmethyl)-2,3-dihydro-1H-benzimidazole-1-carboxamide;
N-[(1S)-1-(aminocarbonyl)-2,2-dimethylpropyl]-2-oxo-3-(pyrimidin-2-ylmethyl)-2,3-dihydro-1H-benzimidazole-1-carboxamide;
N-[(1S)-1-(aminocarbonyl)-2,2-dimethylpropyl]-2-oxo-3-(pyridazin-3-ylmethyl)-2,3-dihydro-1H-benzimidazole-1-carboxamide;
N-[(1S)-1-(aminocarbonyl)-2,2-dimethylpropyl]-2-oxo-3-(pyrimidin-4-ylmethyl)-2,3-dihydro-1H-benzimidazole-1-carboxamide;
N-[(1S)-1-(aminocarbonyl)-2,2-dimethylpropyl]-3-[(5-methylisoxazol-3-yl)methyl]-2-oxo-2,3-dihydro-1H-benzimidazole-1-carboxamide;
N-[(1S)-1-(aminocarbonyl)-2,2-dimethylpropyl]-3-[(5-methyl-1H-pyrazol-3-yl)methyl]-2-oxo-2,3-dihydro-1H-benzimidazole-1-carboxamide;
N-[(1S)-1-(aminocarbonyl)-2,2-dimethylpropyl]-3-[(5-methyl-1,2,4-oxadiazol-3-yl)methyl]-2-oxo-2,3-dihydro-1H-benzimidazole-1-carboxamide;
N-[(1S)-1-(aminocarbonyl)-2,2-dimethylpropyl]-3-[(5-methyl-1,3,4-oxadiazol-2-yl)methyl]-2-oxo-2,3-dihydro-1H-benzimidazole-1-carboxamide;
N-[(1S)-1-(aminocarbonyl)-2,2-dimethylpropyl]-3-[(1-methyl-1H-pyrazol-4-yl)methyl]-2-oxo-2,3-dihydro-1H-benzimidazole-1-carboxamide;
N-[(1S)-1-(aminocarbonyl)-2,2-dimethylpropyl]-3-[(1-methyl-1H-pyrazol-3-yl)methyl]-2-oxo-2,3-dihydro-1H-benzimidazole-1-carboxamide;
N-[(1S)-1-(aminocarbonyl)-2,2-dimethylpropyl]-3-[(2-methyl-1,3-oxazol-5-yl)methyl]-2-oxo-2,3-dihydro-1H-benzimidazole-1-carboxamide;
N-[(1S)-1-(aminocarbonyl)-2,2-dimethylpropyl]-3-[(2-methyl-1,3-thiazol-5-yl)methyl]-2-oxo-2,3-dihydro-1H-benzimidazole-t-carboxamide;
N-[(1S)-1-aminocarbonyl)-2,2-dimethylpropyl]-3-[(5-methyl-1,3,4-thiadiazol-2-yl)methyl]-2-oxo-2,3-dihydro-1H-benzimidazole-1-carboxamide;
N-[(1S)-1-(aminocarbonyl)-2,2-dimethylpropyl]-3-(3-cyanopropyl)-2-oxo-2,3-dihydro-1H-benzimidazole-1-carboxamide;
N-[(1S)-1-(aminocarbonyl)-2,2-dimethylpropyl]-3-(4-cyanobutyl)-2-oxo-2,3-dihydro-1H-benzimidazole-1-carboxamide;
N-[(1S)-1-(aminocarbonyl)-2,2-dimethylpropyl]-2-oxo-3-(tetrahydro-2H-pyran-2-ylmethyl)-2,3-dihydro-1H-benzimidazole-1-carboxamide;
N-[(1S)-1-(aminocarbonyl)-2,2-dimethylpropyl]-2-oxo-3-(tetrahydrofuran-2-ylmethyl)-2,3-dihydro-1H-benzimidazole-1-carboxamide;
N-(2-amino-1,1-dimethyl-2-oxoethyl)-3-(2-cyclobutylethyl)-2-oxo-2,3-dihydro-1H-benzimidazole-1-carboxamide;
N-(2-amino-1,1-dimethyl-2-oxoethyl)-3-[(5-methyl-1,3,4-oxadiazol-2-yl)methyl]-2-oxo-2,3-dihydro-1H-benzimidazole-1-carboxamide;
N-(2-amino-1,1-dimethyl-2-oxoethyl)-3-(3-cyanopropyl)-2-oxo-2,3-dihydro-1H-benzimidazole-1-carboxamide;
N-[(1S)-2-amino-2-oxo-1-phenylethyl]-2-oxo-3-(pyridin-2-ylmethyl)-2,3-dihydro-1H-benzimidazole-1-carboxamide;
N-[(1S)-2-amino-2-oxo-1-phenylethyl]-2-oxo-3-(pyrazin-2-ylmethyl)-2,3-dihydro-1H-benzimidazole-1-carboxamide;
N-[(1S)-2-amino-2-oxo-1-phenylethyl]-3-[(5-methylisoxazol-3-yl)methyl]-2-oxo-2,3-dihydro-1H-benzimidazole-1-carboxamide;
N-alpha-({3-[(1-methyl-1H-pyrazol-3-yl)methyl]-2-oxo-2,3-dihydro-1H-benzimidazol-1-yl}carbonyl)-L-phenylalaninamide;
N-alpha-({3-[(5-methyl-1,3,4-thiadiazol-2-yl)methyl]-2-oxo-2,3-dihydro-1H-benzimidazol-1-yl}carbonyl)-L-phenylalaninamide;
N-alpha-({2-oxo-3-[(5-oxotetrahydrofuran-2-yl)methyl]-2,3-dihydro-1H-benzimidazol-1-yl}carbonyl)-L-phenylalaninamide;
N-alpha-({2-oxo-3-[(5-oxopyrrolidin-2-yl)methyl]-2,3-dihydro-1H-benzimidazol-1-yl)carbon yl}-L-phenylalaninamide;
N-[(1S)-1-(aminocarbonyl)-2,2-dimethylpropyl]-3-(cyclohexylmethyl)-4-methoxy-2-oxo-2,3-dihydro-1H-benzimidazole-1-carboxamide;
N-[(1S)-1-(aminocarbonyl)-2,2-dimethylpropyl]-3-(cyclohexylmethyl)-5-methoxy-2-oxo-2,3-dihydro-1H-benzimidazole-1-carboxamide;
N-(1S)-1-(aminocarbonyl)-2,2-dimethylpropyl)-3-(cyclohexylmethyl)-2-oxo-5-(trifluoromethyl)-2,3-dihydro-1H-benzimidazole-1-carboxamide;
N-[(1S)-1-(aminocarbonyl)-2,2-dimethylpropyl]-3-(cyclohexylmethyl)-2-oxo-2,3-dihydro-1H-imidazo[4,5-c]pyridine-1-carboxamide;
N-[(1S)-1-aminocarbonyl)-2,2-dimethylpropyl]-3-(cyclohexylmethyl)-2-oxo-2,3-dihydro-1H-imidazo[4,6b]pyridine-1-carboxamide;
N-[(1S)-2-amino-1-cyclohexyl-2-oxoethyl]-3-(cyclohexylmethyl)-2-oxo-2,3-dihydro-1H-benzimidazole-1-carboxamide;
N-[(1S)-2-amino-2-oxo-1-(tetrahydro-2H-pyran-4-yl)ethyl]-3-(cyclohexylmethyl)-2-oxo-2,3-dihydro-1H-benzimidazole-1-carboxamide;
N-[(1S)-1-(aminocarbonyl)-2,2-dimethylpropyl]-3-[(1-hydroxycyclohexyl)methyl]-2-oxo-2,3-dihydro-1H-benzimidazole-1-carboxamide;
3-(cyclohexylmethyl)-N-[(1S)-1-{[(2-hydroxyethyl)amino]carbonyl}-2,2-dimethylpropyl]-2-oxo-2,3-dihydro-1H-benzimidazole-1-carboxamide;
3-(cyclohexylmethyl)-N-[(1S)-1-{[(2-methoxyethyl)amino]carbonyl}-2,2-dimethylpropyl]-2-oxo-2,3-dihydro-1H-benzimidazole-1-carboxamide;
N-{(1S)-1-[(benzylamino)carbonyl]-2,2-dimethylpropyl}-3-(cyclohexylmethyl)-2-oxo-2,3-dihydro-1H-benzimidazole-1-carboxamide;
3-(cyclohexylmethyl)-N-[(1S)-2,2-dimethyl-1-{[(pyridin-2-ylmethyl)amino]carbonyl}propyl]-2-oxo-2,3-dihydro-1H-benzimidazole-1-carboxamide;
N-{[3-(cyclohexylmethyl)-2-oxo-2,3-dihydro-1H-benzimidazol-1-yl]carbonyl}-3-methyl-L-valyl-beta-alaninamide;
N-[(1S)-1-(aminocarbonyl-2,2-dimethylpropyl]-3-(cyclohexylmethyl)-5-(methylthio)-2-oxo-2,3-dihydro-1H-benzimidazole-1-carboxamide;
N-[(1S)-1 aminocarbonyl)-2,2-dimethylpropyl]-3-(cyclohexylmethyl)-5(methylsulfinyl)-2-oxo-2,3-dihydro-1H-benzimidazole-1-carboxamide;
N-[(1S)-1-(aminocarbonyl)-2,2-dimethylpropyl]-3-(cyclohexylmethyl)-5-(methylsulfonyl)-2-oxo-2,3-dihydro-1H-benzimidazole-1-carboxamide;
N-[(1S)-1-(aminocarbonyl)-2,2-dimethylpropyl]-5-aminosulfonyl)-3-(cyclohexylmethyl)-2-oxo-2,3-dihydro-1H-benzimidazole-1-carboxamide; and
N-[(1S)-1-(aminocarbonyl)-2,2-dimethylpropyl]-2-oxo-3-(3,3,3-trifluoropropyl)-2,3-dihydro-1H-benzimidazole-1-carboxamide,
In another embodiment a compound of formula (I) or a pharmaceutically acceptable salt thereof, wherein:

A is a carbon atom or a nitrogen atom;

B is a carbon atom or a nitrogen atom;

R¹ is selected from the group consisting of H, CH₃—(CH₂)₄—, CH₃—(CH₂)₃—, cyano-(CH₂)—, cyano-(CH₂)₄—, CF₃—(CH₂)₂—, cyclobutyl-CH₂—, cyclobutyl-(CH₂)₂—, cyclopropyl-CH₂)₃—, cyclopropyl-C(O)—CH₂—, CH₃—CH₂—NH—C(O)—CH₂—, (CH₃)₃—C—C(O)—CH₂—, cyclohexyl-CH₂—, OH-cyclohexyl-CH₂—, F₂-cyclohexyl-CH₂—, F-cyclohexenyl-CH₂—, tetrahydrofuranyl-CH₂—, tetrahydropyranyl-CH₂—, fluoro-benzyl, CH₃—O-benzyl, cyano-benzyl, methyl-benzyl, chloro-benzyl, oxo-tetrahydrofuranyl-CH₂—, oxo-pyrrolidinyl-CH₂—, pyridinyl-CH₂—, pyrazinyl-CH₂—, pyrimidinyl-CH₂—, CH₃-pyrazolyl-CH₂—, CH₃-oxazolyl-CH₂—, CH₃-isoxazolyl-CH₂—, CH₃-oxadiazolyl-CH₂—, CH₃-thiazolyl-CH₂—, and CH₃-thiadiazolyl-CH₂—;

R² is selected from the group consisting of H, NR⁴R⁵—C(O)—CR⁶R⁷—, CR⁸R⁹R¹⁰—, (CH₃)₂—N—CH₂—C(CH₃)₂—CH₂—, tetrahydronaphthalenyl, OH-dihydroindenyl, OH-cyclohexyl, CH₃—CH₂-pyrrolidinyl-CH₂—, oxadiazolyl-CR¹¹R¹²— optionally substituted with CH₃, NH₂, (CH₃)₂—N—C(O)—, CH₃—NH—C(O), tetrahydronaphthalenyl-NH—C(O)—, azepanyl-C(O)—, oxopyrrolidinyl-(CH₂)—NH—C(O)—, CH₃—O—(CH₂)₂—NH—C(O)—, OH-cyclohexyl-NH—C(O)—, OH—CH₂-piperidinyl-C(O)—, —CH₃—CH₂—, (CH₃)₂—CH—(CH₂)₂—NH—C(O)—, or (CH₃)₂—CH—; isoxazolyl-CR¹¹R¹²— optionally substituted with CH₃; furyl-CR¹¹R¹²— optionally substituted with CH₃ or CF₃; pyrazolyl-CR¹¹R¹²— optionally substituted with CH₃, (CH₃)₂—CH—, or CH₃—CH₂—; thiazolyl-CR¹¹R¹²— optionally substituted with CH₃, CH₃—CH₂—, or CF₃; and dihydroisochromenyl-CR¹¹R¹²—;

R³ is selected from the group consisting of H, F, Cl, bromo, difluoro, methyl, cyano, methoxy, trifluoromethyl, methylthio, methylsulfinyl, methylsulfonyl and aminosulfonyl;

R⁴ and R⁵ are H, OH—(CH₂)₂—, NH₂—C(O)—(CH₂)₂—, CH₃—O—(CH₂)₂—, benzyl, pyridinyl, cyclobutyl, (CH₃)₃—C—, cyclopropyl, CH₃, OH—(CH₂)₃—, or (OH)₂—CH₂—CH—CH₂—;

R⁶ and R⁷ are H, (CH₃)₃—C—, CH₃, benzyl, phenyl, tetrahydropyranyl, cyclohexyl, (CH₃)₂—CH—CH₂—, (CH₃)₂—CH—, or (OH)(CH₃)—CH—;

R⁸, R⁹ and R¹⁰ are H, (CH₃)₃—C—, NH₂—C(O)—, OH—CH₂—, (CH₃CH₂)₂—N—CH₂—, CH₃, (OH) (CH₃)₂—CH—, CH₃—NH—C(O)—CH₂—, cyclopropyl-NH—C(O)—CH₂—, NH₂—C(O)—CH₂—NH—C(O)—CH₂—, or COOH—CH₂—; or two of R⁸, R⁹, or R¹⁰ form a cyclohexyl, and

R¹¹ and R¹² are H, CH₃, or (CH₃)₃—C—.

In another embodiment the compound or a pharmaceutically acceptable salt thereof, wherein:

A is a carbon atom;

B is a carbon atom;

R¹ is selected from the group consisting of H, CH₃—CH₂)₄—, CH₃—(CH₂)—, cyano-(CH₂)₃—, cyano-(CH₂)₄—, CF₃—(CH₂)₂—, cyclobutyl-CH₂—, cyclobutyl-(CH₂)₂—, cyclopropyl-(CH₂)₃—, cyclopropyl-C(O)—CH₂—, CH₃—CH₂—NH—C(O)—CH₂—, (CH₃)₃—C—C(O)—CH₂—, cyclohexyl-CH₂—, OH-cyclohexyl-CH₂—, F₂-cyclohexyl-CH₂—, F-cyclohexenyl-CH₂—, tetrahydrofuranyl-CH₂—, tetrahydropyranyl-CH₂—, fluoro-benzyl, CH₃—O-benzyl, cyano-benzyl, methyl-benzyl, chloro-benzyl, oxo-tetrahydrofuranyl-CH₂—, oxo-pyrrolidinyl-CH₂—, pyridinyl-CH₂—, pyrazinyl-CH₂—, pyrimidinyl-CH₂—, CH₃-pyrazolyl-CH₂—, CH₃-oxazolyl-CH₂—, CH₃-isoxazolyl-CH₂—, CH₃-oxadiazolyl-CH₂—, CH₃-thiazolyl-CH₂—, and CH₃-thiadiazolyl-CH₂—;

R² is selected from the group consisting of H, NR⁴R⁵—C(O)—CR⁶R⁷—, CR⁸R⁹R¹⁰—, (CH₃)₂—N—CH₂—C(CH₃)₂—CH₂—, tetrahydronaphthalenyl, OH-dihydroindenyl, OH-cyclohexyl, CH₃—CH₂-pyrrolidinyl-CH₂—, oxadiazolyl-CR¹¹R¹²— optionally substituted with CH₃, NH₂, (CH₃)₂—N—C(O)—, CH₃—NH—C(O)—, tetrahydronaphthalenyl-NH—C(O)—, azepanyl-C(O)—, oxopyrrolidinyl-CH₂)₃—NH—C(O)—, CH₃—O—(CH₂)₂—NH—C(O)—, OH-cyclohexyl-NH—C(O)—, OH—CH₂-piperidinyl-C(O)—, CH₃—CH₂—, (CH₃)₂—CH₃)₂—CH₂)₂—NH—C(O)—, or (CH₃)₂—CH—; isoxazolyl-CR¹¹R¹²— optionally substituted with CH₃; furyl-CR¹¹R¹²— optionally substituted with CH₃ or CF₃; pyrazolyl-CR¹¹R¹²— optionally substituted with CH₃, (CH₃)₂—CH—, or CH₃—CH₂—; thiazolyl-CR¹¹R¹²— optionally substituted with CH₃, CH₃—CH₂—, or CF₃; and dihydroisochromenyl-CR¹¹R¹²—;

R³ is selected from the group consisting of H, F, Cl, bromo, difluoro, methyl, cyano, methoxy, trifluoromethyl, methylthio, methylsulfinyl, methylsulfonyl and aminosulfonyl;

R⁴ and R⁵ are H, OH—(CH₂)₂—, NH₂—C(O)—(CH₂)₂—, CH₃—O—(CH₂)₂—, benzyl, pyridinyl, cyclobutyl, (CH₃)₃—C—, cyclopropyl, CH₃, OH—(CH₂)₃—, or (OH)₂—CH₂—CH—CH₂—;

R⁶ and R⁷ are H, (CH₃)₃—C—, CH₃, benzyl, phenyl, tetrahydropyranyl, cyclohexyl, (CH₃)₂—CH—CH₂—, (CH₃)₂—CH—, or (OH)(CH₃)—CH—;

R⁸, R⁹ and R¹⁰ are H, (CH₃)₃—C—, NH₂—C(O)—, OH—CH₂—, (CH₃CH₂)₂—N—CH₂—, CH₃, (OH) (CH₃)₂—CH—, CH₃—NH—C(O)—CH₂—, cyclopropyl-NH—C(O)CH₂—, NH₂—C(O)—CH₂—NH—C(O)—CH₂—, or COOH—CH₂—; or two of R⁸, R⁹, or R¹⁰ form a cyclohexyl, and

R¹¹ and R¹² are H, CH₃, or (CH₃)₃—C—.

In another embodiment the compound or a pharmaceutically acceptable salt thereof, wherein R¹ is selected from the group consisting of

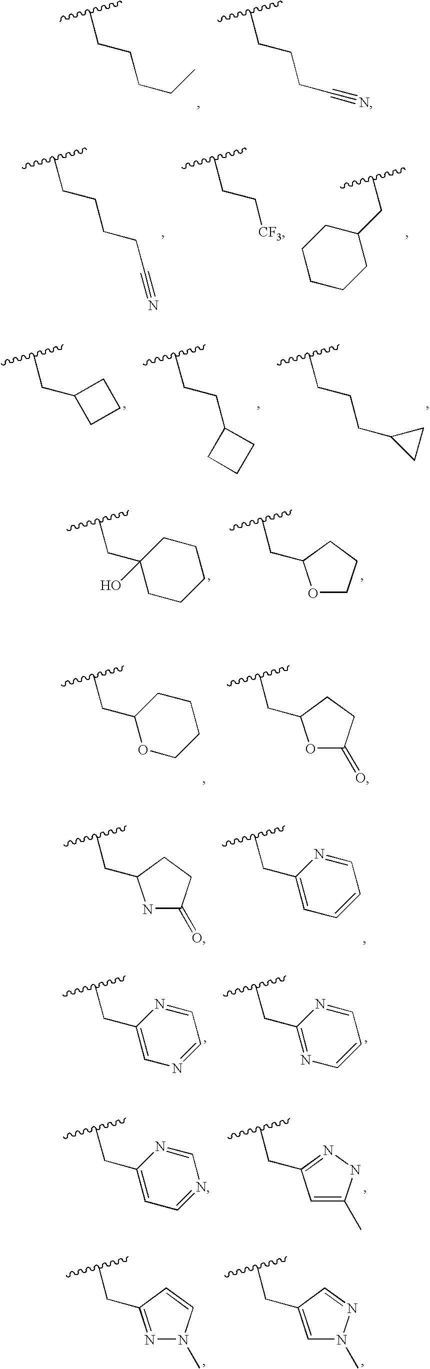

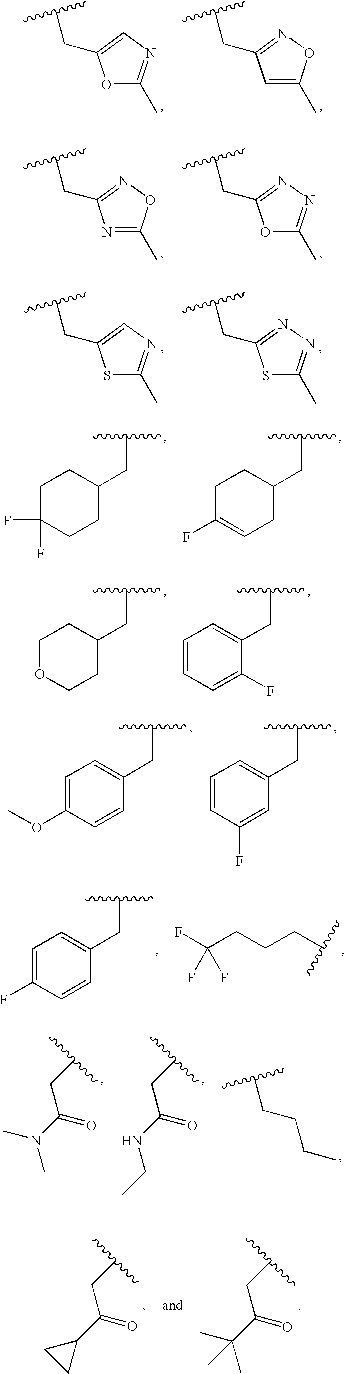

In one embodiment the compound or a pharmaceutically acceptable salt thereof, wherein R¹ is selected from the group consisting of

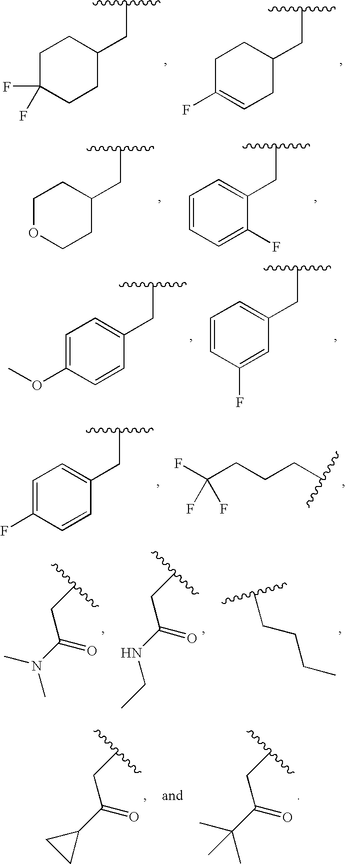

In one embodiment the compound or a pharmaceutically acceptable salt thereof, wherein R² is selected from the group consisting of

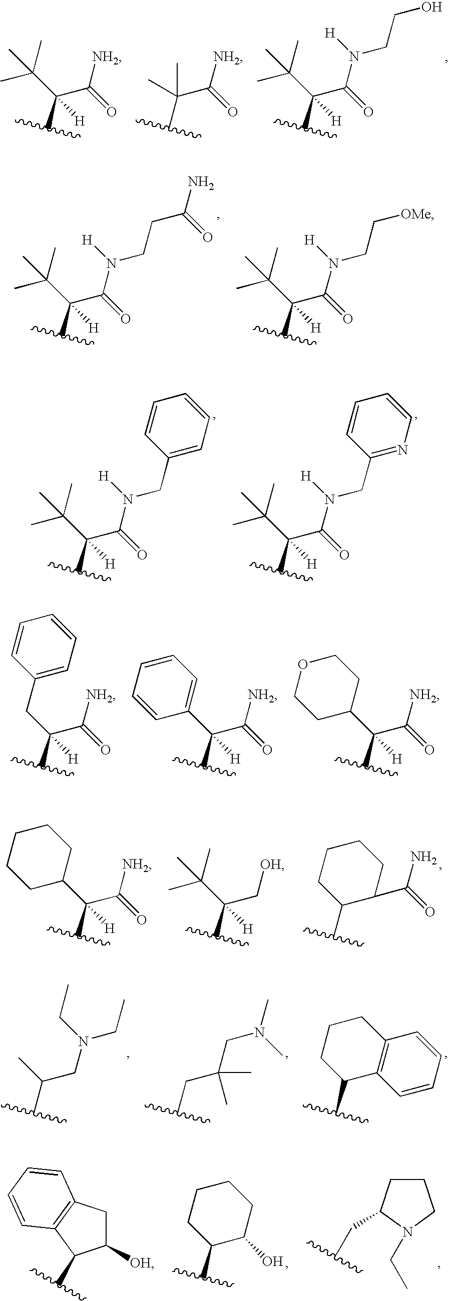

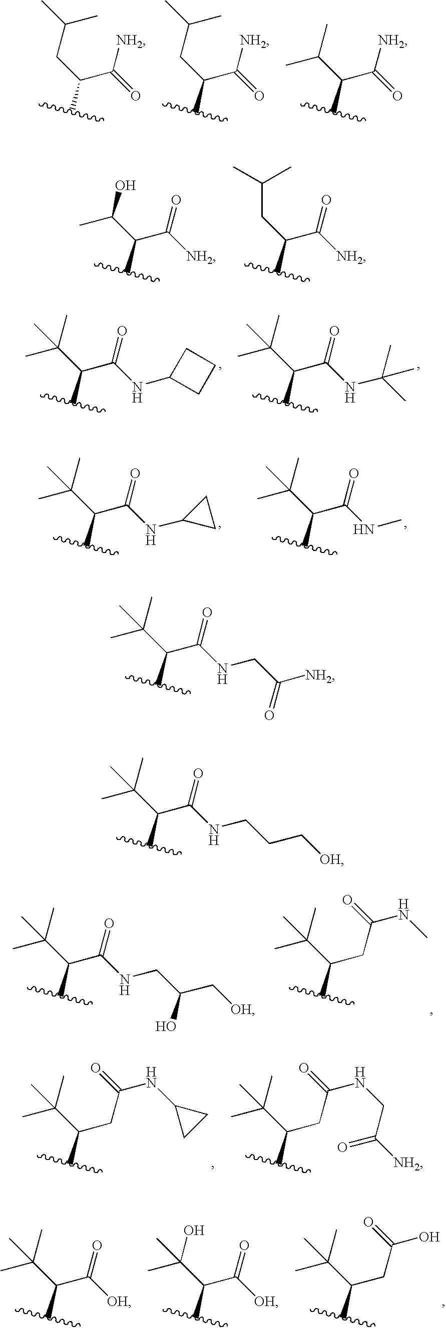

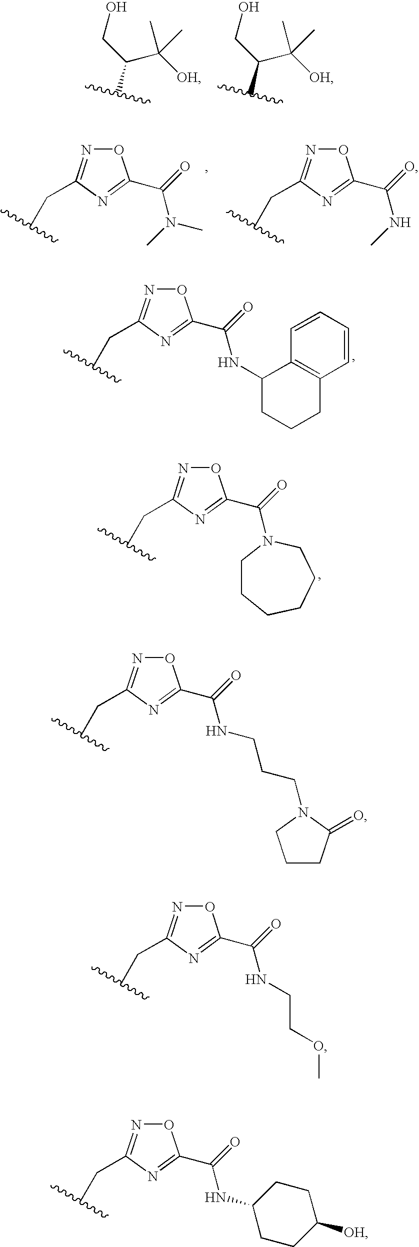

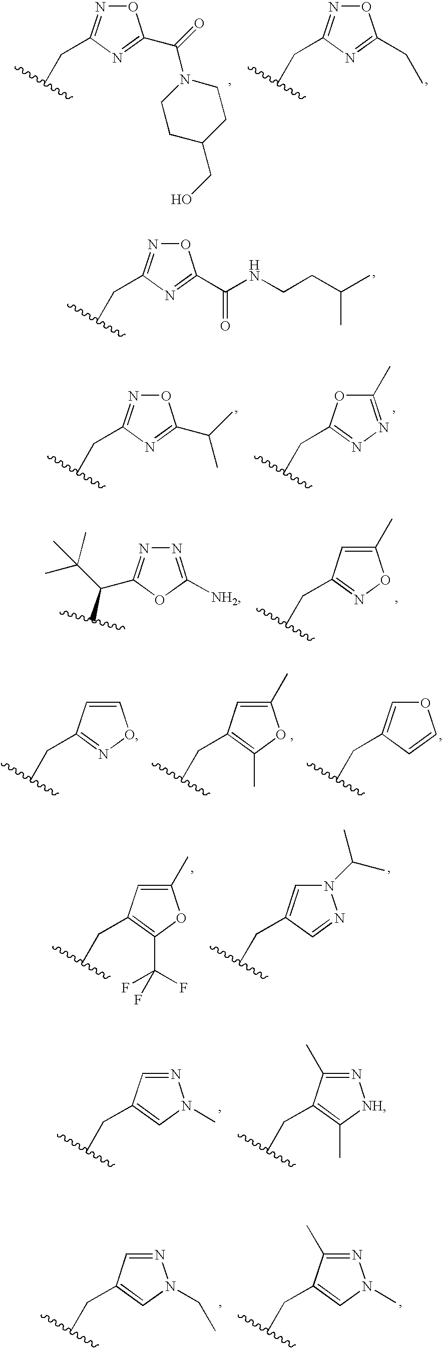

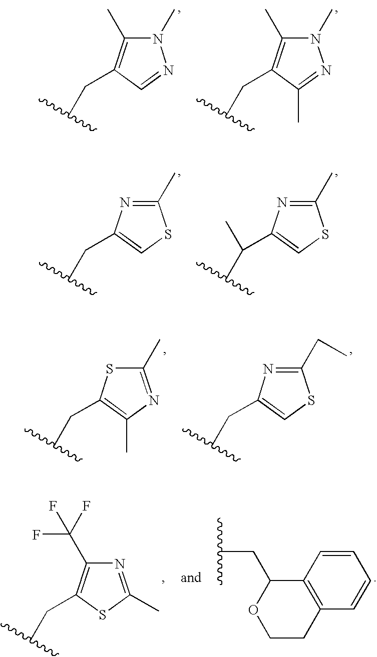

In one embodiment the compound or a pharmaceutically acceptable salt thereof, wherein R² is selected from the group consisting of

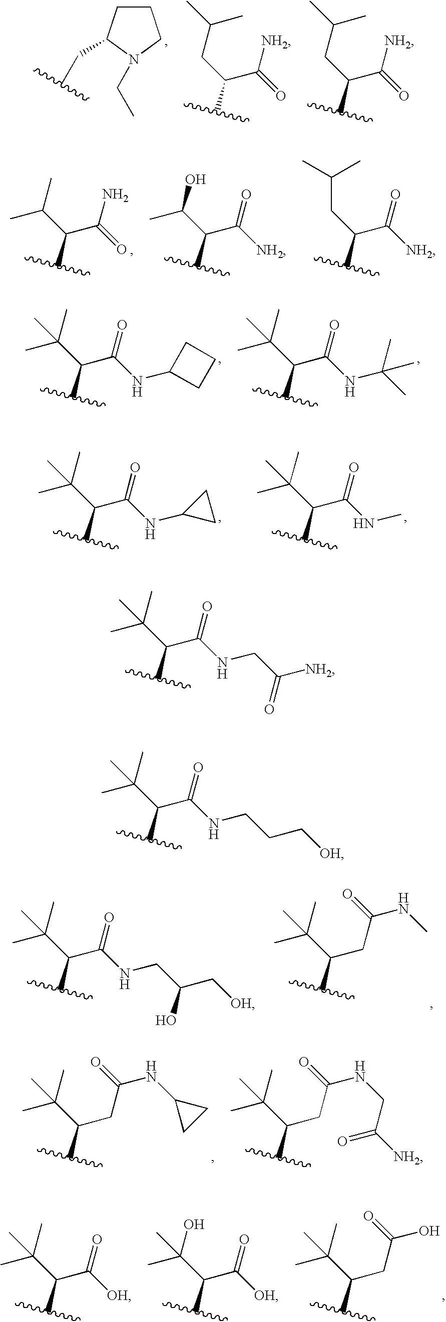

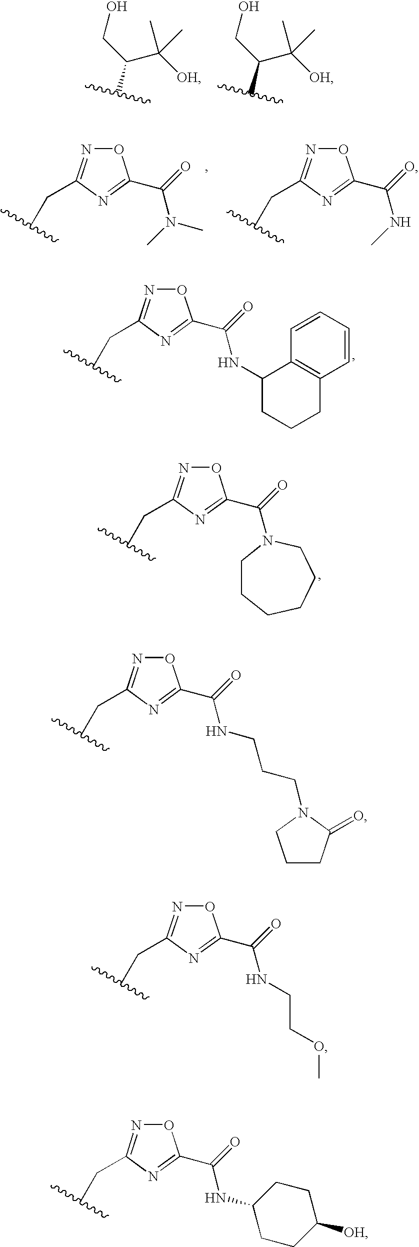

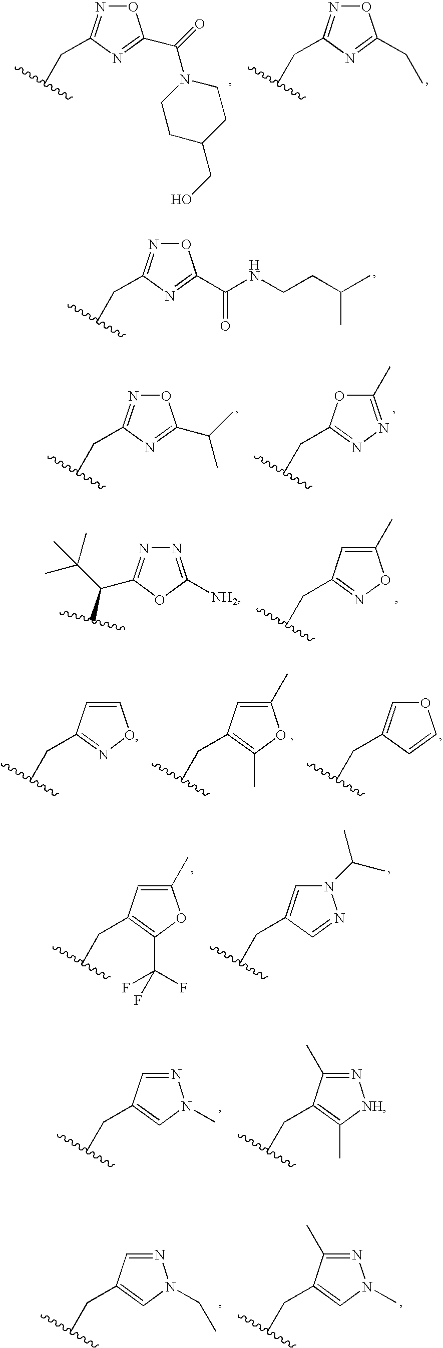

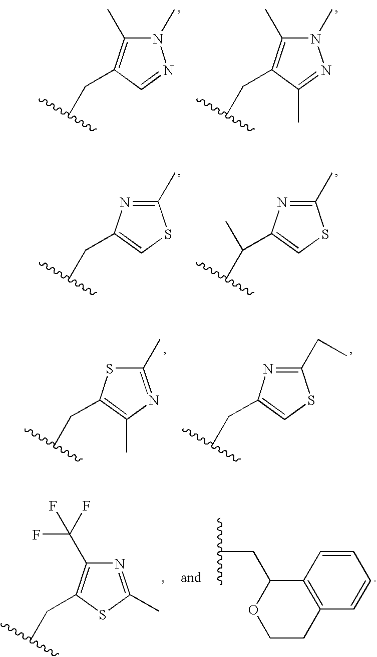

In one embodiment the compound selected from the group consisting of
N-[1-(aminocarbonyl)cyclohexyl]-3(cyclohexylmethyl)-5-methyl-2-oxo-2,3-dihydro-1H-benzimidazole-1-carboxamide;
N-[(1R)-1-(aminocarbonyl)-3-methylbutyl]-3-cyclohexylmethyl)-5-methyl-2-oxo-2,3-dihydro-1H-benzimidazole-1-carboxamide;
Nalpha-{[3-(cyclohexylmethyl)-5-methyl-2-oxo-2,3-dihydro-1H-benzimidazol-1-yl]carbonyl}-L-phenylalaninamide;
N-[(1S)-1-(aminocarbonyl)-2-methylpropyl]-3-(cyclohexylmethyl)-5-methyl-2-oxo-2,3-dihydro-1H-benzimidazole-1-carboxamide;
N-[(1 S)-1-(aminocarbonyl)-3-methylbutyl]-3-(cyclohexylmethyl)-5-methyl-2-oxo-2,3-dihydro-1H-benzimidazole-1-carboxamide;
N-[(1S)-1-(aminocarbonyl)-2,2-dimethylpropyl]-3-(cyclohexylmethyl)-7-fluoro-2-oxo-2,3-di hydro-1H-benzimidazole-1-carboxamide;
N-[(1S)-1-(aminocarbonyl)-2,2-dimethylpropyl]-5-cyano-2-oxo-3-(tetrahydro-2H-pyran-4-ylmethyl)-2,3-dihydro-1H-benzimidazole-1-carboxamide
N-[(1S)-2-amino-1-cyclohexyl-2-oxoethyl]-3-(cyclohexylmethyl)-5-methyl-2-oxo-2,3-dihydro-1H-benzimidazole-1-carboxamide;
N-[(1S,2R)-1-(aminocarbonyl)-2-hydroxypropyl]-2-oxo-3-(tetrahydro-2H-pyran-4-ylmethyl)-2,3-dihydro-1H-benzimidazole-1-carboxamide;
N-[(1S)-1-(aminocarbonyl)-2-methylpropyl]-2-oxo-3-(tetrahydro-2H-pyran-4-ylmethyl)-2,3-dihydro-1H-benzimidazole-1-carboxamide;
2-oxo-N-[(1S)-1,2,3,4-tetrahydronaphthalen-1-yl]-3-(tetrahydro-2H-pyran-4-ylmethyl)-2,3-dihydro-1H-benzimidazole-1-carboxamide;
N-[1)-2-amino-2-oxo-1-phenylethyl]-2-oxo-3-(tetrahydro-2H-pyran-4-ylmethyl)-2,3-dihydro-1H-benzimidazole-1-carboxamide;
Nalpha-{[2-oxo-3-(tetrahydro-2H-pyran-4-ylmethyl)-2,3-dihydro-1H-benzimidazol-1-yl]carbonyl}-L-phenylalaninamide;
N-[(1S)-1-(aminocarbonyl)-3-methylbutyl]-2-oxo-3-(tetrahydro-2H-pyran-4-ylmethyl)-2,3-dihydro-1H-benzimidazole-1-carboxamide;
N-[(1S,2R)-2-hydroxy-2,3-dihydro-1H-inden-1-yl]-2-oxo-3-(tetrahydro-2H-pyran-4-ylmethyl)-2,3-dihydro-1H-benzimidazole-1-carboxamide;
N-[(1S,2S)-2-hydroxycyclohexyl]-2-oxo-3-(tetrahydro-2H-pyran-4-ylmethyl)-2,3-dihydro-1H-benzimidazole-1-carboxamide;
N-[(1S)-1-(hydroxymethyl)-2,2-dimethylpropyl]-2-oxo-3-(tetrahydro-2H-pyran-4-ylmethyl)-2,3-dihydro-1H-benzimidazole-1-carboxamide;
N-[3-(dimethylamino-2,2-dimethylpropyl]-2-oxo-3-tetrahydro-2H-pyran-4-ylmethyl)-2,3-dihydro-1H-benzimidazole-1-carboxamide;
N-[(1S)-2-amino-1-cyclohexyl-2-oxoethyl]-2-oxo-3-(tetrahydro-2H-pyran-4-ylmethyl)-2,3-dihydro-1H-benzimidazole-1-carboxamide;
3-methyl-N-{[2-oxo-3-(tetrahydro-2H-pyran-4-ylmethyl)-2,3-dihydro-1H-benzimidazol-1-yl]carbonyl}-L-valine;
3-hydroxy-N-{[2-oxo-3-(tetrahydro-2H-pyran-4-ylmethyl)-2,3-dihydro-1H-benzimidazol-1-yl]carbonyl}-L-valine;
N-[(1S)-1-(aminocarbonyl)-2-hydroxy-2-methylpropyl]-2-oxo-3-(tetrahydro-2H-pyran-4-ylmethyl)-2,3-dihydro-1H-benzimidazole-1-carboxamide;
N-[(1S)-2-hydroxy-1-(hydroxymethyl)-2-methylpropyl]-2-oxo-3-(tetrahydro-2H-pyran-4-ylmethyl)-2,3-dihydro-1H-benzimidazole-1-carboxamide;
N-[(1R)-2-hydroxy-1-(hydroxymethyl)-2-methylpropyl]-2-oxo-3-(tetrahydro-2H-pyran yl methyl)-2,3-dihydro-1H-benzimidazole-1-carboxamide;
N-[(1S)-1-(aminocarbonyl)-2,2-dimethylpropyl]-5-fluoro-2-oxo-3-(tetrahydro-2H-pyran-4-ylmethyl)-2,3-dihydro-1H-benzimidazole-1-carboxamide;
(3R)-4,4-dimethyl-3-({[2-oxo-3-(tetrahydro-2H-pyran-4-ylmethyl)-2,3-dihydro-1H-benzimidazol-1-yl]carbonyl}amino)pentanoic acid;
N-[(1S)-1-aminocarbonyl)-2,2-dimethylpropyl]-3-butyl-2-oxo-2,3-dihydro-1H-benzimidazole-1-carboxamide;
N-[(1S)-1-(aminocarbonyl)-2,2-dimethylpropyl]-2-oxo-3-(4,4,4-trifluorobutyl)-2,3-dihydro-1H-benzimidazole-1-carboxamide;
N-[(1S)-1-(aminocarbonyl)-2,2-dimethylpropyl]-5,6-difluoro-2-oxo-3-(tetrahydro-2H-pyran-4-ylmethyl)-2,3-dihydro-1H-benzimidazole-1-carboxamide;
N-[(1R)-1-(2-amino-2-oxoethyl)-2,2-dimethylpropyl]-2-oxo-3-(tetrahydro-2H-pyran-4-ylmethyl)-2,3-dihydro-1H-benzimidazole-1-carboxamide;
N-(1R)-1-[2-(cyclopropylamino)-2-oxoethyl]-2,2-dimethylpropyl)-2-oxo-3-(tetrahydro-2H-pyran-4-ylmethyl)-2,3-dihydro-1H-benzimidazole-1-carboxamide;
N-[(1R)-1-{2-[2-(2-amino-2-oxoethyl)amino]-2-oxoethyl}-2,2-dimethylpropyl]-2-oxo-3-(tetrahydro-2H-pyran-4-ylmethyl)-2,3-dihydro-1H-benzimidazole-1-carboxamide;
N-{(1S)-1-[(cyclopropylamino)carbonyl]-2,2-dimethylpropyl}-2-oxo-3-(tetrahydro-2H-pyran-4-ylmethyl)-2,3-dihydro-1H-benzimidazole-1-carboxamide;
N-{(1S)-1-[(tert-butylamino)carbonyl]-2,2-dimethylpropyl}-2-oxo-3-tetrahydro-2H-pyran-4-ylmethyl)-2,3-dihydro-1H-benzimidazole-1-carboxamide;
N-[(1S)-1-(cyclobutylamino)carbonyl]-2,2-dimethylpropyl)-2-oxo-3-(tetrahydro-2H-pyran-4-ylmethyl)-2,3-dihydro-1H-benzimidazole-1-carboxamide;
5-fluoro-N-[(1S)-1-(hydroxymethyl)-2,2-dimethylpropyl]-2-oxo-3-(tetrahydro-2H-pyran-4-ylmethyl)-2,3-dihydro-1H-benzimidazole-1-carboxamide;
3-methyl-N-{[2-oxo-3-(tetrahydro-2H-pyran-4-ylmethyl)-2,3-dihydro-1H-benzimidazol-1-yl ]carbonyl}-L-valylglycinamide;
N-(1S)-2,2-dimethyl-1-[(methylamino)carbonyl]propyl)-2-oxo-3-(tetrahydro-2H-pyran-4-ylmethyl)-2,3-dihydro-1H-benzimidazole-1-carboxamide;
N-[(1S)-1-(aminocarbonyl)-2,2-dimethylpropyl]-3-[(4-fluorocyclohex-3-en-1-yl)methyl]-2-oxo-2,3-dihydro-1H-benzimidazole-1-carboxamide (diastereomer 1);
N-[(1S)-1-(aminocarbonyl)-2,2-dimethylpropyl]-3-[(4-fluorocyclohex-3-en-1-yl)methyl]-2-oxo-2,3-dihydro-1H-benzimidazole-1-carboxamide (diastereomer 2);
N-[(1S)-1-(aminocarbonyl)-2,2-dimethylpropyl]-3-[(4,4-difluorocyclohexyl)methyl]-2-oxo-2,3-dihydro-1H-benzimidazole-1-carboxamide;
N-[(1S)-1-(aminocarbonyl)-2,2-dimethylpropyl]-5-bromo-3-(cyclohexylmethyl)-2-oxo-2,3-dihydro-1H-benzimidazole-1-carboxamide;
5-bromo-3-(cyclohexylmethyl)-N-[(1S)-1-(hydroxymethyl)-2,2-dimethylpropyl]-2-oxo-2,3-dihydro-1H-benzimidazole-1-carboxamide;
N-{(1R)-2,2-dimethyl-1-[2-(methylamino)-2-oxoethyl]propyl}-2-oxo-3-(tetrahydro-2H-pyran-4-ylmethyl)-2,3-dihydro-1H-benzimidazole-1-carboxamide;
N-{(1S)-1-[(cyclopropylamino)carbonyl]-2,2-dimethylpropyl}-5-fluoro-2-oxo-3-(tetrahydro-2H-pyran-4-ylmethyl)-2,3-dihydro-1H-benzimidazole-1-carboxamide;
N-[(2,5-dimethyl-3-furyl)methyl]-2-oxo-3-(tetrahydro-2H-pyran-4-ylmethyl)-2,3-dihydro-1H-benzimidazole-1-carboxamide;
N-[(5-methylisoxazol-3-yl)methyl]-2-oxo-3(tetrahydro-2H-pyran-4-ylmethyl)-2,3-dihydro-1H-benzimidazole-1 carboxamide;
N-[(1S)-1-(aminocarbonyl)-2,2-dimethylpropyl]-3-(2-(dimethylamino)-2-oxoethyl)-2-oxo-2,3-dihydro-1H-benzimidazole-1-carboxamide;
N-[(1S)-1-(aminocarbonyl)-2,2-dimethylpropyl]-3-[2-(ethylamino)-2-oxoethyl]-2-oxo-2,3-dihydro-1H-benzimidazole-1-carboxamide;
N-[(1S)-1-(aminocarbonyl)-2,2-dimethylpropyl]-3-(2-cyclopropyl-2-oxoethyl)-2-oxo-2,3-dihydro-1H-benzimidazole-1-carboxamide;
N-[(1S)-1-(aminocarbonyl)-2,2-dimethylpropyl]-3-(3,3-dimethyl-2-oxobutyl)-2-oxo-2,3-dihydro-1H-benzimidazole-1-carboxamide;
N-[(1S)-1-{[(2-hydroxyethyl)amino]carbonyl}-2,2-dimethylpropyl]-2-oxo-3-(tetrahydro-2H-pyran-4-ylmethyl)-2,3-dihydro-1H-benzimidazole-1-carboxamide;
N-[(1S)-1-{[(3-hydroxypropyl)amino]carbonyl}-2,2-dimethylpropyl]-2-oxo-3-(tetrahydro-2H-pyran-4-ylmethyl)-2,3-dihydro-1H-benzimidazole-1-carboxamide;
N-[(1S)-1-(5-amino-1,3,4-oxadiazol-2-yl)-2,2-dimethylpropyl]-2-oxo-3-(tetrahydro-2H-pyran-4-ylmethyl)-2,3-dihydro-1H-benzimidazole-1-carboxamide;
N-{[5-methyl-2-(trifluoromethyl)-3-furyl]methyl}-2-oxo-3-(tetrahydro-2H-pyran-4-ylmethyl)-2,3-dihydro-1H-benzimidazole-1-carboxamide;
N-[(5-{[4-(hydroxymethyl)piperidin-1-yl]carbonyl}-1,2,4-oxadiazol-3-yl)methyl]-2-oxo-3-(tetrahydro-2H-pyran-4-ylmethyl)-2,3-dihydro-1H-benzimidazole-1-carboxamide;
N-[(1,5-dimethyl-1H-pyrazol-4-yl)methyl]-2-oxo-3-tetrahydro-2H-pyran-4-ylmethyl)-2,3-dihydro-1H-benzimidazole-1-carboxamide;
N-[(5-{[(3-methylbutyl)amino]carbonyl}-1,2,4-oxadiazol-3-yl)methyl]-2-oxo-3-(tetrahydro-2H-pyran-4-ylmethyl)-2,3-dihydro-1H-benzimidazole-1-carboxamide;
N-[(5-isopropyl-1,2,4-oxadiazol-3-yl)methyl]-2-oxo-3-(tetrahydro-2H-pyran-4-ylmethyl-2,3-dihydro-1H-benzimidazole-1-carboxamide;
2-oxo-3-(tetrahydro-2H-pyran-4-ylmethyl)-N-[(1,3,5-trimethyl-1H-pyrazol-4-yl)methyl]-2,3-dihydro-1H-benzimidazole-1-carboxamide;
N-(1,3-oxazol-4-ylmethyl)-2-oxo-3-(tetrahydro-2H-pyran-4-ylmethyl)-2,3-dihydro-1H-benzimidazole-1-carboxamide;
N-[(5-ethyl-1,2,4-oxadiazol-3-yl)methyl]-2-oxo-3-(tetrahydro-2H-pyran-4-ylmethyl)-2,3-dihydro-1H-benzimidazole-1-carboxamide;
N-[(2-ethyl-1,3-thiazol-4-yl)methyl]-2-oxo-3(tetrahydro-2H pyran-4-ylmethyl)-2,3-dihydro-1H-benzimidazole-1-carboxamide;
N-(3-furylmethyl)-2-oxo-3-(tetrahydro-2H-pyran-4-ylmethyl)-2,3-dihydro-1H-benzimidazole-1-carboxamide;
N-[(5-{[(3,4-dihydro-1H-isochromen-1-ylmethyl)amino]carbonyl}-1,2,4-oxadiazol-3-yl)methyl]-2-oxo-3-(tetrahydro-2H-pyran-4-ylmethyl)-2,3-dihydro-1H-benzimidazole-1-carboxamide;
N-{[5-(azepan-1-ylcarbonyl)-1,2,4-oxadiazol-3-yl]methyl}-2-oxo-3-(tetrahydro-2H-pyran-4-ylmethyl)-2,3-dihydro-1H-benzimidazole-1-carboxamide;
2-oxo-N-{[5-({[3-(2-oxopyrrolidin-1-yl)propyl]amino}carbonyl)-1,2,4-oxadiazol-3-yl]methyl}-3-(tetrahydro-2H-pyran-4-ylmethyl)-2,3-dihydro-1H-benzimidazole-1-carboxamide;
N-[(1-ethyl-1H-pyrazol-4-yl)methyl]-2-oxo-3-(tetrahydro-2H-pyran-4-ylmethyl)-2,3-dihydro-1H-benzimidazole-1-carboxamide;
2-oxo-N-({5-[(1,2,3,4-tetrahydronaphthalen-1-ylamino)carbonyl]-1,2,4-oxadiazol-3-yl}methyl)-3-(tetrahydro-2H-pyran-4-ylmethyl)-2,3-dihydro-1H-benzimidazole-1-carboxamide;
N-({5-[(dimethylamino)carbonyl]-1,2,4-oxadiazol-3-yl}methyl)-2-oxo-3-(tetrahydro-2H-pyran-4-ylmethyl)-2,3-dihydro-1H-benzimidazole-1-carboxamide;
N-[(5-{[(2-methoxyethyl)amino]carbonyl}-1,2,4-oxadiazol-3-yl)methyl]-2-oxo-3-(tetrahydro-2H-pyrany-4-ylmethyl)-2,3-dihydro-1H-benzimidazole-1-carboxamide;
N-[(2,4-dim ethyl-1,3-thiazol-5-yl)methyl]-2-oxo-3-(tetrahydro-2H-pyran-4-ylmethyl)-2,3-dihydro-1H-benzimidazole-1-carboxamide;
N-({5-[(methylamino)carbonyl]-1,2,4-oxadiazol-3-yl}methyl)-2-oxo-3-(tetrahydro-2H-pyran-4-ylmethyl)-2,3-dihydro-1H-benzimidazole-1-carboxamide;
N-[(1-isopropyl-1H-pyrazol-4-yl)methyl]-2-oxo-3-(tetrahydro-2H-pyran-4-ylmethyl)-2,3-dihydro-1H-benzimidazole-1-carboxamide;
N-[(1,3-dimethyl-1H-pyrazol-4-yl)methyl]-2-oxo-3-(tetrahydro-2H-pyran-4-ylmethyl)-2,3-di hydro-1H-benzimidazole-1-carboxamide;
N-[(2-methyl-1,3-thiazol-4-yl)methyl]-2-oxo-3-(tetrahydro-2H-pyran-4-ylmethyl)-2,3-dihydro-1H-benzimidazole-1-carboxamide;
N-[(5-{[(trans-4-hydroxycyclohexyl)amino]carbonyl}-1,2,4-oxadiazol-3-yl)methyl]-2-oxo-3-(tetrahydro-2H-pyran-4-ylmethyl)-2,3-dihydro-1H-benzimidazole-1-carboxamide;
N-[(3,5-dimethyl-1H-pyrazol-4-yl)methyl]-2-oxo-3-(tetrahydro-2H-pyran-4-ylmethyl)-2,3-dihydro-1H-benzimidazole-1-carboxamide;
N-[(1-methyl-1H-pyrazol-4-yl)methyl]-2-oxo-3-(tetrahydro-2H-pyran-4-ylmethyl)-2,3-dihydro-1H-benzimidazole-1-carboxamide;
N-[(1S)-1-{([(2S-2,3-dihydroxypropyl]amino}carbonyl)-2,2-dimethylpropyl]-2-oxo-3-(tetrahydro-2H-pyran-4-methyl)-2,3-dihydro-1H-benzimidazole-1-carboxamide;
N-[(5-methyl-1,3,4-oxadiazol-2-yl)methyl]-2-oxo-3-(tetrahydro-2H-pyran-4-ylmethyl)-2,3-dihydro-1H-benzimidazole-1-carboxamide;
N-[(1S)-1-(5-amino-1,3,4-oxadiazol-2-yl)-2,2-dimethylpropyl]-5-fluoro-2-oxo-3-(tetrahydro-2H-pyran-4-ylmethyl)-2,3-dihydro-1H-benzimidazole-1-carboxamide;
N-[(1S)-1-(aminocarbonyl)-2,2-dimethylpropyl]-3-benzyl-2-oxo-2,3-dihydro-1H-benzimidazole-1-carboxamide;
N-{[2-methyl-4-(trifluoromethyl)-1,3-thiazol-5-yl]methyl}-2-oxo-3-(tetrahydro-2H-pyran-4-ylmethyl)-2,3-dihydro-1H-benzimidazole-1-carboxamide;
N-[1-(2-methyl-1,3-thiazol-4-yl)ethyl]-2-oxo-3-(tetrahydro-2H-pyran-4-ylmethyl)-2,3-dihydro-1H-benzimidazole-1-carboxamide;
N-[(1S)-1-(aminocarbonyl)-2,2-dimethylpropyl]-2-oxo-3-{[(2R)-5-oxotetrahydrofuran-2-yl]methyl}-2,3-dihydro-1H-benzimidazole-1-carboxamide,
N-[(1S)-1-(5-amino-1,3,4-oxadiazol-2-yl)-2,2-dimethylpropyl]-2-oxo-3-{[(2R)-5-oxotetrahydrofuran-2-yl]methyl}-2,3-dihydro-1H-benzimidazole-1-carboxamide;
N-[(1S)-1-{[(3-hydroxypropyl)amino]carbonyl}-2,2-dimethylpropyl]-2-oxo-3-{[(2R)-5-oxotetrahydrofuran-2-yl]methyl}-2,3-dihydro-1H-benzimidazole-1-carboxamide;
N-[(1S)-1-{[(2-hydroxyethyl)amino]carbonyl}-2,2-dimethylpropyl]-2-oxo-3-{[(2R)-5-oxotetrahydrofuran-2-yl]methyl}-2,3-dihydro-1H-benzimidazole-1-carboxamide;
5-fluoro-N-[(1S)-1-{[(3-hydroxypropyl)amino]carbonyl}-2,2-dimethylpropyl]-2-oxo-3-(tetrahydro-2H-pyran-4-ylmethyl)-2,3-dihydro-1H-benzimidazole-1-carboxamide;
5-fluoro-N-[(1S)-1-{[(2-hydroxyethyl)amino]carbonyl}-2,2-dimethylpropyl]-2-oxo-3-(tetrahydro-2H-pyran-4-ylmethyl)-2,3-dihydro-1H-benzimidazole-1-carboxamide;
N-[(1S)-1-(aminocarbonyl)-2,2-dimethylpropyl]-3-(2-fluorobenzyl)-2-oxo-2,3-dihydro-1H-benzimidazole-1-carboxamide;
N-[(1S)-1-(aminocarbonyl)-2,2-dimethylpropyl]-3-(4-methoxybenzyl)-2-oxo-2,3-dihydro-1H-benzimidazole-1-carboxamide;
N-[(1S)-1-(5-amino-1,3,4-oxadiazol-2-yl)-2,2-dimethylpropyl]-3-benzyl-2-oxo-2,3-dihydro-1H-benzimidazole-1-carboxamide;
3-benzyl-N-[(1S)-1-{[(2-hydroxyethyl)amino]carbonyl}-2,2-dimethylpropyl]-2-oxo-2,3-dihydro-1H-benzimidazole-1-carboxamide;
3-benzyl-N-[(1S)-1-{[(3-hydroxypropyl)amino]carbonyl}-2,2-dimethylpropyl]-2-oxo-2,3-dihydro-1H-benzimidazole-1-carboxamide;
N-[(1S)-1-(aminocarbonyl)-2,2-dimethylpropyl]-3-(3-fluorobenzyl)-2-oxo-2,3-dihydro-1H-benzimidazole-1-carboxamide;
N-[(1S)-1-(aminocarbonyl)-2,2-dimethylpropyl]-3-(4-fluorobenzyl)-2-oxo-2,3-dihydro-1H-benzimidazole-1-carboxamide;
N-[(1S)-1-(aminocarbonyl)-2,2-dimethylpropyl]-2-oxo-3-(tetrahydro-2H-pyran-4-ylmethyl)-2,3-dihydro-1H-imidazo[4,5-b]pyridine-1-carboxamide;
N-[(1S)-1-(aminocarbonyl)-2,2-dimethylpropyl]-2-oxo-3-pentyl-2,3-dihydro-1H-imidazo[4,5-b]pyridine-1-carboxamide;
N-[(1S)-1-(aminocarbonyl)-2,2-dimethylpropyl]-3-benzyl-2-oxo-2,3-dihydro-1H-benzimidazole-1-carboxamide;
N-[(1S)-1-(aminocarbonyl)-2,2-dimethylpropyl]-3-(2-fluorobenzyl)-2-oxo-2,3-dihydro-1H-benzimidazole-1-carboxamide;
N-[(1S)-1-(aminocarbonyl)-2,2-dimethylpropyl]-3-(4-methoxybenzyl)-2-oxo-2,3-dihydro-1H-benzimidazole-1-carboxamide;
N-[(1S)-1-(5-amino-1,3,4-oxadiazol-2-yl)-2,2-dimethylpropyl]-3-benzyl-2-oxo-2,3-dihydro-1H-benzimidazole-1-carboxamide;
3-benzyl-N-[(1S)-1-[(2-hydroxyethyl)amino]carbonyl)-2,2-dimethylpropyl]-2-oxo-2,3-dihydro-1H-benzimidazole-1-carboxamide;
3-benzyl-N-[(1S)-1-{[(3-hydroxypropyl)amino]carbonyl}-2,2-dimethylpropyl]-2-oxo-2,3-dihydro-1H-benzimidazole-1-carboxamide;
N-[(1S)-1-(aminocarbonyl)-2,2-dimethylpropyl]-3-(4-cyanobenzyl)-2-oxo-2,3-dihydro-1H-benzimidazole-1-carboxamide;
N-[(1S)-1-(aminocarbonyl)-2,2-dimethylpropyl]-3-(2-cyanobenzyl)-2-oxo-2,3-dihydro-1H-benzimidazole-1-carboxamide;
N-[(1S)-1-(aminocarbonyl)-2,2-dimethylpropyl]-3(3-fluorobenzyl)-2-oxo-2,3-dihydro-1H-benzimidazole-1-carboxamide;
N-[(1S)-1-aminocarbonyl)-2,2-dimethylpropyl]-3-(4-fluorobenzyl)-2-oxo-2,3-dihydro-1H-benzimidazole-1-carboxamide;
3-(4-fluorobenzyl)-N-[(1S)-1-{[(2-hydroxyethyl)amino]carbonyl}-2,2-dimethylpropyl]-2-oxo-2,3-dihydro-1H-benzimidazole-1-carboxamide;
3(4-fluorobenzyl)-N-[(1S)-1-{[(3-hydroxypropyl)amino]carbonyl}-2,2-dimethylpropyl]-2-oxo-2,3-dihydro-1H-benzimidazole-1-carboxamide;
N-{[3-(4-fluorobenzyl)-2-oxo-2,3-dihydro-1H-benzimidazol-1-yl]carbonyl}-3-methyl-L-valylglycinamide; and
N-[(1S)-1-(5-amino-1,3,4-oxadiazol-2-yl)-2,2-dimethylpropyl]-(4-fluorobenzyl)-2-oxo-2,3-dihydro-1H-benzimidazole-1-carboxamide;

or a pharmaceutically acceptable salt thereof.

As used herein, the terms “treating”, “treatment”, “treated”, or “to treat,” can be used interchangeably. Treatment includes palliative treatment, preventive treatment and restorative treatment. Palliative treatment includes alleviation, elimination of causation of pain and/or inflammation associated with a CB1 mediated disorder. Preventative treatment means to prevent or to slow the appearance of symptoms associated with a CB1 mediated disorder. For methods of prevention, the subject is any subject, and preferably is a subject that is in need of prevention of a CB1 mediated disorder.

Pharmaceutically acceptable salts of a compound of formula (I) include the acid addition and base salts (including disalts) thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

For a review on suitable salts, see “Handbook of Pharmaceutical Salts: Properties, Selection, and Use” by Stahl and Wermuth (Wiley-VCH, Weinheim, Germany, 2002). A pharmaceutically acceptable salt of a compound of formula (I) may be readily prepared by mixing together solutions of the compound of formula (I) and the desired acid or base, as appropriate. The salt may precipitate from solution and be collected by filtration or may be recovered by evaporation of the solvent. The degree of ionisation in the salt may vary from completely ionised to almost non-ionised.

The compounds useful in the present invention may exist in both unsolvated and solvated forms. The term ‘solvate’ is used herein to describe a molecular complex comprising a compound of the invention and one or more pharmaceutically acceptable solvent molecules, for example, ethanol. The term ‘hydrate’ is employed when said solvent is water.

Pharmaceutically acceptable solvates in accordance with the invention include hydrates and solvates wherein the solvent of crystallization may be isotopically substituted, e.g. D₂O, d₆-acetone, d₆-DMSO

Included within the scope of the invention are complexes such as clathrates, drug-host inclusion complexes wherein, in contrast to the aforementioned solvates, the drug and host are present in stoichiometric or nor-stoichiometric amounts. Also included are complexes of the drug containing two or more organic and/or inorganic components which may be in stoichiometric or non-stoichiometric amounts. The resulting complexes may be ionised, partially ionised, or non-ionised. For a review of such complexes, see J Pharm Sci, 84 (8), 1269-1288 by Haleblian (August 1975).

Hereinafter all references to a compound of formula (I) include references to salts and complexes thereof and to solvates and complexes of salts thereof.

The term “compound of the invention” or “compounds of the invention” refers to, unless indicated otherwise, a compound of formula (I) as hereinbefore defined, polymorphs, prodrugs, and isomers thereof (including optical, geometric and tautomeric isomers) as hereinafter defined and isotopically-labeled compounds of formula (I).

Also within the scope of the invention are so-called ‘prodrugs’ of the compounds of formula (I). Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further Information on the use of prodrugs may be found in ‘Pro-drugs as Novel Delivery Systems, Vol. 14, ACS Symposium Series (T Higuchi and W Stella) and ‘Bioreversible Carriers In Drug Design’, Pergamon Press, 1987 (ed. E B Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in “Design of Prodrugs” by H Bundgaard (Elsevier, 1985). Some examples of prodrugs in accordance with the invention include:
(i) where the compound of formula (I) contains an alcohol functionality (—OH), an ether thereof, for example, replacement of the hydrogen with (C₁-C₆)alkanoyloxymethyl;
(ii) where the compound of formula (I) contains carboxy group, an ester thereof, for example, replacement of the OH of the carboxy with C₁-C₈ alkyl; and
(ii) where the compound of formula (I) contains a primary or secondary amino functionality (—NH₂ or —NHR where R≠H), an amide thereof, for example, replacement of one or both hydrogens with (C₁-C₁₀)alkanoyl.

Further examples of replacement groups in accordance with the foregoing examples and examples of other prodrug types may be found in the aforementioned references.

Finally, certain compounds of formula (I) may themselves act as prodrugs of other compounds of formula (I).

Compounds of formula (I) containing one or more asymmetric carbon atoms can exist as two or more stereoisomers. Where the compound contains, for example, a keto or oxime group or an aromatic moiety, tautomeric isomerism (‘tautomerism’) can occur. It follows that a single compound may exhibit more than one type of isomerism.

Included within the scope of the present invention are all stereoisomers, geometric Isomers and tautomeric forms of the compounds of formula (I), including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Stereoisomeric conglomerates may be separated by conventional techniques known to those skilled in the art—see, for example, “Stereochemistry of Organic Compounds” by E L Eliel (Wiley, New York, 1994).

The present invention includes all pharmaceutically acceptable isotopically-labelled compounds of formula (I) wherein one or more atoms are replaced by atoms having the same atomic number, but an atomic mass or mass number different from the atomic mass or mass number usually found in nature.

Examples of isotopes suitable for inclusion in the compounds of the Invention include isotopes of hydrogen, such as ²H and ³H, carbon, such as ¹¹C, ¹³C and ¹⁴C, chlorine, such as ³⁸Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

All of the compounds of the formula (I) can be prepared by the procedures described in the general methods presented below or by the specific methods described in the Examples section and the Preparations section, or by routine modifications thereof. The present invention also encompasses any one or more of these processes for preparing the compounds of formula (I), in addition to any novel intermediates used therein.

### General Synthesis

The compounds of the present invention may be prepared by a variety of processes well known for the preparation of compounds of this type, for example as shown in the following Methods A to D.

The following Method A illustrates the preparation of compounds of formula (I). Methods B through D illustrate the preparation of various intermediates.

Unless otherwise indicated, R¹, R², R³, A and B in the following Methods are as defined above. The term “protecting group”, as used hereinafter, means a hydroxy, carboxy or amino-protecting group which is selected from typical hydroxy, carboxy or amino-protecting groups described in Protective Groups in Organic Synthesis edited by T. W. Greene et al. (John Wiley & Sons, 1999). All starting materials in the following general syntheses may be commercially available or obtained by conventional methods known to those skilled in the art, such as Meth-Cohn, O.; Smith, D. I. J.C.S., Perkin Trans. 1, 1982, 261; Vernin, G.; Domlog, H.; Siv, C.; Metzger, J. J. Heterocyclic Chem. 1981, 18, 85; Emily, M. S. et al. Tetrahedron 2001, 57, 5303-5320; Kubo, K. et al. J. Med. Chem. 1993, 36, 1772-1784; Israel, M.; Jones, L. C. J. Heterocyclic Chem. 1971, 8, 797; Sebok, P.; Levai, A.; Timar, T. Heterocyclic Commun. 1998, 4, 547-552.); and the disclosures of which are incorporated herein by references.

### Method A

This illustrates the preparation of compounds of formula (I).

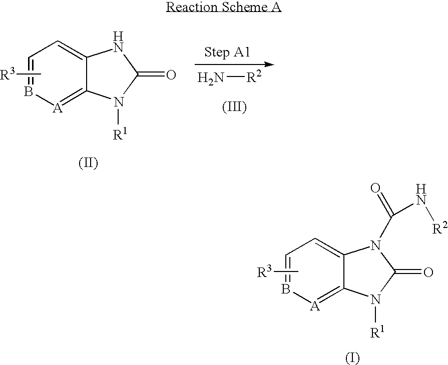

### Step A1

In this step, the desired compound of formula (I) of the present invention is prepared by carbonylation of the compound of formula (II) with the compound of formula (III). The compound of formula (II) is commercially available or can be prepared according to the Methods B and C set forth below. The compound of formula (III) is commercially available.

The reaction is normally and preferably effected in the presence of solvent. There is no particular restriction on the nature of the solvent to be employed, provided that it has no adverse effect on the reaction or the reagents involved and that it can dissolve reagents, at least to some extent. Examples of suitable solvents include, but are not limited to: halogenated hydrocarbons, such as dichloromethane, chloroform, carbon tetrachloride and 1,2-dichloroethane; aromatic hydrocarbons, such as benzene, toluene and nitrobenzene; ethers, such as diethyl ether, diisopropyl ether, tetrahydrofuran and dioxane; and amides, such as N,N-dimethylformamide and N,N-dimethylacetamide. Of these solvents, dichloromethane is preferred.

There is likewise no particular restriction on the nature of the carbonylating agents used, and any carbonylating agent commonly used in reactions of this type may equally be used here. Examples of such carbonylating agents include, but are not limited to: an imidazole derivative such as N,N′-carbonyldiimidazole (CDI); a chloroformate such as trichloromethyl chloroformate and 4-nitrophenyl chloroformate; urea; and triphosgene. Of these, 4-nitrophenyl chloroformate is preferred.

The reaction can take place over a wide range of temperatures, and the precise reaction temperature is not critical to the invention. The preferred reaction temperature will depend upon such factors as the nature of the solvent, and the starting materials. However, in general, it is convenient to carry out the reaction at a temperature of from about 0 degrees Celsius to about 100 degrees Celsius. The time required for the reaction may also vary widely, depending on many factors, notably the reaction temperature and the nature of the starting materials and solvent employed. However, provided that the reaction is effected under the preferred conditions outlined above, a period of from about 5 minutes to about 24 hours will usually suffice.

### Method B

This illustrates the preparation of compounds of formula (II).

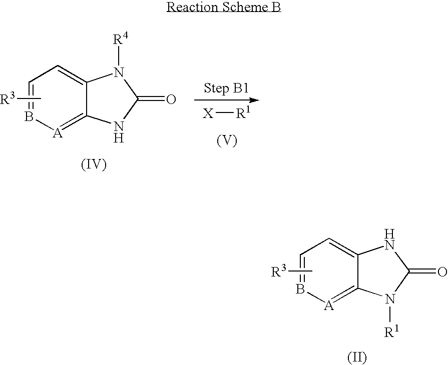

In Reaction Scheme B, R⁴ is an amide-protecting group; X is a leaving group.

The term “amide-protecting group”, as used herein, signifies a protecting group capable of being cleaved by chemical means, such as hydrogenolysis, hydrolysis, electrolysis or photolysis and such amide-protecting groups are described in Protective Groups in Organic Synthesis edited by T. W. Greene et al., (John Wiley & Sons, 1999). Typical amide-protecting groups include, but are not limited to, allyl, isopropenyl, t-butyl, methoxymethyl, benzyloxy and t-butyldimethylsilyl. Of these groups, isopropenyl is preferred.

The term “leaving group”, as used herein, signifies a group capable of being substituted by nucleophilic groups, such as a hydroxy group, amines or carboanions and examples of such leaving groups include halogen atoms, a alkylsulfonyl group and a phenylsulfonyl group. Of these, a bromine atom, a chlorine atom and a methylsulfonyl group are preferred.

### Step B1

In this step, the compound of formula (II) is prepared by the nucleophilic substitution (B1-a) with the compound of formula (V) followed by deprotection (B1-b). The compound of formula (IV) is commercially available or can be prepared according to the methods described in Israel, M.; Jones, L. C. J. Heterocyclic Chem. 1971, 8, 797. The compound of formula (V) is commercially available.

### (B1-a) Nucleophilic Substitution

The reaction is normally and preferably effected in the presence of solvent. There is no particular restriction on the nature of the solvent to be employed, provided that it has no adverse effect on the reaction or the reagents involved and that it can dissolve reagents, at least to some extent. Examples of suitable solvents include: ethers, such as diethyl ether, diisopropyl ether, tetrahydrofuran and dioxane; amides, such as formamide, N,N-dimethylformamide, N,N-dimethylacetamide and hexamethylphosphoric triamide; nitrites, such as acetonitrile and benzonitrile; and sulfoxides, such as dimethyl sulfoxide and sulfolane. Of these solvents, N,N-dimethylformamide is preferred.

The reaction is carried out in the presence of a base. There is likewise no particular restriction on the nature of the bases used, and any base commonly used in reactions of this type may equally be used here. Examples of such bases include: alkali metal hydrides, such as lithium hydride, sodium hydride and potassium hydride; and alkali metal amides, such as lithium amide, sodium amide, potassium amide, lithium diisopropyl amide, potassium diisopropyl amide, sodium diisopropyl amide, lithium bis(trimethylsilyl)amide and potassium bis(trimethylsilyl)amide. Of these, sodium hydride is preferred.

The reaction can take place over a wide range of temperatures, and the precise reaction temperature is not critical to the invention. The preferred reaction temperature will depend upon such factors as the nature of the solvent, and the starting materials. However, in general, it is convenient to carry out the reaction at a temperature of from about −20 degrees Celsius to about 50 degrees Celsius. The time required for the reaction may also vary widely, depending on many factors, notably the reaction temperature and the nature of the starting materials and solvent employed. However, provided that the reaction is effected under the preferred conditions outlined above, a period of from about 30 minutes to about 24 hours, will usually suffice.

### (B1-b) Deprotection

The deprotection method is described in detail by T. W. Greene et al. [Protective Groups in Organic Synthesis, 494-653, (1999)], the disclosures of which are incorporated herein by reference. The following exemplifies a typical method involving the protecting group is isopropenyl.

The reaction is normally and preferably effected in the presence of solvent. There is no particular restriction on the nature of the solvent to be employed, provided that it has no adverse effect on the reaction or the reagents involved and that it can dissolve reagents, at least to some extent. Examples of suitable solvents include, but are not limited to: ethers, such as diethyl ether, diisopropyl ether, tetrahydrofuran and dioxane; water; and alcohols, such as methanol, ethanol, propanol, 2-propanol and butanol. Of these solvents, water or alcohols are preferred.

The reaction is carried out in the presence of excess amount of an acid. There is likewise no particular restriction on the nature of the acids used, and any acid commonly used in reactions of this type may equally be used here. Examples of such acids include, but are not limited to: acids, such as hydrochloric acid, sulfuric acid or trifluoroacetic acid. Of these, hydrochloric acid is preferred.

The reaction can take place over a wide range of temperatures, and the precise reaction temperature is not critical to the invention. The preferred reaction temperature will depend upon such factors as the nature of the solvent, and the starting materials. However, in general, it is convenient to carry out the reaction at a temperature of from about 25 degrees Celsius to about 120 degrees Celsius. The time required for the reaction may also vary widely, depending on many factors, notably the reaction temperature and the nature of the starting materials and solvent employed. However, provided that the reaction is effected under the preferred conditions outlined above, a period of from about 15 minutes to about 12 hours, will usually suffice.

### Method C

This illustrates the preparation of compounds of formula (II).

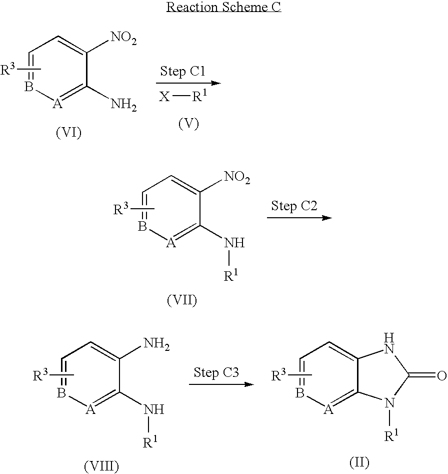

In Reaction Scheme C, X is as defined above.

### Step C1

In this step, the compound of formula (VII) is prepared by the nucleophilic substitution of the compound of formula (VI) with the compound of formula (V). The compound of formula (VI) is commercially available or can be prepared according to the methods described in Kubo, K. et al. J. Med. Chem. 1993, 36, 1772-1784. The compound of formula (V) is commercially available. The reaction may be carried out under the same conditions as described in Step B1-a of Method B.

### Step C2

In this step, the compound of formula (VIII) is prepared by the reduction of the nitro group.

The reaction is normally and preferably effected in the presence of solvent. There is no particular restriction on the nature of the solvent to be employed, provided that it has no adverse effect on the reaction or the reagents involved and that it can dissolve reagents, at least to some extent. Examples of suitable solvents include: ethers, such as diethyl ether, diisopropyl ether, tetrahydrofuran and dioxane; aromatic hydrocarbons, such as benzene and toluene; alcohols, such as methanol, ethanol, propanol, 2-propanol and butanol; and esters, such as ethyl acetate. Of these solvents, tetrahydrofuran is preferred.

The reaction is carried out in the presence of a reducing agent. There is likewise no particular restriction on the nature of the reducing agents used, and any reducing agent commonly used in reactions of this type may equally be used here. Examples of such reducing agents include: hydride compounds such as lithium aluminum hydride, sodium borohydride and diisobutyl aluminum hydride; combinations of hydrogen gas and a catalyst such as palladium-carbon, platinum and Raney nickel; and a combination of metals, such as zinc and iron, and acids, such as hydrochloric acid, acetic acid and acetic acid-ammonium chloride complex. Of these, lithium aluminum hydride is preferred.

The reaction can take place over a wide range of temperatures, and the precise reaction temperature is not critical to the invention. The preferred reaction temperature will depend upon such factors as the nature of the solvent, and the starting materials. However, in general, it is convenient to carry out the reaction at a temperature of from about 25 degrees Celsius to about 120 degrees Celsius. The time required for the reaction may also vary widely, depending on many factors, notably the reaction temperature and the nature of the starting materials and solvent employed. However, provided that the reaction is effected under the preferred conditions outlined above, a period of from about 15 minutes to about 24 hours will usually suffice.

### Step C3

In this step, the compound of formula (II) is prepared by the formation of the cyclic urea of the compound of formula (VIII).

The reaction is normally and preferably effected in the presence of solvent. There is no particular restriction on the nature of the solvent to be employed, provided that it has no adverse effect on the reaction or the reagents involved and that it can dissolve reagents, at least to some extent. Examples of suitable solvents include, but are not limited to: halogenated hydrocarbons, such as dichloromethane, chloroform, carbon tetrachloride and 1,2-dichloroethane; aromatic hydrocarbons, such as benzene, toluene and nitrobenzene; ethers, such as diethyl ether, diisopropyl ether, tetrahydrofuran and dioxane; and amides, such as N,N-dimethylformamide and N,N-dimethylacetamide. Of these solvents, tetrahydrofuran is preferred.

There is likewise no particular restriction on the nature of the carbonylating agents used, and any carbonylating agent commonly used in reactions of this type may equally be used here. Examples of such carbonylating agents include, but are not limited to: an imidazole derivative such as N,N′-carbonyldiimidazole (CDI); a chloroformate such as trichloromethyl chloroformate and 4-nitrophenyl chloroformate; urea; and triphosgene. Of these, CDI or urea is preferred.

The reaction can take place over a wide range of temperatures, and the precise reaction temperature is not critical to the invention. The preferred reaction temperature will depend upon such factors as the nature of the solvent, and the starting materials. However, in general, it is convenient to carry out the reaction at a temperature of from about 0 degrees Celsius to about 100 degrees Celsius. The time required for the reaction may also vary widely, depending on many factors, notably the reaction temperature and the nature of the starting materials and solvent employed. However, provided that the reaction is effected under the preferred conditions outlined above, a period of from about 30 minutes to about 12 hours will usually suffice.

### Method D

This illustrates the preparation of compounds of formula (II).

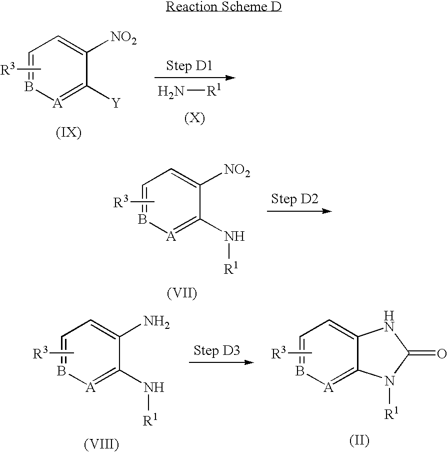

In Reaction Scheme-C, Y is a chlorine atom or fluorine atom.

### Step D1

In this step, the compound of formula (VII) is prepared by the nucleophilic substitution of the compound of formula (IX) with the compound of formula (X). The compound of formula (IX) is commercially available or can be prepared according to the methods described in Orjales, A. et al. J. Med. Chem. 1999, 42, 2870-2880. The compound of formula (X) is commercially available.

The reaction is normally and preferably effected in the presence of solvent. There is no particular restriction on the nature of the solvent to be employed, provided that it has no adverse effect on the reaction or the reagents involved and that it can dissolve reagents, at least to some extent. Examples of suitable solvents include, but are not limited to: halogenated hydrocarbons, such as dichloromethane, chloroform, carbon tetrachloride and 1,2-dichloroethane; aromatic hydrocarbons, such as benzene, toluene and nitrobenzene; ethers, such as diethyl ether, diisopropyl ether, tetrahydrofuran and dioxane; alcohols, such as methanol, ethanol, propanol, 2-propanol and butanol; and amides, such as N,N-dimethylformamide and N,N-dimethylacetamide. Of these solvents, tetrahydrofuran is preferred.

The reaction is carried out in the presence of a base. There is likewise no particular restriction on the nature of the bases used, and any base commonly used in reactions of this type may equally be used here. Examples of such bases include: alkali metal alkoxides, such as sodium methoxide, sodium ethoxide and potassium t-butoxide; alkali metal carbonates, such as lithium carbonate, sodium carbonate and potassium carbonate; amines, such as N-methylmorpholine, triethylamine, tripropylamine, tributylamine, diisopropylethylamine, dicyclohexylamine, N-methylpiperidine, pyridine, 4-pyrrolidinopyridine, picoline, 4-(N,N-dimethylamino)pyridine, 2,6-di(t-butyl)-4-methylpyridine, quinoline, N,N-dimethylaniline, N,N-diethylaniline, 1,5-diazabicyclo[4.3.0]non-5-ene (DBN), 1,4-diazabicyclo[2.2.2]octane (DABCO) and 1,8-diazabicyclo[5.4.0]undec-7-ene (DBU); and alkali metal hydrogencarbonates, such as lithium hydrogencarbonate, hydrogensodium carbonate and potassium hydrogencarbonate. Of these, potassium carbonate is preferred.

The reaction can take place over a wide range of temperatures, and the precise reaction temperature is not critical to the invention. The preferred reaction temperature will depend upon such factors as the nature of the solvent, and the starting materials. However, in general, it is convenient to carry out the reaction at a temperature of from about −20 degrees Celsius to about 120 degrees Celsius. The time required for the reaction may also vary widely, depending on many factors, notably the reaction temperature and the nature of the starting materials and solvent employed. However, provided that the reaction is effected under the preferred conditions outlined above, a period of from about 1 hour to about 36 hours will usually suffice. In this reaction, microwave can be employed to accelerate the reaction. In the case of employing microwave, the reaction at a temperature may be from about 50 degrees Celsius to about 220 degrees Celsius and the reaction time from about 5 minutes to about 6 hours will usually suffice.

### Steps D2 and D3

The reactions may be carried out under the same conditions as described in Steps C2 and C3.

The compounds of formula (I), and the intermediates above-mentioned preparation methods can be isolated and purified by conventional procedures, such as distillation, recrystallization or chromatographic purification.

Compounds of the invention intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a pharmaceutical composition or formulation in association with one or more pharmaceutically acceptable carriers or excipients. The term “carrier” or “excipient” is used herein to describe any ingredient other than the compound(s) of the invention. The choice of carrier or excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

### Oral Administration

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as, for example, tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include, for example, suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used In fast-dissolving, fast-disintegrating dosage forms such as those described in Expert Opinion in Therapeutic Patents, 11 (6), 981-986 by Liang and Chen (2001).

For tablet dosage forms, depending on dose, the drug may make up from about 1 wt % to about 80 wt % of the dosage form, more typically from about 5 wt % to about 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from about 1 wt % to about 25 wt %, preferably from about 5 wt % to about 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from about 0.2 wt % to about 5 wt % of the tablet, and glidants may comprise from about 0.2 wt % to about 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from about 0.25 wt % to about 10 wt %, preferably from about 0.5 wt % to about 3 wt % of the tablet.

Other possible ingredients include anti-oxidants, colourants, flavouring agents, preservatives and taste-masking agents.

Exemplary tablets contain up to about 80% drug, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Verma et al, Pharmaceutical Technology On-line, 25(2), 1-14 (2001). The use of chewing gum to achieve controlled release is described in WO 00/35298.

### Parenteral Administration

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from about 3 to about 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and PGLA microspheres.

### Topical Administration

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955958 by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

### Inhaled/Intranasal Administration

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurized container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed Jet milling, supercritical fluid processing to form nanoparticles, high pressure homogenization, or spray drying.

Capsules (made, for example, from gelatin or HPMC), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from about 1 μg to about 20 mg of the compound of the invention per actuation and the actuation volume may vary from about 1 μl to about 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration. Formulations for inhaled/intranasal administration may be formulated to be immediate and/or modified release using, for example, poly(DL-lactic-coglycolic acid (PGLA). Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from about 1 to about 100 μg of the compound of formula (I). The overall daily dose will typically be in the range about 50 μg to about 20 mg which may be administered in a single dose or, more usually, as divided doses throughout the day.

### Rectal/Intravaginal Administration

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

### Ocular/Aural Administration

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

### Other Technologies

The compounds of the invention may be combined with soluble macromolecular entitles, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in. WO 91/11172, WO 94/02518 and WO 98/55148.

### Kit-of-Parts

Inasmuch as it may be desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus the kit of the invention comprises two or more separate pharmaceutical compositions, at least one of which contains a compound of formula (I) in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

### Dosage

For administration to human patients, the total daily dose of the compounds of the invention is typically in the range of about 0.05 mg to about 100 mg depending, of course, on the mode of administration, preferred in the range of about 0.1 mg to about 50 mg and more preferred in the range of about 0.5 mg to about 20 mg. For example, oral administration may require a total daily dose of from about 1 mg to about 20 mg, while an intravenous dose may only require from about 0.5 mg to about 10 mg. The total daily dose may be administered in single or divided doses.

These dosages are based on an average human subject having a weight of about 65 kg to about 70 kg. The physician will readily be able to determine doses for subjects whose weight falls outside this range, such as infants and the elderly.

### Combination

As discussed above, a compound of the invention exhibits CB1 receptor binding activity. A CB1 ligand of the present invention may be usefully combined with another pharmacologically active compound, or with two or more other pharmacologically active compounds, particularly in the treatment of the cancer, inflammatory diseases, immunomodulatory diseases and gastrointestinal disorder. For example, a CB1 ligands, particularly a compound of the formula (I), or a pharmaceutically acceptable salt thereof, as defined above, may be administered simultaneously, sequentially or separately in combination with one or more agents selected from:
(i) 5-HT₃ antagonists, e.g. dolasetron, palonosetron, alosetron, azasetron and ramosetron, mitrazapine, granisetron, tropisetron, E-3620, ondansetron and indisetron;
(ii) 5-HT₄ agonists, e.g. tegaserod, mosapride, cinitapride and oxtriptane;
(iii) opioid analgesics, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine Modulon® (trimebutine malate), Imodium® (loperamide) and pentazocine;
(iv) tricyclic antidepressants, e.g. imipramine, amitriptyline, clomipramine, amoxapine and lofepramine;
(v) somatostatin analogues, e.g. octreotide, AN238 and PTR-3173;
(vi) anticholinergics, e.g. dicyclomine and hyoscyamine, ipratroplum bromide, ipratroplum, tiotropium bromide;
(vii) laxatives, e.g. Trifyba®, Fybogel®, Konsyl®, Isoget®, Regulan®, Celevac® and Normacol®;
(viii) fiber products, e.g. Metamucil®;
(ix) antispasmodics, e.g.: mebeverine;
(x) dopamine antagonists, e.g. metoclopramide, domperidone and levosulpiride;
(xi) cholinergics, e.g. neostigmine, pilocarpine, carbachol
(xii) calcium channel blockers, e.g. aranidipine, lacidipine, falodipine, azelnidipine, clinidipine, lomerizine, diltiazem, gallopamil, efonidipine, nisoldipine, amlodipine, lercanidipine, bevantolol, nicardipine, isradipine, benidipine, verapamil, nitrendipine, barnidipine, propafenone, manidipine, bepridil, nifedipine, nilvadipine, nimodipine and fasudil;
(xiii) Cl Channel activator: e.g. lubiprostone;
(xiv) selective serotonin reuptake inhibitors, e.g. sertraline, escitalopram, fluoxetine, nefazodone, fluvoxamine, citalopram, milnacipran, paroxetine, venlafaxine, tramadol, sibutramine, duloxetine, desvenlafaxine and depoxetine;
(xv) GABA agonists, e.g. gabapentin, topiramate, cinolazepam, clonazepam, progabide, brotizolam, zopiclone, pregabalin and eszopiclone;
(xvi) tachykinin (NK) antagonists, particularly NK-3, NK-2 and NK-1 antagonists, e.g.: nepadutant, saredutant, talnetant, (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]naphthridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]methyl]-1,2-dihydro-3H-1,2,4-triazol-3-one (MK-869), lanepitant, dapitant and 3-[[2-methoxy-5-(trifluoromethoxy)phenyl]methylamino]-2-phenyl-piperidine (2S,3S).
(xvii). α2 agonists, e.g. clonidine, medetomidine, lofexidine, moxonidine, tizanidine, guanfacine, guanabenz, talipexole and dexmedetomidine;
(xviii) benzodiazepine agonists, e.g. diazepam, zaleplon, zolpidem, haloxazolam, clonazepam, prazepam, quazepam, flutazolam, triazolam, lormetazepam, midazolam, tofisopam, clobazam, flunitrazepam and flutoprazepam;
(xix) prostaglandin analogues, e.g. Prostaglandin, misoprostol, treprostinil, esoprostenol, latanoprost, iloprost, beraprost, enprostil, ibudilast and ozagrel;
(xx) histamine H₃ agonists, e.g. R-alpha-methylhistamine and BP-294;
(xxi) anti-gastric agents, e.g. Anti-gastrin vaccine, itriglumide and Z-360;
(xxii) disease modifying anti-rheumatic drugs (DMARDS), e.g. methotrexate, leflunomide, penicillamine aurothiopropanol sulfonate, sulfasalazine, mesalamine, olsalazine, balsalazide, Hylan G-F 20, glucosamine, chondroitin sulfate, hydroxychloroquine and diacerein.
(xxiii) Tumor Necrosis Factor-Alpha (TNF-α) modulators, e.g. etanercept, infliximab, adalimumab, CDP-870, pegsunercept, ISIS-104838, RDP-58 and thalidomide;
(xxiv) interleukin-based therapies, e.g. anakinra, atlizumabi RGN-303, denileukindiftitox, ilodecakin, oprelvekin and mepolizumab;
(xxv) nonsteroidal antiinflammatory drugs (NSAIDs), e.g. piroxicam, naproxen, indomethacin, ibuprofen, diclofenac, ketorolac, flurbiprofen, aspirin, diflusinal, etodolac, fenbufen, fenoprofen, flufenisal, ketoprofen, meclofenamic acid, mefenamic acid, nabumetone, oxaprozin, phenylbutazone, sulindac, tolmetin and zomepirac;
(xxvi) selective COX-2 inhibitors, e.g. celecoxib, rofecoxib, valdecoxib, etoricoxib, lumiracoxib and LAS-34475;
(xxvii) Centrally Acting Analgesics, e.g. tramadol and oxymorphone ER;
(xxviii) immunosupressives, e.g. cyclosporine, tacrolimus, rapamycin, azathioprine and mycophenolate mofetil;
(xxix) Multiple Sclerosis (MS) treatments, e.g. interferonβ-1b, interferonβ-1α, glatiramer gcetate, mitoxantrone, cyclophosphamide, MBP-8298, AG-284, tiplimotide, BX-471, E-2007, recombinant glial growth factor-2 and natalizumab;
(xxx) Monoclonal Antibodies, e.g. natalizumab, daclizumab, alemtuzumab, omalizumab, TNX-100 and SGN-40;
(xxxi) insulin secretagogues, e.g. glyburide, glipizide, repaglinide and glimiperide;
(xxxii) biguanides, e.g. metformin;
(xxxiii) alpha-glucosidase inhibitors, e.g. acarbose, voglibose and miglitol;
(xxxiv) PPAR γ agonists, e.g. ploglitazone and rosiglitazone;
(xxxv) antibiotics, e.g. sulfacetamide, erythromycin, gentamicin, tobramycin, ciprofloxacin and ofloxacin
(xxxvi) cell adhesion molecule inhibitors, e.g. alicaforsen, MLN-02, alefacept, efalizumab, R-411 and IVL-745;
(xxxvii) anti-allergy drugs, e.g. levocabastine, olopatadine, cromolyn, iodoxamide, pheniramine, ketotifen, mizolastine and epinastine;
(xxxviii) opthalmologic anti-virals, e.g. adenine arabinoside and idoxuridine;
(xxxix) glaucoma treatments, e.g. timolol, metipranolol, carteolol, betaxolol, levobunolol, brimonidine, iopidine, dorzolamide, epinephrine and dipivefrin;
(xl) alkylating anti-tumor agents, e.g. busulfan, carboplatin, chlorambucil, cisplatin, cyclophosphamide, dacarbazine, ifosfamide, mechlorethamine, melphalan, procarbazine, thiotepa, and uracil mustard;
(xli) nitrosoureas, e.g. carmustine, lumustine and streptozocin;
(xlii) antimetabolites, e.g. 5-fluorouracil, 6-mercaptopurine, capecitabine, cytosine arabinoside, floxuridine, fludarabine, gemcitabine, rhethotrexate, thioguanine and azathioprine;
(xliii) antitumor biotics, e.g. dactinomycin, daunorubicin, doxorubicin, idarubicin, mitomycin-C, and mitoxantrone;
(xliv) anti-microtuble agents, e.g. vinblastine, vincristine, vindesine, vinorelbine, paclitaxel and docetaxel;
(xlv) vitamine derivatives, e.g., calcipotriol and tacalcitol;
(xivi) leukotriene antagonists, e.g. montelukast, zafirlukast and praniukast;
(xlvii) β2 Agonists, e.g. albuterol, levalbuterol, salmeterol, formrotero and arformoterol;
(xlviii) corticosteroids, e.g. prednisone, ciclesonide, budesonide, fluticasone methylprednisolone, hydrocortisone and BP-1011;
(xlix) methylxanthines, e.g. theophylline, aminophylline and doxofylilne; and
(l) asthma and/or COPD treatments, e.g. roflumilast, tiotropium, israpafant, N-acetylcysteine and α1-antitrypsin.
(li) a vanilloid receptor agonist (e.g. resinferatoxin) or antagonist (e.g. capsazepine);
(lii) an alpha-2-delta ligand such as gabapentin, pregabalin, 3-methylgabapentin, (1α,3α,5α)(3-amino-methyl-bicyclo[3.2.0]hept-3-yl)-acetic acid, (3S,5R)-3-aminomethyl-5-methyl-heptanoic acid, (3S,5R)-3-amino-5-methyl-heptanoic acid, (3S,5R)-3-amino-5-methyl-octanoic acid, (2S,4S)-4(3-chlorophenoxy)proline, (2S,4S)-4-(3-fluorobenzyl)-proline, [(1R,5R,6S)-6-aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)acetic acid, (3S,5R)-3-aminomethyl-5-methyl-octanoic acid, (3S,5R)-3-amino-5-methyl-nonanoic acid, (3S,5R)-3-amino-5-methyl-octanoic acid, (3R,4R,5R)-3-amino-4,5-dimethyl-heptanoic acid and (3R,4R,5R)-3-amino-4,5-dimethyl-octanoic acid; and
(liii) a prostaglandin E₂ subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(1S)-1-({[5-chloro-2-(3-fluorophenoxy)pyridin-3-yl]carbonyl}amino)ethyl]benzoic acid.

### Biological Evaluation

### Method for Assessing Biological Activities:

The Human CB1 receptor binding affinity and other biological activities of the compounds of this invention are determined by the following procedures.

Membrane preparation: Human Embryonic Kidney (HEK) Cells expressing the human CB1 receptor under transcriptional regulation of a tetracycline inducible promoter were grown in Dulbecco's Modified Essential Medium with sodium pyruvate (Invitrogen, Carlsbad, Calif.) containing 10% tetracycline free fetal bovine serum (Clonetech, Mountain View, Calif.) 100 μg/ml hygromycin (Calbiochem, San Diego, Calif.), 5 μg/ml blasticidin (Invitrogen). CB1 receptor expression was induced by addition of 1 μg/ml doxycycline (Calbiochem) and incubation for an additional 24 hours. Cells were released from flasks using Cell Dissociation Buffer (Invitrogen). Cells were pelleted by centrifugation at 500×G for 5 minutes. Membranes were prepared by resuspending cells in ice cold TEE Buffer (25 mM Tris pH 7.4, 5 mM EDTA, 5 mM EGTA, Complete Protease Inhibitor (Roche, Basel, Switzerland)). Cells were lysed with 12 strokes of a dounce homogenizer. Unlysed cells were pelleted by centrifugation at 500×G for 5 minutes. Membranes were pelleted by centrifugation at 25,000×G for 30 minutes. Membranes were resuspended in TEE, dounced 12 strokes, and pelleted a second time at 25,000×G for 30 minutes. Membrane pellet was resuspended in 50 mM Tris pH 7.4, 100 mM NaCl, 3 mM MgCl₂, 0.2 mM EGTA, Complete Protease Inhibitor (Roche). Protein concentration was determined using the Micro-BCA Protein Assay Kit (Pierce, Rockford, Ill.) using BSA as a standard. Membranes were quick frozen and stored at −80 degrees Celsius until use.
Binding experiments: 50 μl of test compound was incubated with 50 μl, of [³H] CP-55,940 (Perkin Elmer, Boston, Mass.) (final concentration 500 pM) and 150 μl of membrane homogenate (1 μg/well) in polypropylene 96-well plates (Corning, Acton, Mass.). Final reaction conditions were 50 mM Tris pH 7.4, 100 mM NaCl, 3 mM MgCl₂, 0.2 mM EGTA, 0.04% BSA. Nonspecific binding was determined by incubation with 50 μM WIN-55,212-2 (Tocris, Ellisville, Mo.). After incubation at room temperature for 60 minutes reactions were harvested by vacuum filtration through Unifilter GF/B-96 filters (Perkin Elmer) that had been presoaked in assay buffer containing 0.5% BSA (Sigma, St. Louis, Mo.) using a FilterMate Plate Harvester (Perkin Elmer). Filters were rinsed 4 times with 50 mM Tris pH 7.4, 0.025% Tween20 and dried at 50 degrees Celsius for at least 30 minutes. 40 μl of Microscint-20 (Perkin Elmer) was added per well, and plates were counted using a Top-Count Microplate Scintillation Counter (Perkin Elmer). Binding data were analyzed and EC₅₀ and Kᵢ values calculated using Graph Pad Prism 4.0 Software.

### GTPγS Binding:

Membrane preparation: CHO cells expressing the human CB1 receptor were grown to 80% confluence in Ham's F-12 Nutrient Medium (Invitrogen) containing 10% fetal bovine serum (Invitrogen), 1% pen/strep (Invitrogen), 1% Nonessential amino acids (Invitrogen) and 500 μg/ml G418 (Invitrogen). Cells were released from flasks using Cell Dissociation Buffer (Invitrogen). Cells were pelleted by centrifugation at 500×G for 5 minutes. Membranes were prepared by resuspending cells in ice cold Assay Buffer (25 mM Tris pH 7.4, 5 mM EDTA, 5 mM EGTA, Complete Protease Inhibitor (Roche)). Cells were lysed with 12 strokes of a dounce homogenizer. Unlysed cells were pelleted by centrifugation at 500×G for 5 minutes. Membranes were pelleted by centrifugation at 25,000×G for 30 minutes. Membranes were resuspended in TEE, dounced 12 strokes, and pelleted a second time at 25,000×G for 30 minutes. Membrane pellet was resuspended in 50 mM Tris pH 7.4, 100 mM NaCl, 3 mM MgCl₂, 0.2 mM EGTA, Complete Protease Inhibitor (Roche). Protein concentration was determined using the Micro-BCA Protein Assay Kit (Pierce) using BSA as a standard. Membranes were frozen and stored at −80 degrees Celsius until use.
GTPγS Binding: 40 μl of test compound was incubated with 20 μl of [³⁵S] GTPγS (Perkin Elmer) (1250 Ci/millimole) and 140 μl of membrane homogenate (5 ug/well) in polypropylene 96-well plates (Corning). Final reaction conditions were 50 mM Tris pH 7.4, 100 mM NaCl, 3 mM MgCl₅, 0.2 mM EGTA, 0.04% BSA. After incubation at 37 degrees Celsius for 45 minutes reactions were harvested by vacuum filtration through Unifilter GF/B-96 filters (Perkin Elmer) using a FilterMate Plate Harvester (Perkin Elmer). Filters were rinsed 4 times with ice cold 50 mM Tris pH 7.4, 3 mM MgCl₂, 0.2 mM EGTA and dried at 50 degrees Celsius for at least 30 minutes. 40 μl of Microscint-20 (Perkin Elmer) was added per well, and plates were counted using a Top-Count Microplate Scintillation Counter (Perkin Elmer). Binding data were analyzed and EC₅₀ values were calculated using Graph Pad Prism 4.0 Software.

The above protocol assays were used to determine biological activity. The Ki towards human CB1 receptors for certain compounds of the invention are measured to be 0.01-1000 nM. The EC50 towards human CB1 receptors in the GTPγS assay for certain compounds of the invention are measured to be 0.1-5000 nM. Table 1 shows certain biological activities for some of the exemplified compounds.

TABLE

| Example | | |
|---|---|---|
| Number | CB1 Ki (nM) | GTPγS EC50 (nM) |
| 132 | 3.4 | 12.7 |
| 162 | 0.95 | 15.0 |
| 165 | 0.82 | 27.4 |
| 166 | 2.52 | 31.47 |
| 167 | 2.66 | 44.9 |
| 169 | 0.28 | 1.00 |
| 170 | 0.28 | 1.86 |
| 172 | 0.77 | 7.80 |
| 173 | 0.34 | 2.50 |
| 174 | 0.32 | 8.3 |
| 176 | 2.22 | 82.4 |
| 178 | 8.45 | 137 |
| 182 | 17 | 267 |
| 188 | 1.9 | 87.7 |
| 189 | 88.4 | 1020 |
| 190 | 8.09 | 406 |
| 191 | 10.5 | 394 |
| 193 | 67.4 | 622 |
| 197 | 0.78 | 13.2 |
| 198 | 5.79 | 46.5 |
| 199 | 0.9 | 40.1 |
| 200 | 0.52 | 96.9 |
| 201 | 4.94 | 45.3 |
| 202 | 4.52 | 20.1 |
| 203 | 8.23 | 74.1 |
| 205 | 69 | 422 |
| 217 | 1.28 | 3.2 |
| 218 | 1.8 | 13.5 |
| 223 | 102 | 1860 |
| 224 | 29.9 | 176 |
| 226 | 5.34 | 62.1 |
| 227 | 10.2 | 245 |
| 266 | 109 | >2850 |
| 267 | 18.4 | >10000 |
| 268 | 130 | 2580 |
| 269 | 2.95 | 24 |
| 270 | 17.2 | 399 |
| 271 | 1.28 | 6.53 |
| 272 | 58 | 45.4 |
| 273 | 57.5 | 244 |
| 276 | 0.374 | 5.8 |
| 280 | 31 | 136 |
| 290 | 19.2 | 25.1 |
| 292 | 2.09 | 75.1 |
| 293 | 1.45 | 32.4 |
| 298 | 5.27 | 88.6 |
| 300 | 41 | 260 |
| 301 | 5.25 | 268 |
| 302 | 82.3 | 407 |
| 303 | 5.71 | 36.6 |
| 304 | 0.15 | 0.87 |
| 305 | 0.18 | 1.74 |
| 306 | 0.31 | 3.45 |
| 307 | 1.85 | 49.5 |
| 308 | 5.38 | 20.5 |
| 310 | 30.2 | 173 |
| 311 | 2.03 | 57 |
| 312 | 69.5 | 380 |
| 317 | 12 | 83.6 |
| 318 | 23.9 | 230 |
| 319 | 5.17 | 96.3 |
| 320 | 4.2 | 98.6 |
| 327 | 36 | >10000 |
| 328 | 5.94 | 167 |
| 329 | 28 | 606 |
| 337 | 12.1 | 6490 |
| 341 | 24.7 | 355 |
| 345 | 52.8 | 1150 |
| 347 | 9.5 | 229 |
| 348 | 5.64 | 60.1 |
| 349 | 19.1 | 157 |
| 350 | 13.4 | 131 |
| 355 | 6.72 | 361 |
| 356 | 16.6 | 219 |
| 357 | 2.4 | 123 |
| 359 | 3.06 | 79.2 |
| 360 | 19.3 | 149 |
| 361 | 24.7 | 249 |
| 362 | 3.7 | 106 |
| 363 | 0.36 | 4.74 |
| 365 | 95.1 | 1030 |

### EXAMPLES

The invention is illustrated in the following non-limiting examples in which, unless stated otherwise: all operations were carried out at room or ambient temperature, that is, in the range of 18-25 degrees Celsius; evaporation of solvent was carried out using a rotary evaporator under reduced pressure with a bath temperature of up to 60 degrees Celsius; reactions were monitored by thin layer chromatography (TLC) and reaction times are given for illustration only; melting points (mp) given are uncorrected (polymorphism may result in different melting points); the structure and purity of all isolated compounds were assured by at least one of the following techniques: TLC (Merck silica gel 60 F₂₅₄ precoated TLC plates or Merck NH₂ gel (an amine coated silica gel) F₂₅₄, precoated TLC plates), mass spectrometry, nuclear magnetic resonance spectra (NMR), infrared absorption spectra (IR) or microanalysis. Yields are given for illustrative purposes only. Workup with a cation-exchange column was carried out using SCX cartridge (Varian BondElute), which was preconditioned with methanol. Flash column chromatography was carried out using Merck silica gel 60 (63-200 μm), Wako silica gel 300HG (40-60 μm), Fuji Silysia NH gel (an amine coated silica gel) (30-50 μm), Biotage KP-SIL (32-63 μm) or Biotage AMINOSILICA (an amine coated silica gel) (40-75 μm). Preparative TLC was carried out using Merck silica gel 60 F₂₅₄ precoated TLC plates (0.5 or 1.0 mm thickness). Low-resolution mass spectral data (EI) were obtained on an Integrity (Waters) mass spectrometer. Low-resolution mass spectral data (ESI) were obtained on ZMD™ or ZQ™ (Waters) and mass spectrometer. NMR data were determined at 270 MHz (JEOL JNM-LA 270 spectrometer), 300 MHz (JEOL JNM-LA300 spectrometer) or 600 MHz (Bruker AVANCE 600 spectrometer) using deuterated chloroform (99.8% D) or dimethylsulfoxide (99.9% D) as solvent unless indicated otherwise, relative to tetramethylsilane (TMS) as internal standard in parts per million (ppm); conventional abbreviations used are: s=singlet, d=doublet, t=triplet, q=quartet, quint=quintet, m=multiplet, bs=broad singlet, etc. IR spectra were measured by a Fourier transform infrared spectrophotometer (Shimazu FTIR-8300). Chemical symbols have their usual meanings; bp (boiling point), mp (melting point), rt (room temperature), L (liter(s)), mL (milliliter(s)), g (gram(s)), mg (milligram(s)), mol (moles), mmol (millimoles), eq. (equivalent(s)), quant. (quantitative yield). Following abbreviations may be used in examples: CDI (N,N′-carbonyldiimidazole), DMF (N,N-dimethylformamide), DMSO (dimethylsulfoxide), EDAPC (1-ethyl-3-(3-dimethylaminopropyl)carbodiimide hydrochloride), EtOH (ethanol), HOBt (1-Hydroxy-1H-benzotriazole), MeOH (methanol), and THF (tetrahydrofuran). Rt means retention time measured by LC/MS (Waters 2790) under the following condition;

Column: Xterra, C18, 5 μm, 4.6×50 mm (40 degrees Celsius)

flow: 2.0 mL/min

Gradient: Water/MeOH/1% HCO₂H aq.=90/5/5 to 0/95/5

Total run time: 2.5 minutes.

### Example 1

### N-[(1S,2S)-1-(Aminocarbonyl)-2-methylbutyl]-3-(2-morpholin-4-ylethyl)-2-oxo-2,3-dihydro-1H-benzimidazole-1-carboxamide hydrochloride

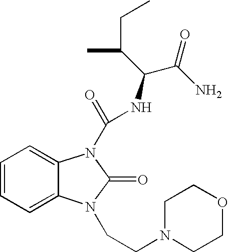

### STEP 1. N-(2-Morpholin-4-ylethyl)-2-nitroaniline

To a mixture of 1-fluoro-2-nitrobenzene (6 g, 43.0 mmol) and potassium carbonate (12 g, 86 mmol) in THF (80 mL) was added 4-(2-aminoethyl)morpholine (6.8 mL, 52.0 mmol) at 0 degrees Celsius. The mixture was stirred for 25 hours at room temperature. Then the mixture was filtered through a pad of Celite and concentrated in vacuo. The residue was purified by column chromatography on silica get eluting with hexane/ethyl acetate (2/1) to afford 10.4 g (97%) of the title compound.

¹H-NMR (270 MHz, CDCl₃) δ 8.50 (bs, 1H), 8.18 (dd, J=8.6, 1.49 Hz, 1H), 7.47-7.41 (m, 1H), 6.82 (d, J=8.6 Hz, 1H), 6.67-6.62 (m, 1H), 3.78-3.74 (m, 4H), 3.40-3.34 (m, 2H), 2.73 (t, J=6.1 Hz, 2H), 2.55-2.52 (m, 4H).

MS (ESI) m/z 252 (M+H)⁺.

### STEP 2. N-(2-Morpholin-4-ylethyl)benzene-1,2-dimanine

To a solution of N-(2-morpholin-4-ylethyl)-2-nitroaniline (10 g, 42 mmol) in THF (100 mL) was added 10% Pd/C (1 g). The flask was evacuated and flushed with H₂ gas and this process was repeated three times. The flask was filled with H₂ gas (4 atm) and stirred for 4 hours at room temperature. Then the reaction mixture was filtered through a pad of Celite and concentrated in vacuo to give the title compound (crude; 9.0 g)

¹H-NMR (300 MHz, CDCl₃) δ 6.82-6.64 (m, 4H), 3.71 (t, J=4.6 Hz, 4H), 3.40 (bs, 2H), 3.19-3.15 (m, 2H), 2.69-2.65 (m, 2H), 2.48 (t, J=4.6 Hz, 4H).

MS (ESI) m/z 222 (M+H)⁺.

### STEP 3, 1-(2-Morpholin-4-ylethyl)-1,3-dihydro-2H-benzimidazol-2-one

To a solution of N-(2-morpholin-4-ylethyl)benzene-1,2-dimanine in THF (100 mL) was added CDI (10 g, 62 mmol) and the mixture was stirred at room temperature. After 23 hours, the mixture was evaporated in vacuo and to the residue was added water (100 mL) at 0 degrees Celsius. The mixture was extracted with ethyl acetate (100 mL×2) and the combined organic layers were washed with water (50 mL), brine (50 mL), dried over sodium sulfate, filtered and concentrated in vacuo. The residue was purified by column chromatography on silica gel eluting with dichloromethane/methanol (30/1) to afford 8.5 g (83%) of the title compound.

¹H-NMR (300 MHz, CDCl₃) δ 10.4 (s, 1H), 7.13-7.01 (m, 4H), 4.03 (t, J=6.8 Hz, 2H), 3.70 (t, J=4.6 Hz, 4H), 2.72 (t, J=6.8 Hz, 2H), 2.57 (t, J=4.6 Hz, 4H).

MS (ESI) m/z 248 (M+H)⁺, 246 (M−H)⁻.

IR (KBr)νₘₐₓ 2851, 1697, 1491, 1402, 1117 cm⁻¹.

mp 131.0 degrees Celsius.

### STEP 4. N-[(1S,2S)-1-(Aminocarbonyl)-2-methylbutyl]-3-(2-morpholin-4-ylethyl)-2-oxo-2,3-dihydro-1H-benzimidazole-1-carboxamide hydrochloride

To a solution of 1-(2-morpholin-4-ylethyl)-1,3-dihydro-2H-benzimidazol-2-one (530 mg, 2.1 mmol) in dichloromethane (8 mL) were added triethylamine (1.0 mL, 7.0 mmol) and 4-nitrophenyl chloroformate (470 mg, 2.3 mmol) at 0 degrees Celsius and the mixture was stirred for 3 hours at room temperature Then to this mixture was added a mixture of L-isoleucinamide hydrochloride (430 mg, 2.6 mmol) and triethylamine (0.6 mL, 4.3 mmol) in dichloromethane (4 mL) at 0 degrees Celsius and stirred room temperature. After 22 hours, the reaction was quenched by addition of water (50 mL) and extracted with dichloromethane (50 mL×2). The combined organic layers were washed with water (20 mL×3), brine (20 mL) and dried over sodium sulfate, filtered and concentrated. The residue was purified by column chromatography on silica gel eluting with hexane/ethyl acetate (1/4) to afford 600 mg (70%) of free form of the title compound. The obtained compound was dissolved in ethyl acetate (1 mL) and to this solution was added 4N HCl in ethyl acetate (0.4 mL) to form white solid which was filtered and dried in vacuo to give the title compound (600 mg).

¹H-NMR (300 MHz, CDCl₃) δ 10.91 (bs, 1H), 9.00 (d, J=8.1 Hz, 1H), 8.06 (d, J=7.8 Hz, 1H), 7.68 (s, 1H), 7.50 (d, J=8.1 Hz, 1H), 7.31-7.18 (m, 3H), 4.38-4.30 (m, −3H), 4.04-3.99 (m, 2H), 3.78-3.70 (m, 2H), 3.65-3.57 (m, 2H), 3.53-3.45 (m, 2H), 3.21-3.17 (m, 2H), 1.89-1.83 (m, 1H), 1.56-1.45 (m, 1H), 1.17-1.03 (m, 1H), 0.94 (d, J=6.9 Hz, 3H), 0.89 (t, J=7.2 Hz, 3H).

MS (ESI) m/z 404 (M+H)⁺.

Anal. calcd. for C₂₀H₂₉N₅O₄ (+0.8H₂O, 1.0 HCl): C, 52.87; H, 7.01; N, 15.41; O, 16.90; Cl, 7.80.

Found: C, 53.00; H, 7.23; N, 15.01.

### Example 2

### Methyl N-{[3-(2-Morpholin-4-ylethyl)-2-oxo-2,3-dihydro-1H-benzimidazole-1-yl]carbonyl}-L-isoleucinate

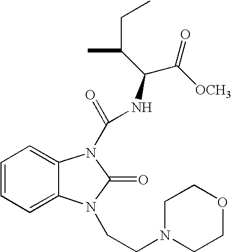

The titled compound was prepared according to the procedure described in Step 4 of example 1 from methyl L-isoleucinate hydrochloride.

¹H-NMR (300 MHz, CDCl₃) δ 9.29 (d, J=8.1 Hz, 1H), 8.20-8.17 (m, 1H), 7.24-7.13 (m, 2H), 7.05-7.02 (m, 1H), 4.62 (dd, J=8.1, 4.8 Hz, 1H), 4.02 (t, J=6.9 Hz, 2H), 3.78 (s, 3H), 3.69-3.66 (m, 4H), 2.70 (t, J=6.6 Hz, 2H), 2.54 (bs, 4H), 2.12-2.05 (m, 1H), 1.61-1.48 (m, 1H), 1.35-1.24 (m, 1H), 1.03 (d, J=6.9 Hz, 3H), 0.97 (t, J=7.2 Hz, 3H).

MS (ESI) m/z 419 (M+H)⁺.

### Example 3

### N-{(1S,2S)-1-[(Dimethylamino)carbonyl]-2-methylbutyl}-3-(2-morpholin-4-ylethyl)-oxo-2,3-dihydro-1H-benzimidazol-1-carboxamide

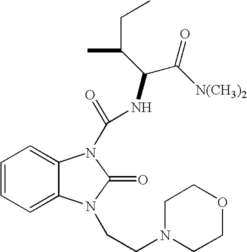

### STEP 1. N-{[3-(2-Morpholin-4-ylethyl)-2-oxo-2,3-dihydro-1H-benzimidazol-1-yl]carbonyl}-L-isoleucine hydrochloride

A suspension of methyl N-{[3-(2-morpholin-4-ylethyl)-2-oxo-2,3-dihydro-1H-benzimidazol-1-yl]carbonyl}-L-isoleucinate (Example 2) in 4N HCl (4 mL) and acetic acid (4 mL) was refluxed for 24 hours. Then it was cooled to room temperature and evaporated to dryness. Recrystallization from ethyl acetate and hexane followed by filtration gave 510 mg (81%) of the title compound as white solid.

¹H-NMR (300 MHz, DMSO-d₆) δ 9.08 (d, J=8.1 Hz, 1H), 8.35 (bs, 1H), 8.05 (d, J=7.2 Hz, 1H), 7.53 (d, J=7.5 Hz, 1H), 7.29 (t, J=7.5 Hz, 1H), 7.21 (t, J=7.2 Hz, 1H), 4.43-4.39 (m, 3H), 4.07-3.95 (m, 2H), 3.30-3.05 (m, 10H), 2.00-1.95 (m, 1H), 1.53-1.44 (m, 1H), 1.28-1.15 (m, 1H), 0.95 (s, 3H), 0.93 (s, 3H).

MS (ESI) m/z 405 (M+H)⁺.

IR (KBr)νₘₐₓ 1732, 1639, 1387, 1184 cm⁻¹

[α]_{D}²⁷+24.0° (c 0.275, methanol).

### STEP 2. N-{(1S,2S)-1-[(Dimethylamino)carbonyl]-2-methylbutyl}-3(2-morpholin-4-ylethyl)-2-oxo-2,3-dihydro-1H-benzimidazole-1-carboxamide

To a solution of N-{[3-(2-morpholin-4-ylethyl)-2-oxo-2,3-dihydro-1 benzimidazol-1-yl]carbonyl}-L-isoleucine hydrochloride (Step 1, 62 mg, 0.14 mmol) in DMF (1 mL) was added CDI (27 mg, 0.17 mol) at room temperature. After 2 hours, to the mixture was added aq. dimethylamine (40%, 20 μL) and stirred for further 14 hours. Then to the mixture was added water (10 mL). The mixture was extracted with ethyl acetate (20 mL×2) and the combined organic layers were washed with brine (10 mL), dried over sodium sulfate, filtered and concentrated. The residue was purified by preparative TLC eluting with dichloromethane/methanol (10/1) to afford 33 mg (54%) of the title compound.

¹H-NMR (300 MHz, DMSO-d₆) δ 9.14 (d, J=8.4 Hz, 1H), 8.02 (d, J=7.5 Hz, 1H), 7.35 (d, J=7.8 Hz, 1H), 7.24 (t, J=7.2 Hz, 1H), 7.15 (t, J=7.8 Hz, 1H), 4.83 (dd, J=8.4, 6.3 Hz, 1H), 4.02 (t, J=6.0 Hz, 2H), 3.49 (t, J=4.8 Hz, 4H), 3.12 (s, 3H), 2.87 (s, 3H), 2.63-2.58 (m, 2H), 2.46-2.43 (m, 4H), 1.85-1.75 (m, 1H), 1.57-1.48 (m, 1H), 1.17-1.07 (m, 1H), 0.93 (d, J=6.6 Hz, 3H), 0.87 (t, J=7.5 Hz, 3H).

MS (ESI) m/z 432 (M+H)⁺.

### Example 4

### N-[(1S)-1-Aminocarbonyl)-2,2-dimethylpropyl]-3-(2-morpholin-4-ylethyl)-2-oxo-2,3-dihydro-1H-benzimidazole-1-carboxamide

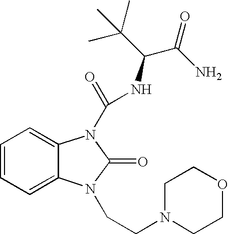

### STEP 1, Benzyl [(1S)-1-(aminocarbonyl)-2,2-dimethylpropyl]carbamate

To a solution of N-[(benzyloxy)carbonyl]-tert-leucine (prepared according to the procedure in the literature; Emily, M. S. et al. Tetrahedron 2001, 57, 5303-5320.; 3.7 g, 14 mmol) in DMF (80 mL) were added ammonium chloride (900 mg, 17 mmol), triethylamine (5.9 mL, 42 mmol), HOBt (2.8 g, 18 mmol) and EDAPC (3.1 g, 18 mmol) and stirred at room temperature. After 17 hours, the reaction mixture was quenched by addition of saturated aqueous sodium bicarbonate (100 mL) and extracted with ethyl acetate (100 mL×3). The combined organic layers were washed with water (100 mL×3), brine (50 mL), dried over sodium sulfate, filtered and concentrated in vacuo. The residue was purified by column chromatography on silica gel eluting with hexane/ethyl acetate (211-1/1) to afford 3.0 g (82%) of the title compound.

MS (ESI) m/z 265 (M+H)⁺.

### STEP 2, tert-Leucinamide

To a solution of benzyl [(1S)-1-(aminocarbonyl)-2,2-dimethylpropyl]carbamate (Step 1, 3.7 g, 14 mmol) in THF (40 mL) was added 10% Pd/C (710 mg). The flask was evacuated and flushed with H₂ gas and this process was repeated three times. The flask was filled with H₂ gas (4 atm) and stirred for 3 hours at room temperature. Then the reaction mixture was filtered through a pad of Celite and concentrated in vacuo to give the title compound as white solid (crude; 1.8 g)

¹H-NMR (300 MHz, DMSO-d₆) δ 6.59 (bs, 1H), 5.92 (bs, 1H), 3.12 (s, 1H), 1.02 (s, 1H).

MS (ESI) m/z 131 (M+H).

### STEP 3. N-[(1S)-1-(Aminocarbonyl)-2,2-dimethylpropyl]-3-(2-morpholin-4-ylethyl)-2-oxo-2,3-dihydro-1H-benzimidazole-1-carboxamide hydrochloride

The title compound was prepared according to the procedure described in Step 4 of Example 1 from L-tert-leucinamide.

¹H-NMR (270 MHz, CDCl₃, the value of free form of the title compound) δ 9.45 (d, J=7.8 Hz, 1H), 8.19-8.16 (m, 1H), 7.25-7.14 (m, 2H), 7.05 (d, J=7.6 Hz, 1H), 5.83 (bs, 1H), 5.53 (bs, 1H), 4.22 (d, J=8.1 Hz, 1H), 4.02 (t, J=6.8 Hz, 2H), 3.68 (t, J=4.6 Hz, 4H), 2.73-2.68 (m, 2H), 2.60-2.49 (m, 4H), 1.15 (s, 9H).

MS (ESI) m/z 404 (M+H)⁺.

Anal. calcd. for C₂₀H₂₉N₅O₄ (+1.0H₂O, 1.0 HCl): C, 52.45; H, 7.043; N, 15.29; O, 17.47; Cl, 7.74.

Found: C, 52.41; H, 7.21; N, 14.98.

[α]_{D}²⁵+29.5° (c 0.325, methanol).

### Example 5

### Methyl 3-methyl-N-{[3-(2-morpholin-4-ylethyl)-2-oxo-2,3-dihydro-1H-benzimidazol-1-yl]carbonyl}-L-valinate

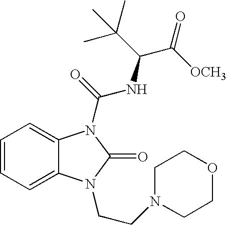

The title compound was prepared according to the procedure described in Step 4 of Example 1 from methyl L-tert-leucinate. The obtained compound was further purified by recrystallization from hexane/ethyl acetate.

¹H-NMR (300 MHz, CDCl₃) δ 9.40 (d, J=8.4 Hz, 1H), 8.20-8.17 (m, 1H), 7.25-7.08 (m, 3H), 4.44 (d, J=8.4 Hz, 1H), 4.10-3.99 (m, 2H), 3.77 (s, 3H), 3.73-3.65 (m, 4H), 2.77-2.66 (m, 2H), 2.62-2.52 (m, 4H), 1.09 (s, 9H).

MS (ESI) m/z 419 (M+H)⁺.

Anal. calcd. for C₂₁H₃₀N₄O₅ (+0.5H₂O): C, 59.00; H, 7.31; N, 13.11; O, 20.58. Found: C, 59.24; H, 7.23; N, 13.15.

IR (KBr)νₘₐₓ 1728, 1553, 1398, 1159 cm⁻¹.

### Example 6

### N-{(1S)-2,2-Dimethyl-1-[(methylamino)carbonyl]propyl-3-(2-morpholin-4-ylethyl)-2-oxo-2,3-dihydro-1H-benzimidazole-1-carboxamide

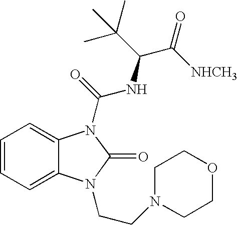

The title compound was prepared according to the procedure described in Example 3 from Methyl 3-methyl-N-{[3-(2-morpholin-4-ylethyl)-2-oxo-2,3-dihydro-1H-benzimidazol-1-yl]carbonyl}-L-valinate (Example 5) and aqueous methylamine (40%).

¹H-NMR (270 MHz, CDCl₃) δ 9.43 (d, J=8.4 Hz, 1H), 8.18-8.15 (m, 1H), 7.25-7.07 (m, 3H), 5.85-5.75 (bs, 1H), 4.15 (d, J=8.4 Hz, 1H), 4.10-3.95 (m, 2H), 3.75-3.62 (m, 4H), 2.84 (d, J=4.59 Hz, 3H), 2.78-2.45 (m, 6H), 1.12 (s, 9H).

MS (ESI) m/z 418 (M+H)⁺.

### Example 7

### N-{(1S)-1-[(Dimethylamino)carbonyl]-2,2-dimethylpropyl}-3-(2-morpholin-4-ylethyl)-2-oxo-2,3-dihydro-1H-benzimidazole-1-carboxamide

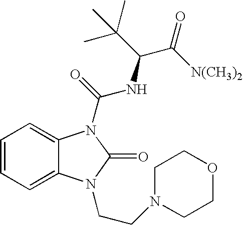

The title compound was prepared according to the procedure described in Example 3 from Methyl-3-methyl-N-{[3-(2-morpholin-4-ylethyl)-2-oxo-2,3-dihydro-1H-benzimidazol-1-yl]carbonyl}-L-valinate (Example 5).

¹H-NMR (270 MHz, CDCl₃) δ 9.48 (d, J=9.2 Hz, 1H), 8.18-8.15 (m, 1H), 7.23-7.12 (m, 2H), 7.03 (d, J=7.6 Hz, 1H), 4.93 (d, J=9.2 Hz, 1H), 4.05-3.99 (m, 2H), 3.69-3.66 (m, 4H), 3.23 (s, 3H), 3.00 (s, 3H), 2.74-2.66 (m, 2H), 2.58-2.48 (m, 4H), 1.11 (s, 9H)

MS (ESI) m/z 432 (M+H)⁺.

### Example 8

### N-[(1S,2S)-1-(Aminocarbonyl)-2-methylbutyl]-2-oxo-3-(2-piperidin-1-ylethyl)-2,3-dihydro-1H-benzimidazole-1-carboxamide hydrochloride

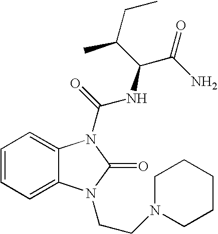

### STEP 1. 1-(2-Piperidin-1-ylethyl)-1,3-dihydro-2H-benzimidazol-2-one

The title compound was prepared according to the procedure described in Steps 1 to 3 of Example 1 from 1-(2-aminoethyl)piperidine.

¹H-NMR (300 MHz, CDCl₃) δ 10.82-10.72 (m, 1H), 7.07-7.04 (m, 4H), 4.06-4.01 (m, 2H), 2.71-2.65 (m, 2H), 2.55-2.50 (m, 4H), 1.62-1.58 (m, 4H), 1.45-1.43 (m, 2H).

MS (ESI) m/z 246 (M+H)⁺.

### STEP 2. N-[(1S,2S)-1-(Aminocarbonyl)-2-methylbutyl]-2-oxo-3-(2-piperidin-1-ylethyl)-2,3-dihydro-1H-benzimidazole-1-carboxamide hydrochloride

The title compound was prepared according to the procedure described in Steps 4 of Example 1 from 1-(2-Piperidin-1-ylethyl)-1,3-dihydro-2H-benzimidazol-2-one (Step1).

¹H-NMR (270 MHz, CDCl₃) δ 12.47 (bs, 1H), 9.06 (d, J=8.1 Hz, 1H), 8.10 (d, J=7.6 Hz, 1H), 7.53 (d, J=7.6 Hz, 1H), 7.24-7.11 (m, 2H), 6.48 (bs, 1H), 5.69 (bs, 1H), 4.55-4.53 (m, 2H), 4.42 (dd; J=8.1, 5.4 Hz, 1H), 3.80-3.50 (m, 2H), 3.40-3.10 (m, 2H), 2.86-2.65 (m, 2H), 2.26-1.48 (m, 7H), 1.34-1.17 (m, 2H), 1.05 (d, J=7.0 Hz, 3H), 0.97 (t, J=7.3 Hz, 3H).

MS (ESI) m/z 402.4 (M+H)⁺.

Anal. calcd. for C₂₁H₃₁N₅O₃ (+0.5H₂O, 1 HCl, 0.2 C₄H₈O₂): C, 56.36; H, 7.51; N, 15.07; O, 13.43; Cl, 7.63. Found: C, 56.28; H, 7.72; N, 14.96.

mp 217.1 degrees Celsius

### Example 9

### N-[(1S,2S)-1-(Aminocarbonyl)-2-methylbutyl]-4-methoxy-3-(2-morpholin-4-ylethyl)-2-oxo-2,3-dihydro-1H-benzimidazole-1-carboxamide hydrochloride

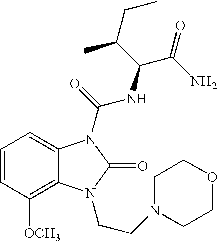

### STEP 1. 2-Bromo-N-(2-methoxy-6-nitrophenyl)acetamide

To a flask was added sodium hydride (60% dispersion in mineral oil, 610 mg, 15 mmol) and hexane (2 mL) at 0 degrees Celsius. The supernatant liquid was decanted and the residue was dried under reduced pressure. To this was added THF (20 mL) and a solution of 2-methoxy-6-nitroaniline (2 g, 12 mmol, Kubo, K. et al. J. Med. Chem. 1993, 36, 1772-1784) in THF (20 mL) at 0 degrees Celsius and stirred at room temperature for 2 hours. To this mixture was added bromoacetyl bromide (1.2 mL, 14 mmol) at 0 degrees Celsius and stirred at room temperature for 3 hours. Then the reaction mixture was quenched by addition of water (100 mL) and extracted with ethyl acetate (100 mL×2). The combined organic layers were washed with brine (100 mL), dried over sodium sulfate, filtered and concentrated in vacuo. The residue was purified by column chromatography on silica gel eluting with hexane/ethyl acetate (2/1-1/1) to afford 2.9 g (85%) of the title compound.

¹H-NMR (300 MHz, CDCl₃) δ 8.62 (bs, 1H), 7.56 (dd, J=8.2, 1.1 Hz, 1H), 7.34 (dd, J=8.4, 8.2 Hz, 1H), 7.19 (dd, J=8.4, 1.1 Hz, 1H), 4.04 (s, 2H), 3.96 (s, 3H).

### STEP 2. N-(2-Methoxy-6-nitrophenyl)-2-morpholin-4-ylacetamide

To a solution of 2-bromo-N-(2-methoxy-6-nitrophenyl)acetamide (Step1, 8.8 g, 30 mmol) in THF (240 mL) was added morpholin (11 mL, 122 mmol) at 0 degrees Celsius and warmed to room temperature. After 2.5 hours, the reaction mixture was quenched by addition of water (200 mL) and extracted with ethyl acetate (200 mL×2). The combined organic layers were washed with water (200 mL), brine (100 mL) dried over sodium sulfate, filtered and concentrated in vacuo. The residue was purified by column chromatography on silica gel eluting with hexane/ethyl acetate (2/1) to afford 6.7 g (75%) of the title compound.

¹H-NMR (270 MHz, CDCl₃) δ 9.51 (bs, 1H), 7.53 (dd, J=8.4, 8.1 Hz, 1H), 7.29 (dd, J=8.4, 8.1 Hz, 1H), 7.17 (dd, J=8.4, 1.1 Hz, 1H), 3.94 (s, 3H), 3.84-3.81 (m, 4H), 3.18 (s, 2H), 2.68-2.65 (m, 4H).

MS (ESI) m/z 296 (M+H)⁺, 294 (M−H)⁻.

### STEP 3. 3-Methoxy-N²-(2-morpholin-4-ylethyl)benzene-1,2-diamine

To a suspension of lithium aluminum hydride (5.2 g, 136 mmol) in THF (35 mL) was added a solution of N-(2-methoxy-6-nitrophenyl)-2-morpholin-4-ylacetamide (Step 2, 6.7 g, 23 mmol) in THF (40 mL) at 0 degrees Celsius and stirred at reflux for 2 hours. Then to this mixture was added water (5.2 mL) followed by addition of 15% sodium hydroxide (5.2 mL), water (15.6 mL) at 0 degrees Celsius. The mixture was diluted with ethyl acetate (100 mL) and stirred for 3 hours at room temperature. The resultant mixture was filtered and concentrated in vacuo. The residue was purified by column chromatography on silica gel eluting with dichloromethane/methanol (30/1) to afford 2.5 g (44%) of the title compound.

¹H-NMR (300 MHz, CDCl₃) δ 6.82 (t, J=8.1 Hz, 1H), 6.39-6.31 (m, 2H), 3.79 (s, 3H), 3.77-3.69 (m, 4H), 3.02-2.98 (m, 2H), 2.53-2.50 (m, 6H).

MS (ESI) m/z 252 (M+H)⁺.

### STEP 4. 7-Methoxy-1-(2-morpholin-4-ylethyl)-1,3-dihydro-2-H-benzimidazol-2-one

The title compound was prepared according to the procedure described in Step 3 of Example 1 from 3-Methoxy-N²-(2-morpholin-4-ylethyl)benzene-1,2-diamine (Step 3).

¹H-NMR (300 MHz, CDCl₃) δ 8.98 (bs, 1H), 6.98 (dd, J=8.3, 7.9 Hz, 1H), 6.72 (dd, J=7.9, 0.7 Hz, 1H), 6.63 (d, J=8.2 Hz, 1H), 4.21 (t, J=7.1 Hz, 2H), 3.90 (s, 3H), 3.71-3.68 (m, 4H), 2.71 (t, J=7.1 Hz, 2H), 2.58-2.55 (m, 4H).

MS (ESI) m/z 278 (M+H)⁺.

### STEP 5. N-[(1S,2S)-1-(Aminocarbonyl)-2-methylbutyl]-4-methoxy-3-(2-morpholin-4-ylethyl)-2-oxo-2,3-dihydro-1H-benzimidazole-1-carboxamide hydrochloride

The title compound was prepared according to the procedure described in Sep 4 of Example 1 from 7-Methoxy-1-(2-morpholin-4-ylethyl)-1,3-dihydro-2-H-benzimidazol-2-one (Step 4) and L-isoleucinamide.

¹H-NMR (300 MHz, CDCl₃) δ 12.70 (bs, 1H), 9.12 (d, J=8.1 Hz, 1H), 7.84 (d, J=8.4 Hz, 1H), 7.19 (bs, 1H), 7.09 (t, J=8.4 Hz, 1H), 6.74 (d, J=8.4 Hz, 1H), 5.69 (bs, 1H), 4.69-4.55 (m, 2H), 4.44 (dd, J=8.4, 4.5 Hz, 4H), 4.37-4.03 (m, 5H), 3.94 (s, 3H), 3.60-3.40 (m, 2H), 3.30-3.15 (m, 1H), 3.10-2.40 (m, 2H), 2.27-2.16 (m, 1H), 1.67-1.54 (m, 1H), 1.36-1.16 (m, 1H), 1.06 (d, J=6.9 Hz, 3H), 0.97 (t, J=7.5 Hz, 3H).

MS (ESI) m/z 434 (M+H)⁺.

Anal. calcd. for C₂₁H₃₁N₅O₅ (+0.5H₂O, 1 HCl, 0.1 C₄H₈O₂): C, 52.69; H, 6.98; N, 14.36; O, 18.70; Cl, 7.27. Found: C, 52.33; H, 7.20; N, 14.01.

### Example 10

### N-[(1S)-1-(Aminocarbonyl)-2,2-dimethylpropyl]-2-oxo-3-[2-tetrahydro-2H-pyran-4-yl)ethyl]-2,3-dihydro-1H-benzimidazole-1-carboxamide

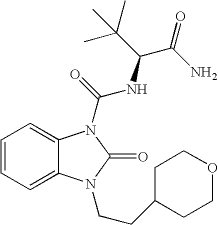

### STEP 1. 1-[2-(Tetrahydro-2H-pyran)-4-ylethyl]-1,3-dihydro-2H-benzimidazol-2-one

The title compound was prepared according to the procedure described in Steps 1 to 3 of Example 1 from 2-(tetrahydro-2H-pyran-4-yl)ethanamine.

MS (ESI) m/z 247 (M+H)⁺, 245 (M−H)⁻.

### STEP 2. N-[(1S)-1-(Aminocarbonyl)-2,2-dimethylpropyl]-2-oxo-3-[2-(tetrahydro-2-pyran-4-yl)ethyl]-2,3-dihydro-1H-benzimidazole-1-carboxamide

The title compound was prepared according to the procedure described in Step 4 of Example 1 from 1-(2-(Tetrahydro-2H-pyran-4-ylethyl)-1,3-dihydro-2H-benzimidazol-2-one (Step 1).

¹H-NMR (270 MHz, CDCl₃) δ 9.45 (d, J=8.1 Hz, 1H), 8.17 (d, J=7.56 Hz, 1H), 7.26-7.14 (m, 2H), 7.00 (dd, J=8.1, 1.6 Hz, 1H), 5.99 (bs, 1H), 5.23 (bs, 1H), 4.24 (d, J=8.1 Hz, 1H), 4.00-3.88 (m, 4H), 3.38 (t, J=11.6 Hz, 2H), 1.77-1.69 (m, 4H), 1.64-1.53 (m, 1H), 1.45-1.30 (m, 2H), 1.15 (s, 9H).

MS (ESI) m/z 403 (M+H)⁺.

Anal. calcd. for C₂₁H₃₀N₄O₄ (+0.1H₂O): C, 62.39; H, 7.53; N, 13.86; O, 16.23. Found: C, 62.21; H, 7.59; N, 13.70.

### Example 11

### N-[(1S)-(Aminocarbonyl)-2,2-dimethylpropyl]-3-(cyclopropylmethyl)-2-oxo-2,3-dihydro-1H-benzimidazole-1-carboxamide

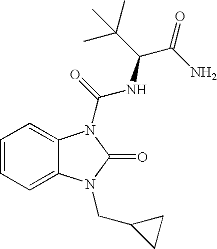

### STEP 1. 1-(cyclopropylmethyl)-1,3-dihydro-2H-benzimidazol-2-one

The title compound was prepared according to the procedure described in Steps 1 to 3 of Example 1 from 1-cyclopropylmethanamine.

¹H-NMR (300 MHz, CDCl₃) δ 7.15-7.03 (m, 4H), 3.79 (d, J=7.0 Hz, 2H), 1.30-1.21 (m, 1H), 0.59-0.50 (m, 2H), 0.48-0.39 (m, 2H).

MS (ESI) m/z 189 (M+H)⁺.

### STEP 2. N-[(1S)-1-(Aminocarbonyl)-2,2-dimethylpropyl]-3-(cyclopropylmethyl)-2-oxo-2,3-dihydro-1H-benzimidazole-1-carboxamide

The title compound was prepared according to the procedure described in step 4 of Example 1 from 1-(cyclopropylmethyl)-1,3-dihydro-2H-benzimidazol-2-one (Step 1).

¹H-NMR (270 MHz, CDCl₃) δ 9.48 (d, J=7.8 Hz, 1H), 8.17 (d, J=7.83 Hz, 1H), 7.25-7.13 (m, 2H), 7.10-7.06 (m, 1H), 5.96 (bs, 1H), 5.65 (bs, 1H), 4.23 (d, J=7.8 Hz, 1H), 3.79 (d, J=7.02 Hz, 2H), 1.33-1.21 (m, 1H), 1.15 (s, 9H), 0.62-0.55 (m, 2H), 0.50-0.42 (m, 2H).

MS (ESI) m/z 345 (M+H)⁺.

Anal. calcd. for C₁₈H₂₄N₃O₃ (+0.1H₂O): C, 62.45; H, 7.05; N, 16.18; O, 14.33. Found: C, 62.26; H, 7.06; N, 16.08.

### Example 12

### N-[(1S)-1-(Aminocarbonyl)-2,2-dimethylpropyl]-3-(3-methylbutyl)-2-oxo-2,3-dihydro-1H-benzimidazole-1-carboxamide

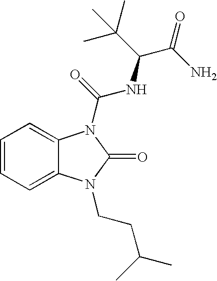

### STEP 1. 1-(3-methylbutyl)-1,3-dihydro-2H-benzimidazol-2-one

The title compound was prepared according to the procedure described in the literature (Meth-Cohn, O.; Smith, D. I. J.C.S. Perkin Trans. 1, 1982, 261-270; Vernin G. et al. J. Heterocyclic Chem. 1981, 18, 85-89.) from 1-bromo-3-methylbutane.

¹H-NMR (300 MHz, CDCl₃) [symbol] 9.86 (br, 1H), 7.14-6.98 (m, 4H), 3.94-3.88 (m, 2H), 1.72-1.62 (m, 3H), 1.00 (d, J=6.1 Hz, 6H).

### STEP 2 N-[(1S)-1-(Aminocarbonyl)-2,2-dimethylpropyl]-3-(3-methylbutyl)-2-oxo-2,3-dihydro-1H-benzimidazole-1-carboxamide

To a solution of 1-(3-methylbutyl)1,2-dihydro-2H-benzimidazol-2-one (Step 1, 140 mg, 0.69 mmol) in dichloromethane (2.5 mL) were added triethylamine (0.32 mL, 2.3 mmol) and 4-nitrophenyl chloroformate (150 mg, 0.76 mmol) at 0 degrees Celsius and the mixture was stirred for 4 hours at room temperature. Then to this mixture was added a solution of L-tert-leucinamide (steps 1 and 2 in example 4, 99 mg, 0.76 mmol) in dichloromethane, (2 mL) at 0 degrees Celsius and stirred rt. After 22 h, the reaction was quenched by addition of water (20 mL) and extracted with ethyl acetate (30 mL×2). The combined organic layers were washed with water (20 mL×3), brine (20 mL) and dried over sodium sulfate, filtered and concentrated. The residue was purified by column chromatography on silica gel eluting with hexane/ethyl acetate (3/1-1/1) to afford 240 mg (96%) of the titled compound. The obtained product was further purified by recrystallization from hexane/ethyl acetate to give 220 mg of the title compound.

¹H-NMR (270 MHz, CDCl₃) δ 9.48 (d, J=7.8 Hz, 1H), 8.16 (d, J=7.56 Hz, 1H), 7.25-7.12 (m, 2H), 7.03-7.00 (m, 1H), 6.01 (bs, 1H), 5.72 (bs, 1H), 4.24 (d, J=7.8 Hz, 1H), 3.99-3.81 (m, 2H), 1.71-1.61 (m, 3H), 1.15 (s, 9H), 1.00 (d, J=6.2 Hz, 6H).

MS (ESI) m/z 361 (M+H)⁺.

Anal. calcd. for C₁₉H₂₈N₄O₃: C, 63.31; H, 7.83; N, 15.54; O, 13.32. Found: C, 62.94; H, 7.86; N, 15.62.

### Example 13

### N-[(1S)-(Aminocarbonyl)-2,2-dimethylpropyl]-3-(3,3-dimethylbutyl)-2-oxo-2,3-dihydro-1H-benzimidazole-1-carboxamide

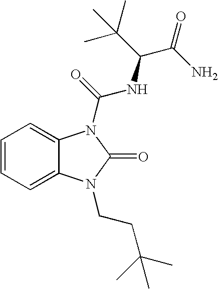

### STEP 1. 1(3,3-dimethylbutyl)-1,2-dihydro-2H-benzimidazol-2-one

The title compound was prepared according to the procedure described in the literature (Meth-Cohn, O.; Smith, D. I. J.C.S. Perkin Trans. 1, 1982, 261-270; Vernin G. et al. J. Heterocyclic Chem. 1981, 18, 85-89.) from 1-bromo-3,3-dimethylbutane.

¹H-NMR (300 MHz, CDCl₃) δ 9.7-9.5 (br, 1H), 7.14-6.96 (m, 4H), 3.94-3.88 (m, 2H), 1.71-1.63 (m, 2H), 1.04 (s, 9H).

### STEP 2. N-[(1S)-1-(Aminocarbonyl)-2,2-dimethylpropyl]-3-(3,3-dimethylbutyl)-2-oxo-2,3-dihydro-1H-benzimidazole-1-carboxamide

The title compound was prepared according to the procedure described in Step 2 of Example 12 from 1-(3-methylbutyl)1,2-dihydro-2H-benzimidazol-2-one (Step 1).

¹H-NMR (270 MHz, CDCl₃) δ 9.47 (d, J=8.1 Hz, 1H), 8.16 (dd, J=7.8, 1.4 Hz, 1H), 7.27-7.12 (m, 2H), 6.99 (dd, J=7.3, 1.6 Hz, 1H), 6.01 (bs, 1H), 5.74 (bs, 1H), 4.25 (d, J=8.1 Hz, 1H), 3.99-3.81 (m, 2H), 1.70-1.62 (m, 2H), 1.15 (s, 9H), 1.04 (s, 9H).

MS (ESI) m/z 375 (M+H)⁺.

Anal. calcd. for C₂₀H₃₀N₄O₃ (+0.1H₂O): C, 63.84; H, 8.09; N, 14.89; O, 13.18. Found: C, 63.47; H, 8.10; N, 14.89.

### Example 14

### N-[(1S)-1-(Aminocarbonyl)-2,2-dimethylpropyl]-3-[(1-methylpiperidin-2-yl)methyl]-2-oxo-2,3-dihydro-1H-benzimidazole-1-carboxamide hydrochloride

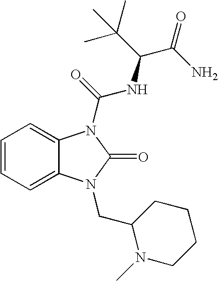

### STEP 1. 1-[(1-methylpiperidin-2-yl)methyl]-1,3-dihydro-2H-benzimidazol-2-one

The title compound was prepared according to the procedure described in Steps 1 to 3 of Example 1 from 1-(1-methylpiperidin-2-yl)methanamine.

MS (ESI) m/z 246 (M+H)⁺.

### STEP 2. N-[(1S)-1-(Aminocarbonyl)-2,2-dimethylpropyl]-3-[(1-methylpiperidin-2-yl)methyl]-2-oxo-2,3-dihydro-1H-benzimidazole-1-carboxamide hydrochloride

The title compound was prepared according to the procedure described in Step 4 of Example 1 from 1-[(1-methylpiperidin-2-yl)methyl]-1,3-dihydro-2H-benzimidazol-2-one.

¹H-NMR (300 MHz, CDCl₃) δ 9.26-9.21 (m, 2H), 8.11 (d, J=8.4 Hz, 2H), 7.41-7.14 (m, 6H), 6.13-5.95 (m, 2H), 5.61-5.56 (m, 2H), 4.71-4.52 (m, 4H), 4.26 (d, J=8.4 Hz, 2H), 3.30-3.28 (m, 4H), 2.96 (s, 3H), 2.91 (s, 3H), 2.18-1.80 (m, 14H), 1.14 (s, 18H).

MS (ESI) m/z 402 (M+H)⁺.

Anal. calcd. for C₂₁H₃₁N₅O₃(+0.8H₂O, 1.5 HCl): C, 53.60; H, 7.30; N, 14.88; O, 12.92; Cl, 11.30.

Found: C, 53.99; H, 7.61; N, 14.86.

### Example 15

### N-[(1S)-1-(Aminocarbonyl)-2,2-dimethylpropyl]-methyl-3-(2-morpholin-4-ylethyl)-2-oxo-2,3-dihydro-1H-benzimidazole-1-carboxamide

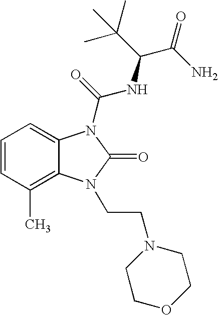

### STEP 1. 2-Methyl-N-(2-morpholin-4-ylethyl)-6-nitroaniline

A solution of 2-chloro-3-nitrotoluene (180 mg, 1.0 mmol), 4-(2-aminoethyl)morpholine (0.54 mL, 4.1 mmol) and triethylamine (0.43 mL, 3.1 mmol) was heated to 180 degrees Celsius by microwave for 20 minutes. The resultant mixture was purified by column chromatography on silica gel eluting with hexane/ethyl acetate (8/1-3/1) to afford 160 mg (59%) of the title compound.

MS (ESI) m/z 266 (M+H)⁺.

### STEP 2. 7-Methyl-1-(2-morpholin-4-ylethyl)-1,3-dihydro-2H-benzimidazol-2-one

The title compound was prepared according to the procedure described in Steps 2 to 3 of Example 1 from 2-Methyl-N-(2-morpholin-4-ylethyl)-6-nitroaniline (Step 1).

¹H-NMR (300 MHz, CDCl₃) δ 9.10 (bs, 1H), 6.98-6.91 (m, 2H), 6.85-6.82 (m, 1H), 4.24-4.19 (m, 2H), 3.72-3.69 (m, 4H), 2.70-2.65 (m, 2H), 2.60 (s, 3H), 2.58-2.55 (m, 4H).

MS (ESI) m/z 262 (M+H)⁺.

### STEP 3. N-[(1S)-1-(Aminocarbonyl)-2,2-dimethylpropyl]-4-methyl-3-(2-morpholin-4-ylethyl)-2-oxo-2,3-dihydro-1H-benzimidazole-1-carboxamide

The title compound was prepared according to the procedure described in Step 4 of example 1 from 7-methyl-1-(2-morpholin-4-ylethyl)-1,3-dihydro-2H-benzimidazol-2-one (Step 2).

¹H-NMR (300 MHz, CDCl₃) δ 9.58 (d, J=7.8 Hz, 1H), 8.10 (d, J=7.8 Hz, 1H), 7.05 (t, J=7.8 Hz, 1H), 6.96 (d, J=7.8 Hz, 1H), 5.91 (bs, 1H), 5.60 (bs, 1H), 4.29-4.18 (m, 3H), 3.73-3.65 (m, 4H), 2.75-2.64 (m, 2H), 2.60 (s, 3H), 2.63-2.45 (m, 4H), 1.15 (s, 9H).

MS (ESI) m/z 418 (M+H)⁺.

Anal. calcd. for C₂₁H₃₁N₅O₄ (+0.5H₂O, 0.1 C₄H₈O₂): C, 59.04; H, 7.59; N, 16.09; O, 17.27.

Found: C, 58.99; H, 7.35; N, 15.88.

### Example 16

### N-[(1S)-1-(Aminocarbonyl)-2,2-dimethylpropyl]-6-methyl-3-(2-morpholin-4-ylethyl)-2-oxo-2,3-dihydro-1H-benzimidazole-1-carboxamide hydrochloride

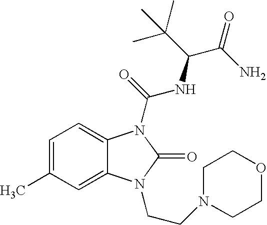

### STEP 1. 6-methyl-1-(2-morpholia-4-ylethyl)-1,3-dihydro-2H-benzimidazol-2-one

The title compound was prepared according to the procedure described in Steps 1 to 3 of Example 1 from 3-fluoro-4-nitrotluene.

MS (ESI) m/z 262 (M+H)⁺, 260 (M−H)⁻.

### STEP 2. N-[(1S)-1-(Aminocarbonyl)-2,2-dimethylpropyl]-5-methyl-3-(2-morpholin-4-ylethyl)-2-oxo-2,3-dihydro-1H-benzimidazole-1-carboxamide hydrochloride

The title compound was prepared according to the procedure described in Step 4 of Example 1 from 6-methyl-1-(2-morpholin-4-ylethyl)-1,3-dihydro-2H-benzimidazol-2-one (Step 1).

¹H-NMR (300 MHz, DMSO-d₆) δ 11.47 (bs, 1H), 9.09 (d, J=9.0 Hz, 1H), 7.90 (d, J=7.5 Hz, 1H), 7.70 (s, 1H), 7.37 (s, 1H), 7.22 (s, 1H), 7.00 (d, J=7.5 Hz, 1H), 4.43-4.32 (m, 2H), 4.25 (d, J=9.0 Hz, 1H), 4.07-3.95 (m, 2H), 3.88-3.72 (m, 2H), 3.68-3.52 (m, 2H), 3.51-3.42 (m, 2H), 3.27-3.10 (m, 2H), 2.38 (s, 3H), 1.00 (s, 9H).

MS (ESI) m/z 418 (M+H)⁺.

Anal. calcd. for C₂₁H₃₁N₅O₄ (+1.0H₂O, 1.0 HCl): C, 53.44; H, 7.26; N, 14.84; O, 16.95; Cl, 7.51.

Found: C, 53.77; H, 7.32; N, 14.64.

### Example 17

### N-[(1S)-1-(Aminocarbonyl)-2,2-dimethylpropyl]-3-(3-methylbutyl)-2-oxo-2,3-dihydro-1H-imidazo[4,5-c]pyridine-1-carboxamide

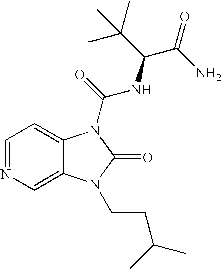

### STEP 1. 3-(3-methylbutyl)-1,3-dihydro-2H-imidazo[4,5-c]pyridin-2-one

The title compound was prepared according to the procedure described in the literature (Meth-Cohn, O.; Smith, D. I. J.C.S. Perkin Trans. 1, 1982, 261-270; Vernin G. et al. J. Heterocyclic Chem. 1981, 18, 85-89.) from 1-bromo-3-methylbutane and 1-isopropenyl-1,3-dihydro-2H-imidazo[4,5-c]pyridin-2-one (Israel, M.; Jones, L. C. J. Heterocyclic Chem. 1971, 8, 797.

¹H-NMR (300 MHz, CDCl₃) [symbol] 10.15 (br, 1H), 8.33 (d, J=5.3 Hz, 1H), 8.33 (s, 1H), 7.09 (d, J=5.3 Hz, 1H), 3.95 (t, J=7.3 Hz, 2H), 1.76-1.63 (m, 3H), 1.02 (d, J=7.0 Hz, 6H).

### STEP 2. N-[(1S)-1-(Aminocarbonyl)-2,2-dimethylpropyl]-3-(3-methylbutyl)-2-oxo-2,3-dihydro-1H-imidazo[4,5-c]pyridine-1-carboxamide

The title compound was prepared according to the procedure described in Step 4 of Example 1 from 3-(3-methylbutyl)-1,3-dihydro-2H-imidazo[4,5-c]pyridin-2-one.

¹H-NMR (300 MHz, CDCl₃) δ 9.34 (d, J=8.1 Hz, 1H), 8.43 (d, J=5.1 Hz, 1H), 8.38 (s, 1H), 8.04 (d, J=5.1 Hz, 1H), 5.80 (bs, 1H), 5.59 (bs, 1H), 4.20 (d, J=8.1. Hz, 1H), 4.00-3.90 (m, 2H), 1.76-1.65 (m, 3H), 1.15 (s, 9H), 1.02 (d, J=5.7 Hz, 6H).

MS (ESI) m/z 362 (M+H)+

Anal. calcd. for C₁₈H₂₇N₆O₃ (+0.5H₂O): C, 58.36; H, 7.62; N, 18.91; O, 15.12. Found: C, 58.60; H, 7.45; N, 18.94.

### Example 18

### N-[(1S)-(Aminocarbonyl)-2,2-dimethylpropyl]-3-[2-(dimethylamino)ethyl]-4-methyl-2-oxo-2,3-dihydro-1H-benzimidazole-1-carboxamide

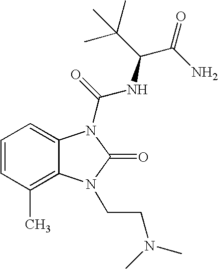

### STEP 1. 1-[2-(dimethylamino)ethyl]-7-methyl-1,3-dihydro-2H-benzimidazol-2-one

The title compound was prepared according to the procedure described in Steps 1 to 3 of Example 1 from N,N-dimethylethylenediamine and 2-chloro-3-nitrotoluene.

MS (ESI) m/z 220 (M+H)⁺.

### STEP 2. N-[(1S)-1-(Aminocarbonyl)-2,2-dimethylpropyl]-3-[2-(dimethylamino)ethyl]-4-methyl-2-oxo-2,3-dihydro-1H-benzimidazole-1-carboxamide

The title compound was prepared according to the procedure described in Step 4 of Example 1 from 7-methyl-1-[2-dimethylamino)ethyl-1,3-dihydro-2H-benzimidazol-2-one (Step 1) and L-tert-leucinamide.

¹H-NMR (270 MHz, CDCl₃) δ 9.57 (d, J=7.8 Hz, 1H), 8.10 (d, J=7.8 Hz, 1H), 7.05 (t, J=7.8 Hz, 1H), 6.96 (d, J=8.1 Hz, 1H), 5.93 (bs, 1H), 5.62 (bs, 1H), 4.27-4.17 (m, 3H), 2.68-2.58 (m, 5H), 2.35 (s, 6H), 1.15 (s, 9H).

MS (ESI) m/z 376 (M+H)+

Anal. calcd. for C₁₉H₂₉N₅O₃: C, 60.78; H, 7.79; N, 18.65; O, 12.78. Found: C, 60.67; H. 7.89; N, 18.48.

### Example 19

### N-[(1S)-1-(Aminocarbonyl)-2-methylpropyl]-3-[2-(dimethylamino)ethyl]-4-methyl-2-oxo-2,3-dihydro-1H-benzimidazole-1-carboxamide

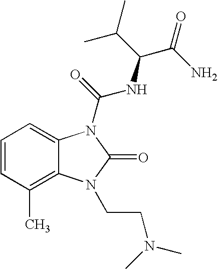

The title compound was prepared according to the procedure described in Step 4 of Example 1 from 7-methyl-1-[2-(dimethylamino)ethyl-1,3-dihydro-2H-benzimidazol-2-one (Step 1 of Example 18) and L-valinamide hydrochloride.

¹H-NMR (300 MHz, CDCl₃) δ 9.39 (d, J=8.1 Hz, 1H), 8.11 (d, J=8.1 Hz, 1H), 7.06 (d, J=8.1, Hz, 1H), 6.97 (d, J=7.2 Hz, 1H), 6.12 (bs, 1H), 5.50 (bs, 1H), 4.36 (dd, J=8.1, 5.1 Hz, 1H), 4.28-4.11 (m, 2H), 2.65-2.56 (m, 5H), 2.49-2.37 (m, 1H), 2.34 (s, 6H), 1.08 (d, J=3.0 Hz, 3H), 1.06 (d, J=3.0 Hz, 3H).

MS (ESI) m/z 362 (M+H)⁺

Anal. calcd. for C₁₈H₂₇N₆O₃(+0.7H₂O): C, 57.80; H, 7.65; N, 18.72; O, 15.83. Found: C, 57.96; H, 7.71; N, 18.35.

### Example 20

### N-{(1S)-1-{[(2-Hydroxyethyl)amino]carbonyl}-2,2-dimethylpropyl)-3-(2-morpholin-4-ylethyl)-2-oxo-2,3-dihydro-1H-benzimidazole-1-carboxamide hydrochloride

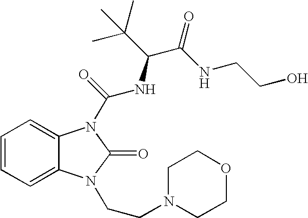

The title compound was prepared according to the procedure described in Step 4 of Example 1 from ethanolamine.

¹H-NMR (300 MHz, DMSO-d₆) δ 11.05 (bs, 1H), 9.14 (d, J=9.0 Hz, 1H), 8.30 (d, J=5.4 Hz, 1H), 8.06 (d, J=7.8, Hz, 1H), 7.51 (d, J=7.8 Hz, 1H), 7.30-7.17 (m, 2H), 4.42-4.37 (m, 2H), 4.32 (d, J=9.0 Hz, 1H), 3.80-3.70 (m, 2H), 3.94-3.65 (m, 6H), 3.25-3.06 (m, 4H), 0.98 (s, 9H).

MS (ESI) m/z 448 (M+H)⁺

Anal. calcd. for C₂₂H₃₃N₅O₅(+1.0 H₂O, 1.0 HCl): C, 52.64; H, 7.23; N, 13.95; O, 19.12; Cl, 7.06.

Found: C, 52.40; H, 7.48; N, 13.81.

### Example 21

### N-[(1S)-1-(Aminocarbonyl)-2,2-dimethylpropyl]-3-(2-morpholin-4-ylethyl)-2-oxo-2,3-dihydro-1H-imidazo[4,5-b]pyridine-1-carboxamide hydrochloride

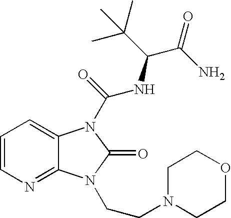

### STEP 1. 3-(2-morpholin-4-ylethyl)-2-oxo-2,3-dihydro-1H-imidazo[4,5-b]pyridine

The title compound was prepared according to the procedure described in Steps 1 to 3 of Example 1 from 2-chloro-3-nitropyridine.

¹H-NMR (300 MHz, CDCl₃) δ 9.98 (bs, 1H), 8.07-8.03 (m, 1H), 7.28-7.21 (m, 1H), 6.98-6.94 (m, 1H), 4.17-4.13 (m, 2H), 3.67-3.64 (m, 4H), 2.83-2.79 (m, 2H), 2.60-2.57 (m, 4H).

MS (ESI) T/z 249 (M+H)⁺, 247 (M−H)⁻.

### STEP 2. N-[(1S)-1-(Aminocarbonyl)-2,2-dimethylpropyl]-3-(2-morpholin-4-ylethyl)-2-oxo-2,3-dihydro-1H-imidazo[4,5-b]pyridine-1-carboxamide hydrochloride

The title compound was prepared according to the procedure described in Step 4 of Example 1 from 3-(2-morpholin-4-ylethyl)-2-oxo-2,3-dihydro-1H-imidazo[4,5-b]pyridine (Step 1) and L-tert-leucinamide.

¹H-NMR (300 MHz, DMSO-d₆) δ 11.40 (bs, 1H), 10.9 (bs, 1H), 8.93 (d, J=9.3 Hz, 1H), 8.22-8.14 (m, 1H), 7.72 (bs, 1H), 7.25-7.20 (m, 2H), 4.35 (t, J=5.4 Hz, 2H), 4.28 (d, J=9.0 Hz, 1H), 4.05-3.90 (m, 2H), 3.88-3.50 (m, 5H), 3.45-3.40 (m, 2H), 3.25-3.08 (m, 2H), 1.00 (s, 9H).

MS (ESI) m/z 405 (M+H)⁺

Anal. calcd. for C₁₉H₂₈N₆O₄(+1.0H₂O, 1.0 HCl): C, 49.72; H, 6.81; N, 18.31; O, 17.43; Cl, 7.72.

Found: C, 50.05; H, 7.01; N, 18.04.

### Example 22

### N-[(1S)-(Aminocarbonyl)-2-methylpropyl]-3-(2-morpholin-4-ylethyl)-2-oxo-2,3-dihydro-1H-benzimidazole-1-carboxamide hydrochloride

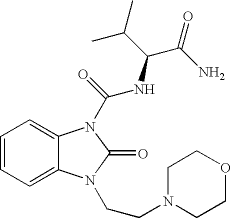

The title compound was prepared according to the procedure described in Step 4 of Example 1 from L-valinamide hydrochloride.

¹H-NMR (300 MHz, DMSO-d₆) δ 11.21 (bs, 1H), 9.00 (d, J=8.1 Hz, 1H), 8.04 (d, J=8.1 Hz, 1H), 7.67 (bs, 1H), 7.50 (d, J=7.2 Hz, 1H), 7.29-7.16 (m, 3H), 4.45-4.35 (m, 2H), 4.30 (dd, J=9.0, 5.1 Hz, 1H), 4.05-3.90 (m, 2H), 3.85-3.66 (m, 2H), 3.65-3.51 (m, 2H), 3.50-3.40 (m, 2H), 3.25-3.05 (m, 2H), 2.19-2.08 (m, 1H), 0.94 (d, J=6.6 Hz, 3H), 0.88 (d, J=6.6 Hz, 3H).

MS (ESI) m/z 390 (M+H)⁺.

Anal. calcd. for C₁₉H₂₇N₅O₄ (+0.5H₂O, 1 HCl, 0.2 C₄H₈O₂): C, 52.55; H, 6.82; N, 15.48; O, 17.32, Cl, 7.83. Found: C, 52.58; H, 6.81; N, 15.15.

### Example 23

### N-[(1S,2S)-1-(hydroxymethyl)-2-methylbutyl]-3-(2-morpholin-4-ylethyl)-2-oxo-2,3-dihydro-1H-benzimidazole-1-carboxamide

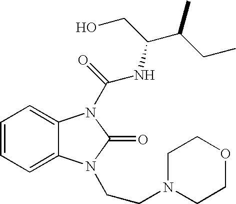

The title compound was prepared according to the procedure described in Step 4 of Example 1 from N-[1S,2S)-1-(hydroxymethyl)-2-methylbutyl]amine.

MS (ESI) m/z 391 (M+H)⁺.

Rt=1.09 min

### Example 24

### N-[(1S)-1-(hydroxymethyl)-2,2-dimethylpropyl]-3-(2-morpholin-4-ylethyl)-2-oxo-2,3-dihydro-1H-benzimidazole-1-carboxamide

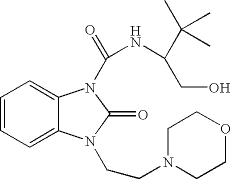

The title compound was prepared according to the procedure described in Step 4 of Example 1 from N-[(1S)-1-hydroxymethyl)-2,2-dimethylpropyl]amine.

MS (ESI) m/z 391 (M+H)⁺.

Rt=1.67 min

### Example 25

### N-[(1S)-(hydroxymethyl)methylbutyl]-3-(2-morpholin-4-ylethyl)-2-oxo-2,3-dihydro-1H-benzimidazole-1-carboxamide

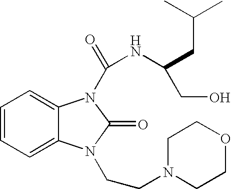

The title compound was prepared according to the procedure described in Step 4 of Example 1 from N-[(1S)-1-(hydroxymethyl)-3-methylbutyl]amine.

MS (ESI) m/z 391 (M+H)⁺.

Rt=1.76 min

### Example 26

### N-(1-[(dimethylamino)carbonyl]-1,3-dimethylbutyl)-3-(2-morpholin-4-ylethyl)-2-oxo-2,3-dihydro-1H-benzimidazole-1-carboxamide

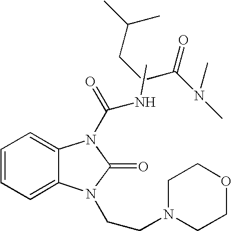

The title compound was prepared according to the procedure described in Step 4 of Example 1 from N-{1-[(dimethylamino)carbonyl]-1,3-dimethylbutyl}amine.

MS (ESI) m/z 446 (M+H)⁺.

Rt=1.74 min

### Example 27

### N-[(1S)-1-(Aminocarbonyl)-2,2-dimethylpropyl]-4-chloro-3-(2-morpholin-4-ylethyl)-2-oxo-2,3-dihydro-1H-benzimidazole-1-carboxamide

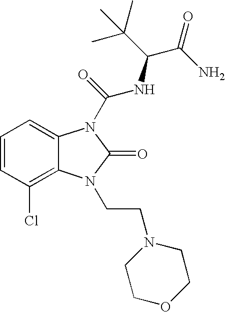

### STEP 1. 7-Chloro-1-(2-morpholin-4-ylethyl)-1,3-dihydro-2H-benzimidazol-2-one

The title compound was prepared according to the procedure described in Steps 1 to 3 of Example 1 from 1,2-dichloro-3-nitrobenzene.

¹H-NMR (300 MHz, DMSO-d₆) δ 11.2 (s, 1H), 7.05-6.87 (m, 3H), 4.23-4.10 (m, 2H), 3.59-3.48 (m, 4H), 2.62-2.37 (m, 6H).

### STEP 2. N-[(1S)-1-(Aminocarbonyl)-2,2-dimethylpropyl]-4-chloro-3-(2-morpholin-4-ylethyl)-2-oxo-2,3-dihydro-1H-benzimidazole-1-carboxamide

The title compound was prepared according to the procedure described in Step 4 of Example 1 from 7-Chloro-1-(2-morpholin-4-ylethyl)-1,3-dihydro-2H-benzimidazol-2-one (Step 1).

¹H-NMR (300 MHz, CDCl₃) δ 9.51 (d, J=8.1 Hz, 1H), 8.18 (d, J=9.2 Hz, 1H), 7.20-7.03 (m, 2H), 5.89 (bs, 1H), 5.71 (bs, 1H), 4.44-4.34 (m, 2H), 4.21 (d, J=8.1 Hz, 1H), 3.70-3.60 (m, 4H), 2.83-2.44 (m, 6H), 1.14 (s, 9H).

### Example 28

### N-[(1S)-1-(Aminocarbonyl)-2,2-dimethylpropyl]-4-chloro-3 (2-morpholin-4-ylethyl)-2-oxo-2,3-dihydro-1H-benzimidazole-1-carboxamide

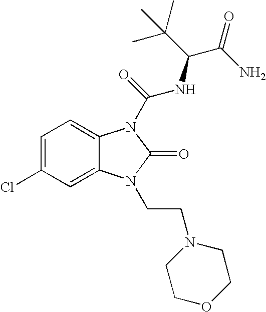

### STEP 1. 6-Chloro-1-(2-morpholin-4-ylethyl)-1,3-dihydro-2H-benzimidazol-2-one

The title compound was prepared according to the procedure described in Steps 1 to 3 of Example 1 from 4-chloro-2-flubronitrobenzene.

¹H-NMR (300 MHz, CDCl₃) δ 10.1 (s, 1H), 7.12-6.93 (m, 3H), 4.06-3.89 (m, 2H), 3.79-3.60 (m, 4H), 2.79-2.47 (m, 6H).

### STEP 2. N-(1S)-1-[Aminocarbonyl)-2,2-dimethylpropyl]-5-chloro-3-(2-morpholin-4-ylethyl)-2-oxo-2,3-dihydro-1H-benzimidazole-1-carboxamide

The title compound was prepared according to the procedure described in Step 4 of Example 1 from 6-Chloro-1-(2-morpholin-4-ylethyl)-1,3-dihydro-2H-benzimidazol-2-one (Step 1).

¹H-NMR (300 MHz, DMSO-d₆) δ 9.64 (d, J=9.2 Hz, 1H), 8.03 (d, J=8.6 Hz, 1H), 7.74-7.65 (m, 2H), 7.29-7.19 (m, 2H), 4.42-4.31 (m, 2H), 4.07 (d, J=8.6 Hz, 1H), 4.08-3.94 (m, 2H), 3.82-3.07 (m, 8H), 0.99 (s, 9H).

### Example 29

### N-[(1S)-1-(Aminocarbonyl)-2,2-dimethylpropyl]-3-[(4-hydroxytetrahydro-2H-pyran-4-yl)methyl]-2-oxo-2,3-dihydro-1H-benzimidazole-1-carboxamide

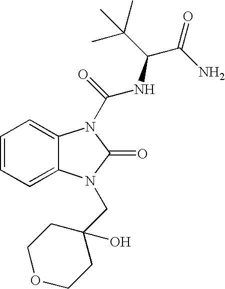

### STEP 1. 1-[(4-Hydroxytetrahydro-2H-pyran-4-yl)methyl]-1,3-dihydro-2H-benzimidazol-2-one

The title compound was prepared according to the procedure described in Steps 1 to 3 of Example 1 from 4-{[(2-Nitrophenyl)amino]methyl}tetrahydro-2H-pyran-4-ol (WO 2004029026).

¹H-NMR (300 MHz, DMSO-d₆) δ 10.9 (bs, 1H), 7.28-7.20 (m, 1H), 7.01-6.94 (m, 3H), 4.76 (s, 1H), 3.73 (s, 2H), 3.66-3.51 (m, 4H), 1.69-1.35 (m, 4H).

### STEP 2. N-[(1S)-1-(Aminocarbonyl)-2,2-dimethylpropyl]-3-[(4-hydroxytetrahydro-2H-pyran-4-yl)methyl]-2-oxo-2,3-dihydro-1H-benzimidazole-1-carboxamide

The title compound was prepared according to the procedure described in Step 4 of Example 1 from 1-[(4-Hydroxytetrahydro-2H-pyran-4-yl)methyl]-1,3-dihydro-2H-benzimidazol-2-one (Step 1).

¹H-NMR (300 MHz, DMSO-d₆) δ 9.23 (d, J=8.8 Hz, 1H), 8.04 (d, J=7.5 Hz, 1H), 7.71-7.63 (m, 1H), 7.44-7.36 (m, 1H), 7.26-7.07 (m, 3H), 4.78 (s, 1H), 4.27 (d, J=9.0 Hz, 1H), 3.91-3.47 (m, 6H), 1.74-1.36 (m, 4H), 0.99 (s, 9H).

### Example 30

### N-[(1S)-1-(Aminocarbonyl)-2,2-dimethylpropyl]-3-[2-(4-hydroxytetrahydro-2H-pyran-4-yl)ethyl]-2-oxo-2,3-dihydro-1H-benzimidazole-1-carboxamide

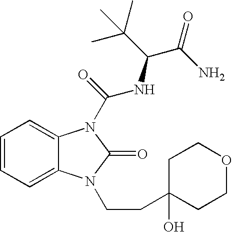

### STEP 1. 1,6-Dioxaspiro[2.5]octane-2-carbonitrile

To a mixture of tetrahydro-4H-pyran-4-one (5.0 g, 50 mmol) and chloroacetonitrile (3.8 g, 50 mmol) was dropwise added a solution of potassium tert-butoxide in tert-butanol (1.0 M, 50 mL). The reaction mixture was stirred overnight and quenched with water (100 mL). The whole was extracted with ethyl acetate (200 mL). The organic layer was washed with water and brine, dried over magnesium sulfate, filtered and concentrated in vacuo to afford the titled compound. (5.65 g)

¹H-NMR (300 MHz, CDCl₃) δ 3.94-3.79 (m, 4H), 3.35 (s, 1H), 2.17-2.03 (m, 1H), 1.97-1.76 (m, 2H), 1.65-1.53 (m, 1H).

### STEP 2. 4-(2-Aminoethyl)tetrahydro-2H-pyran-4-ol hydrochloride

A mixture of 1,6-dioxaspiro[2.5]octane-2-carbonitrile (3.0 g, 22 mmol) and 5% Pd on C (0.3 g) in methanol (40 mL) was stirred for 2 h. under hydrogen (3 kg/cm²). After filtration through a pad of celite, the filtrate was concentrated in vacuo. The residue was dissolved with THF (50 mL). The solution was added dropwise to a mixture of lithium aluminum hydride (1.6 g, 43 mmol) and THF (100 mL) and the mixture was stirred for 2 hours at reflux temperature. After cooling to 0 degrees Celsius, Na₂SO₄-10H₂O (16 g) and KF (2.5 g) were added and the mixture was stirred overnight. After filtration, the filtrate was concentrated in vacuo. The residue was acidified with 4N—HCl in ethyl acetate and concentrated in vacuo. The residue was crystallized from ethanol-ether. The precipitate was filtered to afford the titled compound. (2.1 g)

¹H-NMR (300 MHz, DMSO-d₆) δ 8.19-7.78 (m, 4H), 3.81-3.35 (m, 4H), 2.98-2.77 (m, 2H), 1.81-1.34 (m, 6H).

### STEP 3. 1-[2-(4-Hydroxytetrahydro-2H-pyran-4-yl)ethyl]-1,3-dihydro-2H-benzimidazol-2-one

The title compound was prepared according to the procedure described in Steps 1 to 3 of Example 1 from 4-(2-Aminoethyl)tetrahydro-2H-pyran-4-ol hydrochloride (Step 2).

¹H-NMR (300 MHz, DMSO-d₆) δ 10.8 (bs, 1H), 7.14-6.91 (m, 4H), 4.57 (s, 1H), 3.95-3.81 (m, 2H,), 3.70-3.44 (m, 4H), 1.75-1.42 (m, 6H).

### STEP 4. N-[(1S)-1-(Aminocarbonyl)-2,2-dimethylpropyl]-3-[2-(4-hydroxytetrahydro-2H-pyran-4-yl)ethyl]-2-oxo-2,3-dihydro-1H-benzimidazole-1-carboxamide

The title compound was prepared according to the procedure described in Step 4 of Example 1 from 1-[2-(4-Hydroxytetrahydro-2H-pyran-4-yl)ethyl]-1,3-dihydro-2H-benzimidazol-2-one (Step 3).

¹H-NMR (300 MHz, DMSO-d₆) δ 9.23 (d, J=9.2 Hz, 1H). 8.05 (d, J=7.9 Hz, 1H), 7.72-7.64 (m, 1H), 7.35-7.12 (m, 4H), 4.61 (s, 1H), 4.26 (d, Jo-9.2 Hz, 1H), 4.08-3.91 (m, 2H), 3.75-3.47 (m, 4H), 1.87-1.44 (m, 4H), 1.00 (s, 9H).

### Example 31

### N-[(1S)-1-(Aminocarbonyl)-2,2-dimethylpropyl]-3-[2-(ethylthio)ethyl]-2-oxo-2,3-dihydro-1H-benzimidazole-1-carboxamide

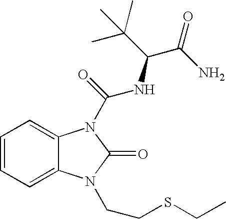

### STEP 1. 1-[2-(Ethylthio)ethyl]-1,3-dihydro-2H-benzimidazol-2-one

The title compound was prepared according to the procedure described in Steps 1 to 3 of Example 1 from 2-(ethylthio)ethanamine hydrochloride.

¹H-NMR (300 MHz, CDCl₃) δ 8.97 (bs, 1H), 7.17-6.96 (m, 4H), 4.14-4.02 (m, 2H), 2.95-2.84 (m, 2H), 1.69-1.35 (m, 4H), 2.63 (q, J=7.3 Hz, 2H), 1.28 (t, J=7.3 Hz, 3H)

### STEP 2. N-[(1S)-1-(Aminocarbonyl)-2,2-dimethylpropyl]-3-[2-(ethylthio)ethyl]-2-oxo-2,3-dihydro-1H-benzimidazole-1-carboxamide

The title compound was prepared according to the procedure described in Step 4 of Example 1 from 1-[2-(Ethylthio)ethyl]-1,3-dihydro-2H-benzimidazol-2-one (Step 1).

¹H-NMR (300 MHz, DMSO-d₆) δ 9.20 (d, J=8.8 Hz, 1H), 8.05 (d, J=7.9 Hz, 1H), 7.73-7.65 (m, 1H), 7.42-7.11 (m, 4H), 4.27 (d, J=8.8 Hz, 1H), 4.15-4.04 (m, 2H), 2.95-2.83 (m, 2H), 2.65-2.54 (m, 2H), 1.21-1.13 (m, 3H), 0.99 (s, 9H).

### Example 32

### N-[(1S)-1-(Aminocarbonyl)-2,2-dimethylpropyl]-3-[2-(methylthio)ethyl]-2-oxo-2,3-dihydro-1H-benzimidazole-1-carboxamide

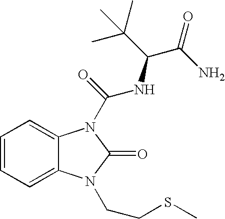

### STEP 1. 1-[2-(Methylthio)ethyl]-1,3-dihydro-2H-benzimidazol-2-one

The title compound was prepared according to the procedure described in Steps 1 to 3 of Example 1 from 2-(methylthio)ethanamine.

¹H-NMR (300 MHz, CDCl3) δ 9.36 (bs, 1H), 7.17-6.99 (m, 4H), 4.18-4.04 (m, 2H), 2.94-2.81 (m, 2H), 2.20 (s, 3H).

### STEP 2. N-[(1S)-1-(Aminocarbonyl)-2,2-dimethylpropyl]-3-[2-(methylthio)ethyl]-2-oxo-2,3-dihydro-1H-benzimidazole-1-carboxamide

The title compound was prepared according to the procedure described in Step 4 of Example 1 from 1-[2-(Methylthio)ethyl]-1,3-dihydro-2H-benzimidazol-2-one (Step 1).

H-NMR (300 MHz, DMSO-d₆) δ 9.19 (d, J=9.2 Hz, 1H), 8.06 (d, J=7.9 Hz, 1H), 7.72-7.64 (m, 1H), 7.42-7.12 (m, 4H), 4.27 (d, J=8.6 Hz, 1H), 4.17-4.06 (m, 2H), 2.90-2.81 (m, 2H), 2.14 (s, 3H), 1.00 (s, 9H).

### Example 33

N-[(1S)-1-(aminocarbonyl)-2,2-dimethylpropyl]-3-[2-(methylsulfinyl)ethyl]-2-oxo-2,3-dihydro-1H-benzimidazole-1-carboxamide

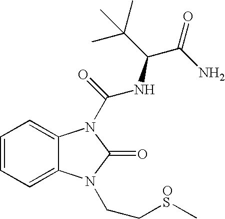

A mixture of N-[(1S)-1-(aminocarbonyl)-2,2-dimethylpropyl]-3-[2-methylthio)ethyl]-2-oxo-2,3-dihydro-1H-benzimidazole-1-carboxamide (Example 32, 150 mg), m-chloroperbenzoic acid (70%, 170 mg) and NaHCO3 (150 mg) in dichloromethane (5 mL) was stirred overnight and quenched with sat. Na₂S₂O₃ aq. (25 mL) The whole was extracted with ethyl acetate (25 mL×2). The combined organic layers were washed with brine, dried over magnesium sulfate, filtrated and concentrated in vacuo. The residue was purified by preparative TLC to yield the titled compound. (180 mg)

¹H-NMR (300 MHz, CDCl3) δ 9.39-9.29 (m, 1H), 8.12 (d, J=7.9 Hz, 1H), 7.31-7.11 (m, 3H), 6.46-6.36 (m, 1H), 6.19-6.07 (m, 1H), 4.43-4.32 (m, 2H), 4.28 (d, J=8.6 Hz, 1H), 3.33-2.99 (m, 2H), 2.67 (s, 3H), 1.13 (s, 9H).

### Example 34

N-[(1S)-1-(Aminocarbonyl)-2,2-dimethylpropyl]-3-[2-(methylsulfonyl)ethyl]-2-oxo-2,3-dihydro-1H-benzimidazole-1-carboxamide

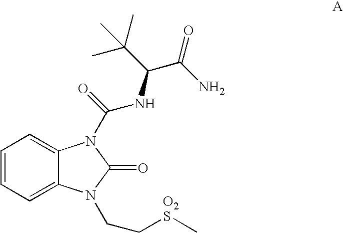

mixture of N-[(1S)-1-(aminocarbonyl)-2,2-dimethylpropyl]-3-[2-(methylsulfinyl)ethyl]-2-oxo-2,3-dihydro-1H-benzimidazole-1-carboxamide (Example 33, 150 mg), m-chloroperbenzoic acid (70%, 170 mg) and NaHCO₃ (150 mg) in dichloromethane (5 mL) was stirred overnight and quenched with sat. Na2S2O3 aq. (25 mL) The whole was extracted with ethyl acetate (25 mL×2). The combined organic layers were washed with brine, dried over magnesium sulfate, filtrated and concentrated in vacuo. The residue was purified by preparative TLC to yield the titled compound. (100 mg)

¹H-NMR (300 MHz, DMSO-d₆) δ 9.14 (d, J=8.6 Hz, 1H), 8.06 (d, J=7.9 Hz, 1H), 7.74-7.65 (m, 1H), 7.43-7.15 (m, 4H), 4.40-4.24 (m, 3H), 3.72-3.53 (m, 2H), 3.11 (s, 3H), 1.00 (s, 9H).

Following Examples 35 to 90 were prepared according to the procedure described in Step 4 of Example 1.

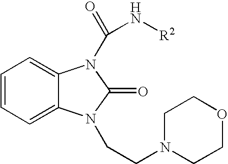

| | |
|---|---|
| Example 35 | Methyl N-{[3-(2-Morpholin-4-ylethyl)-2-oxo-2,3-dihydro-1H-benzimidazol-1-yl] |
| | carbonyl}-L-phenylalaninate |
| 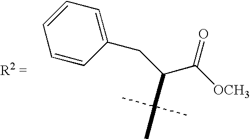 | ¹H-NMR (300 MHz, CDCl₃) δ 9.25 (d, J = 7.5 Hz, 1 H), 8.17-8.14 (m, 1 H), 7.33-7.12 (m, 7 H), 7.03-7.00 (m, 1 H), 4.89 (dt, J = 7.5, 5.7 Hz, 1 H), 4.04-3.94 (m, 2 H), 3.74 (s, 3 H), 3.66 (t, J = 4.8 Hz, 4 H), 3.28 (dd, J = 13.8, 5.4 Hz, 1 H), 3.15 (dd, J = 13.8, 7.5 Hz, 1 H), 2.72-2.62 (m, 2 H), 2.57-2.47 (m, 4 H). MS (ESI) m/z 453 (M + H)⁺. |
| Example 36 | N-(3,4-Dihydro-2H-chromen-4-yl)-3-(2-morpholin-4-ylethyl)-2-oxo-2,3-dihydro- |
| | 1H-benzimidazole-1-carboxamide hydrochloride |
| 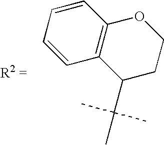 | ¹H-NMR (300 MHz, CDCl₃) δ 13.81 (bs, 1 H), 8.87 (d, J = 7.5 Hz, 1 H), 8.24 (d, J = 7.5 Hz, 1 H), 7.57 (d, J = 7.5 Hz, 1 H), 7.34-7.18 (m, 4 H), 6.95-6.85 (m, 2 H), 5.26-5.20 (m, 1 H), 4.65-4.50 (m, 2 H), 4.37-3.92 (m, 6 H), 3.57-3.40 (m, 2 H), 3.35-3.22 (m, 2 H), 3.10-2.85 (m, 2 H), 2.40-2.30 (m, 1 H), 2.23-2.14 (m, 1 H). MS (ESI) m/z 423 (M + H)⁺. Anal. calcd. for C₂₃H₂₆N₄O₄ (+0.6 H₂O, 1 HCl): C, 58.81; H, 6.05; N, 11.93; O, 15.67, Cl, 7.75. Found: C, 59.13; H, 6.23; N; 11.53. IR (KBr)νₘₐₓ 1728, 1537, 1489, 1385 cm⁻¹. |
| Example 37 | N-cyclohexyl-3-(2-morpholin-4-ylethyl)-2-oxo-2,3-dihydro-1H-benzimidazole- |
| | 1-carboxamide |
| 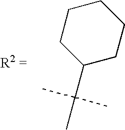 | MS (ESI) m/z 373 (M + H)⁺. Rt = 1.96 min |
| Example 38 | N-(2-methylcyclohexyl)-3-(2-morpholin-4-ylethyl)-2-oxo-2,3-dihydro-1H- |
| | benzimidazole-1-carboxamide |
| 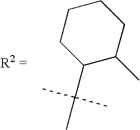 | MS (ESI) m/z 387 (M + H)⁺. Rt = 1.24 min |
| Example 39 | N-(2,3-dihydro-1H-inden-1-yl)-3-(2-morpholin-4-ylethyl)-2-oxo-2,3-dihydro- |
| | 1H-benzimidazole-1-carboxamide |
| 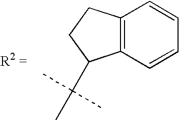 | MS (ESI) m/z 407 (M + H)⁺. Rt = 1.22 min |
| Example 40 | N-(2,3-dimethylcyclohexyl)-3-(2-morpholin-4-ylethyl)-2-oxo-2,3-dihydro-1H- |
| | benzimidazole-1-carboxamide |
| 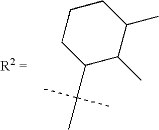 | MS (ESI) m/z 401 (M + H)⁺. Rt = 1.32 min |
| Example 41 | 3-(2-morpholin-4-ylethyl)-2-oxo-N-(3,3,5-trimethylcyclohexyl)-2,3-dihydro- |
| | 1H-benzimidazole-1-carboxamide |
| 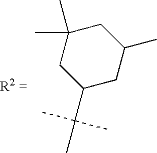 | MS (ESI) m/z 415 (M + H)⁺. Rt = 1.37 min |
| Example 42 | N-(4-methylcyclohexyl)-3-(2-morpholin-4-ylethyl)-2-oxo-2,3-dihydro-1H- |
| | benzimidazole-1-carboxamide |
| 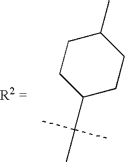 | MS (ESI) m/z 387 (M + H)⁺. Rt = 1.26 min |
| Example 43 | N-(1,3-dimethylbutyl)-3-(2-morpholin-4-ylethyl)-2-oxo-2,3-dihydro-1H- |
| | benzimidazole-1-carboxamide |
| 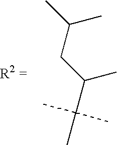 | MS (ESI) m/z 375 (M + H)⁺. Rt = 1.24 min |
| Example 44 | N-(1-methylpentyl)-3-(2-morpholin-4-ylethyl)-2-oxo-2,3-dihydro-1H- |
| | benzimidazole-1-carboxamide |
| 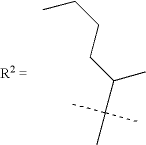 | MS (ESI) m/z 375 (M + H)⁺. Rt = 1.24 min |
| Example 45 | 3-(2-morpholin-4-ylethyl)-2-oxo-N-[(1R)-1,2,2-trimethylpropyl]-2,3-dihydro- |
| | 1H-benzimidazole-1-carboxamide |
| 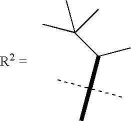 | MS (ESI) m/z 375 (M + H)⁺. Rt = 1.21 min |
| Example 46 | N-cyclooctyl-3-(2-morpholin-4-ylethyl)-2-oxo-2,3-dihydro-1H-benzimidazole- |
| | 1-carboxamide |
| 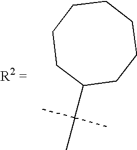 | MS (ESI) m/z 401 (M + H)⁺. Rt = 1.31 min |
| Example 47 | N-(1-methyl-1-phenylethyl)-3-(2-morpholin-4-ylethyl)-2-oxo-2,3-dihydro-1H- |
| | benzimidazole-1-carboxamide |
| 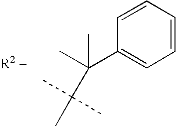 | MS (ESI) m/z 409 (M + H)⁺. Rt = 1.21 min |
| Example 48 | N-[(1R)-1,2-dimethylpropyl]-3-(2-morpholin-4-ylethyl)-2-oxo-2,3-dihydro-1H- |
| | benzimidazole-1-carboxamide |
| 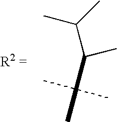 | MS (ESI) m/z 361 (M + H)⁺. Rt = 1.17 min |
| Example 49 | N-[(1S)-2,3-dihydro-1H-inden-1-yl]-3-(2-morpholin-4-ylethyl)-2-oxo-2,3-dihdyro- |
| | 1H-benzimidazole-1-carboxamide |
| 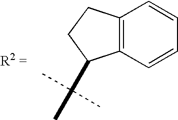 | MS (ESI) m/z 407 (M + H)⁺. Rt = 1.22 min |
| Example 50 | N-[(1S)-1-cyclohexylethyl]-3-(2-morpholin-4-ylethyl)-2-oxo-2,3-dihydro-1H- |
| | benzimidazole-1-carboxamide |
| 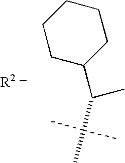 | MS (ESI) m/z 401 (M + H)⁺. Rt = 1.32 min |
| Example 51 | 3-(2-morpholin-4-ylethyl)-2-oxo-N-(1-propylbutyl)-2,3-dihydro-1H-benzimidazole- |
| | 1-carboxamide |
| 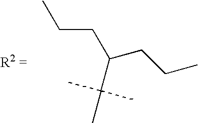 | MS (ESI) m/z 389 (M + H)⁺. Rt = 1.29 min |
| Example 52 | N-[(1R)-1-cyclohexylethyl]-3-(2-morpholin-4-ylethyl)-2-oxo-2,3-dihydro-1H- |
| | benzimidazole-1-carboxamide |
| 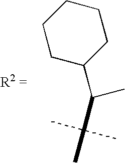 | MS (ESI) m/z 401 (M + H)⁺. Rt = 1.32 min |
| Example 53 | 3-(2-morpholin-4-ylethyl)-2-oxo-N-[(1S)-1,2,2-trimethylpropyl]-2,3-dihydro- |
| | 1H-benzimidazole-1-carboxamide |
| 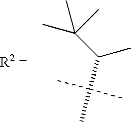 | MS (ESI) m/z 375 (M + H)⁺. Rt = 1.21 min |
| Example 54 | N-[(3S,5S,7S)-1-adamantyl]-3-(2-morpholin-4-ylethyl)-2-oxo- |
| | 2,3-dihydro-1H-benzimidazole-1-carboxamide |
| 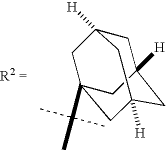 | MS (ESI) m/z 425 (M + H)⁺. Rt = 1.22 min |
| Example 55 | N-(1-ethynylcyclohexyl)-3-(2-morpholin-4-ylethyl)-2-oxo-2,3-dihdyro-1H- |
| | benzimidazole-1-carboxamide |
| 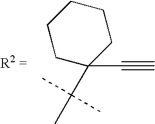 | MS (ESI) m/z 397 (M + H)⁺. Rt = 1.20 min |
| Example 56 | 3-(2-morpholin-4-ylethyl)-2-oxo-N-[(1S,2R,3S,5R)-2,6,6-trimethylbicyclo |
| | [3.1.1]hept-3-yl]-2,3-dihydro-1H-benzimidazole-1-carboxamide |
| 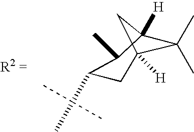 | MS (ESI) m/z 427 (M + H)⁺. Rt = 1.38 min |
| Example 57 | N-(dicyclopropylmethyl)-3-(2-morpholin-4-ylethyl)-2-oxo-2,3-dihydro-1H- |
| | benzimidazole-1-carboxamide |
| 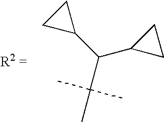 | MS (ESI) m/z 385 (M + H)⁺. Rt = 1.20 min |
| Example 58 | N-(2-hydroxy-1,2,3,4-tetrahydronaphthalen-1-yl)-3-(2-morpholin-4-ylethyl)-2- |
| | oxo-2,3-dihydro-1H-benzimidazole-1-carboxamide |
| 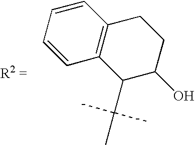 | MS (ESI) m/z 437 (M + H)⁺. Rt = 1.13 min |
| Example 59 | methyl N-{[3-(2-morpholin-4-ylethyl)-2-oxo-2,3-dihydro-1H-benzimidazole-1- |
| | yl]carbonyl}-L-leucinate |
| 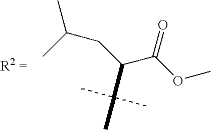 | MS (ESI) m/z 419 (M + H)⁺. Rt = 1.18 min |
| Example 60 | N-[1-(1-adamantyl)ethyl]-3-(2-morpholin-4-ylethyl)-2-oxo-2,3-dihydro-1H- |
| | benzimidazole-1-carboxamide |
| 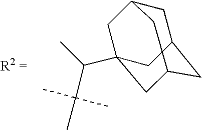 | MS (ESI) m/z 453 (M + H)⁺. Rt = 1.44 min |
| Example 61 | N-(1,1-diethylprop-2-yn-1-yl)-3-(2-morpholin-4-ylethyl)-2-oxo-2,3-dihydro-1H- |
| | benzimidazole-1-carboxamide |
| 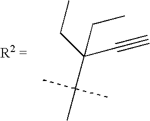 | MS (ESI) m/z 385 (M + H)⁺. Rt = 1.21 min |
| Example 62 | N-{[(1S,2R,5S)-6,6-dimethylbicyclo[3.1.1]hept-2-yl]methyl}-3-(2-morpholin-4- |
| | ylethyl)-2-oxo-2,3-dihydro-1H-benzimidazole-1-carboxamide |
| 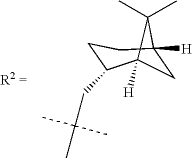 | MS (ESI) m/z 427 (M + H)⁺. Rt = 1.38 min |
| Example 63 | 3-(2-morpholin-4-ylethyl)-2-oxo-N-(1,2,3,4-tetrahydronaphthalen-1-yl)-2,3- |
| | dihydro-1H-benzimidazole-1-carboxamide |
| 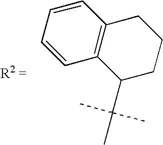 | MS (ESI) m/z 421 (M + H)⁺. Rt = 2.16 min |
| Example 64 | ethyl (1R,2S)-2-({[3-(2-morpholin-4-ylethyl)-2-oxo-2,3-dihydro-1H- |
| | benzimidazole-1-yl]carbonyl}amino)cyclohexanecarboxylate |
| 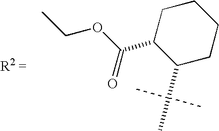 | MS (ESI) m/z 445 (M + H)⁺. Rt = 1.98 min |
| Example 65 | methyl 1-({[3-(2-morpholin-4-ylethyl)-2-oxo-2,3-dihydro-1H-benzimidazol-1- |
| | yl]carbonyl}amino)cyclohexanecarboxylate |
| 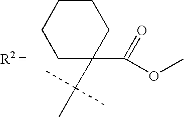 | MS (ESI) m/z 431 (M + H)⁺. Rt = 1.88 min |
| Example 66 | 3-(2-morpholin-4-ylethyl)-2-oxo-N-[(1R,2R)-2-(phenylthio)cyclopentyl]-2,3- |
| | dihydro-1H-benzimidazole-1-carboxamide |
| 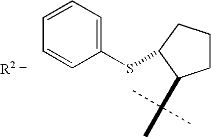 | MS (ESI) m/z 467 (M + H)⁺. Rt = 2.25 min |
| Example 67 | N-(1-ethyl-1-methylpropyl)-3-(2-morpholin-4-ylethyl)-2-oxo-2,3-dihydro-1H- |
| | benzimidazole-1-carboxamide |
| 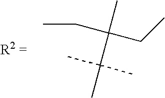 | MS (ESI) m/z 375 (M + H)⁺. Rt = 1.36 min |
| Example 68 | N-[(1R,2R,4S)-bicyclo[2.2.1]hept-2-yl]-3-(2-morpholin-4-ylethyl)-2-oxo-2,3- |
| | dihydro-1H-benzimidazole-1-carboxamide |
| 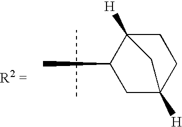 | MS (ESI) m/z 385 (M + H)⁺. Rt = 2.02 min |
| Example 69 | 3-(2-morpholin-4-ylethyl)-2-oxo-N-(3,3,5,5-tetramethylcyclohexyl)-2,3- |
| | dihydro-1H-benzimidazole-1-carboxamide |
| 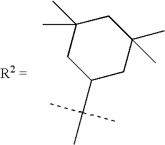 | MS (ESI) m/z 429 (M + H)⁺. Rt = 2.48 min |
| Example 70 | 3-(2-morpholin-4-ylethyl)-2-oxo-N-(1,1,3,3-tetramethyl)-2,3-dihydro-1H- |
| | benzimidazole-1-carboxamide |
| 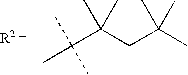 | MS (ESI) m/z 303 (M + H)⁺. Rt = 2.35 min |
| Example 71 | N-(1-isopropyl-2-methylpropyl)-3-(2-morpholin-4-ylethyl)-2-oxo-2,3-dihydro- |
| | 1H-benzimidazole-1-carboxamide |
| 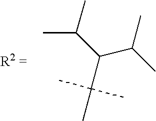 | MS (ESI) m/z 389 (M + H)⁺. Rt = 1.36 min |
| Example 72 | N-[(1S,4R)-bicyclo[2.2.1]hept-2-yl]-3-(2-morpholin-4-ylethyl)-2-oxo-2,3- |
| | dihydro-1H-benzimidazole-1-carboxamide |
| 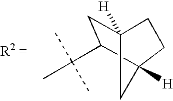 | MS (ESI) m/z 385 (M + H)⁺. Rt = 2.00 min |
| Example 73 | 3-(2-morpholin-4-ylethyl)-N-1-naphthyl-2-oxo-2,3-dihydro-1H-benzimidazole- |
| | 1-carboxamide |
| 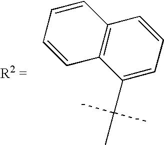 | ¹H-NMR (300 MHz, CDCl₃). δ 11.50 (bs, 1 H), 8.37-8.34 (m, 1 H), 8.26 (d, J = 7.5 Hz, 1 H), 8.16 (d, J = 8.4 Hz, 1 H), 7.91-7.89 (m, 1 H), 7.71 (d, J = 8.3 Hz, 1 H), 7.63-7.51 (m, 3 H), 7.30-7.22 (m, 2 H), 7.13-7.10 (m, 1 H), 4.12 (t, J = 6.6 Hz, 2 H), 3.72-3.68 (m, 4 H), 2.77 (t, J = 6.6 Hz, 2 H), 2.57-2.60 (m, 4 H). MS (ESI) m/z 417.2 (M + H)⁺. Anal. calcd. for C₂₄H₂₄N₄O₃: C, 69.21; H, 5.81; N, 13.45; O, 11.52. Found: C, 69.35; H, 5.84; N; 13.49. IR (KBr)νₘₐₓ 2849, 1730, 1690, 1564, 1489, 1385 cm⁻¹. mp 137.3, 128.3 degrees Celsius. |
| Example 74 | 3-(2-morpholin-4-ylethyl)-2-oxo-N-(5,6,7,8-tetrahydonaphthalen-1-yl)-2,3- |
| | dihydro-1H-benzimidazole-1-carboxamide |
| 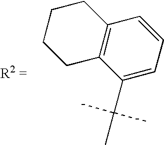 | ¹H-NMR (300 MHz, CDCl₃). δ 10.69 (bs, 1 H), 8.34-8.31 (m, 1 H), 7.93 (d, J = 8.0 Hz, 1 H), 7.27-7.15 (m, 3 H), 7.09-7.06 (m, 1 H), 6.93 (d, J = 7.7 Hz, 1 H), 4.06 (t, J = 6.8 Hz, 2 H), 3.69-3.66 (m, 4 H), 2.83-2.70 (m, 6 H), 2.57-2.54 (m, 4 H), 1.94-1.86 (m, 2 H), 1.82-1.74 (m, 2 H). MS (ESI) m/z 421.2 (M + H)⁺. Anal. calcd. for C₂₄H₂₈N₄O₃: C, 68.55; H, 6.71; N, 13.32; O, 11.41. Found: C, 68.34; H, 6.73; N; 13.12. IR (KBr)νₘₐₓ 2945, 1732, 1609, 1568, 1387, 1302, 1159, 1117 cm⁻¹. mp 141.5 degrees Celsius. |
| Example 75 | N-(1-adamantylmethyl)-3-(2-morpholin-4-ylethyl)-2-oxo-2,3-dihydro-1H- |
| | benzimidazole-1-carboxamide |
| 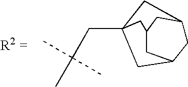 | ¹H-NMR (300 MHz, CDCl₃). δ 8.86-8.82 (m, 1 H), 8.26-8.23 (m, 1 H), 7.24-7.14 (m, 2 H), 7.05-7.02 (m, 1 H), 4.02 (t, J = 6.8 Hz, 2 H), 3.69-3.66 (m, 4 H), 3.13 (d, J = 6.0 Hz, 2 H), 2.70 (t, J = 6.8 Hz, 2 H), 2.55-2.52 (m, 4 H), 2.00 (bs, 3 H), 1.75-1.63 (m, 6 H), 1.59-1.58 (m, 6 H). MS (ESI) m/z 439.3 (M + H)⁺. Anal. calcd. for C₂₅H₃₄N₄O₃: C, 68.47; H, 7.81; N, 12.78; O, 10.94. Found: C, 68.66; H, 7.82; N; 12.69. IR (KBr)νₘₐₓ 2907, 1730, 1558, 1393 cm⁻¹. mp 142.0 degrees Celsius. |
| Example 76 | 3-(2-morpholin-4-ylethyl)-2-oxo-N-[(1S)-1,2,3,4-tetrahydronaphthalen-1-yl]-2, |
| | 3-dihydro-1H-benzimidazole-1-carboxamide hydrochloride |
| 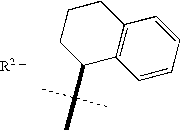 | ¹H-NMR (300 MHz, CDCl₃). δ 13.86 (bs, 1 H), 8.82 (d, J = 8.4 Hz, 1 H), 8.27 (dd, J = 8.1, 1.5 Hz, 1 H), 7.58-7.55 (m, 1 H), 7.41-7.36 (m, 1 H), 7.32-7.11 (m, 4 H), 5.29-5.23 (m, 1 H), 4.61-4.57 (m, 2 H), 4.28-4.20 (m, 2 H), 3.99 (d, J = 11.7 Hz, 2 H), 3.49-3.45 (m, 2 H), 3.30-3.25 (m, 2 H), 2.93-2.75 (m, 4 H), 2.22-2.12 (m, 1 H), 2.03-1.87 (m, 3 H). MS (ESI) m/z 421.3(M + H)⁺. Anal. calcd. for C₂₄H₂₈N₄O₃ (+0.4 H₂O, 1.0 HCl): C, 62.10; H, 6.47; N, 12.07; O, 11.72, Cl, 7.64. Found: C, 62.42; H, 6.56; N; 11.75. |
| Example 77 | 3-(2-morpholin-4-ylethyl)-2-oxo-N-[(1R)-1,2,3,4-tetrahydronaphthalen-1-yl]-2, |
| | 3-dihydro-1H-benzimidazole-1-carboxamide hydrochloride |
| 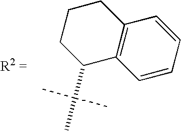 | ¹H-NMR (300 MHz, CDCl₃). δ 13.90 (bs, 1 H), 8.82 (d, J = 8.1 Hz, 1 H), 8.27 (dd, J = 8.1, 1.2 Hz, 1 H), 7.59-7.56 (m, 1 H), 7.41-7.36 (m, 1 H), 7.32-7.11 (m, 4 H), 5.29-5.23 (m, 1 H), 4.62-4.57 (m, 2 H), 4.29-4.21 (m, 2 H), 4.01-3.98 (m, 2 H), 3.49-3.45 (m, 2 H), 3.30-3.25 (m, 2 H), 2.93-2.75 (m, 4 H), 2.22-2.12 (m, 1 H), 2.03-1.87 (m, 3 H). MS (ESI) m/z 421.3 (M + H)⁺. Anal. calcd. for C₂₄H₂₈N₄O₃ (+0.2 H₂O, 1.0 HCl): C, 62.59; H, 6.43; N, 12.16; O, 11.12, Cl, 7.70. Found: C, 62.36; H, 6.59; N; 11.80. |
| Example 78 | N-isoquinolin-1-yl-3-(2-morpholin-4-ylethyl)-2-oxo-2,3-dihydro-1H- |
| | benzimidazole-1-carboxamide |
| 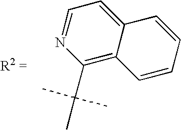 | ¹H-NMR (300 MHz, CDCl₃). δ 11.97 (s, 1 H), 8.47 (d, J = 5.7 Hz, 1 H), 8.44-8.39 (m, 1 H), 8.23 (d, J = 8.7 Hz, 1 H), 7.88-7.85 (m, 1 H), 7.76-7.65 (m, 2 H), 7.53 (d, J = 5.7 Hz, 1 H), 7.31-7.21 (m, 2 H), 7.14-7.09 (m, 1 H), 4.12 (t, J = 6.6 Hz, 2 H), 3.71-3.68 (m, 4 H), 2.77 (t, J = 6.6 Hz, 2 H), 2.60-2.57 (m, 4 H). MS (ESI) m/z 418.3 (M + H)⁺. Anal. calcd. for C₂₄H₂₃N₅O₃: C, 66.17; H, 5.55; N, 16.78; O, 11.50. Found: C, 66.13; H, 5.56; N; 16.62. IR (KBr)νₘₐₓ 2827, 1753, 1634, 1547, 1377 cm⁻¹. mp 139.0 degress Celsius. |
| Example 79 | Methyl N-{[4-methoxy-3-(2-morpholin-4-ylethyl)-2-oxo-2,3-dihydro-1H- |
| | benzimidazol-1-yl]carbonyl}-3-methyl-L-valinate |
| 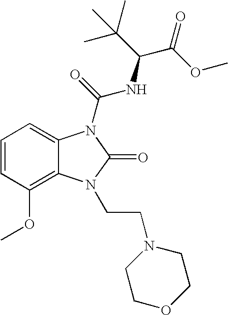 | ¹H-NMR (300 MHz, CDCl₃) δ 9.54 (d, J = 8.4 Hz, 1 H), 7.86 (dd, J = 8.1, 0.6 Hz, 1 H), 7.08 (t, J = 8.1 Hz, 1 H), 6.76 (d, J = 8.1 Hz, 1 H), 4.43 (d, J = 8.4 Hz, 1 H), 4.31-4.17 (m, 2 H), 3.91 (s, 3 H), 3.76 (s, 3 H), 3.67 (t, J = 4.5 Hz, 4 H), 2.75-2.64 (m, 2 H), 2.59-2.50 (m, 4 H), 1.09 (s, 9 H), MS (ESI) m/z 449 (M + H)⁺. Anal. calcd. for C₂₂H₃₂N₄O₆: C, 58.91; H, 7.19; N, 12.49; O, 21.40. Found: C, 58.78; H, 7.12 N; 12.35. |
| Example 80 | N-[(1S)-1-(Aminocarbonyl)-2,2-dimethylpropyl]-4-methoxy-3-(2-morpholin-4- |
| | ylethyl)-2-oxo-2,3-dihydro-1H-benzimidazole-1-carboxamide |
| 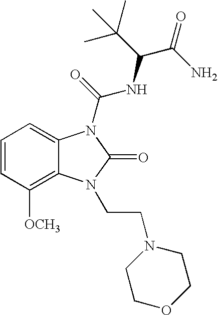 | ¹H-NMR (300 MHz, CDCl3) δ 9.58 (d, J = 7.8 Hz, 1 H), 7.84 (dd, J = 8.4, 0.9 Hz, 1 H), 7.08 (d, J = 8.4 Hz, 1 H), 6.77 (d, J = 8.4 Hz, 2 H), 5.96 (bs, 1 H), 5.66 (bs, 1 H), 4.32-4.15 (m, 3 H), 3.91 (s, 3 H), 3.67 (t, J = 4.5 Hz, 2 H), 2.76-2.62 (m, 2 H), 2.60-2.49 (m, 4 H), 1.14 (s, 9 H). MS (ESI) m/z 434.4 (M + H)⁺. Anal. calcd. for C₂₁H₃₁N₅O₅ (+1 H₂O): C, 57.94; H, 7.22; N, 16.09; O, 18.74. Found: C, 57.77; H, 7.19; N; 15.72. mp 210.9 degrees Celsius |
| Example 81 | N-[(1S)-1-(Aminocarbonyl)-2,2-dimethylpropyl]-2-oxo-3-(2-piperidin-1-ylethyl)- |
| | 2,3-dihydro-1H-benzimidazole-1-carboxamide hydrochloride |
| 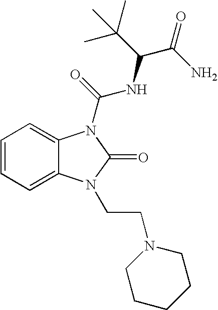 | ¹H-NMR (300 MHz, DMSO-d₆) δ 10.51 (bs, 1 H), 9.13 (d, J = 9.0 Hz, 1 H), 8.06 (dd, J = 7.8, 0.9 Hz, 1 H), 7.70 (bs, 1 H), 7.53 (d, J = 7.8 Hz, 1 H), 7.31-7.16 (m, 3 H), 4.35-4.45 (m, 2 H), 4.27 (d, J = 9.0 Hz, 1 H), 3.70-3.51 (m, 2 H), 3.43-3.31 (m, 2 H), 3.05-2.87 (m, 2 H), 1.88-1.65 (m, 5 H), 1.47-1.30 (m, 1 H), 1.00 (s, 9 H). MS (ESI) m/z 4.02.3 (M + H)⁺. Anal. calcd. for C₂₁H₃₁N₅O₃ (+1.0 H₂O, 1.0 HCl): C, 55.32; H, 7.52; N, 15.36; O, 14.04; Cl, 7.78. Found: C, 55.70; H, 7.69; N; 15.30. |
| Example 82 | N-{(1S,2S)-1-[(dimethylamino)carbonyl]-2-methylbutyl}-2-oxo-3-(2-piperidin- |
| | 1-ylethyl)-2,3-dihydro-1H-benzimidazole-1-carboxamide |
| 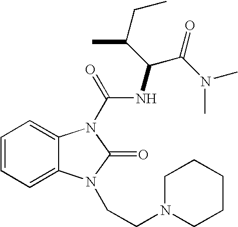 | MS (ESI) m/z 430 (M + H)⁺. Rt = 1.07 min |
| Example 83 | N-{(1S,2S)-1-[(dimethylamino)carbonyl]-2-methylbutyl}-2-oxo-3-(2- |
| | thiomorpholin-4-ylethyl)-2,3-dihydro-1H-benzimidazol-1-carboxamide |
| 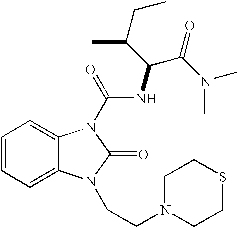 | MS (ESI) m/z 448 (M + H)⁺. Rt = 1.06 min |
| Example 84 | 4-methoxy-3-(2-morpholin-4-ylethyl)-N-1-naphthyl-2-oxo-2,3-dihydro-1H- |
| | benzimidazole-1-carboxamide |
| 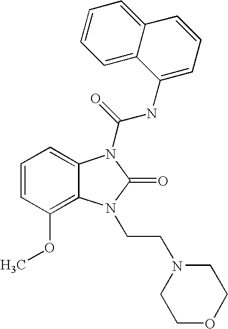 | ¹H-NMR (300 MHz, CDCl₃). δ 11.64 (bs, 1 H), 8.25 (dd, J = 7.5, 0.9 Hz, 1 H), 8.16 (d, J = 8.4 Hz, 1 H), 8.03 (dd, J = 8.25, 0.9 Hz, 1 H), 7.90-7.88 (m, 1 H), 7.70 (d, J = 8.4 Hz, 1 H), 7.63-7.50 (m, 3 H), 7.16 (t, J = 8.3 Hz, 1 H), 6.83-6.81 (m, 1 H), 4.34 (t, J = 6.8 Hz, 2 H), 3.94 (s, 3 H), 3.71-3.68 (m, 4 H), 2.75 (t, J = 6.8 Hz, 2 H), 2.60-2.57 (m, 4 H). MS (ESI) m/z 447.2 (M + H)⁺. Anal. calcd. for C₂₅H₂₆N₄O₄: C, 67.25; H, 5.87; N, 12.55; O, 14.33. Found: C, 67.30; H, 6.01; N; 12.48. IR (KBr)νₘₐₓ 2959, 1734, 1690, 1572, 1387, 1234 cm⁻¹. mp 170.9 degrees Celsius. |
| Example 85 | N-1-naphthyl-2-oxo-3-(2-piperidin-1-ylethyl)-2,3-dihydro-1H-benzimidazole-1- |
| | carboxamide |
| 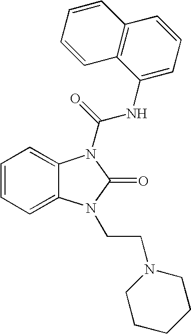 | ¹H-NMR (300 MHz, CDCl₃). δ 11.53 (bs, 1 H), 8.36-8.33 (m, 1 H), 8.26 (dd, J = 7.5, 0.9 Hz, 1 H), 8.17 (d, J = 8.7 Hz, 1 H), 7.91-7.88 (m, 1 H), 7.70 (d, J = 8.1 Hz, 1 H), 7.63-7.51 (m, 3 H), 7.30-7.20 (m, 2 H), 7.16-7.13 (m, 1 H), 4.16-4.07 (m, 2 H), 2.77-2.67 (m, 2 H), 2.58-2.46 (m, 4 H), 1.65-1.52 (m, 4 H), 1.50-1.39 (m, 2 H). MS (ESI) m/z 415.3 (M + H)⁺. Anal. calcd. for C₂₅H₂₆N₄O₂: C, 72.44; H, 6.32; N, 13.52; O, 7.72. Found C, 72.51; H, 6.41; N; 13.26. IR (KBr)νₘₐₓ 2941, 1734, 1572, 1379, 1261, 1163 cm⁻¹. mp 145.7 degrees Celsius. |
| Example 86 | 3-(2-morpholin-4-ylethyl)-2-oxo-N-(1,2,3,4-tetrahydronaphthalen-1-yl)-2,3- |
| | dihydro-1H-imidazo[4,5-b]pyridine-1-carboxamide hydrochloride |
| 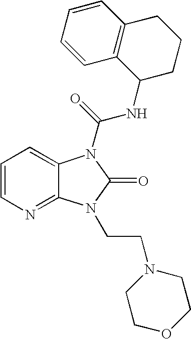 | ¹H-NMR (300 MHz, CDCl₃). δ 13.59 (bs, 1 H), 8.57-8.52 (m, 2 H), 8.13 (d, J = 4.8 Hz, 1 H), 7.45-7.42 (m, 1 H), 7.29-7.12 (m, 3 H), 5.27-5.20 (m, 1 H), 4.63 (bs, 2 H), 4.32-4.22 (m, 2 H), 3.98-3.93 (m, 2 H), 3.74-3.70 (m, 2 H), 3.53 (bs, 2 H), 2.94-2.75 (m, 4 H), 2.25-2.15 (m, 1 H), 2.01-1.89 (m, 3 H). MS (ESI) m/z 422.2 (M + H)⁺. Anal. calcd. for C₂₃H₂₇N₅O₂ (+2.0 HCl): C, 55.87; H, 5.91; N, 14.17; O, 9.71; Cl, 14.34. Found: C, 55.87; H, 5.99; N; 14.23. IR (KBr)νₘₐₓ 1736, 1535, 1394 cm⁻¹. |
| Example 87 | 4-methyl-3-(2-morpholin-4-ylethyl)-2-oxo-N-(1,2,3,4-tetrahydronaphthalen-1- |
| | yl),2,3-dihydro-1H-benzimidazole-1-carboxamide hydrochloride |
| 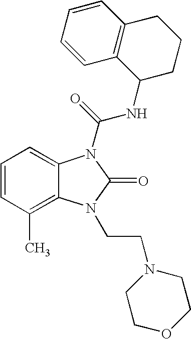 | ¹H-NMR (300 MHz, CDCl₃). δ 14.98 (bs, 1 H), 8.94-8.92 (m, 1 H), 8.23 (d, J = 8.1 Hz, 1 H), 7.40-7.37 (m, 1 H), 7.21-7.17 (m, 2 H), 7.15-7.10 (m, 2 H), 7.01 (d, J = 7.2 Hz, 1 H), 5.29-5.23 (m, 1 H), 4.80-4.68 (m, 2 H), 4.34-4.26 (m, 2 H), 4.01-3.97 (m, 2 H), 3.60-3.48 (m, 2 H), 3.30-3.15 (m, 2 H), 3.05-2.90 (m, 2 H), 2.88-2.81 (m, 1 H), 2.72 (s, 3 H), 2.19-2.13 (m, 1 H), 2.02-1.89 (m, 4 H). MS (ESI) m/z 435.1 (M + H)⁺. Anal. calcd. for C₂₅H₃₀N₄O₃ (+1.0 HCl): C, 63.75; H, 6.63; N, 11.90; O, 10.19; Cl, 7.53. Found: C, 63.42; H, 6.64; N; 11.79. IR (KBr)νₘₐₓ 1724, 1537, 1452 cm⁻¹. |
| Example 88 | 3-(2-methoxyethyl)-2-oxo-N-(1,2,3,4-tetrahydronaphthalen-1-yl)-2,3-dihydro- |
| | 1H-benzimidazole-1-carboxamide |
| 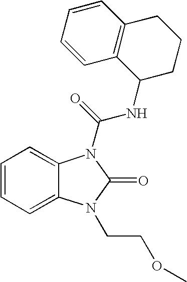 | ¹H-NMR (300 MHz, CDCl₃). δ 9.07 (d, J = 7.5 Hz, 1 H), 8.29-8.26 (m, 1 H), 7.43-7.40 (m, 1 H), 7.28-7.10 (m, 6 H), 5.30-5.24 (m, 1 H), 4.03 (t, J = 5.4 Hz, 2 H), 3.66 (t, J = 5.4 Hz, 2 H), 3.32 (s, 3 H), 2.93-2.74 (m, 2 H), 2.21-2.11 (m, 1 H), 2.05-1.87 (m, 3 H). MS (ESI) m/z 366.1 (M + H)⁺. Anal. calcd. for C₂₁H₂₃N₃O₃: C, 69.02; H, 6.34; N, 11.50; O, 13.13. Found: C, 69.08; H, 6.52; N; 11.51. IR (KBr)νₘₐₓ 1726, 1520, 1383, 1171 cm⁻¹. mp 115.6 degrees Celsius. |
| Example 89 | 2-oxo-3-(2-pyrrolidin-1-ylethyl)-N-(1,2,3,4-tetrahydronaphthalen-1-yl)-2,3- |
| | dihydro-1H-benzimidazole-1-carboxamidehydrochloride |
| 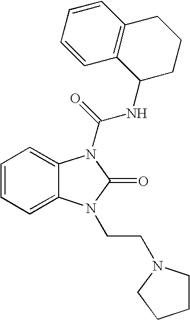 | ¹H-NMR (300 MHz, CDCl₃). δ 13.19 (bs, 1 H), 8.84 (d, J = 8.4 Hz, 1 H), 8.27 (dd, J = 7.8, 0.9 Hz, 1 H), 7.58 (d, J = 7.2 Hz, 1 H), 7.41-7.38 (m, 1 H), 7.32-7.11 (m, 4 H), 5.29-5.23 (m, 1 H), 4.55-4.51 (m, 2 H), 3.78 (bs, 2 H), 3.89 (t, J = 7.2 Hz, 2 H), 2.91-2.77 (m, 4 H), 2.21-2.13 (m, 4 H), 2.03-1.71 (m, 4 H). MS (ESI) m/z 405.2 (M + H)⁺. Anal. calcd. for C₂₄H₂₈N₄O₂ (+1.0 HCl, 0.3 H₂O): C, 64.58; H, 6.68; N, 12.55; O, 8.24; Cl, 7.94. Found: C, 64.67; H, 7.08; N; 12.56. IR (KBr)νₘₐₓ 1728, 1533, 1489, 1383 cm⁻¹. mp 165.6 degrees Celsius. |
| Example 90 | 3-[(1-Methylpiperidin-2-yl)methyl]-N-1-naphthyl-2-oxo-2,3-dihydro-1H- |
| | benzimidazole-1-carboxamide hydrochloride |
| 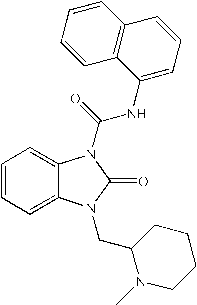 | ¹H-NMR (300 MHz, CDCl₃). δ 12.98 (bs, 1 H), 11.24 (s, 1 H), 8.33 (dd, J = 7.8, 0.9 Hz, 1 H), 8.25 (dd, J = 7.8, 0.9 Hz, 1 H), 8.11 (d, J = 8.7 Hz, 1 H), 7.92-7.89 (m, 1 H), 7.73 (d, J = 8.1 Hz, 1 H), 7.63-7.52 (m, 4 H), 7.37-7.24 (m, 2 H), 4.72 (dd J = 14.7, 5.1 Hz, 1 H), 4.60-4.52 (m, 1 H), 3.57-3.45 (m, 2 H), 2.97 (s, 3 H), 2.90-2.81 (m, 1 H), 2.33-2.21 (m, 2 H), 2.01-1.86 (m, 3 H), 1.51-1.43 (m, 1 H). MS (ESI) m/z 415.1 (M + H)⁺. Anal. calcd. for C₂₅H₂₆N₄O₂ (+0.7 H₂O, 1.0 HCl, 0.2 C₄H₈O₂) C, 64.40; H, 6.28; N, 11.64; O, 10.31; Cl, 7.37. Found: C, 64.56; H, 6.06; N; 11.32. IR (KBr)νₘₐₓ 1740, 1570, 1383 cm⁻¹. |

Following Examples 91 to 92 were prepared according to the procedure described in Example 3.

| | |
|---|---|
| Example 91 | N-[(1S, 2S)-2-Methyl-1-(morpholin-4-ylcarbonyl)butyl]-3-(2-morpholin-4- ylethyl)-2-oxo-2,3-dihydro-1H-benzimidazole-1-carboxamlde hydrochloride |
| 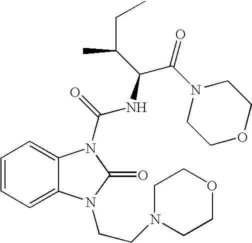 | ¹H-NMR (270 MHz, CDCl₃) δ 9.23 (d, J = 8.9 Hz, lH), 8.15 (d, J = 7.8 Hz, 1H), 7.60-7.48 (m, 1H), 7.31-7.17 (m, 2H), 4.87 (dd, J = 8.37, 6.21 Hz, 1H), 4.76-4.45 (m, 2H), 4.35-3.95 (m, 4H), 3.80-3.25 (m, 12H), 3.07-2.87 (m, 2H), 1.92-1.83 (m, 1H), 1.70-1.55 (m, 1H), 1.28-1.17 (m, 1H), 1.04 (d, J = 6.75 Hz, 3H), 0.94 (t, J = 7.3 Hz, 3H). MS (ESI) m/z 474 (M + H)⁺. Anal. calcd. for C₂₄H₃₅N₅O₅ (+1 H₂O, 1 HCl): C, 54.59; H, 7.25; N, 13.26; O, 18.18, Cl, 6.71. Found: C, 54.52; H, 7.16; N; 12.86. |
| Example 92 | N-[(1S)-2,2-Dimethyl-1-(pyrrolidin-1-ylcarbonyl)propyl]-3-{2-morpholin-4- ylethyl)-2-oxo-2,3-dihydro-1H-benzimidazole-1-carboxamide hydrochloride |
| 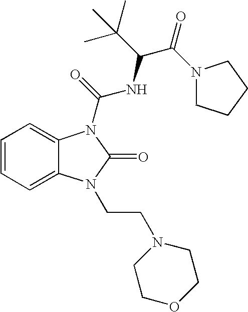 | ¹H-NMR (300 MHz, DMSO-d₆) δ 11.46 (bs, 1H), 9.18 (d, J = 9.0 Hz, 1H), 8.05 (d, J = 7.8 Hz, 1H), 7.53 (d, J = 7.5 Hz, 1H), 7.34-7.17 (m, 2H), 4.60 (d, J = 9.0 Hz, 1H), 4.40 (t, J = 6.3 Hz, 2H), 4.06-3.98 (m, 2H), 3.84-3.72 (m, 2H), 3.70-3.28 (m, 12H), 3.26-3.15 (m, 2H), 1.95-1.87 (m, 2H), 1.83-1.75 (m, 2H), 1.02 (s, 9H). MS (ESI) m/z 458 (M + H)⁺. Anal. calcd. for C₂₄H₃₅N₅O₄ (+1 H₂O, 1 HCl): C, 56.30; H, 7.48; N, 13.68; O, 15.62, Cl, 6.92. Found: C, 56.53; H, 7.60; N; 13.35. |

### Example 93

### N-[(1S)-2,2-dimethyl-(2-methyl-2H-tetrazol-5-yl)propyl]-3-(2-morpholin-4-ylethyl)-2-oxo-2,3-dihydro-1H-benzimidazole-1-carboxamide

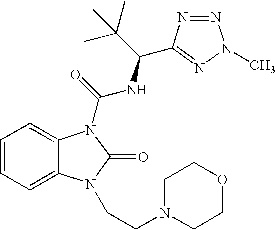

### STEP 1. benzyl [(1S)-2,2-dimethyl-1-(2H-tetrazol-5-yl)propyl]carbamate

The title compound was prepared according to the procedure described in the literature (Demko. Z. P.; Sharpless, K. B. Org. Lett. 2002, 4, 2525-2527.) from benzyl [(1S)-1-(aminocarbonyl)-2,2-dimethylpropyl]carbamate (Step 1 of Example 4).

MS (ESI) m/z 290 (M+H)⁺, 288 (M−H)⁻.

### STEP 2. benzyl [(1S)-2,2-dim ethyl-1-(2-methyl-2H-tetrazol-5-yl)propyl]carbamate and benzyl [(1S)-2,2-dimethyl-1-(1-methyl-1H-tetrazol-5-yl)propyl]carbamate

To a suspension of benzyl [(1S)-2,2-dimethyl-1-(2H-tetrazol-5-yl)propyl]carbamate (280 mg, 0.96 mmol), potassium carbonate (660 mg, 4.8 mmol) and methyl iodide (0.24 mL, 3.8 mmol) in acetone (5 mL) was stirred at 0 degrees Celsius for 10 minutes and warmed to room temperature. After 4 hours, the mixture was filtered and concentrated. The residue was purified by column chromatography on silica gel eluting with hexane/ethyl acetate (8/1-4/1-1/1) to afford 166 mg (57%) of benzyl [(1S)-2,2-dimethyl-1-(2-methyl-2H-tetrazol-5-yl)propyl]carbamate and 82 mg (28%) of benzyl [(1S)-2,2-dimethyl-1-(1-methyl-1H-tetrazol-5-yl)propyl]carbamate.

### benzyl [(1S)-2,2-dimethyl-1,2-methyl-2H-tetrazol-5-yl)propyl]carbamate

¹H-NMR (300 MHz, CDCl₃) δ 7.40-7.28 (m, 5H), 5.59 (d, J=9.3 Hz, 1H), 5.12 (d, J=12.3 Hz, 1H), 5.06 (d, J=12.3 Hz, 1H), 5.00 (d, J=9.3 Hz, 1H), 4.32 (s, 3H), 0.97 (s, 9H).

MS (ESI) m/z 304 (M+H)⁺.

### benzyl [(1S)-2,2-dimethyl-1-(1-methyl-1H-tetrazol-5-yl)propyl]carbamate

¹H-NMR (300 MHz, CDCl₃) δ 7.40-7.28 (m, 5H), 5.55 (d, J=9.6 Hz, 1H), 5.10 (d, J=12.6 Hz, 1H), 5.02 (d, J=12.6 Hz, 1H), 4.84 (d, J=9.6 Hz, 1H), 4.13 (s, 3H), 1.05 (s, 9H).

MS (ESI) m/z 304 (M+H)⁺.

### STEP 3. N-[(1S)-2,2-dimethyl-1-(2-methyl-2H-tetrazol-5-yl)propyl]-3-(2-morpholin-4-ylethyl)-2-oxo-2,3-dihydro-1H-benzimidazole-1-carboxamide

The titled compound was prepared according to the procedure described in Step 4 of example 1 from (1S)-2,2-dimethyl-1-(2-methyl-2H-tetrazol-5-yl)propan-1-amine which was prepared from benzyl [(1S)-2,2-dimethyl-1-(2-methyl-2H-tetrazol-5-yl)propyl]carbamate according to the procedure described in step 2 of example 4.

¹H-NMR (300 MHz, CDCl₃) δ 13.9 (bs, 1H), 9.55-9.32 (m, 1H), 8.22-8.01 (m, 1H), 7.68-7.43 (m, 1H), 7.35-7.05 (m, 1H), 5.24-5.15 (m, 1H), 4.78-4.47 (m, 2H), 4.40-3.85 (m, 7H), 3.62-3.18 (m, 4H), 3.12-2.80 (m, 2H), 1.08 (s, 9H).

MS (ESI) m/z 443 (M+H)⁺.

Anal. calcd. for C₂₁H₃₀N₈O₃ (+0.5H₂O, 1.0 HCl, 0.5 C₄H₈O₂): C, 51.92; H, 6.82; N, 21.06; O, 13.53; Cl, 6.66. Found: C, 51.73; H, 6.79; N, 21.20.

### Example 94

### N-[(1S)-2,2-dimethyl-1-(1-methyl-1H-tetrazol-6-yl)propyl]-3-(2-morpholin-4-ylethyl)-2-oxo-2,3-dihydro-1H-benzimidazole-1-carboxamide

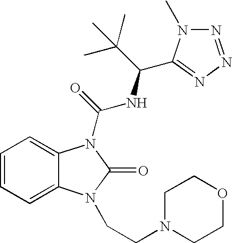

The titled compound was prepared according to the procedure described in Step 2 of Example 12 from (1S)-2,2-dimethyl-1-(1-methyl-1H-tetrazol-5-yl)propan-1-amine which was prepared from benzyl [(1S)-2,2-dimethyl-1-(1-methyl-1H-tetrazol-5-yl)propyl]carbamate according to the procedure described in Step 2 of Example 4.

¹H-NMR (300 MHz, CDCl₃) δ 9.72 (d, J=8.1 Hz, 1H), 8.06 (d, J=7.2 Hz, 1H), 7.23-7.00 (m, 4H), 5.09 (d, J=8.1 Hz, 1H), 4.22 (s, 3H), 4.07-3.95 (m, 2H), 3.73-3.61 (m, 4H), 2.75-2.44 (m, 6H), 1.17 (s, 9H).

MS (ESI) m/z 443 (M+H)⁺.

### Example 95

### N-[(1S)-1-(aminocarbonyl)-2,2-dimethylpropyl]-3-(2-methyl-2-methylpropyl)-2-oxo-2,3-dihydro-1H-benzimidazole-1-carboxamide

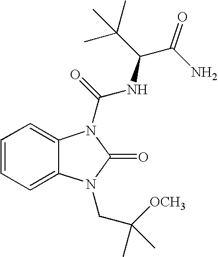

### STEP 1. 2-methoxy-2-methylpropan-1-amine

To a suspension of lithium aluminum hydride (2.1 g, 55 mmol) and diethyl ether (30 mL) at 0 degrees Celsius was added a solution of 2-methoxy-2-methylpropanenitrile (prepared from 2.5 g (29 mmol) of 2-hydroxy-2-methylpropanenitrile according to the procedure in the literature (U.S. Pat. No. 4,864,051)) in diethyl ether (20 mL). The mixture was refluxed for 7 hours. Then the reaction mixture was quenched by addition of water (2.1 mL), 15% NaOH (2.1 mL) and water (6.3 mL) at 0 degrees Celsius and stirred at room temperature for 14 hours. The mixture was filtered and concentrated in vacuo to give crude material (1.5 g).

¹H-NMR (300 MHz, CDCl₃) δ 3.20 (s, 3H), 2.65 (s, 2H), 1.14 (s, 9H).

### STEP 2. 1-(2-methoxy-2-methylpropyl)-1,3-dihydro-2H-benzimidazol-2-one

The title compound was prepared according to the procedure described in Steps 1, 2 and 3 of Example 1 from 2-methoxy-2-methylpropan-1-amine.

MS (ESI) m/z 221 (M+H)⁺, 219 (M−H)⁻.

### STEP 3. N-[(1S)-1-(aminocarbonyl)-2,2-dimethylpropyl]-3-(2-methyl-2-methylpropyl)-2-oxo-2,3-dihydro-1H-benzimidazole-1-carboxamide

The title compound was prepared according to the procedure described in Step 2 of Example 12 without recrystallization.

¹H-NMR (300 MHz, CDCl₃) δ 9.49 (d, J=8.1 Hz, 1H), 8.16-8.11 (m, 1H), 7.31-7.23 (m, 1H), 7.22-7.10 (m, 2H), 5.97 (bs 1H), 5.65 (bs, 1H), 4.24 (d, J=8.1 Hz, 1H), 3.90 (d, J=14.4 Hz, 1H), 3.85 (d, J=14.4 Hz, 1H), 3.20 (s, 3H), 1.27 (s, 3H), 1.26 (s, 3H), 1.15 (s, 9H).

MS (ESI) m/z 377 (M+H)⁺.

### Example 96

### N-[1S)-1-(aminocarbonyl)-2,2-dimethylpropyl]-3-(2-hydroxy-2-methylpropyl)-2-oxo-2,3-dihydro-1H-benzimidazole-1-carboxamide

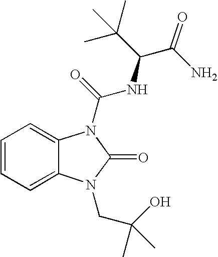

### STEP 1. 1-amino-2-methylpropan-2-ol hydrochloride

The title compound was prepared according to the procedure described in Step1 of Example 95 from 2-hydroxy-2-methylpropanenitrile.

¹H-NMR (300 MHz, CDCl₃, the value of free form of the title compound) δ 4.39-3.96 (s, 1H), 2.61 (s, 2H), 1.17 (s, 6H).

### STEP 2. 1-(2-hydroxy-2-methylpropyl)-1,3-dihydro-2H-benzimidazol-2-one

The title compound was prepared according to the procedure described in Steps 1, 2 and 3 of Example 1 from 1-amino-2-methylpropan-2-ol hydrochloride.

¹H-NMR (300 MHz, CDCl₃) δ 10.43 (bs, 1H), 7.13-7.04 (m, 4H), 3.92 (s, 2H), 3.63 (s, 1H), 1.33 (s 6H).

MS (ESI) m/z 207 (M+H)⁺, 205 (M−H)⁻.

### STEP 3 N-[(1S)-1(aminocarbonyl)-2,2-dimethylpropyl]-3-(2-hydroxy-2-methylpropyl)-2-oxo-2,3-dihydro-1H-benzimidazole-1-carboxamide

The title compound was prepared according to the procedure described in Step 2 of Example 12.

¹H-NMR (300 MHz, CDCl₃) δ 9.40 (d, J=8.1 Hz, 1H), 8.21-8.15 (m, 1H), 7.22-7.15 (m, 3H), 5.84 (bs 1H), 5.54 (bs, 1H), 4.22 (d, J=8.1 Hz, 1H), 2.13 (s, 2H), 2.36 (s, 1H), 1.35 (s, 6H), 1.15 (s, 9H).

MS (ESI) m/z 363 (M+H)⁺.

Anal. calcd. for C₁₈H₂₆N₄O₄ (+0.1H₂O): C, 59.36; H, 7.25; N, 15.38; O, 18.01. Found: C, 59.45; H, 7.25; N, 15.00.

### Example 97

### N-[(1S)-1-cyano-2,2-dimethylpropyl]-3-(2-morpholin-4-ylethyl)-2-oxo-2,3-dihydro H-benzimidazole-1-carboxamide

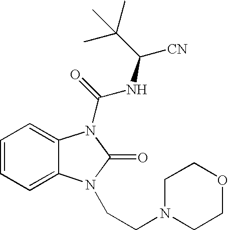

### STEP 1. benzyl [(1S)-1-cyano-2,2-dimethylpropyl]carbamate

The title compound was prepared according to the procedure described in the literature (Demko. Z. P.; Sharpless, K. B. Org. Lett. 2002, 4, 2525-2527.) from benzyl [(1S)-1-(aminocarbonyl)-2,2-dimethylpropyl]carbamate (Step1 of Example 4).

MS (ESI) m/z 247 (M+H)⁺.

### STEP 2. N-[(1S)-1-cyano-2,2-dimethylpropyl]-3-(2-morpholin-4-ylethyl)-2-oxo-2,3-dihydro-1H-benzimidazole-1-carboxamide

The title compound was prepared according to the procedure described in Step 2 of Example 12 from (2S)-2-amino-3,3-dimethylbutanenitrile which was prepared from benzyl [(1S)-cyano-2,2-dimethylpropyl]carbamate according to the procedure described in Step 2 of Example 4.

¹H-NMR (300 MHz, CDCl₃) δ 9.45 (d, J=9.0 Hz, 1H), 8.20-8.16 (m, 1H), 7.28-7.18 (m, 2H), 7.09-7.03 (m, 1H), 4.77 (d, J=9.0 Hz, 1H), 4.04-3.98 (m, 2H), 3.70-3.62 (m, 4H), 2.77-2.63 (m, 2H), 2.60-2.48 (m, 4H), 1.18 (s, 9H).

MS (ESI) m/z 386 (M+H)⁺.

Anal. calcd. for C₂₀H₂₇N₅O₃: C, 62.32; H, 7.06; N, 18.17; O, 12.45. Found: C, 61.99; H, 7.01; N, 17.96.

### Example 98

### N-[(1S)-1-(aminocarbonyl)-2,2-dimethylpropyl]-5-methyl-3-(2-morpholin-4-ylethyl)-2-oxo-2,3-dihydro-1H-imidazo[4,5-b]pyridine-1-carboxamide

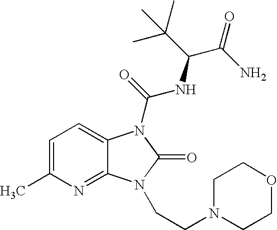

### STEP 1. 5-methyl-3-(2-morpholin-4-ylethyl)-1,3-dihydro-2H-imidazo[4,5-b]pyridin-2-one

The title compound was prepared from 2-chloro-3-nitro-6 picoline according to the procedure described in Steps 1, 2 and 3 of Example 1.

¹H-NMR (300 MHz, CDCl₃) δ 9.94 (bs, 1H), 7.12 (d, J=7.5 Hz, 1H), 6.80 (d, J=7.5 Hz, 1H), 4.18-4.05 (m, 2H), 3.70-3.55 (m, 4H), 2.83-2.71 (m, 2H), 2.65-2.53 (m, 4H), 2.50 (s, 3H).

MS (ESI) m/z 263 (M+H)⁺, 261 (M−H)⁻.

### STEP 2. N-[(1S)-1-(aminocarbonyl)-2,2-dimethylpropyl]-5-methyl-3-(2-morpholin-4-ylethyl)-2-oxo-2,3-dihydro-1H-imidazo[4,5-b]pyridine-1-carboxamide (PF-03407918-01)

The title compound was prepared from 5-methyl-3-(2-morpholin-4-ylethyl)-1,3-dihydro-2H-imidazo[4,5b]pyridin-2-one and L-tert-leucinamide according to the procedure described in Step 4 of Example 1.

¹H-NMR (300 MHz, DMSO-d₆) δ 11.29 (bs, 1H), 8.93 (d, J=8.7 Hz, 1H), 8.07 (d, J=7.8 Hz, 1H), 7.73 (bs, 1H), 7.26 (bs, 1H), 7.06 (d, J=7.8 Hz, 1H), 4.38-4.22 (m, 3H), 4.06-3.59 (m, 8H), 3.25-3.08 (m, 2H), 2.48 (s, 3H), 0.99 (s, 9H).

MS (ESI) m/z 419 (M+H)⁺.

Anal. calcd. for C₂₀H₃₀N₆O₄ (0.6H₂O, 1.0 HCl, 0.1 C₄H₈O₂): C, 51.63; H, 7.01; N, 17.71; O, 16.18; Cl, 7.47. Found: C, 51.88; H, 7.14; N, 17.48.

### Example 99

### N-[(1S)-1-(aminocarbonyl)-2-methylpropyl]-5-methyl-3-(2-morpholin-4-ylethyl)-2-oxo-2,3-dihydro-1H-imidazo[4,5-b]pyridine-1-carboxamide

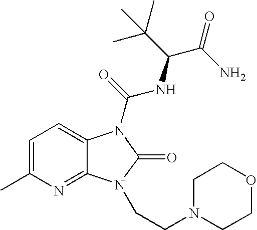

The title compound was prepared from 5-methyl-3-(2-morpholin-4-ylethyl)-1,3-dihydro-2H-imidazo[4,5-b]pyridin-2-one and L-valinamide hydrochloride according to the procedure described in Step 4 of Example 1.

¹H-NMR (300 MHz, DMSO-d₆) δ 11.22 (bs, 1H), 8.82 (d, J=8.7 Hz, 1H), 8.07 (d, J=7.8 Hz, 1H), 7.73 (bs, 1H), 7.28 (bs, 1H), 7.07 (d, J=7.8 Hz, 1H), 4.38-4.26 (m, 3H), 4.06-3.60 (m, 8H), 3.27-3.08 (m, 2H), 2.48 (s, 3H), 2.19-2.08 (m, 1H), 0.96 (d, J=7.2 Hz, 3H), 0.89 (d, J=6.6 Hz, 3H).

MS (ESI) m/z 405 (M+H)⁺.

Anal. calcd. for C₁₉H₂₈N₆O₄ (0.5H₂O, 1.0 HCl, 0.1 C₄H₈O₂): C, 50.79; H, 6.77; N, 18.32; O, 16.39; Cl, 7.73. Found: C, 50.46; H, 6.90; N, 17.93.

### Example 100

### N-[(1S)-2,2-dimethyl-1-(3-methyl-1,2,4-oxadiazol-5-yl)propyl]-3-[2-(4-morpholinyl)ethyl]-2-oxo-2,3-dihydro-1H-benzimidazole-1-carboxamide

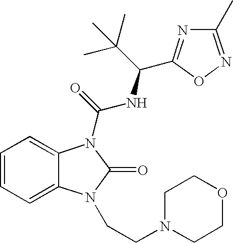

### STEP 1. E,Z mixture of N-{(1S)-1-[({[1-aminoethylidene]amino}oxy)carbonyl]-2,2-dimethylpropyl}-3-[2-(4-morpholinyl)ethyl]-2-oxo-2,3-dihydro-1H-benzimidazole-1-carboxamide

To a solution of N-({3-[2-(4-morpholinyl)ethyl]-oxo-2,3-dihydro-1H-benzimidazol-1-yl}carbonyl)-L-tert-leucine (prepared according to the procedure described in step 1 of example 3 from methyl L-tert-leucinate hydrochloride) (0.18 g, 0.45 mmol) in DMF (1 mL) were added a solution of N-hydroxyethanimidamide (37 mg, 0.50 mmol, Hamze, A.; Hernandez, J.-F.; Fulcrand, P.; Martinez, J. J. Org. Chem. 2003, 68, 7316-7321.) In DMF (1 mL), triethylamine (0.26 mL, 1.8 mmol), HOBt (83 mg, 0.54 mmol) and WSC (0.10 g, 0.54 mmol) at room temperature. After 9 h, to this mixture were added N-hydroxyethanimidamide (20 mg, 0.26 mmol), triethylamine (0.10 mL, 0.70 mmol), HOBt (10 mg, 0.06 mmol) and WSC (20 mg, 0.10 mmol). After 13 hours, the reaction was quenched by addition of sat. aq. sodium bicarbonate (10 mL) and extracted with ethyl acetate (20 mL×2). The combined organic layers were washed with water (10 mL×2), brine (20 mL) and dried over sodium sulfate, filtered and concentrated. The residue was purified by column chromatography on silica gel eluting with dichloromethane/methanol (10/1) to afford 0.12 g (57%) of the title compound.

MS (ESI) m/z 461 (M+H)⁺.

### STEP 2. N-[(1S)-2,2-dimethyl-1-(3-methyl-1,2,4-oxadiazol-5-yl)propyl]-3-[2-(4-morpholinyl)ethyl]-2-oxo-2,3-dihydro-1H-benzimidazole-1-carboxamide

To a solution of N-{(1S)-1-[({[aminoethylidene]amino}oxy)carbonyl]-2,2-dimethylpropyl}-3-[2-(4-morpholinyl)ethyl]-2-oxo-2,3-dihydro-1H-benzimidazole-1-carboxamide (0.11 g, 0.24 mmol) in toluene (5 mL) was added p-toluenesulfonic acid monohydrate (4 mg, 0.02 mmol) and the mixture was refluxed for 6 hours. Then the reaction was cooled to room temperature and quenched by addition of water (10 mL) and extracted with ethyl acetate (20 mL×2). The combined organic layers were washed with brine (20 mL) and dried over sodium sulfate, filtered and concentrated. The residue was purified by preparative TLC eluting with dichloromethane/methanol (10/1) to afford 62 mg (58%) of the title compound. The subsequent recrystallization from ethyl acetate and hexane followed by filtration gave 48 mg of the title compound as white solid.

¹H-NMR (300 MHz, CDCl₃) [symbol] 9.68 (d, J=8.1 Hz, 1H), 8.15 (d, J=8.1. Hz, 1H), 7.30-7.11 (m, 2H), 7.08-7.01 (m, 1H), 5.18 (d, J=8.1 Hz, 1H), 4.09-4.00 (m, 2H), 3.72-3.63 (m, 4H), 2.75-2.67 (m, 2H), 2.61-2.48 (m, 4H), 2.41 (s, 3H), 1.12 (s, 9H).

MS (ES I) m/z 443 (M+H)⁺.

Anal. calcd. for C₂₂H₃₀N₆O₄(+0.2H₂O): C, 59.23; H, 6.87; N, 18.84; O, 15.06. Found: C, 59.14; H, 7.00; N, 18.50.

### Example 101

### N-[(1S)-1-(aminocarbonyl)-2,2-dimethylpropyl]-3-(3-hydroxy-3-methylbutyl)-2-oxo-2,3-dihydro-1H-benzimidazole-1 carboxamide

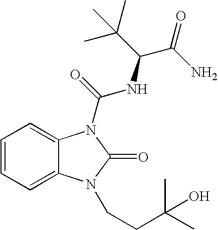

### STEP 1. 3-hydroxy-3-methylbutanenitrile

To a solution of 1-chloro-2-methylpropan-2-ol (17 g, 0.16 mol) in ethanol (320 mL) and water (55 mL) was added sodium cyanide (9.4 g, 0.19 mol) and the mixture was refluxed. After 3 hours, the mixture was cooled to room temperature and concentrated in vacuo. To the residue was added water and extracted with ethyl acetate. The combined organic layers were dried over magnesium sulfate and concentrated to give 14 g (90%) of the title compound.

¹H-NMR (300 MHz, CDCl₃) δ 2.54 (s, 2H), 2.03 (s, 1H), 1.42 (s, 6H).

### STEP 2. 4-amino-2-methyl-2-butanol

To a solution of 3-hydroxy-3-methylbutanenitrile (16 g, 0.16 mol) in THF (350 mL) was added lithium aluminumhydride (12 g, 0.33 mol) slowly at 0 degrees Celsius and the mixture was stirred for 4 hours at 50 degrees Celsius. After cooling to 0 degrees Celsius, to the mixture were added sodium sulfate decahydrate and potassium fluoride. The mixture was stirred for 30 minutes at room temperature and filtered through a pad of celite. The filtrate was concentrated in vacuo to give 14 g (84%) of the title compound.

¹H-NMR (300 MHz, CDCl₃) [symbol] 3.03 (t, J=6.8 Hz, 2H), 1.58 (t, J=6.8 Hz, 2H), 1.24 (s, 6H).

### STEP 3. 2-methyl-4-[(2-nitrophenyl)amino]-2-butanol

A solution of 1-fluoro-2-nitrobenzene (1.9 mL, 18 mmol), 4-amino-2-methyl-2-butanol (1.6 g, 15 mmol) and triethylamine (6.4 mL, 46 mmol) in THF (30 mL) was refluxed for 8 h. Then the reaction was quenched by addition of water (50 mL) and extracted with ethyl acetate (50 mL×2). The combined organic layers were washed with brine (50 mL) and dried over sodium sulfate, filtered and concentrated. The residue was purified by column chromatography on silica gel eluting with hexane/ethyl acetate (5/1-2/1) to afford 2.8 g (82%) of the title compound.

MS (ESI) m/z 225 (M+H)⁺.

### STEP 4. 4-[(2-aminophenyl amino]-2-methyl-2-butanol

The title compound was prepared according to the procedure described in step 2 of example 1 from 2-methyl-4-[(2-nitrophenyl)amino]-2-butanol.

MS (ESI) m/z 195 (M+H)⁺.

### STEP 5. 1-(3-hydroxy-3-methylbutyl)-1,3-dihydro-2H-benzimidazol-2-one

The title compound was prepared according to the procedure described in step 3 of acetate (0.10 L) and washed with water (50 mL×2), brine (50 mL), dried over sodium sulfate, filtered and concentrated to give a crude material. The another batch starting from 1.3 g of 2-chloro-3-nitrotoluene was combined to this crude material and the combined crude products were purified by column chromatography on silica gel eluting with hexane/ethyl acetate (3/1) to afford 2.6 g (77%) of the title compound.

MS (ESI) m/z 227 (M+H)⁺.

### STEP 2. 7-methyl-1-[2-(methylthio)ethyl]-1,3-dihydro-2H-benzimidazol-2-one

To a solution of 2-methyl-N-[2-(methylthio)ethyl]-6-nitroaniline (2.6 g, 12 mmol) in ethanol (6.0 mL) was added a solution of Tin(II) chloride dihydrate (7.9 g, 35 mmol) in concd. hydrochloric acid (8.0 mL) at 0 degrees Celsius and warmed to room temperature. After 4 hours, the reaction was quenched by addition of 6N sodium hydroxide (100 mL) and extracted with ethyl acetate (100 mL×2), dried over sodium sulfate, filtered and concentrated. The crude material was dissolved in THF (50 mL) and to this solution was added CDI (2.3 g, 14 mmol) and the mixture was stirred at room temperature. After 12 hours, to the mixture was added CDI (1.5 g, 6.7 mmol) and the reaction mixture was refluxed for 5 hours. Then the mixture was cooled to room temperature and evaporated to dryness. To this was added water (100 mL) and extracted with ethyl acetate (100 mL×2). The combined organic layers were washed with water (50 mL), brine (50 mL), dried over sodium sulfate, filtered and concentrated. The obtained material was dissolved in methanol (30 mL) and to this solution was added 2N sodium hydroxide (3 mL) and stirred at room temperature for 2 hours. Then the mixture was quenched by addition of sat. aq. sodium bicarbonate (50 mL) and extracted with ethyl acetate (100 mL×2). The combined organic layers were washed with water (50 mL), brine (50 mL), dried over sodium sulfate, filtered and concentrated. The residue was purified by column chromatography on silica gel eluting with dichloromethane/methanol (20/1-10/1) to afford 1.8 g (69%) of the title compound.

MS (ESI) m/z 223 (M+H)⁺.

### STEP 3. N-{(1S)-1-[(dimethylamino)carbonyl]-2,2-dimethylpropyl}-4-methyl-3-[2-methylthio)ethyl]-2-oxo-2,3-dihydro-1H-benzimidazole-1-carboxamide

To a solution of 7-methyl-1-[2-(methylthio)ethyl]-1,3-dihydro-2H-benzimidazol-2-one (0.21 g, 0.93 mmol) in 1,2-dichloroethane (5 mL) were added triethylamine (0.43 mL, 3.1 mmol) and 4-nitrophenyl chloroformate (0.23 g, 1.1 mmol) at 0 degrees Celsius and the mixture was stirred at room temperature for 4 hours. Then to this mixture was added a solution of N,N-dimethyl-tert-leucinamide (ca. 1.4 mmol, prepared according to the procedure described in steps 1 and 2 of example 3 from dimethylamine hydrochloride) in 1,2-dichloroethane (3 mL) at 0 degrees Celsius and stirred at room temperature. After 14 hours, the reaction was quenched by addition of water (50 mL) and extracted with dichloromethane (50 mL×2). The combined organic layers were washed with water (30 mL×4), brine (30 mL), dried over sodium sulfate, filtered and concentrated in vacuo. The residue was purified by preparative TLC eluting with dichloromethane/methanol (10/1) to afford 0.31 g (83%) of the title compound.

¹H-NMR (300 MHz, CDCl₃) [symbol] 9.56 (d, J=9.0 Hz, 1H), 8.10 (d, J=7.8 Hz, 1H), 7.07-6.98 (m, 1H), 6.94 (d, J=7.5 Hz, 1H), 4.92 (d, J=9.0 Hz, 1H), 4.32-4.22 (m, 2H), 3.23 (s, 3H), 3.00 (s, 3H), 2.85-2.77 (m, 2H), 2.59 (s, 3H), 2.22 (s, 3H), 1.10 (s, 9H).

MS (ESI) m/z 407 (M+H)⁺.

Anal. calcd. for C₂₀H₃₀N₄O₃S: C, 59.09; H, 7.44; N, 13.78; O, 11.81; S, 7.89. Found: C, 58.97; H, 7.45; N, 13.67.

### Example 103

### N-{(1S)-1-[(dimethylamino)carbonyl]-2,2-dimethylpropyl}-4-methyl-3-[2-(methylsulfonyl)ethyl]-2-oxo-2,3-dihydro-1H-benzimidazole-1-carboxamide

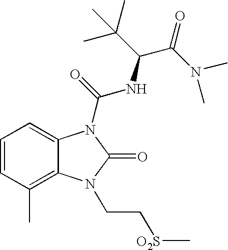

To a solution of N-{(1S)-1-[(dimethylamino)carbonyl]-2,2-dimethylpropyl}-4-methyl-3-[2-methylthio)ethyl]-2-oxo-2,3-dihydro-1H-benzimidazole-1-carboxamide (EXAMPLE 102, 0.27 g, 0.67 mmol) in dichloromethane (22 mL) were added m-CPBA (0.57 g, 2.3 mmol) and sodium bicarbonate (0.15 g, 1.7 mmol) at room temperature and stirred for 14 hours. Then the reaction was quenched by addition of sat. aq. sodium thiosulfate (50 mL) and the aqueous layer was extracted with dichloromethane (50 mL). The combined organic layers were washed with water (30 mL), brine (30 mL), dried over sodium sulfate, filtered and concentrated. The residue was purified by preparative TLC eluting with dichloromethane/methanol (10/1) to afford 0.19 g (66%) of the title compound. The obtained solid was then recrystallized from hexane/ethylacetate to give 164 mg of the title compound.

¹H-NMR (300 MHz, CDCl₃) [symbol] 9.45 (d, J=8.7 Hz, 1H), 8.11 (d, J=7.5 Hz, 1H), 7.10-7.05 (m, 1H), 6.98 (d, J=7.2 Hz, 1H), 4.92 (d, J=8.7 Hz, 1H), 4.66-4.55 (m, 2H), 3.53-3.44 (m, 2H), 3.22 (s; 3H), 3.04 (s, 3H), 2.99 (s, 3H), 2.64 (s, 3H), 1.10 (s, 9H).

MS (ESI) m/z 439 (M+H)⁺.

Anal. calcd. for C₂₀H₃₀N₄O₅S: C, 54.78; H, 6.90; N, 12.78; O, 18.24; S, 7.31. Found: C, 54.42; H, 6.90; N, 12.50.

mp 190.7 degrees Celsius.

### Example 104

### N-[(1S)-(hydroxymethyl)-2,2-dimethylpropyl]-3-[2-(methylsulfonyl)ethyl]-2-oxo-2,3-dihydro-1H-benzimidazole-1-carboxamide

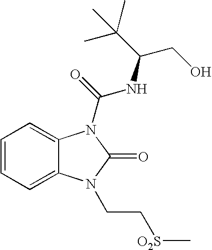

### STEP 1. N-[2-(methylthio)ethyl]-2-nitroaniline

The title compound was prepared according to the procedure described in step 1 of example 1 from 2-(methylthio)ethylamine.

MS (ESI) m/z 213 (M+H)⁺.

### STEP 2. 1-[2-(methylthio)ethyl]-1,3-dihydro-2H-benzimidazol-2-one

The title compound was prepared according to the procedure described in step 2 of EXAMPLE 102.

MS (ESI) m/z 209 (M+H)⁺.

### STEP 3. N-[(1S)-1-(hydroxymethyl)-2,2-dimethylpropyl]-3-[2-(methylsulfonyl)ethyl]-2-oxo-2,3-dihydro-1H-benzimidazole-1-carboxamide

The title compound was prepared according to the procedure described in steps 3 of EXAMPLE 102 and EXAMPLE 103 starting from L-tert-leucinol.

¹H-NMR (270 MHz, CDCl₃) [symbol] 8.89 (d, J=8.4 Hz, 1H), 8.24-8.21 (m, 1H), 7.30-7.14 (m, 3H), 4.41 (t, J=7.3 Hz, 2H), 4.04-3.86 (m, 2H), 3.73-3.61 (m, 1H), 3.49 (t, J=7.3 Hz, 2H), 2.96 (s, 3H), 2.27-2.18 (m, 1H), 1.05 (s, 9H).

MS (ESI) m/z 384 (M+H)⁺.

Anal. calcd. for C₁₇H₂₅N₃OFS (+0.2H₂O): C, 52.75; H, 6.61; N, 10.86; O, 21.49; S, 8.28. Found: C, 52.44; H, 6.61; N, 10.68.

### Example 105

### N-[(1S)-2,2-dimethyl-1-(2-methyl-2H-tetrazol-5-yl)propyl]-3-(3-hydroxy-3-methylbutyl)-2-oxo-2,3-dihydro-1H-benzimidazole-1-carboxamide

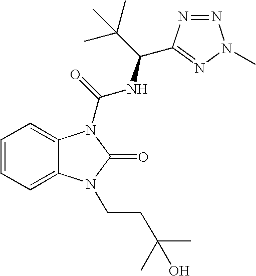

### STEP 1. benzyl[(1S)-2,2-dimethyl-1-(2H-tetrazol-5-yl)propyl]carbamate

The title compound was prepared according to the procedure described the literature (Olah, G. A. et al. Synthesis 1980, 657-658.; Demko, Z. P. and Sharpless, K. B. Org. Lett. 2002, 4, 2525-2527.) starting from benzyl[(1S)-1-(aminocarbonyl)-2,2-dimethylpropyl]carbamate.

MS (ESI) m/z 290 (M+H)⁺.

### STEP 2. benzyl[(1S)-2,2-dimethyl-1-(2-methyl-2H-tetrazol-5-yl)propyl]carbamate

A suspension of benzyl[(1S)-2,2-dimethyl-1-(2H-tetrazol-5-yl)propyl]carbamate (0.41 g, 1.4 mmol), potassium carbonate (1.0 g, 7.0 mmol) and methyl iodide (0.35 mL, 5.6 mmol) in acetone (7 mL) was stirred at 0 degrees Celsius for 10 minutes and warmed to room temperature. After 5 hours, the reaction mixture was filtered and concentrated. The residue was purified by column chromatography on silica gel eluting with hexane/ethyl acetate (8/1-4/1-1/1) to afford 0.29 g (68%) of the title compound.

MS (ESI) m/z 304 (M+H)⁺.

### STEP 3. (1S)-2,2-dimethyl-1-(2-methyl-2H-tetrazol-5-yl)-1-propanamine

The title compound was prepared according to the procedure described in step 2 of example 3 starting from benzyl[(1S-2,2-dimethyl-1-(2-methyl-2H-tetrazol-5-yl)propyl]carbamate.

MS (ESI) m/z 170 (M+H)⁺.

### STEP 4. N-[(1S)-2,2-dimethyl-1-(2-methyl-2H-tetrazol-5-yl)propyl]-3-(3-hydroxy-3-methylbutyl)-2-oxo-2,3-dihydro-1H-benzimidazole-1-carboxamide

The title compound was prepared according to the procedure described in step 4 of EXAMPLE 101 starting from 1-(3-hydroxy-3-methylbutyl)-1,3-dihydro-2H-benzimidazol-2-one and (1S)-2,2-dimethyl-1-(2-methyl-2H-tetrazol-5-yl)-1-propanamine.

¹H-NMR (300 MHz, CDCl₃) [symbol] 9.67 (d, J=9.3 Hz, 1H), 8.16 (dd, J=7.2, 1.5 Hz, 1H), 7.23-7.12 (m, 2H), 7.06 (d, J=7.2 Hz, 1H), 5.30 (d, J=9.3 Hz, 1H), 4.34 (s, 3H), 4.11-4.06 (m, 2H), 1.95-1.90 (m, 2H), 1.34 (s, 6H), 1.08 (s, 9H).

MS (ESI) m/z 416 (M+H)⁺.

Anal. calcd. for C₂₀H₂₉N₇O₃ (+0.1H₂O): C, 57.57; H, 7.05; N, 23.50; O, 11.89. Found: C, 57.29; H, 7.13; N, 23.10.

### Example 106

### N-[(1S)-2,2-dimethyl-1-(5-methyl-1,3,4-thiadiazol-2-yl)propyl]-3-(3-hydroxy-3-methylbutyl)-2-oxo-2,3-dihydro-1H-benzimidazole-a-carboxamide

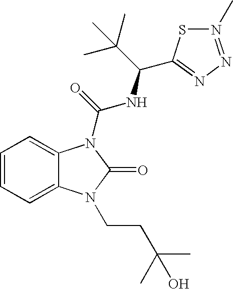

### STEP 1. benzyl{(1S)-2,2-dimethyl-1-(5-methyl-1,3,4-thiadiazol-2-yl)propyl}carbamate

The title compound was prepared according to the procedure described in the literature (Alker, D. et al. J. Med. Chem. 1989, 32, 2381-2388.) starting from N-[(benzyloxy)carbonyl]-tert-leucine.

¹H-NMR (300 MHz, CDCl₃) δ 7.41-7.28 (m, 5H), 5.79-5.75 (m, 1H), 5.14-4.99 (m, 3H), 2.78 (s, 3H), 1.30 (s, 9H).

MS (ESI) m/z 320 (M+H)⁺.

### STEP 2. (1S)-2,2-dimethyl-1-(5-methyl-1,3,4-thiadiazol-2-yl)-1-propSnamine hydrochloride

A solution of benzyl{(1S)-2,2-dimethyl-1-(5-methyl-1,3,4-thiadiazol-2-yl)propyl}carbamate (ca. 0.6 mmol) in anhydrous hydrogen bromide in acetic acid (25% solution, 1 mL) was stirred at room temperature for 4 hours. Then to this mixture was added ether (50 mL) (precipitate was observed.). The supernatant fluid was decanted. The process of wash with ether followed by decantation was repeated twice and the resultant solid was dried in vacuo to give the crude material of the title compound.

¹H-NMR (300 MHz, DMSO-d₆) δ 4.91-4.89 (m, 1H), 2.77 (s, 3H), 1.01 (s, 9H).

### STEP 3. N-[(1S)-2,2-dimethyl-1-(5-methyl-1,3,4-thiadiazol-2-yl)propyl]-3-(3-hydroxy-3-methylbutyl)-2-oxo-2,3-dihydro-1H-benzimidazole-a-carboxamide

To a solution of 1-(3-hydroxy-3-methylbutyl)-1,3-dihydro-2H-benzimidazol-2-one (0.12 g, 0.55 mmol) in 1,2-dichloroethane (18 mL) were added triethylamine (0.25 mL, 1.8 mmol) and 4-nitrophenyl chloroformate (0.13 g, 0.66 mmol) at 0 degrees Celsius and the mixture was stirred at room, temperature for 4 hours. Then to this mixture was added a suspension of (1S)-2,2-dimethyl-1-(5-methyl-1,3,4-thiadiazol-2-yl)-propanamine hydrochloride and triethylamine (0.2 mL, 1.4 mmol) in 1,2-dichloroethane (5 mL) at 0 degrees Celsius and stirred room temperature. After 14 hours, the reaction was quenched by addition of water (30 mL) and extracted with dichloromethane (30 mL×3). The combined organic layers were washed with water (30 mL×5), brine (30 mL), dried over sodium sulfate, filtered and concentrated in vacuo. The residue was purified by preparative TLC twice eluting with THF/hexane (1/1) and dichloromethane/methanol (10/1) respectively to afford 15 mg (6%) of the title compound.

¹H-NMR (300 MHz, CDCl₃) [symbol] 9.70 (d, J=10.2 Hz, 1H), 8.15 (d, J=9.0 Hz, 1H), 7.22-7.13 (m, 2H), 7.07 (d, J=8.7 Hz, 1H), 5.31 (d, J=9.0 Hz, 1H), 4.13-4.05 (m, 2H), 2.74 (s, 3H), 1.98-1.88 (m, 2H), 1.34 (s, 6H), 1.15 (s, 9H).

MS (ESI) m/z 432 (M+H)⁺.

### Example 107

### N-[(1S)-(1-aminocarbonyl)-2,2-dimethylpropyl]-3-(3-amino-3-methylbutyl)-2-oxo-2,3-dihydro-1H-benzimidazole-1-carboxamide hydrochloride

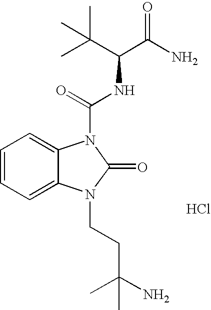

### STEP 1. N-{1,1-dimethyl-3-[(2-nitrophenyl)amino]propyl}formamide

To a mixture of 2-methyl-4-[(2-nitrophenyl)amino]butan-2-ol (1.7 g, 7.8 mmol) and trimethylsilyl cyanide (4.2 mL, 31 mmol) was added conod. sulfuric acid at −30 degrees Celsius and the mixture was warmed up to room temperature. After stirring for 24 hours, the reaction mixture was cooled to 0 degrees Celsius and to the mixture was added water and stirred for 30 minutes at room temperature. The mixture was poured into aq. potassium carbonate and extracted with dichloromethane. The combined organic layers were dried over magnesium sulfate and concentrated in vacuo. The crude product was purified by column chromatography on silica gel eluting with dichloromethane/methanol (25/1) to give 1.3 g (66%) of the title compound.

¹H-NMR (300 MHz, CDCl₃) (a mixture of rotamers) δ 8.34-7.84 (m, 3H), 7.53-7.36 (m, 1H), 6.96-6.57 (m, 2H), 6.02 (bs, 0.2H), 5.35 (bs, 0.8H), 3.53-3.31 (m, 2H), 2.38-1.97 (m, 2H), 1.44 (s, 6H).

MS (ESI) m/z 252 (M+H)⁺.

### STEP 2. N-[1,1-dimethyl-3-(2-oxo-2,3-dihydro-1H-benzimidazol-1-yl)propyl]formamide

A mixture of N-{1,1-dimethyl-3-[(2-nitrophenyl)amino]propyl}formamide (1.3 g, 5.1 mmol) and palladium on charcoal (0.13 g) in THF (20 mL) was stirred under hydrogen atmosphere (4 atm) for 6 hours. The mixture was filtered through a celite pad and the filtrate was concentrated in vacuo.

The obtained crude product was dissolved in THF (20 mL) and to this solution was added CDI (1.0 g, 6.2 mmol). After stirring for 16 hours at room temperature, water was added to the solution. Then it was extracted with ethyl acetate. The combined organic layers were dried over magnesium sulfate and concentrated. The residue was purified by column chromatography on silica gel eluting with dichloromethane/methanol (25/1) to give 1.1 g of a mixture of the title compound and an impurity.

MS (ESI) m/z 248 (M+H)⁺.

### STEP 3. 1-(3-amino-3-methylbutyl)-1,3-dihydro-2H-benzimidazol-2-one

A mixture of N-[1,1-dimethyl-3-(2-oxo-2,3-dihydro-1H-benzimidazol-1-yl)propyl]formamide (1.1 g) and hydrogen chloride-methanol (80-90% methanol, 18 mL) was stirred at rt for 50 h. The mixture was concentrated in vacuo and the residue was basified by aq. potassium carbonate and the mixture was extracted with dichloromethane. The combined organic layers were dried over sodium sulfate and concentrated to give 0.81 g (3.7 mmol, 73% for 3 steps) of the title compound.

¹H-NMR (300 MHz, CDCl₃) δ 7.14-6.97 (m, 4H), 4.02 (t, J=7.5 Hz, 2H), 1.83 (t, J=7.5 Hz, 2H), 1.22 (s, 6H).

MS (ESI) m/z 220 (M+H)⁺.

### STEP 4. tert-butyl [1,1-dimethyl-3-(2-oxo-2,3-dihydro-1H-benzimidazol-1-yl)propyl]carbamate

To a suspension of 1-(3-amino-3-methylbutyl)-1,3-dihydro-2H-benzimidazol-2-one (0.22 g, 1.0 mmol) in dichloromethane (2 mL) and THF (2 mL) were added triethylamine (0.28 mL, 2.0 mmol) and di-tert-butyl dicarbonate (0.24 g, 1.1 mmol) at room temperature. After stirring for 1.5 hours, the mixture was diluted with ethyl acetate and washed with water and brine, dried over magnesium sulfate and concentrated in vacuo. The residue was purified by column chromatography on silica gel eluting with dichloromethane/methanol (25/1) to give 0.32 g (quantitative yield) of the title compound.

¹H-NMR (300 MHz, CDCl₃) δ 9.51 (bs, 1H), 7.19-6.95 (m, 4H), 3.93 (t, J=8.3 Hz, 2H), 3.49 (bs, 1H), 2.18 (t, J=8.3 Hz, 2H), 1.44 (s, 9H), 1.36 (s, 6H).

MS (ESI) m/z 320 (M+H)⁺.

### STEP 5. tert-butyl {3-[3-({[(1S)-1-(aminocarbonyl)-2,2-dimethylpropyl]amino}carbonyl)-2-oxo-2,3-dihydro-1H-benzimidazol-1-yl]-1,1-dimethylpropyl}carbamate

The title compound was prepared according to the procedure described in Step 4 of Example 1 from tert-butyl [1,1-dimethyl-3-(2-oxo-2,3-dihydro-1H-benzimidazol-1-yl)propyl]carbamate.

¹H-NMR (300 MHz, CDCl₃) δ 9.46 (d, J=9.0 Hz, 1H), 8.15 (d, J=6.0 Hz, 1H), 7.37-7.04 (m, 3H), 5.80 (bs, 1H), 5.42 (bs, 1H), 4.54 (s, 1H), 4.22 (d, J=9.0 Hz, 1H), 4.00-3.83 (m, 2H), 2.31-2.08 (m, 2H), 1.42 (s, 6H), 1.36 (s, 9H), 1.16 (s, 9H).

MS (ESI) m/z 476 (M+H)⁺.

### STEP 6. N-[(1S)-1-(aminocarbonyl)-2,2-dimethylpropyl]-3-(3-amino-3-methylbutyl)-2-oxo-2,3-dihydro-1H-benzimidazole-1-carboxamide hydrochloride

To a solution of tert-butyl {3-[3-({[(1S)-1-(aminocarbonyl)-2,2-dimethylpropyl]amino}carbonyl)-2-oxo-2,3-dihydro-1H-benzimidazol-1-yl]-1,1-dimethylpropyl}carbamate (0.23 g, 0.48 mmol) in methanol (1.5 mL) was added hydrogen chloride-methanol (80-90% methanol, 6 mL). After stirring at room temperature for 40 hours, the reaction mixture was concentrated in vacuo. The residue was added a mixture of hexane and ethyl acetate and the precipitate was filtered and dried to give 0.18 g (88%) of the title compound.

¹H-NMR (DMSO-d₆) δ 9.19 (d, J=9.0 Hz, 1H), 8.16 (bs, 2H), 8.06 (d, J=6.0 Hz, 1H), 7.67 (bs, 1H), 7.41-7.10 (m, 4H), 4.27 (d, J=9.0 Hz, 1H), 4.02 (t, J=7.5 Hz, 2H), 1.98 (t, J=7.5 Hz, 2H), 1.36 (s, 6H), 1.00 (s, 9H).

MS (ESI) m/z 376 (M+H)⁺.

Anal. calcd. for C₁₉H₂₉N₅O₃ (+3.0H₂O, 1.3 HCl): C, 47.85; H, 7.67; N, 14.68; O, 20.13; Cl, 9.66.

Found: C, 47.74; H, 7.43; N, 14.71.

Following Examples 108 to 149 were prepared according to the procedure described in step 4 of Example 1.

| | |
|---|---|
| Example 108 | Rel-3-[2-(dimethylamino)ethyl]-N-[(1R,2S)-2-hydroxycyclohexyl]-4-methyl- 2-oxo-2,3-dihydro-1H-benzimidazole-1-carboxamide |
| 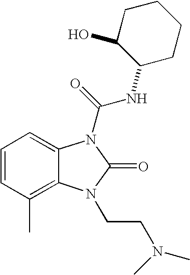 | ¹H-NMR (300 MHz, DMSO) δ 10.63 (bs, 1H), 8.76 (d, J = 7.2 Hz, 1H), 8.01 (d, J = 7.5 Hz, 1H), 7.09-7.00 (m, 1H), 4.88 (bs, 1H), 4.45 (t, J = 6.6 Hz, 2H), 3.55-3.45 (m, 1H), 3.42 (t, J = 6.6 Hz, 2H), 2.87 (s, 6H), 2.59 (s, 3H), 2.10-1.98 (m, 1H), 1.93-1.80 (m, 1H), 1.69-1.51 (m, 2H), 1.36-1.15 (m, 4H). MS (ESI) m/z 361 (M + H)⁺. Anal. calcd. for C₁₉H₂₈N₄O₃ (+1 HCl· 0.2 H₂O): C, 56.98; H, 7.40; N, 13.99; O, 12.78; Cl, 8.85. Found: C, 56.58; H, 7.41; N, 13.81. |
| Example 109 | N-[(1S)-1-(aminocarbonyl)-2,2-dimethylpropyl]-3-{2-[methyl (methylsulfonyl)amino]ethyl}-2-oxo-2,3-dihydro-1H- benzimidazole-1-carboxamide |
| 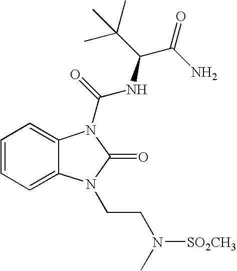 | ¹H-NMR (270 MHz, CDCl₃) δ 9.38 (d, J = 7.8 Hz, 1H), 8.17 (d, J = 7.3 Hz, 1H), 7.31 -7.16 (m, 3H), 5.82 (bs, 1H), 5.48 (bs, 1H), 4.22 (d, J = 7.8 Hz, 1H), 4.19-4.09 (m, 2H), 3.53-3.45 (m, 2H), 2.93 (s, 3H), 2.80 (s, 3H), 1.15 (s, 9H).MS (ESI) m/z 426 (M + H)⁺. |
| Example 110 | N-[(1S)-1-(aminocarbonyl)-2,2-dimethylpropyl]-3- (cyclopropylmethyl)-2-oxo-2,3-dihydro-1H-imidazo[4,5-b]pyridine- 1-carboxamide |
| 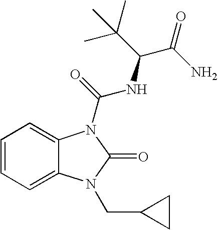 | ¹H-NMR (300 MHz, DMSO-d₆) δ 9.01 (d, J = 9.0 Hz, 1H), 8.21-8.15 (m, 2H), 7.69 (bs, 1H), 7.24-7.17 (m, 2H), 4.26 (d, J = 9.0 Hz, 1H), 3.86-3.65 (m, 2H), 1.34-1.17 (m, 1H), 0.99 (s, 9H), 0.54-0.35 (m, 4H). MS (ESI) m/z 346 (M +H)⁺. Anal. calcd. for C₁₇H₂₃N₅O₃: C, 59.12; H, 6.71; N, 20.28; O, 13.90. Found: C, 58.73; H, 6.77; N; 19.93. |
| Example 111 | N-[(1S)-1-(aminocarbonyl)-2,2-dimethylpropyl]-3-(cyclopropyl- methyl)-4-methyl-2-oxo-2,3-dihydro-1H-Imidazo[4,5-c]pyridine- 1-carboxamide. |
| 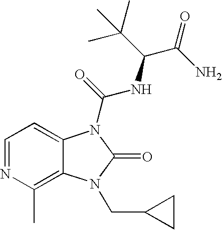 | ¹H-NMR (300 MHz, CDCl₃) δ 9.46 (d, J = 8.1 Hz, 1H), 8.30 (d, J = 5.7 Hz, 1H), 8.02 (d, J = 5.7 Hz, 1H), 5.75 (bs, 1H), 5.52 (bs, 1H), 4.19 (d, J = 8.1 Hz, 1H), 4.08-3.95 (m, 2H), 2.87 (s, 3H), 1.25-1.02 (m, 10H), 0.65-0.46 (m, 4H). MS (ESI) m/z 360 (M + H)⁺. Anal. calcd. for C₁₈H₂₅N₅O₃ (+0.7 H₂O): C, 58.11; H, 7.15; N, 18.82; O, 15.91. Found: C, 58.24; H, 7.12; N, 18.45. |
| Example 112 | N-[(1S)-1-(aminocarbonyl)-2,2-dimethylpropyl]-2- oxo-3-(1,1,3,3-tetramethylbutyl)-2,3-dihydro-1H- benzimidazole-1-carboxamide |
| 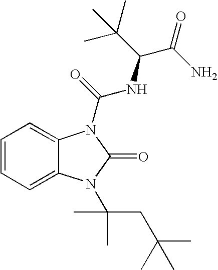 | ¹H-NMR (270 MHz, CDCl₃) δ 9.72 (d, J =7.8 Hz, 1H), 8.29-8.25 (m, 1H), 7.48-7.44 (m, 1H), 7.16-7.09 (m, 2H), 5.88 (bs, 1H), 5.50 (bs, 1H), 4.22 (d, J = 7.8 Hz, 1H), 2.17 (d, J = 15.4 Hz, 1H), 2.03 (d, J = 15.4 Hz, 1H), 1.90 (s, 3H), 1.89 (s, 3H), 1.15 (s, 9H), 0.88 (s, 9H). MS (El) m/z 402 (M)⁺. Anal. calcd. for C₂₂H₃₄N₄O₃ (+0.5 H₂O): C, 64.21; H, 8.57; N, 13.61; O, 13.61. Found: C, 64.54; H, 8.70; N, 13.44. |
| Example 113 | N-[(1S)-1-(aminocarbonyl)-2,2-dimethylpropyl]-6- methyl-3-[2-(4-morpholinyl)ethyl]-2-oxo-2,3-dihydro- 1H-benzimidazole-1-carboxamide |
| 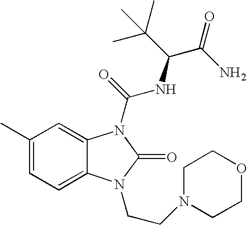 | ¹H-NMR (270 MHz, DMSO-d₆) δ 10.74 (bs, 1H), 9.08 (d, J = 9.2 Hz, 1H), 7.86 (bs, 1H), 7.64 (bs, 1H), 7.31 (d, J = 7.8 Hz, 1H), 7.16 (bs, 1H), 7.04 (d, J = 7.8 Hz, 1H), 4.35-4.25 (m, 2H), 4.19 (d, J = 9.2 Hz, 1H), 4.02-3.84 (m, 2H), 3.80-3.35 (m, OH), 3.20-3.00 (m, 2H), 2.32 (s, 3H), 0.94 (s, 9H). MS (ESI) m/z 418 (M + H)⁺. Anal. calcd. for C₂₁H₃₁N₅O₄ (+1.0 HCl, 0.2 C₄H₈O₂, 1.2 H₂O): C, 53.09; H, 7.36; N, 14.20; O, 18.17; Cl, 7.19. Found: C, 53.04; H, 7.27; N, 14.26. |
| Example 114 | N-[(1S)-1-(aminocarbonyl)-2,2-dimethylpropyl]-3-[2-(dimethyl- amino)ethyl]-2-oxo-4-(trifluoromethyl)-2,3-dihydro-1H- benzimidazole-1-carboxamide |
| 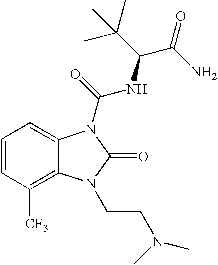 | ¹H-NMR (300 MHz, CDCl₃) δ 9.20 (d, J = 8.7 Hz, 1H), 8.47 (d, J = 8.1 Hz, 1H), 7.68 (bs, 1H), 7.59 (d, J = 8.1 Hz, 1H), 7.37-7.29 (m, 1H), 7.24 (bs, 1H), 4.26 (d, J = 8.7 Hz, 1H), 4.11-3.98 (m, 2H), 3.41-3.25 (m, 2H), 2.20 (s, 6H), 0.98 (s, 9H). MS (ESI) m/z 430 (M + H)⁺. |
| Example 115 | N-[(1S)-1-(aminocarbonyl)-2,2-dimethylpropyl]-5-fluoro- 3-[2-(4-morpholinyl)ethyl]-2-oxo-2,3-dihydro-1H-benzimidazole- 1-carboxamide |
| 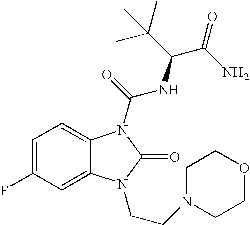 | ¹H-NMR (300 MHz, DMSO-d₆) δ 11.03 (bs, 1H), 9.00 (d, J = 9.6 Hz, 1H), 8.02 (dd, J = 9.6, 5.4 Hz, 1H), 7.69 (bs, 1H), 7.54 (d, J = 7.5 Hz, 1H), 7.22 (bs, 1H), 7.03 (dt, J = 9.6, 3.0 Hz, 1H), 4.42-4.30 (m, 2H), 4,26 (d, J = 9.6 Hz, 1H), 4.09-3.93 (m, 2H), 3.85-3.42 (m, 6H), 3.26-3.08 (m, 2H), 0.99 (s, 9H). MS (ESI) m/z 422 (M + H)⁺. Anal. calcd. for C₂₀H₂₈N₅O₄F (+1.0 HCl, 0.1 C₈H₁₄, 0.5 H₂O): C, 52.03; H, 6.66; N, 14.73; O, 15.14; F, 4.00; Cl, 7.46. Found: C, 51.67; H, 6.81; N, 14.40. |
| Example 116 | N-[(1S)-1-(aminocarbonyl)-2,2-dimethylpropyl]-3-(3-hydroxy- 3-methylbutyl)-4-methyl-2-oxo-2,3-dihydro-1H-benzimldazole- 1-carboxamide |
| 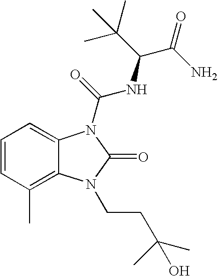 | ¹H-NMR (300 MHz, DMSO-d₆) δ 9.34 (d, J = 9.0 Hz, 1H), 7.98 (dd, J = 7.2, 2.1 Hz, 1H), 7.66 (bs, 1H), 7.20 (bs, 1H), 7.06-6.99 (m, 2H), 4.51 (s, 1H), 24.24 (d, J = 9.0 Hz, 1H), 4.20-4.09 (m, 2H), 2.61 (s, 3H), 1.79-1.70 (m, 2H), 1.20 (s, 6H), 0.99 (s, 9H). MS (ESI) m/z 391 (M + H)⁺. Anal. calcd. for C₂₀H₃₀N₄O₄: C, 61.52; H, 7.74; N) 14.35; O, 16.39. Found: C, 61.17; H, 7.71; N, 14.20. |
| Example 117 | N-[(1S)-1-(aminocarbonyl)-2,2-dimethylpropyl]-3-(1-hydroxy- cyclobutyl)methyl]-2-oxo-2,3-dihydro-1H-benzimidazole-1- carboxamide |
| 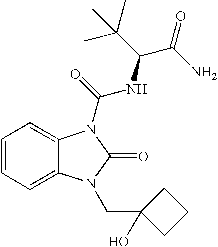 | ¹H-NMR (300 MHz, DMSO-d₆) δ 9.24 (d, J = 8.7 Hz, 1H), 8.03 (d, J = 6.6 Hz, 1H), 7.68 (bs, 1H), 7.40 (bs, J = 8.1 Hz, 1H), 7.22-7.10 (m, 3H), 5.40 (s, 1H), 4.27 (d, J = 8.7 Hz, 1H), 4.01 (d, J = 15.0 Hz, 1H), 3.94 (d, J = 15.0 Hz, 1H), 2.20-2.14 (m, 2H), 2.00-1.90 (m, 2H), 1.71-1.58 (m, 2H), 0.99 (s, 9H). MS (ESI) m/z 375 (M + H)⁺. Anal. calcd. for C₁₉H₂₆N₄O₄ (+0.1 H₂O): C, 60.65; H, 7.02; N, 14.89; O, 17.44. Found: C, 60.44; H, 7.03; N, 14.62. mp 158 degrees Celsius. |
| Example 118 | N-((1S)-2,2-dimethyl-1-{[(methylsulfonyl)amino]methyl}propyl)-3- [2-(4-morpholinyl)ethyl]-2-oxo-2,3-dihydro-1H-benzimidazolone- 1-carboxamide |
| 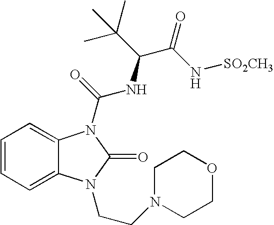 | ¹H-NMR (300 MHz, CDCl₃) δ 9.07 (d, J = 8.7 Hz, 1H), 8.20-8.17 (m, 1H), 7.24-7.17 (m, 2H), 7.07-7.04 (m, 1H), 5.02-4.93 (m, 1H), 4.07-3.89 (m, 3H), 3.68-3.56 (m, 5H), 3.15 (ddd, J = 12.3, 10.2, 4.2 Hz, 1H), 2.97 (s, 3H), 2.77-2.65(m, 2H), 2.63-2.47 (m, 4H), 1.06 (s, 9H). MS (ESI) m/z 468 (M + H)⁺. Anal. calcd. for C₂₁H₃₃N₅O₅S: C, 53.33; H, 7.16; N, 14.81; O, 17.93; S, 6.78. Found: C, 53.12; H, 7.02; N, 14.68. |
| Example 119 | N-[(1S)-1-(aminocarbonyl)-2,2-dimethylpropyl]-3-[2-(dimethyl- amino)ethyl]-4-fluoro-2-oxo-2,3-dihydro-1H-benzimidazole- 1-carboxamide |
| 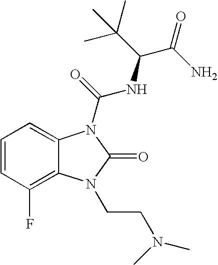 | ¹H-NMR (300 MHz, DMSO-d₆) δ 9.97 (bs, 1H), 9.08 (d, J = 8.7 Hz, 1H), 7.97-7.90 (m, 1H), 7.70 (bs, 1H), 7.28-7.14 (m, 3H), 4.38-4.27 (m, 3H), 3.54-3.42 (m, 2H), 2.89 (s, 6H), 0.99 (s, 9H). MS (ESI) m/z 380 (M + H)⁺. Anal. calcd. for C₁₈H₂₆N₅O₃F (+1.0 HCl, 0.5 IPA, 0.5 H₂O): C, 51.48; H, 7.09; N, 15.39; O, 14.07; F, 4.18; Cl, 7.79. Found: C, 51.71; H, 7.20; N, 15.19. |
| Example 120 | 3-(3-hydroxy-3-methylbutyl)-N-[(1S)-1-(hydroxymethyl)-2,2- dimethylpropyl]-2-oxo-2,3-dihydro-1H-benzimidazole-1-carboxamide |
| 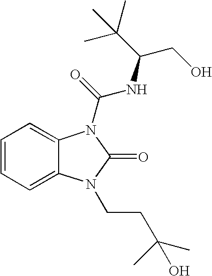 | ¹H-NMR (300 MHz, CDCl₃) δ 9.05 (d, J = 8.7 Hz, 1H), 8.21 (dd, J = 7.2, 1.5 Hz, 1H), 7.24-7.14 (m, 2H), 7.06 (dd, J = 7.2, 1.5 Hz, 1H), 4.09-3.88 (m, 4H), 3.70-3.63 (m, 1H), 2.41 (bs, 1H), 1.94-1.89 (m, 2H), 1.77 (bs, 1H), 1.34 (s, 6H), 1.05 (s, 9H). MS (ESI) m/z 364 (M + H)⁺. Anal. calcd. for C₁₉H₂₉N₃O₄ (+0.3 H₂O): C, 61.87; H, 8.09; N, 11.39; O, 18.65. Found: C, 61.93; H, 8.18; N, 11.37. |
| Example 121 | N-[(1S)-2,2-dimethyl-1-(1-methyl-1H-tetrazol-5-yl)propyl]-3-(3- hydroxy-3-methylbutyl)-2-oxo-2,3-dihydro-1 H-benzimidazole- 1-carboxamide |
| 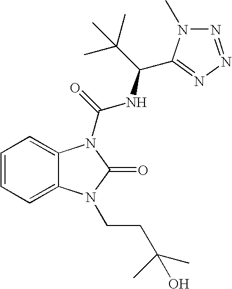 | ¹H-NMR (270 MHz, CDCl₃) δ 9.74-9.70 (m, 1H), 8.06 (d, J = 8.1 Hz, 1H), 7.23-7.05 (m, 3H), 5.09 (d, J = 7.8 Hz, 1H), 4.22 (s, 3H), 4.09-4.03 (m, 2H), 1.94-1.88 (m, 2H), 1.34 (s, 3H), 1.33 (s, 3H), 1.17 (s, 9H). MS (ESI) m/z 416 (M + H)⁺. |
| Example 122 | N-{(1S)-2,2-dimethyl-1-[(methylsulfonyl)methyl]propyl}-3- (3-hydroxy-3-methylbutyl)-2-oxo-2,3-dihydro-1H-benzimidazole- 1-carboxamide |
| 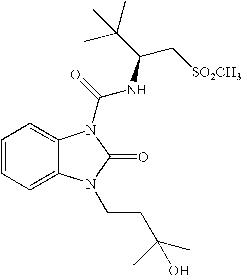 | ¹H-NMR (300 MHz, CDCl₃) δ 9.14 (d, J = 9.6 Hz, 1H), 8.22 (d, J = 6.6 Hz, 1H), 7.24-7.16 (m, 2H), 7.09-7.07 (m, 1H), 4.54-4.40 (m, 1H), 4.15-3.97 (m, 2H), 3.34 (d, J = 14.7 Hz, 1H), 3.11 (dd, J = 14.7, 10.2 Hz, 1H), 3.02 (s, 3H), 1.94-1.89 (m, 2H), 1.33 (s, 6H), 1.05 (s, 9H). MS (ESI) m/z 426 (M + H)⁺. Anal. calcd. for C₂₀H₃₁N₃O₅S (+0.3 H₂O): C, 55.74; H, 7.39; N, 9.75; O, 19.68; S, 7.44. Found: C, 55.56; H, 7.42; N, 9.66. |
| Example 123 | 3-(3-hydroxy-3-methylbutyl)-N-[1-(hydroxymethyl)cyclopentyl- 2-oxo-2,3-dihydro-1H-benzimidazole-1-carboxamide |
| 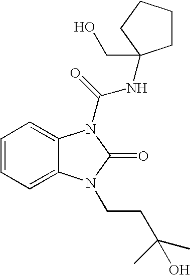 | ¹H-NMR (300 MHz, CDCl₃) δ 9.06 (s, 1H), 8.18 (d, J = 7.2 Hz, 1H), 7.25-7.14 (m, 2H), 7.09-7.02 (m, 1H), 4.09-3.93 (m, 3H), 3.84-3.76 (m, 2H), 1.94-1.66 (m, 10H), 1.33 (s, 6H). MS (ESI) m/z 362 (M + H)⁺. Anal. calcd. for C₁₉H₂₇N₃O₄: C, 62.51; H, 7.57; N, 11.51; O, 18.41. Found: C, 62.41; H, 7.60; N, 11.24. |
| Example 124 | N-[1-(aminocarbonyl)-2,2-dimethylpropyl]-3-[(1-hydroxycyclohexyl) methyl]-2-oxo-2,3-dihydro-1H-benzimidazole-1-carboxamide |
| 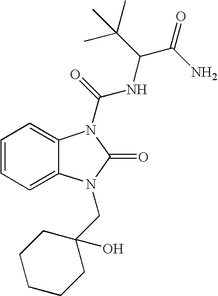 | ¹H-NMR (270 MHz, CDCl₃) δ 9.42 (d, J = 7.9 Hz, 1H), 8.19-8.16 (m, 1H), 7.22-7.07 (m, 3H), 5.90 (br, 1H), 5.58 (br, 1H), 4.22 (d, J = 7.9 Hz, 1H), 3.89 (s, 2H), 2.28 (d, J = 3.3 Hz, 1H), 1.14 (s, 9H), 1.64-1.52 (m, 10H) MS (ESI) m/z 403 (M + H)⁺. |
| Example 125 | N-[1-(aminocarbonyl)-2,2-dimethylpropyl]-2-oxo-3-(tetrahydrofuran- 2-ylmethyl)-2,3-dihydro-1H-benzimidazole-1-carboxamide |
| 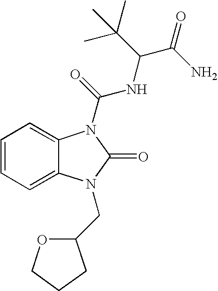 | ¹H-NMR (270 MHz, CDCl₃) δ 9.47-9.44 (m, 1H), 8.17 (d, J = 7.3 Hz, 1H), 7.24-7.13 (m, 3H), 4.30-4.19 (m, 2H), 4.08-3.70 (m, 4H), 2.11-1.68 (m, 4H), 1.15 (s, 9H). MS (ESI) m/z 375 (M + H)⁺. |
| Example 126 | N-[2,2-dimthyel-1-(pyrrolidin-1-ylmethyl)propyl]-3-(2-morpholin- 4-ylethyl)-2-oxo-2,3-dihydro-1H-benzimidazole-1-carboxamide |
| 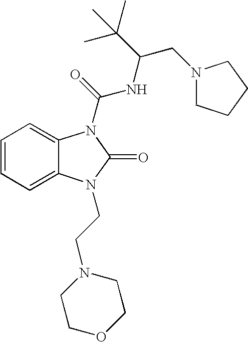 | ¹H-NMR (270 MHz, CDCl₃) δ 8.87 (d, J = 9.9 Hz, 1H), 8.24-8.21 (m, 1H), 7.23-7.13 (m, 2H), 7.05-7.01 (m, 1H), 4.14-3.98 (m, 3H), 3.67 (t, J = 4.6 Hz, 4H), 3.00-2.66 (m, 8H), 2.60-2.47 (m, 4H), 1.76-1.89 (m, 4H), 1.02 (s, 9H). MS (ESI) m/z 444 (M + H)⁺. |
| Example 127 | N-(1-acetyl-2,2-dimethylpropyl)-3-(2-morpholin-4-ylethyl)-2-oxo- 2,3-dihydro-1H-benzimidazole-1-carboxamide |
| 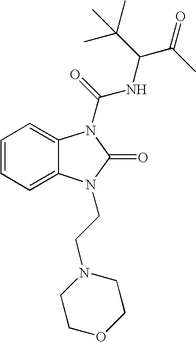 | ¹H-NMR (300 MHz, CDCl₃) δ 9.37 (d, J = 7.3 Hz, 1H), 8.09 (dd, J = 2.0, 7.3 Hz, 1H), 7.18-7.07 (m, 2H), 6.99-6.96 (m, 1H), 4.37 (d, J = 7.3 Hz, 1H), 3.95 (t, J = 6.6 Hz, 2H), 3.62-3.59 (m, 4H), 2.63 (t, J = 6.6 Hz, 2H), 2.54-2.41 (m, 4H), 2.55 (s, 3H), 1.04 (s, 9H). MS (ESI) m/z 403 (M + H)⁺. |
| Example 128 | N-[(1S)-1-(aminocarbonyl)-2,2-dimethylpropyl]-4-methyl-2-oxo-3-(2-pyr- roliden-1-ylethyl)-2,3-dihydro-1H-benzimidazole-1-carboxamide hydrochloride |
| 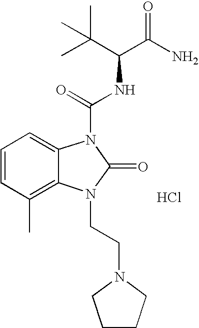 | ¹H-NMR (270 MHz, CDCl₃) δ 13.1-12.8 (m, 1H), 9.41-9.23 (m, 1H), 8.13-7.94 (m, 1H), 7.11-6.77 (m, 2H), 6.31-6.05 (m, 1H), 5.68-5.45 (m, 1H), 4.90-4.40 (m, 2H), 4.27-3.70 (m, 3H), 3.17-2.80 (m, 1H), 2.70 (bs, 3H), 2.47-1.88 (m, 4H), 1.22-0.81 (m, 13H). MS (ESI) m/z 402 (M + H)⁺. Anal. calcd. for C₂₁H₃₁N₅O₃ (+1.0 H₂O, 1.0 HCl): C, 55.32; H, 7.52; N, 15.36; O, 14.04; Cl, 7.78. Found: C, 55.53; H, 7.55; N; 15.08. |
| Example 129 | N-[(1S)-1-(aminocarbonyl)-2,2-dimethylpropyl]-3- (2-hydroxy-2-methylpropyl)4-methyl-2-oxo-2,3-dihydro-1H-benzimidazole- 1-carboxamide |
| 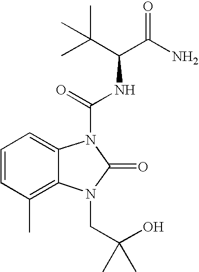 | ¹H-NMR (300 MHz, CDCl₃) δ 9.49 (d, J = 9.0 Hz, 1H), 8.15 (d, J = 8.3 Hz, 1H), 7.07 (dd, J = 8.3, 8.3 Hz, 1H), 6.98 (d, J = 8.3 Hz, 1H), 5.88 (bs, 1H), 5.57 (bs, 1H), 4.23 (s, 2H), 4.21 (d, J = 9.0 Hz, 1H), 3.05 (bs, 1H), 2.63 (s, 3H), 1.33 (s, 6H), 1.14 (s, 9H). MS (ESI) m/z 377 (M + H)⁺. Anal. calcd. for C₁₉H₂₈N₄O₄ (+0.45 H₂O, 0.30 C₃H₆O, 0.10 C₄H₈O₂ HCl): C, 59.35; H, 7.73; N, 13.64; O, 19.28. Found: C, 58.95; H, 7.54; N; 13.61. |
| Example 130 | N-[(1S)-1-(aminocarbonyl)-2,2-dimethylpropyl]-3-[(1-hydroxycyclopentyl) methyl]-2-oxo-2,3-dihydro-1H-benzimidazole-1-carboxamide |
| 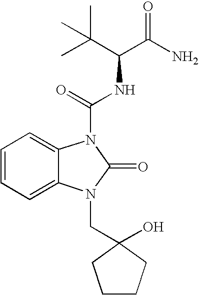 | ¹H-NMR (300 MHz, CDCl₃) δ 9.41 (d, J = 7.5 Hz, 1H), 8.18 (d, J = 6.0 Hz, 1H), 7.27-7.14 (m, 3H), 5.84 (bs, 1H), 5.50 (bs, 1H), 4.22 (d, J = 7.5 Hz, 1H), 4.04 (s, 2H), 1.92-1.66 (m, 8H), 1.14 (s, 9H). MS (ESI) m/z 389 (M + H)⁺. Anal. calcd. for C₂₀H₂₈N₄O₄ (+0.30 H₂O): C, 60.99; H, 7.32; N, 14.22; O, 17.47. Found: C, 60.62; H, 7.31; N; 13.94. |
| Example 131 | N-[(1S)-1-(aminocarbonyl)-2,2-dimethylpropyl]-3-[2-(4-hydroxytetrahy dro-2H-pyran-4-yl)ethyl]-4-methyl-2-oxo-2,3-dihydro-1H-benzimidazole- 1-carboxamide |
| 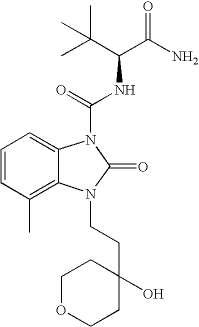 | ¹H-NMR (270 MHz, ODCl₃) δ 9.51 (d, J = 9.5 Hz, 1H), 8.11 (d, J = 7.4 Hz, 1H), 7.05 (dd, J = 7.4, 7.4 Hz, 1H), 6.97 (d, J = 7.4 Hz, 1H), 5.88 (bs, 1H), 5.59 (bs, 1H), 4.30 (m, 2H), 4.22 (d, J = 9.5 Hz, 1H), 3.89-3.67 (m, 4H), 2.62 (s, 3H), 2.17 (s, 1H), 1.94 (t, J = 8.1 Hz, 2H), 1.84-1.50 (m, 4H), 1.14 (s, 9H). MS (ESI) m/z 433 (M + H)⁺. |
| Example 132 | N-[(1S)-1-(aminocarbonyl)-2,2-dimethylpropyl]-2-oxo-3-(tetrahydro-2H- pyran-4-ylmethyl)-2,3-dihydro-1H-benzimidazole-1-carboxamide |
| 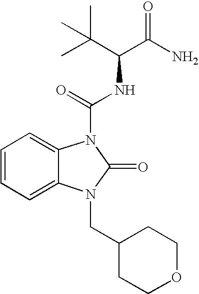 | ¹H-NMR (300 MHz, CDCl₃) δ 9.45 (d, J = 9.0 Hz, 1H), 8.18 (d, J = 9.0 Hz, 1H), 7.25-7.14 (m, 2H), 7.03 (d, J = 9.0 Hz, 1H), 5.98 (bs, 1H), 5.73 (bs, 1H), 4.24 J = 9.0 Hz, 1H) 4.05-3.89 (m, 2H), 3.85-3.67 (m, 2H), 3.41-3.30 (m, 2H), 2.22-2.08 (m, 1H), 1.68-1.35 (m, 4H), 1.16 (s, 9H). MS (ESI) m/z 389 (M + H)⁺. |
| Example 133 | N-[(1S)-1-tert-butyl-3,3,3-trifluoro-2-hydroxypropyl]-3-(2-morpholin-4- ylethyl)-2-oxo-2,3-dihydro-1H-benzimidazole-1-carboxamide hydrochloride |
| 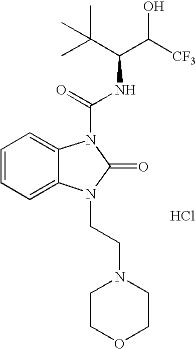 | ¹H-NMR (300 MHz, DMSO-d₆) δ 10.57 (bs, 1H), 8.87 (d, J = 12.0 Hz, 1H), 8.09 (d, J = 9.0 Hz, 1H), 7.50 (d, J = 6.0 Hz, 1H), 7.37-7.16 (m, 2H), 6.59 (bs, 1H), 4.51-3.93 (m, 5H), 3.77-3.05 (m, 9H), 1.02 (s, 9H). MS (ESI) m/z 459 (M + H)⁺. Anal. calcd. for C₂₁H₂₉F₃N₄O₄ (+2.1 H₂O, 1.0 HCl): C, 47.34; H, 6.47; N, 10.52; O, 18.32; F, 10.70; Cl, 6.65. Found: C, 47.03; H, 6.17; N; 10.19. |
| Example 134 | N-[(1S)-1-tert-butyl-3,3,3-trifluoro-2-oxopropyl]-3-(2-morpholin-4-ylethyl)- 2-oxo-2,3-dihydro-1H-benzimidazole-1-carboxamide |
| 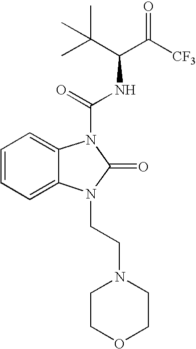 | ¹H-NMR (300 MHz, DMSO-d₆) δ 9.52 (d, J = 7.5 Hz, 1H), 7.96 (d, J = 9.0 Hz, 1H), 7.44-7.10 (m, 3H), 4.88 (d, J = 7.5 Hz, 1H), 4.04 (t, J = 6.0 Hz, 2H), 3.59-3.41 (m, 4H), 2.61 (t, J = 6.0 Hz, 2H), 2.75-2.36 (m, 4H), 1.08 (s, 9H). MS (ESI) m/z 457 (M + H)⁺. Anal. calcd. for C₂₁H₂₇F₃N₄O₄ (+2.1 H₂O, 1.0 HCl): C, 54.82; H, 6.00; N, 12.18; O, 14.61; F, 12.39. Found: C, 54.51; H, 5.95; N; 11.96. |
| Example 135 | N-[(1R)-1-(cyanomethyl)-2,2-dimethylpropyl]-3-(3-hydroxy-3-methylbutyl)- 2-oxo-2,3-dihydro-1H-benzimdazole-1-carboxamide |
| 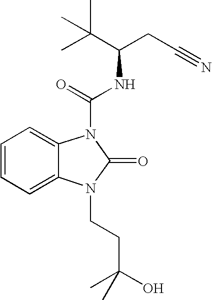 | ¹H-NMR (300 MHz, CDCl₃) δ 9.22 (d, J = 9.0 Hz, 1H), 8.22 (d, J = 9.0 Hz, 1H), 7.30-7.08 (m, 3H), 4.23-4.02 (m, 3H), 2.81 (dd, J = 17.3, 6.0 Hz, 1H), 2.56 (dd, J = 17.3, 7.5 Hz, 1H), 1.93 (t, J = 7.5 Hz, 2H), 1.34 (s, 6H), 1.07 (s, 9H). MS(ESI) m/z 377 (M + H)⁺. Anal. calcd. for C₂₀H₂₈N₄O₃ (+1.0 H₂O): C, 61.52; H, 7.74; N, 14.35; O, 16.39. Found: C, 61.83; H, 7.47; N; 14.36. |
| Example 136 | N-[(1R)-1-(2-amino-2-oxoethyl)-2,2-dimethylpropyl]-3-(3-hydroxy-3- methylbutyl)-2-oxo-2,3-dihydro-1H-benzimidazole-1-carboxamide |
| 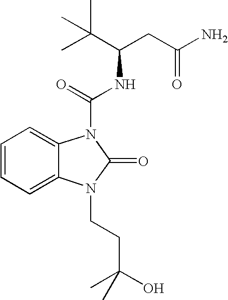 | ¹H-NMR (300 MHz, CDCl₃) δ 9.08 (d, J = 10.8 Hz, 1H), 8.20 (d, J = 8.1 Hz, 1H), 7.41-7.02 (m, 3H), 6.60 (bs, 1H), 5.30 (bs, 1H), 4.22-3.96 (m, 3H), 2.72 (dd, J = 11.5, 4.1 Hz, 1H), 2.40 (dd, J = 11.5, 5.4 Hz, 1H), 1.92 (t, J = 8.1 Hz, 2H), 1.34 (s, 6H), 1.04 (s, 9H). MS (ESI) m/z 391 (M + H)⁺. HR-MS (FAB) Calcd. for C₂₀H₃₁N₄O₄: 391.2345. Found: 391.2343. |
| Example 137 | N-[(1R)-1-(2-hydroxyethyl)-2,2-dimethylpropyl]-3-(3-hydroxy-3-methyl butyl)-2-oxo-2,3-dihydro-1H-benzimidazole-1-carboxamide |
| 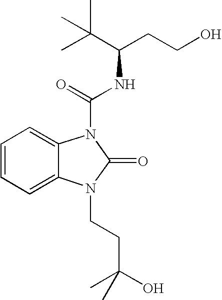 | ¹H-NMR (270 MHz, CDCl₃) δ 8.88 (d, J = 10.8 Hz, 1H), 8.20 (d, J = 5.4 Hz, 1H), 7.28-7.05 (m, 3H), 4.11-3.97 (m, 3H), 3.69-3.65 (m, 2H), 3.31 (bs, 1H), 2.12-1.98 (m, 1H), 1.92 (t, J = 8.1 Hz, 2H), 1.72-1.68 (m, 2H), 1.34 (s, 6H), 1.04 (s, 9H). MS (ESI) m/z 378 (M + H)⁺. Anal. calcd. for C₂₀H₃₁N₃O₄ (+0.8 H₂O): C, 61.30; H, 8.38; N, 10.72; O, 19.60. Found: C, 61.56; H, 8.06; N; 10.73. |
| Example 138 | N-[(1S)-1-tert-butyl-2-hydroxy-2-methylpropyl]-3-(3-hydroxy-3-methylbutyl)- 2-oxo-2,3-dihydro-1H-benzimidazole-1-carboxamide |
| 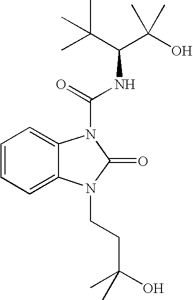 | ¹H-NMR (300 MHz, CDCl₃) δ 8.25 (d, J = 9.0 Hz, 1H), 7.35-7.04 (m, 4H), 4.12-4.07 (m, 2H), 3.84 (d, J = 9.0 Hz, 1H), 1.94 (t, J = 9.0 Hz, 2H), 1.70 (s, 1H), 1.68 (s, 1H), 1.42 (s, 3H), 1.39 (s, 3H), 1.35 (s, 6H), 1.16 (s, 9H). MS (ESI) m/z 392 (M + H)⁺. Anal. calcd. for C₂₁H₃₃N₃O₄ (+0.5 H₂O): C, 62.98; H, 8.56; N, 10.49; O, 17.98. Found: C, 63.02; H, 8.38; N; 10.46. |
| Example 139 | N-[(1S)-1-(aminocarbonyl)-2,2-dimethylpropyl]-3-(2-dimethylamino)-2- methylprpyl]-2-oxo-2,3-dihydro-1H-benzimidazole-1-carboxamide hydrochloride |
| 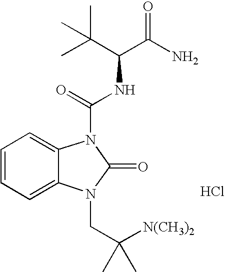 | ¹H-NMR (300 MHz, DMSO-d₆) δ 11.07 (bs, 1H), 9.13 (d, J = 9.0 Hz, 1H), 8.08 (d, J = 6.0 Hz, 1H), 7.72 (bs, 1H), 7.59 (d, J = 6.0 Hz, 1H), 7.41-7.04 (m, 3H), 4.44-4.18 (m, 3H), 2.84 (d, J = 3.0 Hz, 6H), 1.41 (s, 6H), 1.41 (s, 6H), 1.00 (s, 9H). MS (ESI) m/z 390 (M + H)⁺. Anal. calcd. for C₂₀H₃₁N₃O₄ (+1.5 H₂O, 1.0 HCl, 0.2 C₄H₈O₂): C, 53.09; H, 7.84; N, 14.88; O, 16.66; Cl, 7.53. Found: C, 53.14; H, 7.77; N; 14.53. |
| Example 140 | N-[(1S)-2,2-dimethyl-1-(1,3,4-oxadiazol-2-yl)propyl]-3-(3-hydroxy-3- methylbutyl)-2-oxo-2,3-dihydro-1H-benzimidazole-1-carboxamide |
| 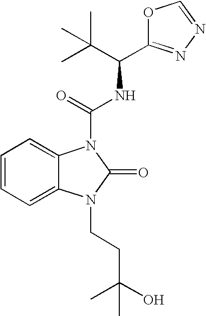 | ¹H-NMR (270 MHz, CDCl₃) δ 9.69 (d, J = 8.1 Hz, 1H), 8.39 (s, 1H), 8.15 (d, J = 8.1 Hz, 1H), 7.35-7.06 (m, 3H), 5.27 (d, J = 8.1 Hz, 1H), 4.16-4.03 (m, 2H), 1.96-1.87 (m, 2H), 1.34 (s, 6H), 1.13 (s, 9H). MS (ESI) m/z 402 (M + H)⁺. HR-MS (FAB) Calcd. for C₂₀H₂₈N₅O₄: 402.2141. Found: 402.2150. |
| Example 141 | N-{(1R)-2,2-dimethyl-1-[(2-methyl-2H-tetrazol-5-yl)methyl]propyl)-3-(3- hydroxy-3-methylbutyl)-2-oxo-2,3-dihydro-1H-benzimidazole-1-carbox- amide |
| 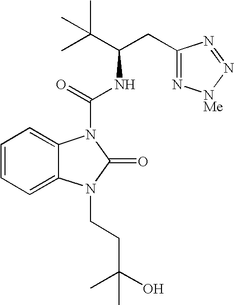 | ¹H-NMR (300 MHz, CDCl₃) δ 8.95 (d, J = 9.0 Hz, 1H), 8.09 (d, J = 9.0 Hz, 1H), 7.36-6.99 (m, 3H), 4.31-4.22 (m, 1H), 4.22 (s, 3H), 4.05 (t, J = 7.5 Hz, 2H), 3.31 (dd, J = 15.0, 3.0 Hz, 1H), 2.96 (dd, J = 15.0, 12.0 Hz, 1H), 1.91 (t, J = 7.5 Hz, 2H), 1.89 (s, 1H), 1.33 (s, 6H), 1.08 (s, 9H). MS (ESI) m/z 430 (M + H)⁺. HR-MS (FAB) Calcd. for C₂₁H₃₂N₇O₃: 430.2567. Found: 430.2580. |
| Example 142 | N-[(1S)-1-tert-butyl-3,3,3-trlfluoro-2-oxopropyl]-3-(3-hydroxy-3-methyl- butyl)-2-oxo-2,3-dihydro-1H-benzimidazole-1-carboxamide |
| 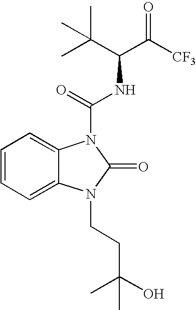 | ¹H-NMR (300 MHz, CDCl₃) δ 9.53 (d, J = 7.5 Hz, I H), 8.15 (d, J = 6.0 Hz, 1H), 7.24-7.02 (m, 3H), 4.90 (d, J = 7.5 Hz, 1H), 4.08 (t, J = 7.5 Hz, 2H), 1.93 (t, J = 7.5 Hz, 2H), 1.34 (s, 6H), 1.15 (s, 9H). MS (ESI) m/z 430 (M + H)⁺. HR-MS (FAB) Calcd. for C₂₀H₂₇F₃N₃O₄: 430.1954. Found: 430.1962. |
| Example 143 | N-[(1S)-1-tert-butyl-3,3,3-trifluoro-2-oxopropyl]-3-[2-(methylsulfonyl) ethyl]-2-oxo-2,3-dihydro-1H-benzimidazole-1-carboxamide |
| 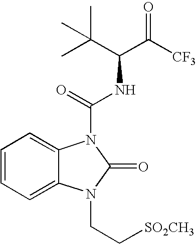 | ¹H-NMR (300 MHz, CDCl₃) δ 9.36 (d, J = 7.5 Hz, 1H), 8.17 (d, J = 9.0 Hz, 1H), 7.35-7.16 (m, 3H), 4.91 (d, J = 7.5 Hz, 1H), 4.42 (t, J = 6.8 Hz, 2H), 3.50 (t, J = 6.8 Hz, 2H), 2.97 (s, 3H), 1.15 (s, 9H). MS (ESI) m/z 450 (M + H)⁺. HR-MS (FAB) Calcd. for C₁₈H₂₃F₃N₃O₅S: 450.1311. Found: 450.1326. |
| Example 144 | N-[2,2-dimethyl-1-(1,3-oxazol-2-yl)propyl]-3-(3-hydroxy-3-methylbutyl)- 2-oxo-2,3-dihydro-1H-benzimidazole-1-carboxamide |
| 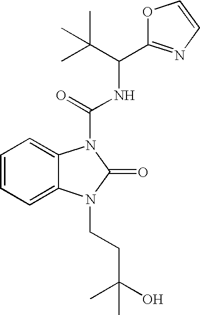 | ¹H-NMR (300 MHz, CDCl₃) δ 9.65 (d, J = 9.0 Hz, 1H), 8.18 (d, J = 9.0 Hz, 1H), 7.62 (s, 1H), 7.27-7.02 (m, 4H), 5.11 (d, J = 9.0 Hz, 1H), 4.08 (t, J = 8.3 1.93 J = 8.3 Hz, 2H), 1.78 (bs, 1H), 1.34 (s, 6H), 1.08 (s, 9H). MS (ESI) m/z 401 (M + H)⁺. HR-MS (FAB) Calcd. for C₂₁H₂₉F₃N₄O₄: 401.2189. Found: 401.2189. |
| Example 145 | N-[(1S)-1-(aminocarbonyl)-2,2-dimethylpropyl]-3-[(4-hydroxytetrahydro- 2H-pyran-4-yl)methyl]-4-methyl]-2-oxo-2,3-dihydro-1H-benzimidarole- 1-carboxamide |
| 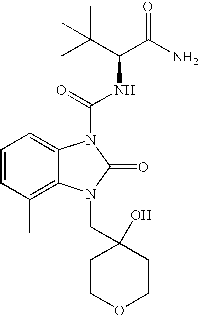 | ¹H-NMR (300 MHz, CDCl₃) δ 9.43 (d, J = 9.0 Hz, 1H), 8.16 (d, J = 7.5 Hz, 1H), 7.09 (dd, J = 7.5, 7.5 Hz, 1H), 6.98 (d, J = 7.5 Hz, 1H), 5.76 (bs, 1H), 5.46 (bs, 1H), 4.25 (s, 2H), 4.20 (d, J = 9.0 Hz, 1H), 3.86-3.71 (m, 5H), 2.64 (s, 3H), 1.91-1.70 (m, 2H), 1.60-1.50 (m, 2H), 1.14 (s, 9H). MS (ESI) m/z 419 (M + H)⁺. Anal. calcd. for C₂₁H₃₀N₄O₅ (+0.2 H₂O, 0.2 C₄H₈O₂, 0.1 C₆H₁₄): C, 58.14; H, 7.62; N, 12.11; 0, 22.13. Found: C, 58.40; H, 7.22; N; 12.48. |
| Example 146 | N-[(1S)-2,2-dimethyl-1-(2-methyl-2H-tetrazol-5-yl)propyl]-3-(3-methoxypropyl)- 2-oxo-2,3-dihydro-1H-benzimidazole-1-carboxamide |
| 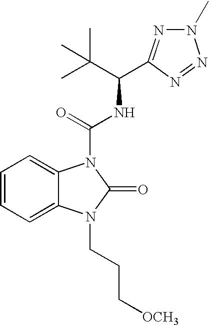 | ¹H-NMR (300 MHz, CDCl₃) δ 9.71 (d, J = 9.0 Hz, 1H), 8.16 (d, J = 6.0 Hz, 1H), 7.23-7.04 (m, 3H), 5.30 (d, J = 9.0 Hz, 1H), 4.34 (s, 3H), 4.02 (t, J = 7.5 Hz, 2H), 3.41 (t, J = 6.0 Hz, 2H), 3.34 (s, 3H), 2.12-1.97 (m, 2H), 1.09 (s, 9H). MS (ESI) m/z 402 (M + H)⁺. |
| Example 147 | N-[(1S)-2,2-dimethyl-1-(2-methyl-2H-tetrazol-5-yl)propyl]-3-(3-hydroxybutyl)-2- oxo-2,3-dihydro-1H-benzimidazole-1-carboxamide (1:1) |
| 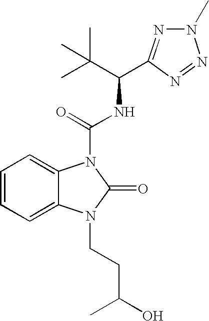 | ¹H-NMR (300 MHz, CDCl₃) δ 9.67-9.52 (m, 1H), 8.18 (d, J = 6.0 Hz, 1H), 7.32-7.05 (m, 3H), 5.30 (d, J = 9.0 Hz, 1H), 4.39-4.17 (m, 4H), 3.97-3.70 (m, 2H), 2.80-2.70 (m, 1H), 1.99-1.65 (m, 2H), 1.29-1.18 (m, 3H), 1.09 (s, 9H). MS (ESI) m/z 402 (M + H)⁺. |
| Example 148 | N-[(1S)-2,2-dimethyl-1-(1,3-oxazol-5-yl)propyl]-3-(3-hydroxy-3-methylbutyl)-2- oxo-2,3-dihydro-1H-benzimidazole-1-carboxamide |
| 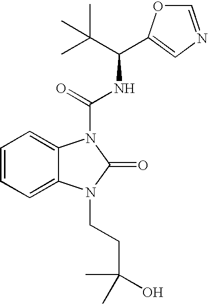 | ¹H-NMR (300 MHz, CDCl₃) δ 9.52 (d, J = 9.0 Hz, 1H), 8.18 (d, J = 9.0 Hz, 1H), 7.84 (s, 1H), 7.32-7.07 (m, 3H), 7.00 (s, 1H), 5.06 (d, J = 9.0 Hz, 1H), 4.09 (t, J = 7.5 Hz, 2H), 1.93 (t, J = 7.5 Hz, 2H), 1.73 (bs, 1H), 1.35 (s, 6H), 1.07 (s, 9H). MS (ESI) m/z 401 (M + H)⁺. |
| Example 149 | N-[(1S)-1-2,2-dimethyl-1-(5-methyl-1,3,4-oxadiazol-2-yl)propyl]- 3-(3-hydroxy-3-methylbutyl)-2-oxo-2,3-dihydro-1H- benzimidazole-1-carboxamide |
| 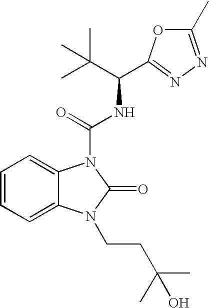 | ¹H-NMR (300 MHz, CDCl₃) δ 9.61 (d, J = 8.7 Hz, 1H), 8.15 (d, J = 7.2 Hz, 1H), 7.23-7.14 (m, 2H), 7.09-7.06 (m, 1H), 5.17 (d, J = 8.7 Hz, 1H), 4.10-4.05 (m, 2H), 2.54 (s, 3H), 1.95-1.90 (m, 2H), 1.34 (s, 6H), 1.12 (s, 9H). MS (ESI) m/z 416 (M + H)⁺. Anal. calcd. for C₂₁H₂₉N₅O₄: C, 60.71; H, 7.04; N, 16.86; O, 15.40. Found: C, 60.55; H, 7.04; N, 16.76. |

Following Examples 150 to 151 were prepared according to the procedure described in Example 107.

| | |
|---|---|
| Example 150 | 1-[3-({[2,2-dimethyl-1-(2-methyl-2H-tetrazol-5-yl)propyl]amino}carbonyl)- 7-methyl-2-oxo-2,3-dihydro-1H-benzimidazol-1-yl]-2-methylpropan-2- aminium formate |
| 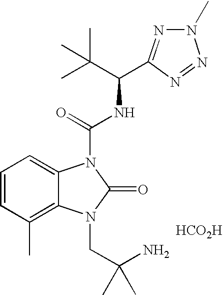 | ¹H-NMR (300 MHz, CDCl₃) δ 9.60 (d, J = 9.0 Hz, 1H), 8.34 (s, 1H), 8.17 (d, J = 8.3 Hz, 1H), 7.07 (dd, J = 8.3, 8.3 Hz, 1H), 6.97 (d, J = 8.3 Hz, 1H), 5.30 (d, J = 9.0 Hz, 1H), 4.33 (bs, 5H), 3.50 (s, 2H), 2.60 (s, 3H), 1.43 (s, 3H), 1.40 (s, 3H), 1.07 (s, 9H). MS (ESI) m/z 415 (M + H)⁺. |
| Example 151 | 4-[3-({(2,2-dimethyl-1-(2-methyl-2H-tetrazol-5-yl)propyl]amino}carbonyl)- 7-methyl-2-oxo-2,3-dihydro-1H-benzimidazol-1-yl]-2-methylbutan-2- aminium formate |
| 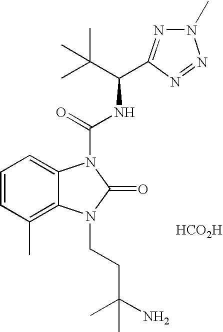 | ¹H-NMR (300 MHz, CDCl₃) δ 9.75 (d, J = 9.0 Hz, 1H), 8.40 (s, 1H), 8.10 (d, J = 6.0 Hz, 1H), 7.06-6.79 (m, 2H), 5.30 (d, J = 9.0 Hz, 1H), 4.34-4.20 (m, 2H), 4.33 (s, 3H), 3.25 (bs, 3H), 2.58 (s, 3H), 2.05 (t, J = 9.0 Hz, 2H), 1.43 (s, 6H), 1.06 (s, 9H). MS (ESI) m/z 429 (M + H)⁺. |

Following Examples 152 to 154 were prepared according to the procedure described in Example 102.

| | |
|---|---|
| Example 152 | N-[(1S)-1-aminocarbonyl)-2,2-dimethylpropyl]-4-methyl-3- [2-(methylthio)ethyl]-2-oxo-2,3-dihydro-1H-benzimidazole- 1-carboxamide |
| 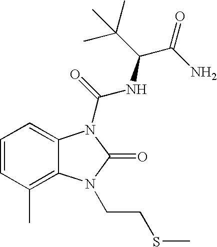 | ¹H-NMR (300 MHz, CDCl₃) δ 9.54 (d, J = 8.1 Hz, 1H), 8.11 (d, J = 7.5 Hz, 1H), 7.06 (t, J = 7.5 Hz, 1H), 6.97 (d, J = 7.5 Hz, 1H), 5.82 (bs, 1H), 5.49 (bs, 1H), 4.33-4.27 (m, 2H), 4.21 (d, J = 8.1 Hz, 1H), 2.85-2.80 (m, 2H), 2.59 (s, 3H), 2.22 (s, 3H), 1.14 (s, 9H). MS (ESI) m/z 379 (M + H)⁺. Anal. calcd. for C₁₈H₂₆N₄O₃S: C, 57.12; H, 6.92; N, 14.80; O, 12.68; S, 8.47. Found: C, 57.19; H, 6.93; N, 14.78. |
| Example 153 | N-{(1S)-2,2-dimethyl-1-[(methylamino)carbonyl]propyl}-4-methyl- 3-[2-(methylthio)ethyl]-2-oxo-2,3-dihydro-1H-benzimidazole-1- carboxamide |
| 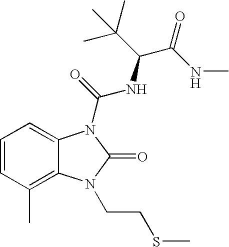 | ¹H-NMR (300 MHz, CDCl₃) δ 9.56-9.45 (m, 1H), 8.09 (d, J = 7.2 Hz, 1H), 7.08-7.03 (m, 1H), 6.96 (d, J = 7.5 Hz, 1H), 5.81 (bs, 1H), 4.32-4.27 (m, 2H), 4.14 (d, J = 8.1 Hz, 1H), 2.85-2.79 (m, 5H), 2.59 (s, 3H), 2.22 (s, 3H), 1.11 (s, 9H). MS (ESI) m/z 393 (M + H)⁺. Anal. calcd. for C₁₉H₂₈N₄O₃S (+0.2 H₂O): C, 57.61; H, 7.23; N, 14.14; O, 12.92; S, 8.10. Found: C, 57.29; H, 7.21; N, 14.01. |
| Example 154 | N-[(1S)-1-(hydroxymethyl)-2,2-dimethylpropyl]-4-methyl- 3-[2-(methylthio)ethyl-2-oxo-2,3-dihydro-1H-benzimidazole- 1-carboxamide |
| 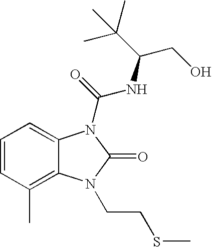 | ¹H-NMR (300 MHz, DMSO-d₆) δ 8.92 (d, J = 9.6 Hz, 1H), 8.04-8.01 (m, 1H), 7.08-7.00 (m, 2H), 4.74-4.66 (m, 1H), 4.27-4.23 (m, 2H), 3.74-3.64 (m, 2H), 3.51-3.43 (m, 1H), 2.84-2.79 (m, 2H), 2.59 (s, 1H), 2.15 (s, 3H), 0.95 (s, 9H). MS (ESI) m/z 366 (M + H)⁺. Anal. calcd. for C₁₈H₂₇N₃O₃S: C, 59.15; H, 7.45; N, 11.50; O, 13.13; S, 8.77. Found: C, 58.76; H, 7.39; N, 11.48. |

Following Examples 155 to 161 were prepared according to the procedure described in Example 103.

| | |
|---|---|
| Example 155 | N-[(1S)-1-(aminocarbonyl)-2,2-dimethylpropyl]-4-methyl-3-[2- (methylsulfonyl)ethyl]-2-oxo-2,3-dihydro-1H-benzimidazole- 1-carboxamide |
| 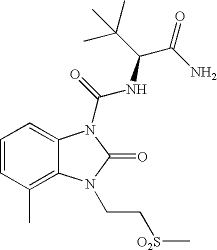 | ¹H-NMR (300 MHz, CDCl₃) δ 9.41 (d, J = 8.7 Hz, 1H), 8.12 (d, J = 8.7 Hz, 1H), 7.12-7.06 (m, 1H), 7.00 (d, J = 8.1 Hz, 1H), 5.75 (bs, 1H), 5.48 (bs, 1H), 4.66-4.61 (m, 2H), 4.21 (d, J = 8.7 Hz, 1H), 3.51-3.46 (m, 2H), 3.04 (s, 2H), 2.65 (s, 3H), 1.14 (s, 9H). MS (ESI) m/z 411 (M + H)⁺. Anal. calcd. for C₁₈H₂₆N₄O₅S (+0.5 H₂O, 0.2 C₄H₈O₂): C, 51.66; H, 6.59; N, 12.82; O, 21.60; S, 7.34. Found: C, 51.96; H, 6.33; N, 12.89. mp 177.5 degrees Celsius, 238.2 degrees Celsius. |
| Example 156 | N-{(1S)-2,2-dimethyl-1-[(methylamino)carbonyl]propyl}-4- methyl-3-[2-(methylsulfonyl)ethyl]-2-oxo-2,3-dihydro-1H- benzimidazole-1-carboxamide |
| 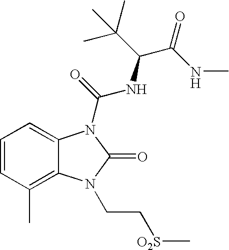 | ¹H-NMR (300 MHz, CDCl₃) δ 9.42 (d, J = 9.0 Hz, 1H), 8.09 (d, J = 7.2 Hz, 1H), 7.07 (t, J = 7.2 Hz, 1H), 6.98 (d, J = 7.2 Hz, 1H), 5.89-5.80 (m, 1H), 4.65-4.57 (m, 2H), 4.16 (d, J = 9.0 Hz, 1H), 3.51-3.46 (m, 2H), 3.04 (s, 3H), 2.84 (d, J = 4.5 Hz, 3H), 2.64 (s, 3H), 1.11 (s, 9H). MS (ESI) m/z 425 (M + H)⁺. Anal. calcd. for C₁₉H₂₈N₄O₆S (+0.2 H₂O): C, 53.08; H, 6.71; N, 13.03; O, 19.72; Cl, 7.46. Found: C, 52.76; H, 6.54; N, 12.64. |
| Example 157 | N-[(1S)-1-(hydroxymethyl)-2,2-dimethylpropyl]-4-methyl- 3-[2-(methylsulfonyl)ethyl-2-oxo-2,3-dihydro-1H-benzimidazole- 1-carboxamide |
| 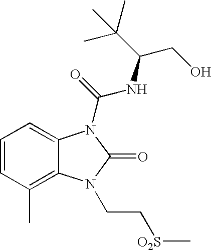 | ¹H-NMR (300 MHZ, CDCl₃) δ 8.87 (d, J = 9.6 Hz, 1H), 8.02 (dd, J = 7.2, 1.2 Hz, 1H), 7.10-7.01 (m, 2H), 4.72-4.69 (m, 1H), 4.53-4.48 (m, 2H), 3.74-3.59 (m, 4H), 3.51-3.41 (m, 1H), 3.12 (s, 3H), 2.62 (s, 3H), 0.95 (s, 9H). MS (ESI) m/z 398 (M + H)⁺. Anal. calcd. for C₁₈H₂₇N₃O₅S (+0.1 H₂O): C, 54.14; H, 6.87; N, 10.52; O, 20.44; S, 8.03. Found: C, 53.90; H, 6.87; N, 10.26. |
| Example 158 | rel-N-[(1R, 2S)-2-hydroxycyclohexyl]-4-methyl-3-[2-(methylsulfonyl)ethyl]- 2-oxo-2,3-dihydro-1H-benzimidazole-1-carboxamide |
| 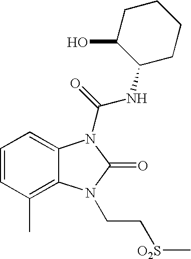 | ¹H-NMR (300 MHz, DMSO-d₆) δ 8.80 (d, J = 7.5 Hz, 1H), 8.02-7.99 (m, 1H), 7.09-7.01 (m, 2H), 4.87 (d, J = 5.1 Hz, 1H), 4.52-4.47 (m, 2H), 3.66-3.45 (m, 3H), 3.11 (s, 3H), 2.61 (s, 3H), 2.10-1.98 (m, 1H), 1.92-1.80 (m, 1H), 1.70-1.56 (m, 2H), 1.36-1.20 (m, 4H). MS (ESI) m/z 396 (M + H)⁺. Anal. calcd. for C₁₈H₂₅N₃O₅S (+0.3 H₂O): C, 53.93; H, 6.44; N, 10.48; O, 21.15; S, 8.00: Found: C, 53.66; H, 6.24; N, 10.36. mp 152.3 degrees Celsius, 238.9 degrees Celsius. |
| Example 159 | N-[(1S)-2,2-dimethyl-1-(3-methyl-1,2,4-oxadiazol-5-yl)propyl]- 3-[2-(methylsulfonyl)ethyl]-2-oxo-2,3-dihydro-1-H-benzimidazole- |
| 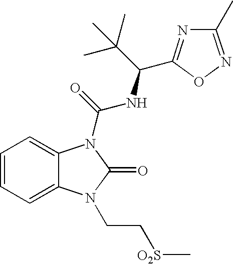 | ¹H-NMR (300 MHz, CDCl₃) δ 9.49 (d, J = 8.1 Hz, 1H), 8.16 (d, J = 8.1 Hz, 1H), 7.30-7.15 (m, 3H), 5.17 (d, J = 8.1 Hz, 1H), 4.45-4.40 (m, 2H), 3.54-3.49 (m, 2H), 2.98 (S, 3H), 2.41 (s, 3H), 1.12 (s, 9H). MS (ESI) m/z 436 (M + H)⁺. Anal. calcd. for C₁₉H₂₅N₅O₅S: C, 52.40; H, 5.79; N, 16.08; O, 18.37; S, 7.36. Found: C, 52.06; H, 5.75; N, 15:79 m.p 124.2 degrees Celsius. |
| Example 160 | N-[(1S)-2,2-dimethyl-1-(2-methyl-2H-tetrazol-5-yl)propyl]- 3-[2-(methylsulfonyl)ethyl]-2-oxo-2,3-dihydro-1H- benzimidazole-1-carboxamide |
| 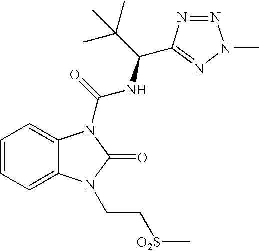 | ¹H-NMR (300 MHz, CDCl₃) δ 9.56 (d, J = 8.7 Hz, 1H), 8.18 (d, J = 7.5 Hz, 1H), 7.28-7.14 (m, 3H), 5.29 (d, J = 8.7 Hz, 1H), 4.44-4.39 (m, 2H), 4.34 (s, 3H), 3.54-3.49 (m, 2H), 2.97 (s, 3H), 1.08 (s, 9H). MS (ESI) m/z 436 (M + H)⁺. Anal. calcd. for C₁₈H₂₅N₇O₄S (+0.1 H₂O): C, 49.44; H, 5.81; N, 22.42; O, 15.00; S, 7.33. Found: C, 49.18; H, 5.76; N, 22.09. |
| Example 161 | N-[(1S)-2,2-dimethyl-1-(5-methyl-1,3,4-oxadiazol-2-yl)propyl]-3- [2-(methylsulfonyl)ethyl)-2-oxo-2,3-dihydro-1H-benzimidazole- 1-carboxamide |
| 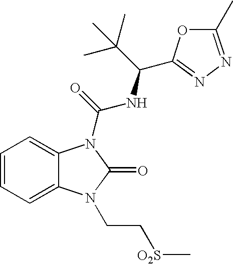 | ¹H-NMR (300 MHz, CDCl₃) δ 9.46 (d, J = 10.2 Hz, 1H), 8.18-8.15 (m, 1H), 7.30-7.15 (m, 3H), 5.15 (d, J = 10.2 Hz, 1H), 4.45-4.39 (m, 2H), 3.54-3.48 (m, 2H), 2.98 (S, 3H), 2.55 (s, 3H), 1.12 (s, 9H). MS (ESI) m/z 436 (M + H)⁺. Anal. calcd. for C₁₉H₂₅N₅O₅S (+0.1 H₂O): C, 52.18; H, 5.81; N, 16.02; O, 18.66; S, 7.33. Found: C, 51.91; H, 5.70; N, 15.91. |

### Example 162

### Same as Example 152 Made by Alternative Procedure

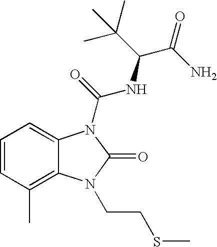

### N-[(1S)-1-aminocarbonyl)-2,2-dimethylpropyl]-4-methyl-3-[2-(methylthio)ethyl]-2-oxo-2,3-dihydro-1H-benzimidazole-1-carboxamide

### STEP 1: 2-methyl-N-[2-(methylthio)ethyl]-6-nitroaniline

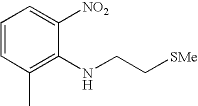

A mixture of 2-chloro-3-nitrotoluene (1.3 g, 7.3 mmol), 2-(methylthio)ethylamine (1.4 mL, 15 mmol) and N,N-diisopropylethylamine (5.0 mL, 29 mmol) in 1-methyl-2-pyrrolidinone (3.7 mL) was heated at 220 degrees Celsius by microwave for 1 h. The mixture was diluted with ethyl acetate (0.10 L) and washed with water (2×50 mL), brine (50 mL), dried over sodium sulfate, filtered, and concentrated to give a crude material. The another batch starting from 1.3 g of 2-chloro-3-nitrotoluene was combined to this crude material and the combined crude materials were purified by column chromatography on silica gel eluting with hexane/ethyl acetate (3/1) to afford 2.6 g (77%) of the title compound: MS (ESI) m/z 227 (M+H)⁺.

### STEP 2: 7-methyl-1-[2-(methylthio)ethyl]-1,3-dihydro-2H-benzimidazol-2-one

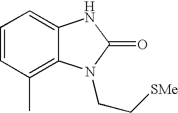

To a solution of 2-methyl-N-[2-(methylthio)ethyl]-nitroaniline (2.6 g, 12 mmol) in ethanol (6.0 mL) was added a solution of Tin(II) chloride dihydrate (7.9 g, 35 mmol) In concd. hydrochloric acid (8.0 mL) at 0 degrees Celsius and then warmed to room temperature. After 4 hours, the reaction was quenched by addition of 6N sodium hydroxide (100 mL) and extracted with ethyl acetate (100 mL×2), dried over sodium sulfate, filtered, and concentrated. The crude material was dissolved in THF (50 mL) and to this solution was added CDI (2.3 g, 14 mmol) and the mixture was stirred at room temperature. After 12 hours, to the mixture was added CDI (1.5 g, 6.7 mmol) and the reaction mixture was refluxed for 5 hours. Then the mixture was cooled to room temperature and evaporated to dryness. To this residue was added water (100 mL) at 0 degrees Celsius and extracted with ethyl acetate (100 mL×2). The combined organic layers were washed with water (50 mL), brine (50 mL), dried over sodium sulfate, filtered, and concentrated. The obtained material was dissolved in methanol (30 mL) and to this solution was added 2N sodium hydroxide (3 mL) and stirred at room temperature for 2 hours. Then the mixture was quenched by addition of sat. aq. sodium bicarbonate (50 mL) and extracted with ethyl acetate (100 mL×2). The combined organic layers were washed with water (50 mL), brine (50 mL), dried over sodium sulfate, filtered and concentrated. The residue was purified by column chromatography on silica gel eluting with dichloromethane/methanol (20/1-10/1) to afford 1.8 g (69%) of the title compound: 6 MS (ESI) m/z 223 (M+H)⁺.

### STEP 3: N-[(1S)-1-(aminocarbonyl)-2,2-dimethylpropyl]-4-methyl-3-[2-(methylthio)ethyl]-2-oxo-2,3-dihydro-1H-benzimidazole-1-carboxamide

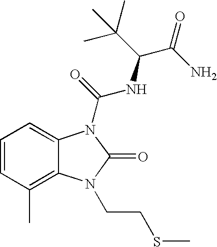

To a solution of 7-methyl-1-[2-(methylthio)ethyl]-1,3-dihydro-2H-benzimidazol-2-one (0.22 g, 1.00 mmol) in 1,2-dichloroethane (5 mL) were added triethylamine (0.5 mL, 3.3 mmol) and 4-nitrophenyl chloroformate (0.24 g, 1.2 mmol) at 0 degrees Celsius and the mixture was stirred at room temperature for 4 hours. Then to this mixture was added a solution of tert-leucinamide (156 mg, 1.2 mmol) in 1,2-dichloroethane (3 mL) at 0 degrees Celsius and stirred at room temperature. After 14 hours, the reaction was quenched by addition of water (50 mL) and extracted with dichloromethane (50 mL×2). The combined organic layers were washed with water (30 mL×4), brine (30 mL), dried over sodium sulfate, filtered, and concentrated in vacuo. The residue was purified by column chromatography on silica gel eluting with dichloromethane/methanol (10/1) to afford 0.33 g (87%) of the title compound: ¹H-NMR (300 MHz, CDCl₃) [symbol] 9.54 (d, J=8.1 Hz, 1H), 8.11 (d, J=7.5 Hz, 1H), 7.06 (t, J=7.5 Hz, 1H), 6.97 (d, J=7.5 Hz, 1H), 5.82 (bs, 1H), 5.49 (bs, 1H), 4.33-4.27 (m, 2H), 4.21 (d, J=8.1 Hz, 1H), 2.85-2.80 (m, 2H), 2.59 (s, 3H), 2.22 (s, 3H), 1.14 (s, 9H); MS (ESI) m/z 379 (M+H)⁺.

Anal. calcd. for C₁₈H₂₆N₄O₃S: C, 57.12; H, 6.92; N, 14.80; O, 12.68; S, 8.47. Found: C, 57.19; H, 6.93; N, 14.78

The tert-Leucinamide reagent used in step 3 above was prepared in two steps as follows:

### STEP 1: Benzyl [(1S)-1-(aminocarbonyl)-2,2-dimethylpropyl]carbamate

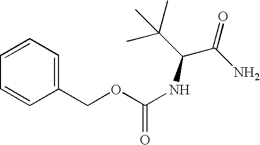

To a solution of N-[(benzyloxy)carbonyl]-tert-leucine (prepared according to the procedure in the literature; Emily, M. S. et al. Tetrahedron 2001, 57, 5303-5320.; 3.7 g, 14 mmol) in DMF (80 mL) were added ammonium chloride (900 mg, 17 mmol), triethylamine (5.9 mL, 42 mmol), HOBt (2.8 g, 18 mmol), and EDC (3.1 g, 18 mmol) at rt. After 17 h, the reaction mixture was quenched by addition of sat. aq. sodium bicarbonate (100 mL) and extracted with ethyl acetate (100 mL×3). The combined organic layers were washed with water (100 mL×3), brine (50 mL), dried over sodium sulfate, filtered and concentrated in vacuo. The residue was purified by column chromatography on silica gel eluting with hexane/ethyl acetate (2/1-1/1) to afford 3.0 g (82%) of the title compound.

MS (ESI) m/z 265 (M+H)⁺.

### STEP 2: tert-Leucinamide

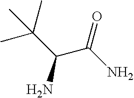

To a solution of benzyl [(1S)-1-(aminocarbonyl)-2,2-dimethylpropyl]carbamate (3.7 g, 14 mmol) in THF (40 mL) was added 10% Pd/C (710 mg). The flask was evacuated and flushed with H₂ gas and this process was repeated three times. The flask was filled with H₂ gas (4 atm) and stirred for 3 hours at room temperature. Then the reaction mixture was filtered through a pad of Celite and concentrated in vacuo to give the title compound as white solid (crude; 1.8 g):

¹H-NMR (300 MHz, DMSO-d₆) [symbol] 6.59 (bs, 1H), 5.92 (bs, 1H), 3.12 (s, 1H), 1.02 (s, 1H); MS (ESI) m/z 131 (M+H)⁺.

### Example 163

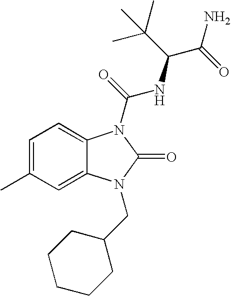

### N-[(1S)-1-(aminocarbonyl)-2,2-dimethylpropyl]-3-(cyclohexylmethyl)-5-methyl-2-oxo-2,3-dihydro-1H-benzimidazole-1-carboxamide (PF-04431715-51)

### Step 1: Preparation of N-(cyclohexylmethyl)-5-methyl-2-nitroaniline

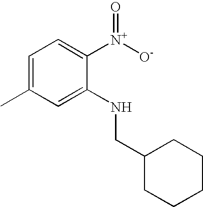

A mixture of cyclohexylmethylamine (1.6 g, 14.2 mmol), 3-fluoro-4-nitrotoluene (2.0 g, 13.0 mmol) and N,N-diisopropylethylamine (1.83 g, 14.2 mmol) was heated to 80 degrees Celsius in acetonitrile (10 mL) for 2 hours. The reaction was cooled to ambient temperature and partitioned between water and ethyl acetate. The layers were separated and aqueous extracted with ethyl acetate (1×30 mL). The organic layers were combined, dried over sodium sulfate and concentrated in vacuo. Purification of the residue by silica gel chromatography eluting from example 1 from 4-[(2-aminophenyl)amino]-2-methyl-2-butanol.

MS (ESI) m/z 221 (M+H)⁺.

### STEP 6. N-[(1S)-1-(aminocarbonyl)-2,2-dimethylpropyl]-3-(3-hydroxy-3-methylbutyl)-2-oxo-2,3-dihydro-1H-benzimidazole-1-carboxamide

To a solution of 1-(3-hydroxy-3-methylbutyl)-1,3-dihydro-2H-benzimidazol-2-one (0.25 g, 1.1 mmol) in 1,2-dichloroethane (30 mL) were added triethylamine (0.52 mL, 3.7 mmol) and 4-nitrophenyl chloroformate (0.27 g, 1.4 mmol) at 0 degrees Celsius and the mixture was stirred for 4 hours at room temperature. Then to this mixture was added a mixture of L-tert-leucinamide (0.18 g, 1.4 mmol) in 1,2-dichloroethane (5 mL) at 0 degrees Celsius and stirred at room temperature. After 14 hours, the reaction was quenched by addition of water (50 mL) and filtered and washed with water (30 mL) and dichloromethane (30 mL). The obtained solid was suspended in water (50 mL) and filtered. This procedure was repeated twice followed by recrystallization from methanol. The obtained solid was suspended in water (50 mL) again and filtered and this procedure was repeated twice. Then the solid was dissolved in methanol/dichloromethane and filtered and concentrated in vacuo. The obtained solid was then recrystallized from acetone to give 0.14 g (33%) of the title compound.

¹H-NMR (300 MHz, DMSO-d₆) δ 9.21 (d, J=9.0 Hz, 1H), 8.03 (d, J=7.2 Hz, 1H), 7.65 (bs, 1H), 7.25-7.12 (m, 4H), 4.50 (s, 1H), 4.33 (d, J=9.0 Hz, 1H), 3.96-3.93 (m, 2H), 1.77-1.71 (m, 2H), 1.17 (s, 6H), 0.98 (s, 9H).

MS (ESI) m/z 377 (M+H)⁺.

Anal. calcd. for C₁₉H₂₈N₄O₄: C, 60.62; H, 7.50; N, 14.88; O, 17.00. Found: C, 60.46; H, 7.51; N, 14.59.

mp 247.7 degrees Celsius

[α]_{D}²³+29.1 (c 0.11, methanol).

>99% ee

### Example 102

### N-{(1S)-1-[(dimethylamino)carbonyl]-2,2-dimethylpropyl}-4-methyl-3-[2-(methylthio)ethyl]-2-oxo-2,3-dihydro-1H-benzimidazole-1-carboxamide

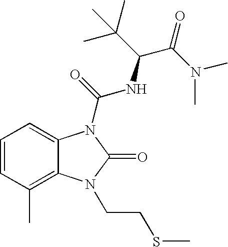

### STEP 1. 2-methyl-N-[2-(methylthio)ethyl]-6-nitroaniline

A mixture of 2-chloro-3-nitrotoluene (1.3 g, 7.3 mmol), 2-(methylthio)ethylamine (1.4 mL, 15 mmol) and N,N-diisopropylethylamine (5.0 mL, 29 mmol) in 1-methyl-2-pyrrolidinone (3.7 mL) was heated to 220 degrees Celsius by microwave for 1 h. The mixture was diluted with ethyl 0-10% ethyl acetate/hexanes gave 3.2 g of oil: ¹H NMR (400 MHz, DMSO-d₆) δ 0.92-1.05 (m, 2H), 1.10-1.24 (m, 3H), 1.58-1.72 (m, 6H), 2.27 (s, 3H), 3.16 (t, J=6.0 Hz, 2H), 6.45 (dd, J=8.8, 1.2 Hz, 1H), 6.82 (s, 1H), 7.91 (d, J=8.4 Hz, 1H), 8.15 (t, J=5.2 Hz, 1H); MS (APES) m/z 249 (M+H).

### Step 2: Preparation of 1-(cyclohexylmethyl)-6-methyl-1-dihydro-2H-benzimidazol-2-one

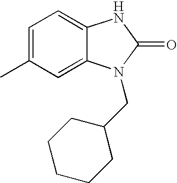

A solution of N-(cyclohexylmethyl)-5-methyl-2-nitroaniline (3.2 g, 13 mmol) in ethanol (6.5 ml) and concentrated hydrochloric acid (8.7 mL) was added tin(II) chloride dehydrate (8.8 g, 39 mmol) as a solid at 0 degrees Celsius. The reaction mixture was allowed to warm to room temperature for 2 hours. The mixture was quenched by the addition of 6 N NaOH (100 mL) and extracted with ethyl acetate (3×100 mL). The organic layers were combined, dried over Na₂SO₄ and concentrated in vacuo. The resultant orange oil was dissolved in tetrahydrofuran (50 mL) and 1,1′-carbonyldiimidazole (2.5 g, 15.6 mmol) was added as a solid. The resultant mixture was stirred at room temperature overnight. The mixture was concentrated in vacuo and the residue was partitioned between water and ethyl acetate. The organic layer was dried over sodium sulfate and evaporated. Purification of the residue by chromatography over silica gel by eluting with 0-10% methanol/dichloromethane gave a yellow material, which was triturated with ethyl acetate and ether to yield 2.37 g of white solid: ¹H NMR (400 MHz, DMSO-d₆) δ 0.92-0.99 (m, 2 H), 1.05-1.15 (m, 3H), 1.50-1.63 (m, 5H), 1.73 (m, 1H), 2.82 (s, 3H), 3.53 (d, J=7.6 Hz, 2H), 6.73 (d, J=8.0 Hz, 1H), 6.80 (d, J=7.6 Hz, 1H), 6.88 (s, 1H), 10.61 (s, 1H); MS (APES) m/z 245 (M+H).

### Step 3: Preparation of N-[(1S)-1-(aminocarbonyl)-2,2-dimethylpropyl]-3-(cyclohexylmethyl)-5-methyl-2-oxo-2,3-dihydro-1H-benzimidazole-1-carboxamide (PF-04431715-51)

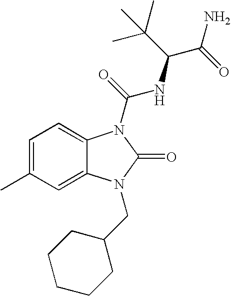

To a solution of 1-(cyclohexylmethyl)-6-methyl-13-dihydro-2H-benzimidazol-2-one (125 mg, 0.51 mmol) in 1,2-dichloroethane (3 mL) was added triethylamine (171 mg, 1.7 mmol) and 4-nitrophenylchloroformate (124 mg, 0.61 mmol) at 0 degrees Celsius. The reaction mixture was then allowed to warm to room temperature and stirred for 4 hours. Tert-leucinamide (80 mg, 0.61 mmol) was added to the reaction mixture and the stirring was continued at room temperature overnight. The reaction was quenched by the addition of water and the organic layer was washed with water (3×3 mL), dried over sodium sulfate, and concentrated to dryness. To a solution of the residue in methanol (2 mL) was added trifluoroacetic acid dropwise to yield a white solid, which was collected by filtration to give 82.3 mg of the title compound as a trifluoroacetate salt: ¹H NMR (400 MHz, DMSO-d₆) δ 0.90-1.09 (m, 12H), 1.17 (m, 3H), 1.58-1.70 (m, 5H), 1.83 (m, 1H), 2.37 (s, 3H), 3.71 (dd, J=8.0, 4.0 Hz, 2H), 4.23 (d, J=8.78 Hz, 1H), 6.95 (d, J=8.05 Hz, 1H), 7.14 (d, J=5.49 Hz, 2H), 7.62 (br. s., 1H), 7.91 (d, J=8.0 Hz, 1H), 9.16 (d, J=9.15 Hz, 1H); MS (ES+) m/z 401.255 (M+H).

### Example 164

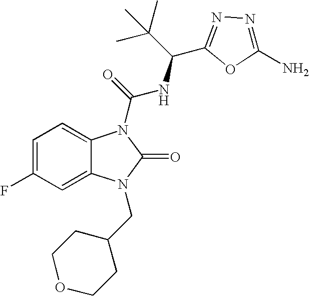

### N-[(1S)-1-(5-amino-1,3,4-oxadiazol-2-yl)-2,2-dimethylpropyl]-5-fluoro-2-oxo-3-(tetrahydro-2H-pyran-4-ylmethyl)-2,3-dihydro-1H-benzimidazole-1-carboxamide

### Step 1: ((S)-1-Hydrazinocarbonyl-2,2-dimethylpropyl)carbamic acid tert-butyl ester

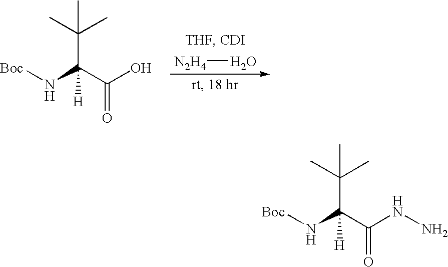

To a solution of N-Boc-L-tert-leucine (2.0 g, 8.647 mmol) in dry THF (20 ml), N,N-carbonyl diimidazole (CDI) (1.54 g, 9.511 mmol) was added and stirred at rt under nitrogen atmosphere for 1.5 h. Hydrazine hydrate (1.3 ml, 26.6 mmol) was then added to it and stirring was continued for 18 h at rt. On completion of reaction (monitored by TLC, R_{f}=0.3; solvent system 40% ethyl acetate in hexane), THF was evaporated up to dryness and the residual mass dissolved in 1,4-dioxane (50 ml) and filtered. The filtrate was concentrated under reduced pressure and the residual mass (as white sticky material) was again dissolved in DCM. The solution was washed with distilled water, brine, dried over anhydrous Na₂SO₄ and concentrated under reduced pressure to afford desired product ((S)-1-hydrazinocarbonyl-2,2-dimethylpropyl)carbamic acid tert-butyl ester (2.3 g) as gummy sticky mass contaminated with imidazole.

¹H NMR (400 MHz, DMSO-d₆) δ: 0.87 (s, 9H), 1.37 (s, 9H), 3.80 (d, J=9.6 Hz, 1H), 6.35 (d, J=9.6 Hz, 1H), 9.10 (s, 1H)+imidazole: 7.01 (s, 2H), 7.63 (s, 1H). ¹H NMR (400 MHz, DMSO-d₆-D₂O exchange) δ: 0.88 (s, 9H), 1.35 (s, 9H), 3.77 (s, (1H), +imidazole: 7.01 (2H, 7.65 (s, 1H). FIA-MS: 246.3 [M+H]⁺, 268.3 [M+H+Na]⁺.

### Step 2

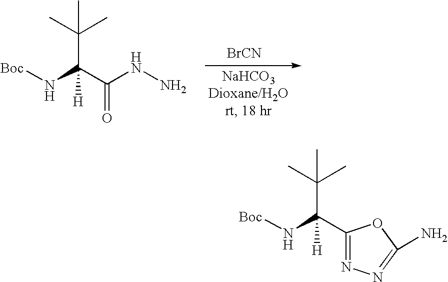

[1-(5 Amino-[1,3,4]oxadiazol-2-yl)-(S)-2,2-dimethylpropyl]carbamic acid tert-butyl ester: To a clear solution of ((S)-1-hydrazinocarbonyl-2,2-dimethylpropyl)carbamic acid tert-butyl ester (1.5 g, 6.117 mmol) in 1,4-dioxane (50 ml), a solution of NaHCO₃ (0.515 g, 6.117 mmol) in distilled water (15 ml) was added to form a white suspension. Cyanogen bromide (0.65 g, 6.117 mmol) was added portion wise to the reaction mixture and stirred for 18 h at rt. On completion of reaction (monitored by TLC, R_{f}=0.5; solvent system 50% ethyl acetate in hexane), the dioxane was evaporated under reduced pressure and ethyl acetate (100 ml) was added. This solution was then washed twice with distilled water (2×100 ml), brine, dried over anhydrous Na₂SO₄ and concentrated under reduced pressure. The residual mass obtained was washed with hexane to afford desired product [1-(5-amino-[1,3,4]oxadiazol-2-yl)-(S)-2,2-dimethylpropyl]carbamic acid tert-butyl ester (0.7 g, yield 42%) as off white solid.

¹H NMR (400 MHz, CDCl₃) δ: 1.01 (s, 9H), 1.27 (s, 9H), 4.65 (d, J=9.6 Hz, 1H), 5.44 (d, J=8.4 Hz, 1H), 8.92 (br s, 2H). LC-MS (10%-90% CH₃CN-0.05% TFA-water gradient over 5 minutes: 3.30 min, 271.4 [M+H]⁺.

### Step 3

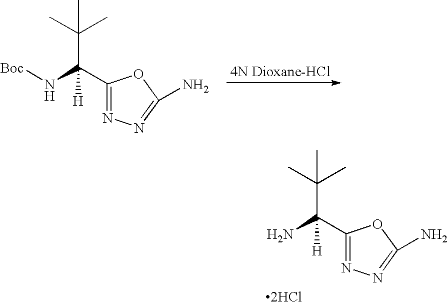

5-((S)-1-Amino-2,2-dimethylpropyl)-[1,3,4]oxadiazol-2-ylamine dihydrochloride: [1-(5-Amino-[1,3,4]oxadiazol-2-yl)-(S)-2,2-dimethylpropyl]carbamic acid tert-butyl ester (4.0 g, 14.81 mmol) was dissolved in 75 ml of 4N 1,4-dioxane-HCl solution and stirred at rt under nitrogen atmosphere for 4 hr. Evaporation of dioxane under reduced pressure gave 5-((S)-1-amino-2,2-dimethylpropyl)-[1,3,4]oxadiazol-2-yl amine dihydrochloride as white solid (3.5 g, yield 98.59%).

¹H NMR (400 MHz, DMSO-d₆) δ: 0.95 (s, 9H), 4.31 (d, J=5.6 Hz, 1H), 6.34 (br s, 3H), 7.60 (br s, 1H), 8.86 (d, J=4.0 Hz, 3H). LC-MS (10%-90% CH₃CN-0.05% TFA-water gradient over 5 minutes: 0.69 min, 171.1 [M+H]⁺.

### Step 4

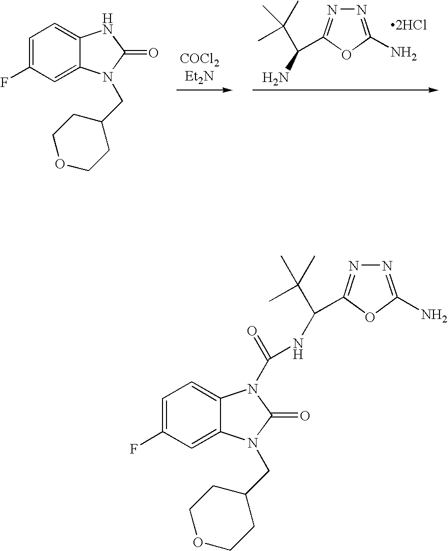

### N-[(1S)-1-(5-amino-1,3,4-oxadiazol-2-yl)-2,2-dimethylpropyl]-5-fluoro-2-oxo-3-(tetrahydro-2H-pyran-4-ylmethyl)-2,3-dihydro-1H-benzimidazole-1-carboxamide (PF-04676113-00)

To a solution of 6-fluoro-1-(tetrahydro-2H-pyran-4-ylmethyl)-1,3-dihydro-2H-benzimidazol-2-one (45 mg, 0.18 mmol) in 1,2-dichloroethane (3 ml) was added triethylamine (100 μl, 73 mg, 0.72 mmol) and phosgene as a 1.8 M solution in toluene (120 μl, 21.3 mg, 0.22 mmol) at ambient temperature. The resultant mixture was stirred for 1 hour at ambient temperature (the mixture turned brown). To this mixture was added 5-[(1S)-1-amino-2,2-dimethylpropyl]-1,3,4-oxadiazol-2-amine hydrochloride (44.6 mg, 0.22 mmol) as a solid. The resultant reaction mixture was stirred at 45° C. overnight. The reaction mixture was cooled to ambient temperature and extracted 3×2 ml water. The organic layer was concentrated, dissolved in 1 ml DMSO and purified by reversed phase HPLC (acetonitrile/water). 23.5 mg, 29% yield.

¹H NMR (400 MHz, DMSO-d₆) □ ppm 1.03 (s, 8H) 1.32 (dd, J=12.44, 4.03 Hz, 2H) 1.54 (d, J=12.44 Hz, 2H) 2.05 (td, J=11.80, 4.57 Hz, 1H) 3.18-3.26 (m, 3H) 3.74 (d, J=6.95 Hz, 1H) 3.79 (d, J=7.32 Hz, 2H) 3.81 (br. s., 3H) 4.87 (d, J=8.78 Hz, 1H) 7.03 (s, 2H) 7.11 (td, J=9.15, 2.56 Hz, 1H) 7.41 (dd, J=8.78, 4.76 Hz, 1H) 7.79 (dd, J=9.52, 2.56 Hz, 1H) 9.38 (d, J=8.78 Hz, 1H). LC/MS 446.2 (M).

Examples 165-175 were prepared according to the procedures described for Examples 162, 163 and 164.

| | Structure | 1H NMR (400 MHz, | |
|---|---|---|---|
| Example No. | chemical name | DMSO-d6) δ ppm | Mass (M) |
| 165 | 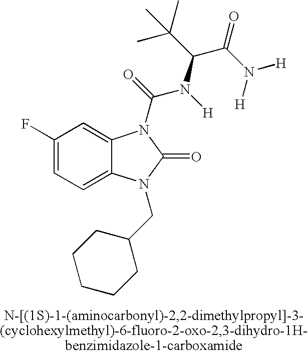 | 0.93-1.60 (m, 12H), 1.15 (m, 3H), 1.64 (m, 5H), 1.81 (m, 1 H), 3.72 (d, J = 4.76 Hz, 2H), 4.24 (d, J = 9.15 Hz, 1H), 7.15 (br. s., 1H), 7.08 (dt, J = 8.0, 4.0 Hz, 1H), 7.33 (d, J = 8.60, 4.57 Hz, 1H), 7.63 (br. s., 1H), 7.81 (dd, J = 8.0, 4.0 Hz, 1H), 9.16 (d, J = 8.78 Hz, 1H) | 404 |
| 166 | 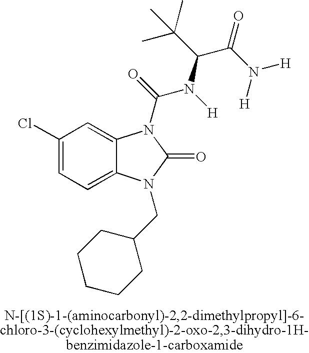 | 0.93-1.15 (m, 13H), 1.59 (m, 4H), 1.77 (m, 2H), 3.69 (dd, J = 7.2, 2.4 Hz, 2H), 4.21 (d, J = 9.2 Hz, 1H), 6.90 (d, J = 8.8 Hz, 1 H), 7.18 (br. s., 1H), 7.26 (dd, J = 8.4, 2.0 Hz, 1H), 7.34 (d, J = 8.4 Hz, 1H), 7.64 (s, 1H), 8.01 (d, J = 2.0 Hz, 1H), 8.09 (d, J = 9.2 Hz, 1H), 9.11 (d, J = 9.2 Hz, 1H) | 420 |
| 167 | 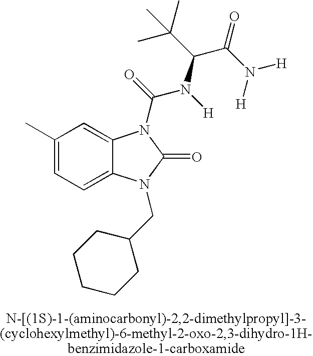 | 0.92-1.07 (m, 12H), 1.14 (m, 3H), 1.58-1.70 (m, 5H), 1.81 (m, 1H), 2.35 (s, 3H), 3.70 (dd, J = 8.0, 4.0 Hz, 2H), 4.23 (d, J = 8.78 Hz, 1H), 7.04 (d, J = 7.69 Hz, 1H), 7.19 (d, J = 7.69 Hz, 1H), 7.13 (br. s., 1H), 7.62 (br. s., 1 H), 7.88 (s, 1H), 9.20 (d, J = 8.78 Hz, 1H) | 400 |
| 168 | 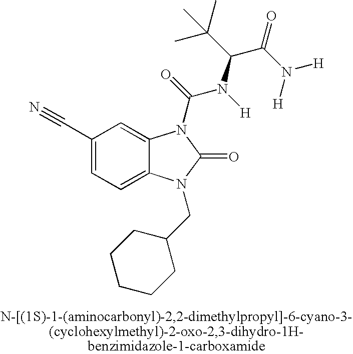 | 0.94-1.07 (m, 12H), 1.16 (m, 3H), 1.58-1.70 (m, 5H), 1.81 (m, 1H), 3.77 (d, J = 6.95 Hz, 2H), 4.27 (d, J = 8.79 Hz, 1H), 7.18 (br. s., 1 H), 7.54 (d, J = 8.42 Hz, 1H), 7.72 (d, J = 8.05 Hz, 1H), 7.66 (br. s., 1H), 8.30 (s, 1 H), 9.05 (d, J = 9.15 Hz, 1H) | 411 |
| 169 | 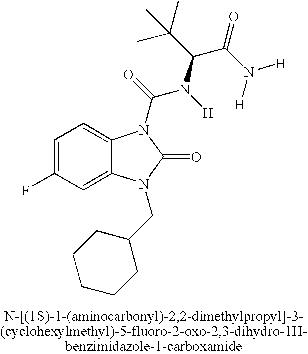 | 0.89-1.03 (m, 9H), 1.10-1.16 (m, 3H), 1.57-1.62 (m, 4H), 1.78 (m, 1H), 3.68 (dd, J = 7.2, 2.0 Hz, 2 H), 4.21 (d, J = 9.2 Hz, 2H), 6.87-6.96 (m, 2H), 7.19 (s, 1 H), 7.32 (dd, J = 8.8, 2.4 Hz, 1H), 7.63 (s, 1H), 7.96 (m, 1H), 8.08 (d, J = 9.2 Hz, 1 H), 9.07 (d, J = 9.2 Hz, 1H) | 404 |
| 170 | 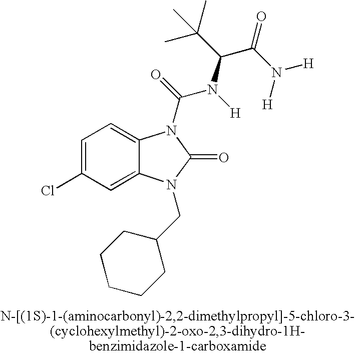 | 0.89-1.16 (m, 12H), 1.62 (m, 4H), 1.78 (m, 1H), 3.70 (d, J = 7.6 Hz, 2H), 4.21 (d, J = 8.8 Hz, 1H), 6.91 (dd, , J = 6.8, 2,0 Hz, 1 H), 7.16 (m, 2H), 7.50 (s, 1H), 7.63 (s, 1H), 7.97 (d, J = 8.4 Hz, 1H), 8.09 (d, J = 9.2 Hz, 1H), 9.09 (d, J = 8.8 Hz, 1H) | 420 |
| 171 | 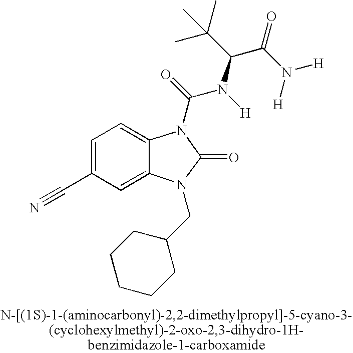 | 0.90-1.09 (m, 11H), 1.16 (m, 3H), 1.58-1.70 (m, 5H), 1.84 (m, 1H), 3.75 (d, J = 7.32 Hz, 2H), 4.26 (d, J = 8.79 Hz, 1H), 7.17 (br. s., 1 H), 7.64 (br. s., 1H), 7.61 (d, J = 8.42 Hz, 1H), 7.93 (s, 1H), 8.16 (d, J = 8.05 Hz, 1H), 9.11 (d, J = 8.79 Hz, 1H) | 411 |
| 172 | 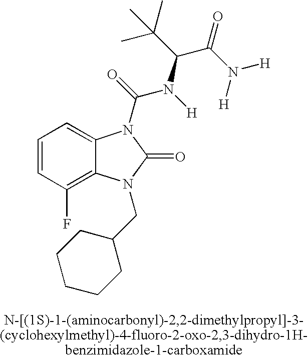 | 0.89-1.16 (m, 13H), 1.58-1.73 (m, 6H), 3.75 (m, 2H), 4.21 (d, J = 9.2 Hz, 1H), 6.89 (d, J = 9.2 Hz, 1H), 7.12 (m, 2H), 7.18 (s, 1H), 7.64 (s, 1H), 7.89 (d, J = 8.8 Hz, 1 H), 8.08 (d, J = 8.8 Hz, 1H), 9.14 (d, J = 8.8 Hz, 1H) | 404 |
| 173 | 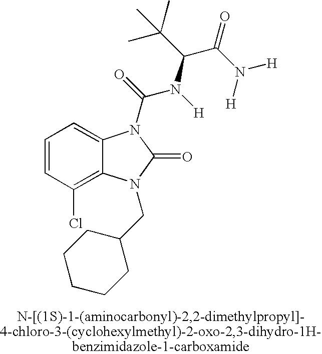 | 0.89-1.11 (m, 13H), 1.58-1.63 (m, 5H), 1.79 (m, 1H), 3.96 (dt, J = 7.2, 1.6 Hz, 2 H), 4.21 (d, J = 9.2 Hz, 2H), 7.13 (t, J = 8.0 Hz, 1H), 7.18 (s, 1H), 7.23 (dd, J = 8.0, 1.2, 1H), 7.64 (s, 1 H), 8.08 (dd, J = 8.0, 1.2 Hz, 1H), 9.18 (d, J = 8.8 Hz, 1H) | 420 |
| 174 | 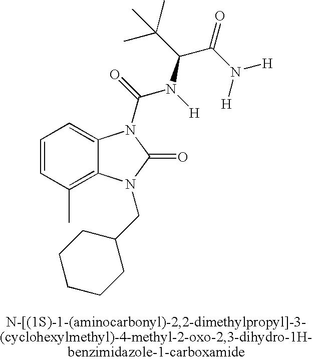 | 0.93-1.00 (m, 12H), 1.17 (m, 3H), 1.60 (m, 3H), 1.68 (m, 2 H), 2.57 (s, 3H), 3.89 (d, J = 6.95 Hz, 2H), 4.25 (d, J = 8.78 Hz, 1H), 7.08-6.98 (m, 1 H), 7.02 (d, J = 4.76 Hz, 1H), 7.13 (br. s., 1H), 7.62 (s, 1H), 8.00 (d, J = 7.69 Hz, 1H), 9.31 (d, J = 8.78 Hz, 1H) | 400 |
| 175 | 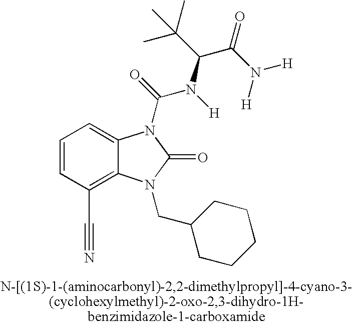 | 0.93-1.12 (m, 11H), 1.17 (m, 4H), 1.63 (m, 1H), 1.71 (d, J = 8.8 Hz, 4H), 1.86 (m, 1H), 3.95 (d, J = 7.32 Hz, 2H), 4.26 (d, J = 9.15 Hz, 1H), 7.18 (s, 1H), 7.30 (t, J = 8.05 Hz, 1H), 7.67-7.62 (m, 1H), 8.37 (d, J = 8.0 Hz, 1H), 9.08 (d, J = 8.8 Hz, 1H) | 411 |

Examples 176-379 can be prepared according to the procedures described above.

| Example | Structure | | Mass |
|---|---|---|---|
| Number | Chemical Name | NMR Date | (M) |
| 176 | 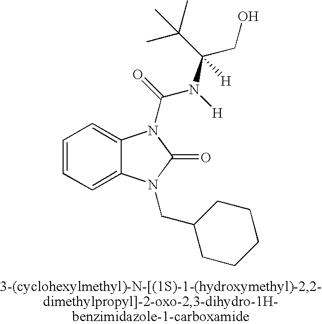 | 1H NMR (400 MHz, DMSO-d6) δ ppm 8.82 (1H, d, J = 9.5 Hz), 8.07 (1H, d, J = 8.1 Hz), 7.32 (1H, d, J = 7.7 Hz), 7.22 (1H, t, J = 7.7 Hz), 7.14 (1H, t, J = 7.7 Hz), 3.73 (2H, dd, J = 6.8, 4.6 Hz), 3.69 (1H, br. s.), 3.66 (1H, d, J = 4.8 Hz), 3.48 (1H, dd, J = 11.3, 4.8 Hz), 1.82 (1H, dd, J = 8.1, 7.0 Hz), 1.63 (5H, d, J = 15.7 Hz), 1.23 (1H, br. s.), 1.15 (3H, d, J = 7.7 Hz), 1.04 (2H, d, J = 11.3 Hz), 1.08 (1H, br. s.), 0.95 (10H, s) | 373.2 |
| 177 | 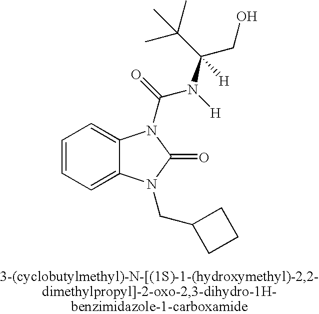 | 1H NMR (400 MHz, DMSO-d6) δ ppm 8.83 (1H, d, J = 9.1 Hz), 8.06 (1H, d, J = 7.7 Hz), 7.34 (1H, d, J = 7.7 Hz), 7.22 (1H, t, J = 7.7 Hz), 7.14 (1H, t, J = 7.7 Hz), 4.65 (1H, t, J = 5.3 Hz), 3.94 (2H, dd, J = 7.0, 4.4 Hz), 3.64- 3.74 (2H, m), 3.44-3.51 (1H, m), 1.98 (2H, d, J = 5.5 Hz), 1.83 (3H, t, J = 10.4 Hz), 0.96 (9H, s) | 345.2 |
| 178 | 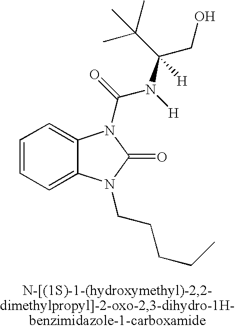 | 1H NMR (400 MHz, DMSO-d6) δ ppm 8.83 (1H, d, J = 9.5 Hz), 8.07 (1H, d, J = 7.7 Hz), 7.32 (1H, d, J = 8.1 Hz), 7.23 (1H, t, J = 7.3 Hz), 7.15 (1H, t, J = 7.7 Hz), 4.66 (1H, t), 3.84-3.93 (2 H, m), 3.89 (0H, d, J = 11.7 Hz), 3.63- 3.74 (2H, m), 3.43-3.52 (1H, m), 3.21 (0H, br. s.), 1.64-1.74 (2H, m), 1.70 (0H, d, J = 6.6 Hz), 1.22-1.37 (4 H, m), 0.95 (10H, s), 0.86 (3H, t, J = 66 Hz) | 347.2 |
| 179 | 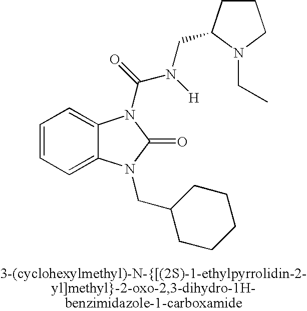 | 1H NMR (400 MHz, DMSO-d6) δ ppm 9.02 (1H, br. s.), 8.11 (1H, d, J = 9.2 Hz), 8.04 (1H, d, J = 7.7 Hz), 7.33 (1H, d, J = 7.7 Hz), 7.24 (1H, t, J = 7.3 Hz), 7.16 (1H, t, J = 7.5 Hz), 6.93 (1H, d, J = 9.2 Hz), 3.74 (3H, d, J = 7.3 Hz), 3.67 (2H, br. s.), 3.58 (1H, d, J = 5.9 Hz), 3.42 (1H, d, J = 4.4 Hz), 3.41 (1H, d, J = 18.7 Hz), 3.31 (1H, br. s.), 3.12 (1H, d, J = 4.4 Hz), 3.07-3.16 (1H, m), 2.18 (1H, d, J = 5.9 Hz), 1.99 (1H, d, J = 8.8 Hz), 1.79-1.87 (2H, m), 1.66 (5H, br. s.), 1.24 (3H, t, J = 7.1 Hz), 1.15 (3H, d, J = 7.3 Hz), 1.04 (1H, d, J = 11.7 Hz) | 384.3 |
| 180 | 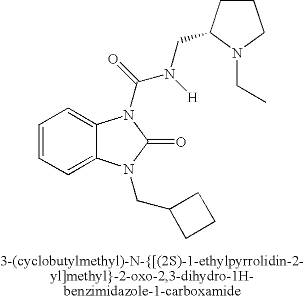 | 1H NMR (400 MHz, DMSO-d6) δ ppm 9.02 (1H, br. s.), 8.03 (1H, d, J = 8.1 Hz), 7.34 (1H, d, J = 8.1 Hz), 7.24 (1H, t, J = 7.7 Hz), 7.16 (1H, t, J = 7.7 Hz), 3.94 (2H, d, J = 7.0 Hz), 3.63 (1H, br. s.), 3.69 (2H, d, J = 14.3 Hz), 3.58 (1 H, br. s.), 3.42 (1H, br. s.), 3.11 (2H, br. s.), 2.77 (1H, dt, J = 14.9, 7.4 Hz), 2.18 (1H, br. s.), 1.93-2.00 (1H, m), 1.98 (2H, d, J = 7.0 Hz), 1.83 (5H, t, J = 10.2 Hz), 1.88 (1H, br. s.), 1.24 (3 H, t, J = 7.0 Hz) | 356.2 |
| 181 | 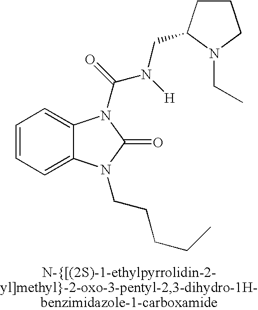 | 1H NMR (400 MHz, DMSO-d6) δ ppm 8.83 (1H, d, J = 4.4 Hz), 8.08 (1H, dd, J = 19.6, 8.6 Hz), 7.27-7.32 (1H, m), 7.23 (1H, q, J = 7.8 Hz), 7.14 (1H, t, J = 7.7 Hz), 6.91 (1H, d, J = 9.1 Hz), 3.88 (2H, t, J = 6.8 Hz), 3.45 (1H, td, J = 6.6, 3.3 Hz), 3.09 (1H, dt, J = 9.2, 4.7 Hz), 2.82 (1H, dd, J = 12.1, 7.3 Hz), 2.63 (1H, d, J = 4.8 Hz), 2.26 (1H, dd, J = 11.9, 6.8 Hz), 2.16 (1H, q, J = 8.4 Hz), 1.85 (1H, dd, J = 11.9, 8.2 Hz), 1.70 (1H, br. s.), 1.67 (3H, d, J = 7.3 Hz), 1.54 (1H, dd, J = 12.1, 6.2 Hz), 1.30 (3H, d, J = 7.0 Hz), 1.24- 1.33 (1H, m), 1.05 (3H, t, J = 7.1 Hz), 0.85 (3H, t, J = 6.6 Hz) | 358.2 |
| 182 | 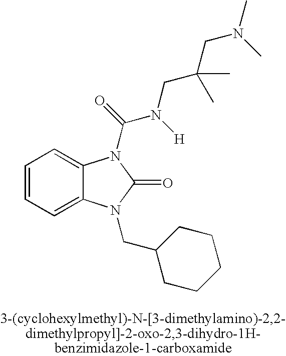 | 1H NMR (400 MHz, DMSO-d6) δ ppm 8.91 (1H, t, J = 6.0 Hz), 8.04 (1H, d, J = 8.1 Hz), 7.33 (1H, d, J = 7.7 Hz), 7.24 (1H, t, J = 7.5 Hz), 7.16 (1H, t, J = 7.7 Hz), 3.73 (2H, d, J = 7.0 Hz), 3.35 (5H, d, J = 6.2 Hz), 3.00 (2H, br. s.), 2.82 (5H, br. s.), 1.81 (1H, br. s.), 1.64 (5H, d, J = 14.6 Hz), 1.24 (1H, br. s.), 1.15 (3H, d, J = 6.6 Hz), 1.05 (8H, br. s.) | 387.3 (M + H) |
| 183 | 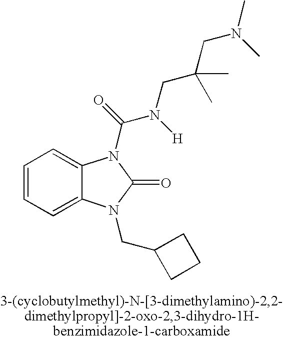 | 1H NMR (400 MHz, DMSO-d6) δ ppm 0.90 (br. s., 6H) 1.83 (br. s., 4H) 1.98 (d, J = 6.22 Hz, 2H) 2.16 (br. s., 1 H) 2.26 (br. s., 6H) 3.31 (br. s., 2H) 3.94 (br. s., 3H) 7.08-7.26 (m, 3H) 7.33 (d, J = 7.69 Hz, 1H) 8.04 (d, J = 6.96 Hz, 1H) 9.01 (br. s., 1H) | 358.2 |
| 184 | 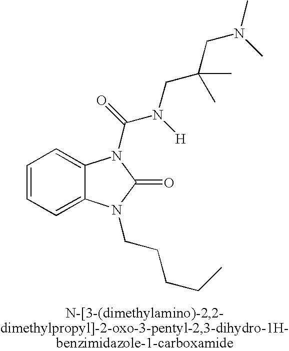 | 1H NMR (400 MHz, DMSO-d6) δ ppm 8.99 (1H, t, J = 4.9 Hz), 8.05 (1H, d, J = 8.1 Hz), 7.28-7.33 (1H, m), 7.23 (1H, t, J = 7.7 Hz), 7.14 (1H, t, J = 7.7 Hz), 6.93 (1H, d, J = 9.1 Hz), 3.88 (2H, t, J = 7.1 Hz), 3.23-3.24 (1H, m), 2.27 (6H, s), 2.18 (2H, s), 2.02 (1H, s), 1.88 (1H, s), 1.83 (1H, s), 1.69 (2H, t, J = 7.0 Hz), 1.30 (2H, d, J = 3.3 Hz), 1.24-1.34 (2H, m), 0.90 (6H, s), 0.85 (3H, t, J = 6.8 Hz) | 360.3 |
| 185 | 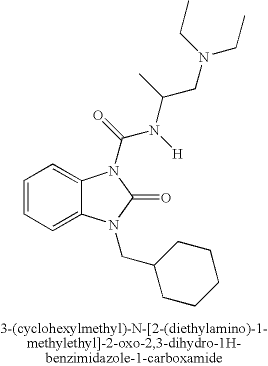 | 1H NMR (400 MHz, DMSO-d6) δ ppm 8.78 (1H, d, J = 8.1 Hz), 8.61 (1 H, dd, J = 10.4, 4.2 Hz), 8.05 (1H, d, J = 8.1 Hz), 7.30-7.36 (1H, m), 7.25 (1H, t, J = 7.7 Hz), 7.17 (1H, t, J = 7.7 Hz), 4.43 (1H, d, J = 4.4 Hz), 3.73 (2H, d, J = 7.0 Hz), 3.17 (3H, br. s.), 2.95 (1 H, br. s.), 1.81 (1H, br. s.), 1.60-1.70 (5H, m), 1.30 (3H, d, J = 6.2 Hz), 1.23 (6H, d, J = 6.2 Hz), 1.15 (3H, d, J = 6.2 Hz), 1.04 (2H, d, J = 8.4 Hz), 1.08 (1H, br. s.) | 386.3 |
| 186 | 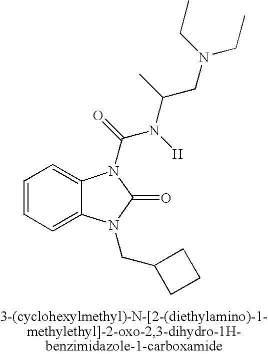 | 1H NMR (400 MHz, DMSO-d6) δ ppm 8.71 (1H, d, J = 6.6 Hz), 7.33 (1 H, d, J = 8.1 Hz), 7.15 (1H, d, J = 8.4 Hz), 7.22 (1H, t, J = 7.3 Hz), 4.01 (1H, br. s.), 3.92 (3H, d, J = 7.0 Hz), 3.30 (3 H, br. s.), 2.77 (1H, d, J = 6.2 Hz), 2.62 (2H, dd, J = 10.4, 5.7 Hz), 2.65 (1H, br. s.), 2.58 (1H, br. s.), 1.98 (1H, d, J = 11.3 Hz), 1.81 (3H, d, J = 5.1 Hz), 1.78-1.87 (1H, m), 1.23 (4H, d, J = 6.6 Hz), 1.01 (5H, t, J = 5.7 Hz) | 358.2 |
| 187 | 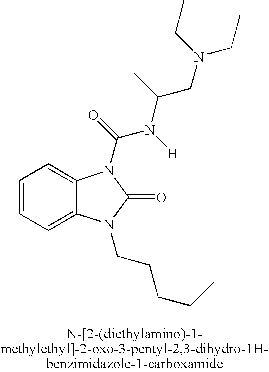 | 1H NMR (400 MHz, DMSO-d6) δ ppm 8.70 (1H, d, J = 6.6 Hz), 7.30 (1 H, d, J = 7.3 Hz), 7.22 (1H, t, J = 8.1 Hz), 7.14 (1H, t, J = 7.3 Hz), 3.87 (3H, t, J = 7.1 Hz), 3.30 (1H, br. s.), 2.59 (2 H, br. s.), 2.45 (1H, br. s.), 1.66 (2H, d, J = 5.9 Hz), 1.69 (1H, br. s.), 1.29 (5 H, d, J = 2.6 Hz), 1.22 (5H, d, J = 6.2 Hz), 0.99 (6H, br. s.), 0.84 (3H, t, J = 6.8 Hz) | 360.3 |
| 188 | 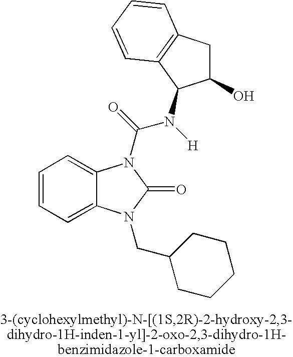 | 1H NMR (400 MHz, DMSO-d6) δ ppm 9.32 (1H, d, J = 8.1 Hz), 8.15 (1 H, d, J = 7.7 Hz), 7.32 (1H, br. s.), 7.25 (2H, d, J = 7.7 Hz), 7.18 (1H, br. s.), 7.20 (1H, d, J = 7.0 Hz), 5.35 (1H, d, J = 4.4 Hz), 5.30 (1H, d, J = 4.8 Hz), 4.54 (1H, d, J = 4.4 Hz), 3.71 (3H, d, J = 7.0 Hz), 3.13 (1H, dd, J = 16.3, 4.6 Hz), 2.85 (1H, d, J = 16.5 Hz), 1.82 (1 H, dd, J = 4.2, 2.7 Hz), 162 (6H, d, J = 15.0 Hz), 1.14 (4H, d, J = 7.3 Hz), 1.03 (2H, t, J = 11.0 Hz) | 405.2 |
| 189 | 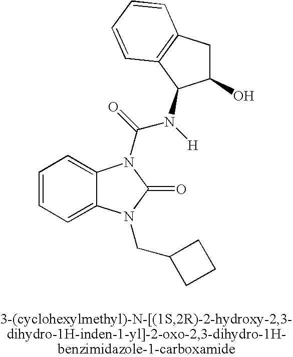 | 1H NMR (400 MHz, DMSO-d6) δ ppm 1.77-1.87 (m, 4H) 1.97 (d, J = 6.59 Hz, 2H) 2.73-2.80 (m, 1H) 2.85 (d, J = 16.11 Hz, 1H) 3.14 (dd, J = 16.47, 4.39 Hz, 1H) 3.92 (d, J = 7.32 Hz, 3H) 4.55 (br. s., 1H) 5.28-5.38 (m, 2H) 7.18-729 (m, 5H) 7.35 (d, J = 7.32 Hz, 1H) 8.15 (d, J = 7.69 Hz, 1 H) 9.32 (d, J = 8.05 Hz, 1H) | 377.2 |
| 190 | 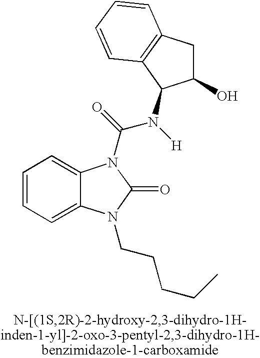 | 1H NMR (400 MHz, DMSO-d6) δ ppm 9.31 (1H, d, J = 8.1 Hz), 8.15 (1 H, d, J = 7.7 Hz), 7.18-7.29 (5H, m), 5.32 (1H, dd, J = 15.7, 4.8 Hz), 4.54 (1 H, d, J = 2.2 Hz), 3.87 (2H, t, J = 7.0 Hz), 3.13 (1H, dd, J = 16.7, 4.6 Hz), 2.85 (1H, d, J = 16.1 Hz), 1.66 (1H, br. s.), 1.69 (2H, d, J = 7.0 Hz), 1.30 (5H, d, J = 3.3 Hz), 1.23 (1H, br. s.), 0.84 (3 H, t, J = 6.8 Hz) | 379.2 |
| 191 | 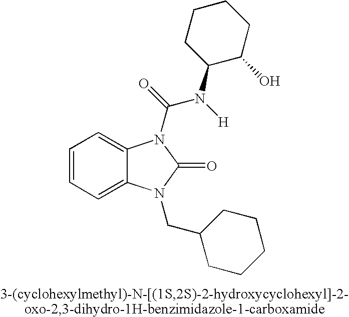 | 1H NMR (400 MHz, DMSO-d6) δ ppm 8.75 (1H, d, J = 7.3 Hz), 8.06 (1 H, d, J = 7.7 Hz), 7.31 (1H, d, J = 7.7 Hz), 7.22 (1H, t, J = 7.5 Hz), 7.14 (1H, t, J = 7.7 Hz), 4.83 (1H, d, J = 4.8 Hz), 3.72 (2H, d, J = 7.0 Hz), 3.52 1H, br. s.), 3.30-3.39 (1H, m), 2.04 (1H, d, J = 2.9 Hz), 1.84 (2H, d, J = 18.3 Hz), 1.83 (7H, d, J = 15.7 Hz), 1.28 (4H, d, J = 7.0 Hz), 1.15 (3H, d, J = 7.7 Hz), 1.03 (2H, d, J = 10.2 Hz), 1.07 (1H, br. s.) | 371.2 |
| 192 | 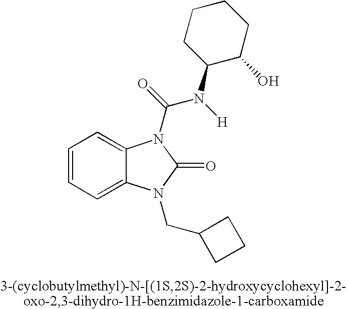 | 1H NMR (400 MHz, DMSO-d6) δ ppm 7.33 (1H, d, J = 7.7 Hz), 7.22 (1 H, t, J = 7.9 Hz), 7.14 (1H, t, J = 7.3 Hz), 4.83 (1H, d, J = 5.1 Hz), 3.92 (2H, d, J = 7.3 Hz), 3.48-3.56 (1H, m), 3.35 (1H, d, J = 4.4 Hz), 3.34 (1H, d, J = 17.2 Hz), 2.74-2.81 (1H, m), 1.94- 2.05 (3H, m), 1.82 (3H, d, J = 5.1 Hz), 1.78-1.89 (2H, m), 1.66 (1H, br. s.), 1.60 (1H, br. s.), 1.28 (5H, d, J = 6.6 Hz), 1.21 (1H, br. s.) | 343.2 |
| 193 | 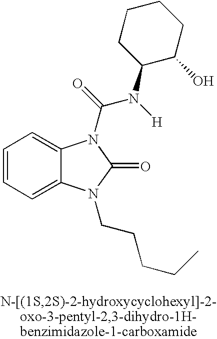 | 1H NMR (400 MHz, DMSO-d6) δ ppm 8.74 (1H, d, J = 7.3 Hz), 8.06 (1 H, d, J = 7.7 Hz), 7.29-7.33 (1H, m), 7.22 (1H, t, J = 7.3 Hz), 7.15 (1H, t, J = 7.7 Hz), 4.82 (1H, d, J = 5.1 Hz), 3.87 (2H, t, J = 7.1 Hz), 3.49-3.56 (1 H, m), 3.35 (1H, d, J = 4.0 Hz), 3.25 (1 H, br. s.), 2.04 (1H, br. s.), 1.87 (1H, d, J = 8.4 Hz), 1.67 (2H, d, J = 7.0 Hz), 1.70 (1H, br. s.), 1.60 (1H, br. s.), 1.23-1.33 (9H, m), 0.85 (3H, t, J = 6.6 Hz) | 345.2 |
| 194 | 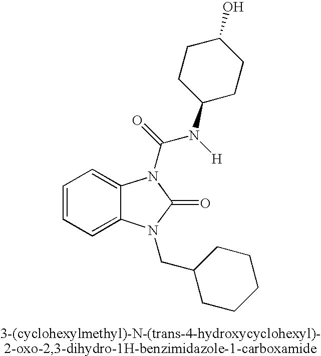 | 1H NMR (400 MHz, DMSO-d6) δ ppm 8.62 (1H, d, J = 7.3 Hz), 8.04 (1 H, d, J = 7.7 Hz), 7.30 (1H, d, J = 8.1 Hz), 7.21 (1H, t, J = 7.5 Hz), 7.14 (1H, t, J = 7.7 Hz), 4.54 (1H, d, J = 3.7 Hz), 3.71 (2H, br. s.), 3.62-3.68 (1H, m), 3.43-3.51 (1H, m), 3.26 (0H, br. s.), 1.92-1.99 (2H, m), 1.76-1.86 (3H, m), 1.57-1.69 (5H, m), 1.32 (3H, d, J = 11.0 Hz), 1.24-1.39 (2H, m), 1.10- 1.19 (3H, m), 0.97-1.08 (2H, m) | 371.2 |
| 195 | 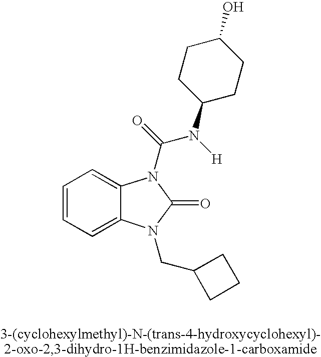 | 1H NMR (400 MHz, DMSO-d6) δ ppm 8.62 (1H, d, J = 7.7 Hz), 7.32 (1 H, d, J = 7.7 Hz), 7.22 (1H, t, J = 7.7 Hz), 7.14 (1H, J = 7.3 Hz), 4.54 (1H, 3.60-3.69 (1H, m), 3.47 (1H, dd, (1H, t, J = 7.5 Hz), 1.97 (3H, d, J = 4.0 Hz), 1.94 (2H, br. s.), 1.83 (3H, d, J = 7.0 Hz), 1.81 (2H, br. s.), 1.76 (1H, br. s.), 1.32 (3H, d, J = 13.2 Hz), 1.27- 1.37 (1H, m) | 343.2 |
| 196 | 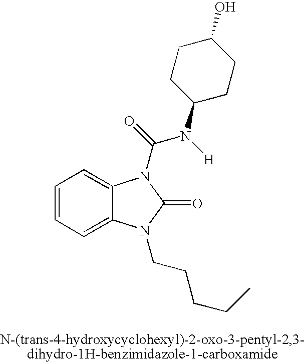 | 1H NMR (400 MHz, DMSO-d6) δ ppm 8.62 (1H, d, J = 7.3 Hz), 8.04 (1 H, d, J = 7.7 Hz), 7.30 (1H, d, J = 7.7 Hz), 7.22 (1H, t, J = 7.7 Hz), 7.14 (1H, t, J = 7.7 Hz), 4.55 (1H, d, J = 4.0 Hz), 3.86 (2H, t, J = 7.1 Hz), 3.60-3.70 (1 H, m), 3.43-3.52 (1H, m), 1.89-2.00 (2H, m), 1.83 (2H, d, J = 10.2 Hz), 1.60-1.75 (1H, m), 1.68 (2H, d, J = 7.0 Hz), 1.29 (5H, d, J = 9.9 Hz), 1.21-1.40 (4H, m), 0.84 (2H, t, J = 6.8 Hz) | 345.2 |
| 197 | 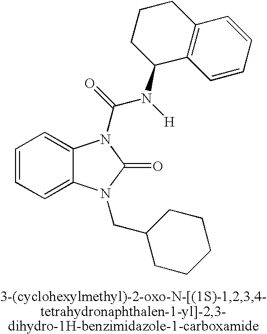 | 1H NMR (400 MHz, DMSO-d6) δ ppm 9.00 (1H, d, J = 8.1 Hz), 8.10 (1 H, d, J = 8.1 Hz), 7.31 (1H, d, J = 8.1 Hz), 7.34 (1H, d, J = 7.3 Hz), 7.13- 7.23 (3H, m), 5.09 (1H, d, J = 6.6 Hz), 3.68 (2H, d, J = 7.0 Hz), 3.27 (1H, s), 2.70-2.87 (2H, m), 2.08 (1H, d, J = 5.9 Hz), 1.91 (1H, dd, J = 12.1, 5.9 Hz), 1.81 (1H, br. s.), 1.63 (2H, d, J = 5.5 Hz), 1.60 (6H, d, J = 13.9 Hz), 1.12 (4H, d, J = 7.7 Hz), 1.03 (1H, br. s.), 0.99 (1H, d, J = 9.2 Hz) | 403.2 |
| 198 | 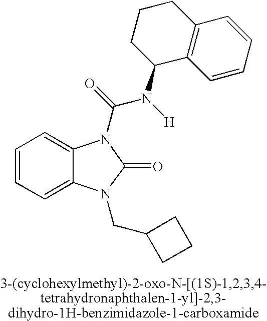 | 1H NMR (400 MHz, DMSO-d6) δ ppm 9.00 (1H, d, J = 7.7 Hz), 8.10 (1 H, d, J = 7.7 Hz), 7.34 (2H, d, J = 7.3 Hz), 7.14-7.26 (4H, m), 5.06-5.14 (1H, m), 3.89 (2H, d, J = 7.0 Hz), 2.82 (0H, br. s.), 2.70-2.79 (3H, m), 2.00- 2.11 (1H, m), 1.87-1.98 (1H, m), 1.94 (2H, d, J = 2.9 Hz), 1.76-1.86 (1 H, m), 1.82 (5H, dd, J = 12.3, 5.7 Hz), 1.74 (1H, br. s.) | 376.2 (M + H) |
| 199 | 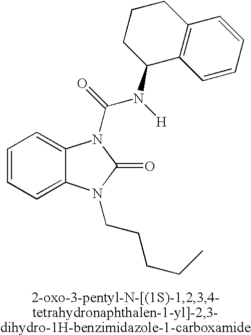 | 1H NMR (400 MHz, DMSO-d6) δ ppm 9.00 (1H, d, J = 8.1 Hz), 8.11 (1 H, d, J = 8.1 Hz), 7.32 (1H, d, J = 8.8 Hz), 7.25 (1H, t, J = 7.5 Hz), 7.17 (2H, d, J = 10.6 Hz), 3.84 (2H, t, J = 7.1 Hz), 2.72-2.87 (2H, m), 2.05 (1H, d, J = 5.9 Hz), 2.02-2.11 (1H, m), 1.91 (1 H, dd, J = 12.3, 6.0 Hz), 1.84 (2H, d, J = 5.5 Hz), 1.81 (1H, br. s.), 1.64 (1H, br. s.), 1.66 (2H, d, J = 7.0 Hz), 1.28 (4 H, d, J = 3.3 Hz), 1.23 (1H, br. s.), 0.83 (2H, t, J = 6.6 Hz) | 377.2 |
| 200 | 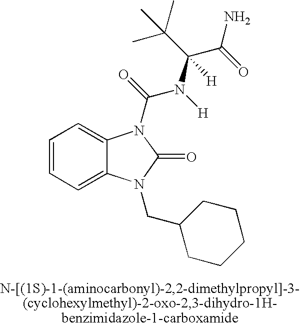 | 1H NMR (400 MHz, DMSO-d6) δ ppm 9.19 (1H, d, J = 8.8 Hz), 8.04 (1 H, d, J = 7.7 Hz), 7.62 (1H, br. s.), 7.32 (1H, d, J = 7.7 Hz), 7.23 (1H, t, J = 7.3 Hz), 7.11-7.19 (2H, m), 4.25 (1H, d, J = 8.8 Hz), 3.73 (1H, d, J = 3.7 Hz), 3.74 (1H, br. s.), 1.83 (1H, br. s.), 1.63 (5H, d, J = 15.4 Hz), 1.15 (4H, d, J = 7.3 Hz), 1.00 (11H, s) | 386.2 |
| 201 | 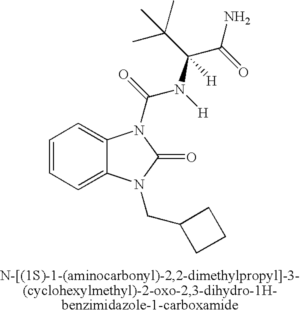 | 1H NMR (400 MHz, DMSO-d6) δ ppm 9.19 (1H, d, J = 8.8 Hz), 8.03 (1 H, d, J = 8.1 Hz), 7.62 (1H, br. s.), 7.33 (1H, d, J = 7.7 Hz), 7.22 (1H, t, J = 7.5 Hz), 7.11-7.18 (2H, m), 4.24 (1H, d, J = 8.8 Hz), 3.93 (2H, dd, J = 6.8, 2.7 Hz), 1.98 (2H, d, J = 6.6 Hz), 1.77- 1.89 (4H, m), 1.00 (10H, s) | 358.2 |
| 202 | 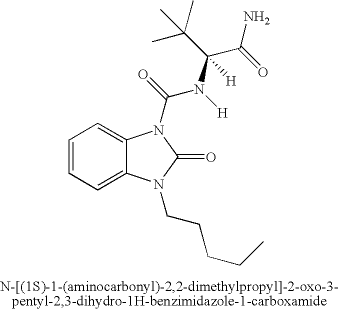 | 1H NMR (400 MHz, DMSO-d6) δ ppm 9.19 (1H, d, J = 8.8 Hz), 8.04 (1 H, d, J = 8.1 Hz), 7.62 (1H, br. s.), 7.31 (1H, d, J = 7.7 Hz), 7.23-7.27 (1H, m), 7.12-7.23 (2H, m), 4.25 (1H, d, J = 8.8 Hz), 3.89 (2H, t, J = 6.6 Hz), 3.22 (0H, br. s.), 1.64-1.74 (1H, m), 1.64-1.74 (1H, m), 1.16-1.37 (4H, m), 1.00 (10 H, s), 0.85 (3 H, t, J = 6.4 Hz) | 360.2 |
| 203 | 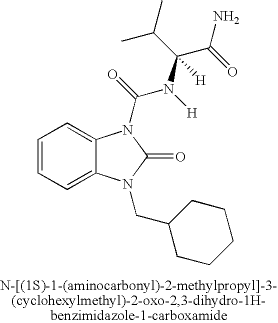 | 1H NMR (400 MHz, DMSO-d6) δ ppm 9.08 (1H, d, J = 8.1 Hz), 8.04 (1 H, d, J = 7.7 Hz), 7.61 (1H, br. s.), 7.32 (1H, d, J = 8.1 Hz), 7.23 (1H, t, J = 7.3 Hz), 7.12-7.18 (1H, m), 4.29 (1H, dd, J = 8.2, 4.9 Hz), 3.73 (2H, d, J = 5.5 Hz), 2.15 (1H, dd, J = 12.3, 6.8 Hz), 1.83 (1H, br. s.), 1.64 (5H, d, J = 16.5 Hz), 1.10-1.20 (3H, m), 1.01-1.10 (2H, m), 0.88-0.98 (6H, m) | 372.2 |
| 204 | 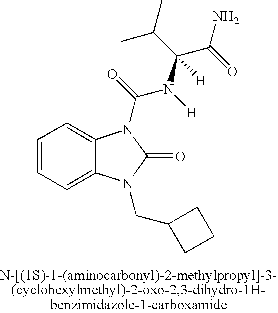 | 1H NMR (400 MHz, DMSO-d6) δ ppm 9.08 (1H, d, J = 8.4 Hz), 8.03 (1 H, d, J = 7.7 Hz), 7.61 (1H, br. s.), 7.34 (1H, d, J = 7.7 Hz), 7.23 (1H, t, J = 7.5 Hz), 7.15 (2H, d, J = 6.6 Hz), 4.29 (1H, dd, J = 8.2, 4.9 Hz), 3.94 (2H, d, J = 7.0 Hz), 2.78 (1H, ddd, J = 14.7, 7.2, 7.0 Hz), 2.15 (1H, dd, J = 11.9, 6.4 Hz), 1.98 (2H, d, J = 5.5 Hz), 1.79-1.89 (4 H, m), 0.88-0.98 (7H, m) | 344.2 |
| 205 | 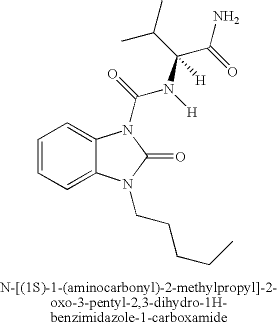 | 1H NMR (400 MHz, DMSO-d6) δ ppm 9.08 (1H, d, J = 8.4 Hz), 8.03 (1 H, d, J = 8.1 Hz), 7.61 (1H, br. s.), 7.31 (1H, d, J = 7.7 Hz), 7.23 (1H, t, J = 7.5 Hz), 7.12-7.18 (2 m), 4.29 (1H, dd, J = 8.2, 4.9 Hz), 3.88 (2H, t, J = 7.0 Hz), 2.10-2.20 (1H, m), 1.68 (0H, br. s.), 1.61-1.77 (2H, m), 1.22-1.41 (4 H, m), 0.86 (2H, t, J = 6.6 Hz), 0.81- 1.00 (7H, m) | 346.2 |
| 206 | 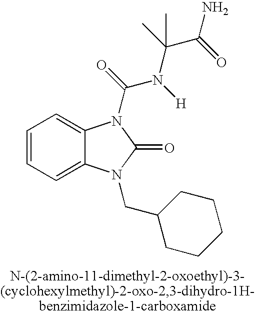 | 1H NMR (400 MHz, DMSO-d6) δ ppm 9.40 (1H, s), 8.04 (1H, d, J = 7.7 Hz), 7.44 (1H, br. s.), 7.26-7.33 (1H, m), 7.21 (1H, t, J = 7.9 Hz), 7.13 (1H, t, J = 7.9 Hz), 7.07 (1H, br. s.), 3.71 (2 H, d, J = 7.3 Hz), 1.81 (1H, br. s.), 1.64 (5H, d, J = 15.7 Hz), 1.55 (6H, s), 1.15 (2H, d, J = 7.3 Hz), 1.04 (2H, q, J = 10.9 Hz) | 358.2 |
| 207 | 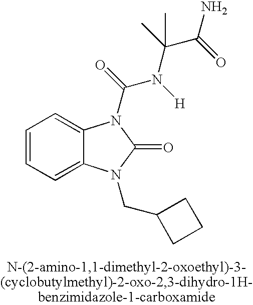 | 1H NMR (400 MHz, DMSO-d6) δ ppm 9.40 (1H, s), 8.04 (1H, d, J = 8.1 Hz), 7.45 (1H, br. s.), 7.31 (1H, d, J = 7.7 Hz), 7.21 (1H, t, J = 7.7 Hz), 7.13 (1H, t, J = 7.7 Hz), 7.09 (1H, br. s.), 3.92 (2H, d, J = 7.0 Hz), 1.98 (2H, d, J = 5.5 Hz), 1.78-1.88 (4H, m), 1.55 (7H, s) | 330.2 |
| 208 | 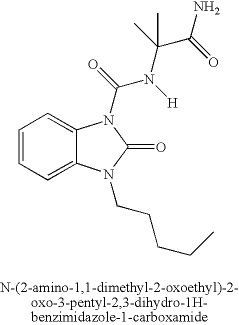 | 1H NMR (400 MHz, DMSO-d6) δ ppm 9.40 (1H, s), 8.04 (1H, d, J = 8.1 Hz), 7.45 (1H, br. s.), 7.30 (1H, d, J = 7.7 Hz), 7.22 (1H, t, J = 7.5 Hz), 7.13 (1H, t, J = 7.7 Hz), 7.08 (1H, br. s.), 3.87 (2H, t, J = 7.0 Hz), 1.67 (1H, br. s.), 1.69 (2H, d, J = 6.6 Hz), 1.55 (7 H, s), 1.23-1.40 (4H, m), 0.81-0.91 (2H, m) | 332.2 |
| 209 | 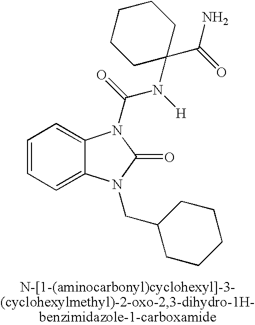 | 1H NMR (400 MHz, DMSO-d6) δ ppm 9.01 (1H, s), 8.02 (1H, d, J = 7.7 Hz), 7.37 (1H, br. s.), 7.31 (1H, s), 7.20-7.26 (1H, m), 7.14 (1H, t, J = 7.7 Hz), 6.83 (1H, br. s.), 3.74 (2H, d, J = 7.3 Hz), 2.05 (2H, d, J = 13.2 Hz), 1.84 (1H, br. s.), 1.76 (3H, t, J = 13.5 Hz), 1.68 (4H, br. s.), 1.60 (4H, br. s.), 1.40 (2H, d, J = 13.2 Hz), 1.32- 1.48 (1H, m), 1.24 (1H, d, J = 3.7 Hz), 1.16 (3H, d, J = 7.0 Hz), 1.09 (1H, br. s.), 1.05 (2H, d, J = 12.1 Hz) | 398.2 |
| 210 | 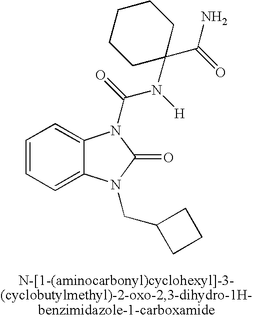 | 1H NMR (400 MHz, DMSO-d6) δ ppm 9.01 (1H, s), 8.01 (1H, d, J = 8.1 Hz), 7.37 (1H, br. s.), 7.34 (1H, d, J = 7.7 Hz), 7.23 (1H, t, J = 7.5 Hz), 7.14 (1H, t, J = 7.7 Hz), 6.83 (1H, br. s.), 3.95 (2H, d, J = 7.0 Hz), 1.95-2.09 (4H, m), 1.76 (2H, t, J = 13.5 Hz), 1.85 (4H, d, J = 2.9 Hz), 1.61 (3H, d, J = 10.2 Hz), 1.40 (2H, q, J = 12.4 Hz), 1.18-1.30 (1H, m) | 370.2 |
| 211 | 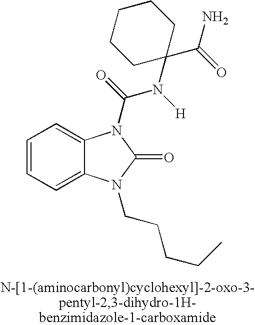 | 1H NMR (400 MHz, DMSO-d6) δ ppm 9.01 (1H, s), 8.02 (1H, d, J = 8.1 Hz), 7.32 (1H, d, J = 7.7 Hz), 7.36 (1H, br. s.), 7.23 (1H, t, J = 7.7 Hz), 7.14 (1 H, t, J = 7.7 Hz), 6.83 (1H, br. s.), 3.90 (2H, t, J = 7.1 Hz), 2.05 (2H, d, J = 13.5 Hz), 1.65-1.80 (4H, m), 1.61 (3H, d, J = 11.0 Hz), 1.33 (4H, d, J = 6.6 Hz), 128-1.48 (2H, m), 1.23 (2H, d, J = 10.2 Hz), 0.82-0.91 (2H, m) | 372.2 |
| 212 | 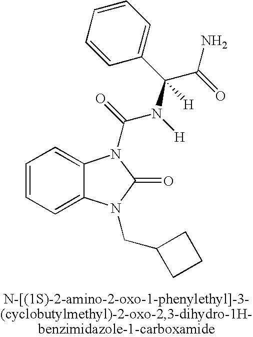 | 1H NMR (400 MHz, DMSO-d6) δ ppm 9.72 (1H, d, J = 7.0 Hz), 7.97 (1 H, d, J = 8.1 Hz), 7.83 (1H, br. s.), 7.48 (2H, d, J = 7.3 Hz), 7.33 (3H, dd, J = 17.6, 9.9 Hz), 7.29-7.40 (2H, m), 7.22 (1H, t, J = 7.5 Hz), 7.12 (1H, t, J = 7.9 Hz), 5.46 (1H, d, J = 7.0 Hz), 3.94 (2H, d, J = 7.0 Hz), 2.77 (1H, d, J = 7.3 Hz), 1.99 (2H, d, J = 7.7 Hz), 1.86 (1H, br. s.), 1.79-1.85 (1H, m), 1.83 (2H, d, J = 4.0 Hz) | 378.2 |
| 213 | 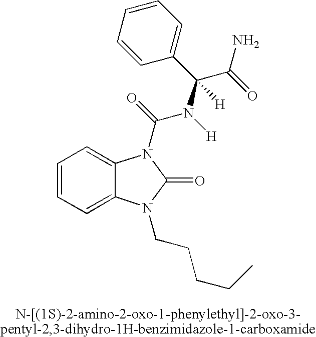 | 1H NMR (400 MHz, DMSO-d6) δ ppm 9.73 (1H, d, J = 7.0 Hz), 7.98 (1 H, d, J = 7.7 Hz), 7.84 (1H, br. s.), 7.48 (2H, d, J = 7.3 Hz), 7.38 (2H, t, J = 7.3 Hz), 7.31 (3H, d, J = 2.9 Hz), 7.22 (1H, t, J = 7.5 Hz), 7.12 (1H, t, J = 7.7 Hz), 5.46 (1H, d, J = 7.0 Hz), 3.89 (2H, t, J = 7.1 Hz), 1.68 (0H, br. s.), 1.65 1.74 (3H, m), 1.32 (4H, d, J = 3.3 Hz), 0.86 (3H, t, J = 6.8 Hz) | 380.2 |
| 214 | 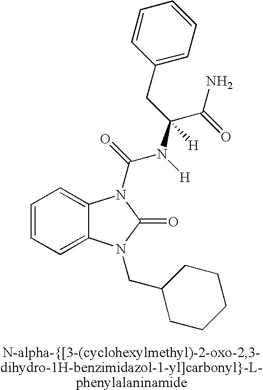 | 1H NMR (400 MHz, DMSO-d6) δ ppm 9.03 (1H, d, J = 7.7 Hz), 7.98 (1 H, d, J = 7.7 Hz), 7.67 (1H, br. s.), 7.29 (1H, d, J = 8.1 Hz), 7.19-7.26 (6H, m), 7.12 (1H, t, J = 7.7 Hz), 4.59-4.66 (1H, m), 3.70 (2H, d, J = 7.0 Hz), 3.16 (1H, dd, J = 13.5, 4.8 Hz), 2.97 (1H, dd, J = 13.5, 7.7 Hz), 1.79 (1H, br. s.), 1.61 (5H, br. s.), 1.11-1.20 (3H, m), 1.03 (2H, t, J = 11.2 Hz) | 420.0 |
| 215 | 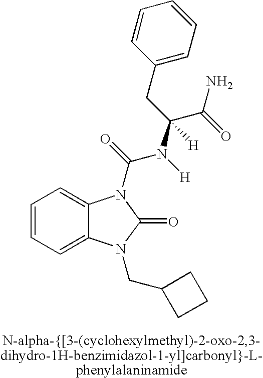 | 1H NMR (400, MHz, DMSO-d6) δ ppm 9.03 (1H, d, J = 7.7 Hz), 7.97 (1 H, d, J = 8.1 Hz), 7.67 (1H, br. s.), 7.31 (1H, d, J = 7.7 Hz), 7.18-7.27 (6H, m), 7.12 (1H, t, J = 7.7 Hz), 4.58-4.67 (1H, m), 3.91 (2H, d, J = 7.3 Hz), 3.16 (1H, dd, J = 13.9, 5.1 Hz), 2.97 (1H, dd, J = 13.9, 8.1 Hz), 2.71-2.81 (1H, m), 1.92-2.01 (2H, m), 1.76-1.87 (4 H, m) | 392.2 |
| 216 | 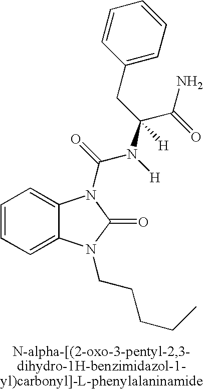 | 1H NMR (400 MHz, DMSO-d6) δ ppm 9.03 (1H, d, J = 7.7 Hz), 7.98 (1 H, d, J = 8.1 Hz), 7.66 (1H, br. s.), 7.19- 7.30 (8H, m), 7.13 (1H, t, J = 7.9 Hz), 4.63 (1H, d, J = 5.5 Hz), 3.86 (2H, t, J = 7.1 Hz), 3.16 (1H, dd, J = 13.9, 5.1 Hz), 2.98 (1H, dd, J = 13.9, 8.1 Hz), 1.67 (2H, qd, J = 7.0, 6.8 Hz), 1.31 (3 H, d, J = 6.2 Hz), 1.25-1.35 (1H, m), 0.86 (3H, t, J = 6.8 Hz) | 394.2 |
| 217 | 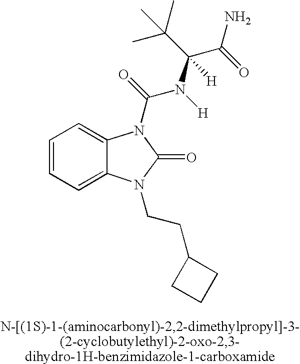 | 1H NMR (400 MHz, DMSO-d6) δ ppm 9.19 (1H, d, J = 9.1 Hz), 8.04 (1 H, d, J = 8.1 Hz), 7.61 (1H, br. s.), 7.27- 7.31 (1H, m), 7.24 (1H, t, J = 7.7 Hz), 7.14 (2H, d, J = 7.7 Hz), 4.26 (1H, d, J = 9.1 Hz), 3.82 (2H, t, J = 6.6 Hz), 2.29 (1H, qd, J = 7.7, 7.5 Hz), 1.99 (1 H, d, J = 3.7 Hz), 1.96-2.03 (1H, m), 1.81 (2H, d, J = 7.3 Hz), 1.79-1.85 (1 H, m), 1.75-1.79 (1H, m), 1.62 (2H, q, J = 8.4 Hz), 1.00 (9H, s) | 372.2 |
| 218 | 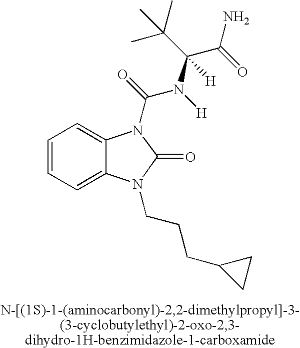 | 1H NMR (400 MHz, DMSO-d6) δ 9.19 (1H, d, J = 8.1 Hz), 8.04 (1 H, d, J = 7.7 Hz), 7.61 (1H, br. s.), 7.29- 7.33 (1H, m), 7.24 (1H, t, J = 7.7 Hz), 7.14 (2H, d, J = 8.1 Hz), 4.25 (1H, d, J = 8.8 Hz), 3.91 (1H, d, J = 3.7 Hz), 3.91 (1H, d, J = 17.2 Hz), 1.75-1.83 (2 H, m, J = 7.2, 7.2, 7.2, 7.2 Hz), 1.25 (2 H, q, J = 7.1 Hz), 1.00 (9H, s), 0.74 (1 H, m), 0.39 (2H, d, J = 7.7 Hz), 0.02 (2H, d, J = 4.4 Hz) | 372.2 |
| 219 | 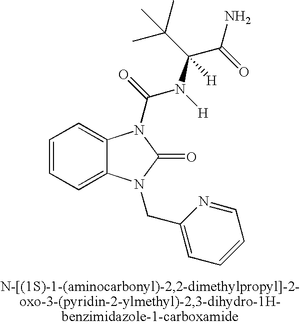 | 1H NMR (400 MHz, DMSO-d6) δ ppm 9.17 (1H, d, J = 8.8 Hz), 7.79 (1 H, t, J = 7.3 Hz), 7.63 (1H, br. s.), 7.41 (1H, d, J = 8.1 Hz), 7.15 (4H, br. s.), 5.24 (2H, br. s.), 4.27 (1H, d, J = 8.8 Hz), 1.00 (11H, s) | 381.2 |
| 220 | 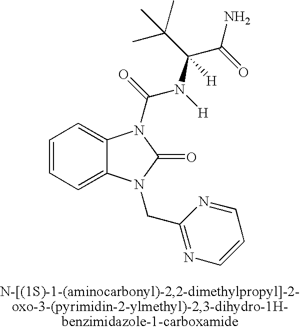 | 1H NMR (400 MHz, DMSO-d6) δ ppm 9.13 (1H, d, J = 8.8 Hz), 8.76 (1 H, d, J = 4.8 Hz), 8.07 (1H, d, J = 5.5 Hz), 7.64 (1H, br. s.), 7.43 (1H, t, J = 4.6 Hz), 7.16 (3H, br. s.), 6.90- 7.00 (1H, m), 5.36 (2H, s), 4.27 (1H, d, J = 8.8 Hz), 0.99 (9H, s), 0.93 (3H, s) | 382.2 |
| 221 | 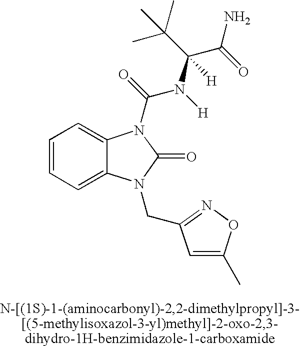 | 1H NMR (400 MHz, DMSO-d6) δ ppm 9.11 (1H, d, J = 8.8 Hz), 8.05 (1 H, d, J = 8.1 Hz), 7.63 (1H, br. s.), 7.09- 7.26 (3H, m), 5.17 (2H, s), 4.26 (1 H, d, J = 9.5 Hz), 2.31-2.40 (4H, m), 2.25 (2H, s), 1.01 (9H, s) | 385.2 |
| 222 | 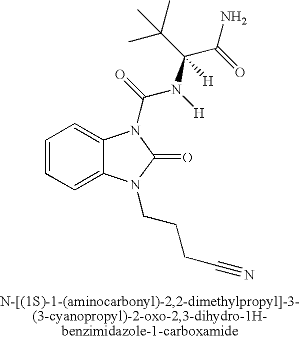 | 1H NMR (400 MHz, DMSO-d6) δ ppm 9.16 (1H, d, J = 8.8 Hz), 8.05 (1 H, d, J = 7.7 Hz), 7.61 (1H, br. s.), 7.33 (1H, d, J = 8.1 Hz), 7.24 (1H, t, J = 7.5 Hz), 7.12-7.19 (1H, m), 4.26 (1H, d, J = 8.8 Hz), 3.98 (2H, t, J = 6.6 Hz), 2.60 (2H, t, J = 7.1 Hz), 2.01 (2H, t, J = 7.0 Hz), 1.00 (9H, s), 0.93 (2H, s) | 357.2 |
| 223 | 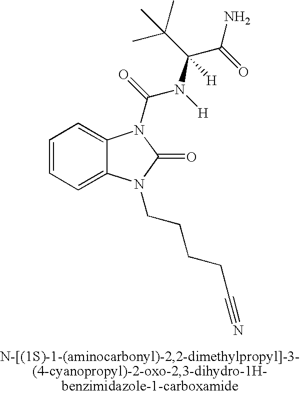 | 1H NMR (400 MHz, DMSO-d6) δ ppm 9.18 (1H, d, J = 8.8 Hz), 8.04 (1 H, d, J = 7.7 Hz), 7.61 (1H, br. s.), 7.35 (1H, d, J = 7.7 Hz), 7.24 (1H, t, j = 7.3 Hz), 7.11-7.19 (2H, m), 4.25 (1H, d, J = 8.8 Hz), 3.94 (2H, t, J = 6.6 Hz), 2.55-2.58 (1H, m), 1.78 (0H, d, J = 7.7 Hz), 1.71-1.84 (2H, m), 1.64 (0H, d, J = 7.7 Hz), 1.55-1.68 (2H, m), 1.00 (9H, s), 0.93 (2H, s) | 371.2 |
| 224 | 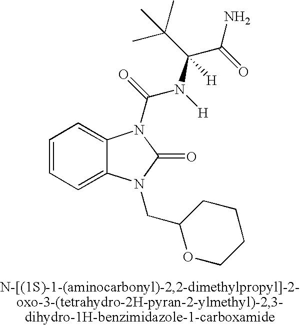 | 1H NMR (400 MHz, DMSO-d6) δ ppm 9.18 (1H, d, J = 8.8 Hz), 7.62 (1 H, br. s.), 7.31 (1H, d, J = 5.5 Hz), 7.21 (1H, t, J = 7.5 Hz), 7.09-7.18 (2H, m), 4.25 (1H, d, J = 8.8 Hz), 3.85-3.97 (2 H, m), 3.77-3.85 (0H, m), 3.77-3.85 (2H, m), 3.63-3.74 (1H, m), 3.22 (0 H, br. s.), 1.73-1.83 (1H, m), 1.65 (1 H, d, J = 12.1 Hz), 1.38-1.54 (3H, m), 1.15-1.35 (1H, m), 1.00 (8H, s), 0.93 (2H, s) | 389.2 (M + H) |
| 225 | 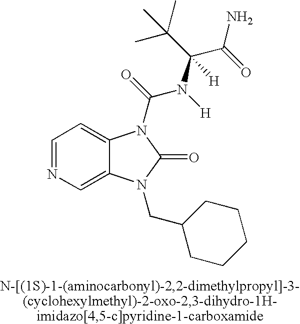 | 1H NMR (400 MHz, DMSO-d6) δ ppm 10.50 (1H, br. s.), 8.34 (1H, s), 8.14 (1H, d, J = 5.1 Hz), 7.91 (1H, d, J = 2.6 Hz), 7.01 (1H, d, J = 5.1 Hz), 3.69 (1H, s), 3.66 (2H, d, J = 7.3 Hz), 1.81 (1H, dd, J = 10.2, 4.4 Hz), 1.66 (2 H, d, J = 4.8 Hz), 1.59 (3H, d, J = 11.0 Hz), 1.15 (3H, t, J = 7.7 Hz), 1.00 (4H, s), 0.93 (9H, s) | 388.3 (M + H) |
| 226 | 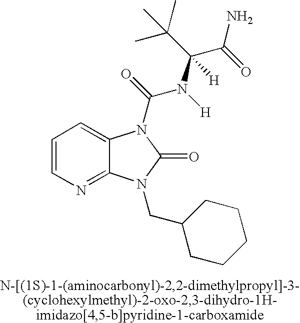 | 1H NMR (400 MHz, DMSO-d6) δ ppm 8.99 (1H, d, J = 9.1 Hz), 8.19 (1 H, d, J = 8.1 Hz), 8.15 (1H, d, J = 4.0 Hz), 7.63 (1H, br. s.), 7.16 (1H, br. s.), 7.18 (1H, d, J = 7.7 Hz), 4.25 (1H, d, J = 8.8 Hz), 3.75 (2H, d, J = 7.0 Hz), 1.87-1.97 (1H, m), 1.59-1.70 (5H, m), 1.12-1.21 (3H, m), 1.00 (11H, s) | 387.2 |
| 227 | 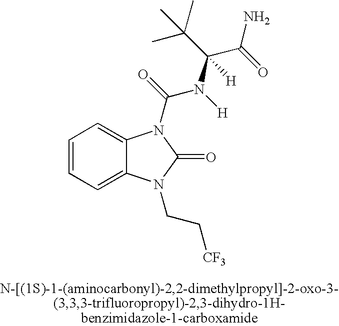 | 1H NMR (400 MHz, DMSO-d6) δ ppm 1.00 (s, 9H) 2.81 (td, J = 10.98, 4.03 Hz, 2H) 4.17 (t, J = 6.77 Hz, 2H) 4.27 (d, J = 9.15 Hz, 1H) 7.19 (d, J = 7.69 Hz, 1H) 7.14 (br. s., 1H) 7.26 (t, J = 7.50 Hz, 1H) 7.36 (d, J = 7.69 Hz, 1H) 7.62 (br. s., 1H) 8.05 (d, J = 7.69 Hz, 1H) 9.12 (d, J = 8.78 Hz, 1H) | 386.1 |

| Example | Structure |
|---|---|
| Number | Chemical Name |
| 228 | 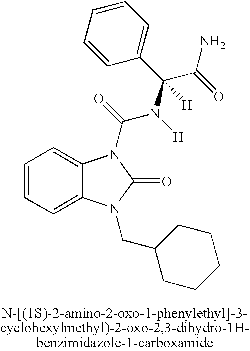 |
| 229 | 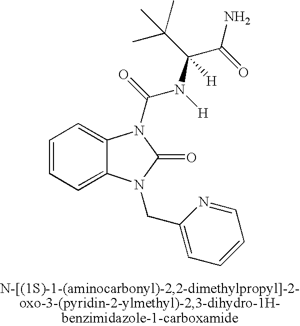 |
| 230 | 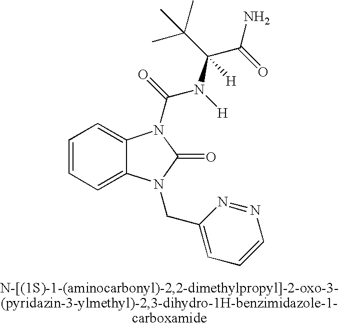 |
| 231 | 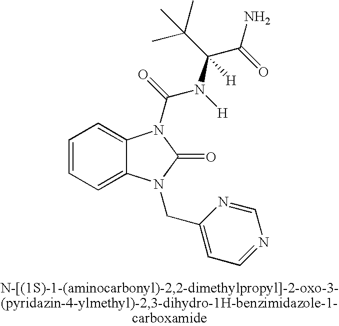 |
| 232 | 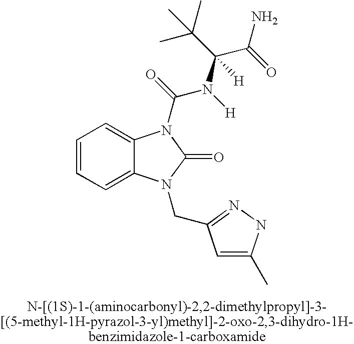 |
| 233 | 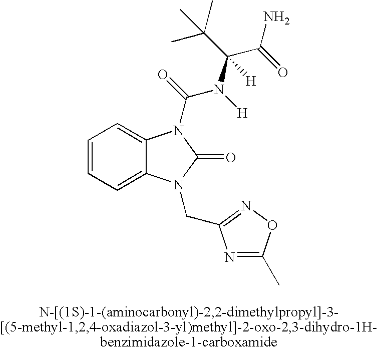 |
| 234 | 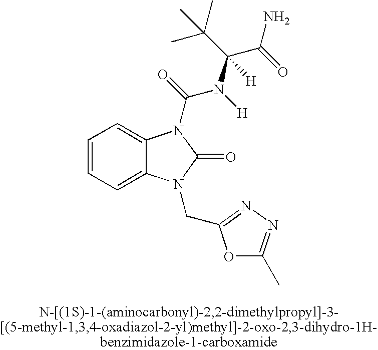 |
| 235 | 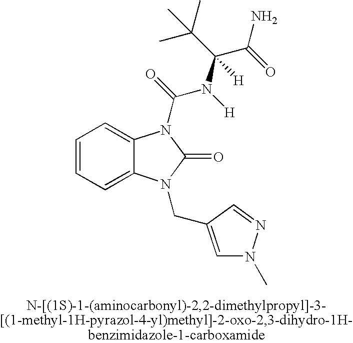 |
| 236 | 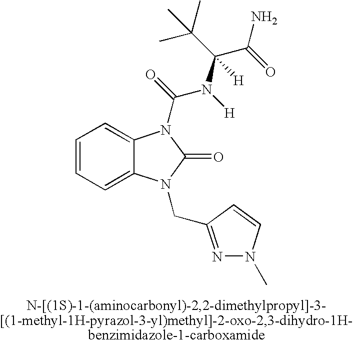 |
| 237 | 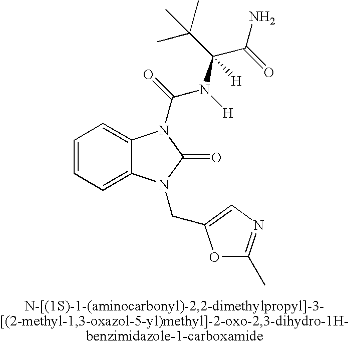 |
| 238 | 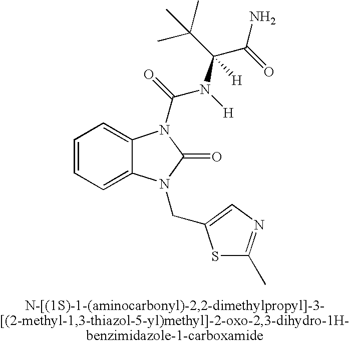 |
| 239 | 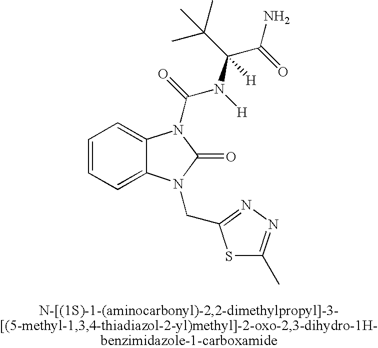 |
| 240 | 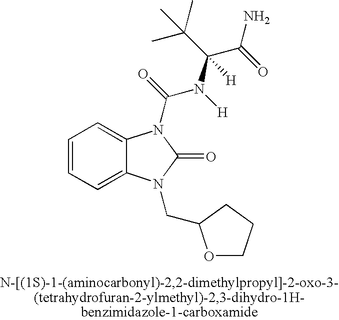 |
| 241 | 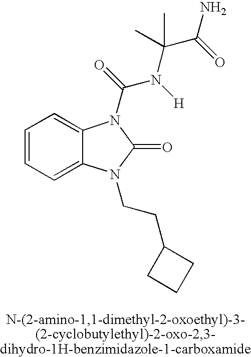 |
| 242 | 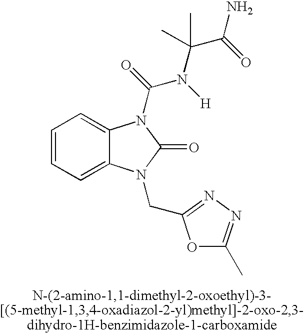 |
| 243 | 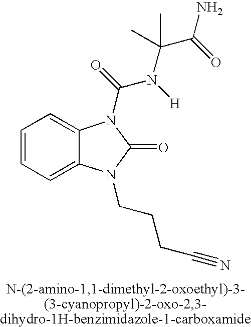 |
| 244 | 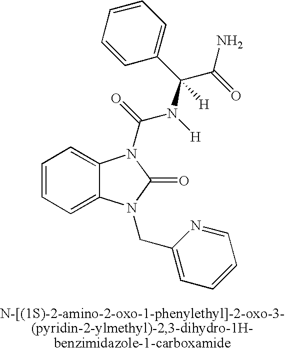 |
| 245 | 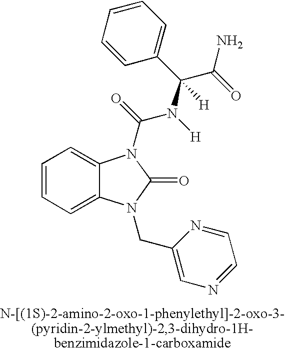 |
| 246 | 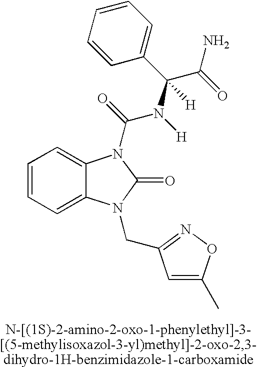 |
| 247 | 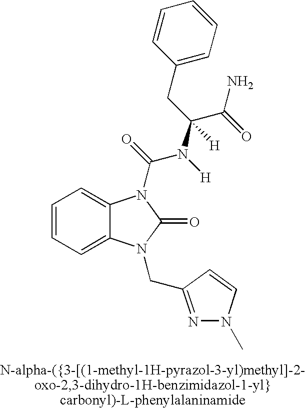 |
| 248 | 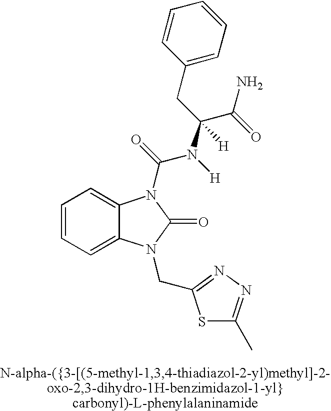 |
| 249 | 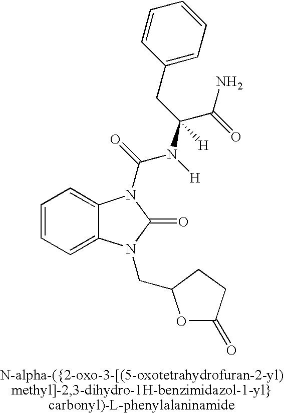 |
| 250 | 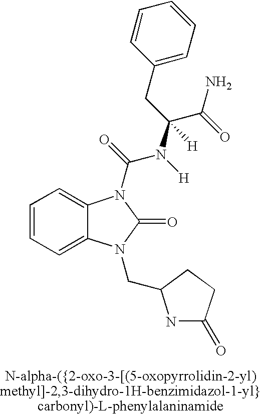 |
| 251 | 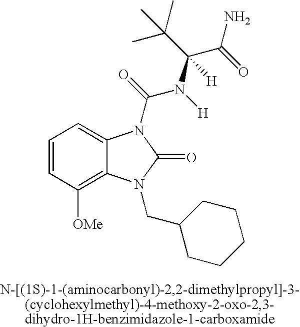 |
| 252 | 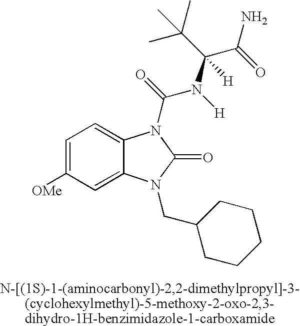 |
| 253 | 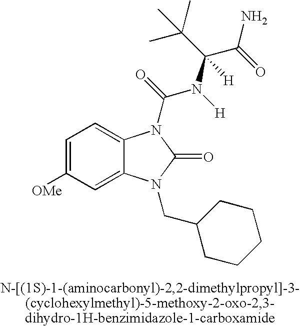 |
| 254 | 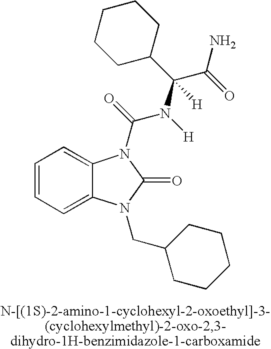 |
| 255 | 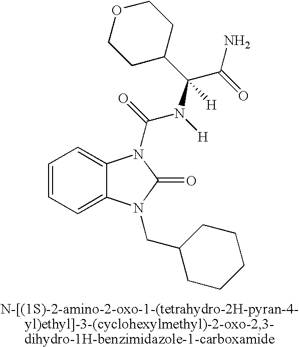 |
| 256 | 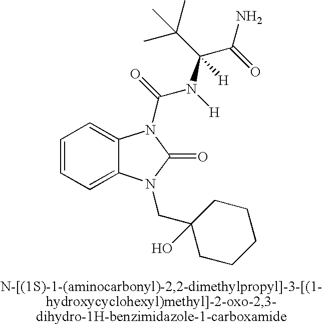 |
| 257 | 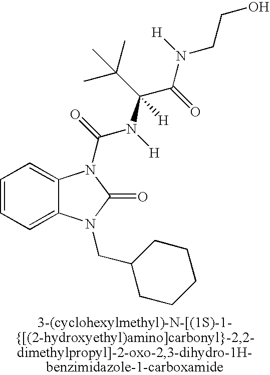 |
| 258 | 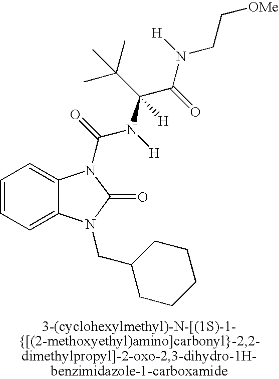 |
| 259 | 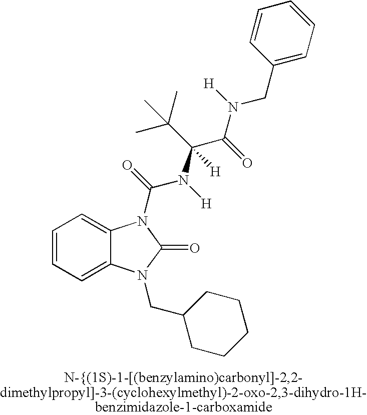 |
| 260 | 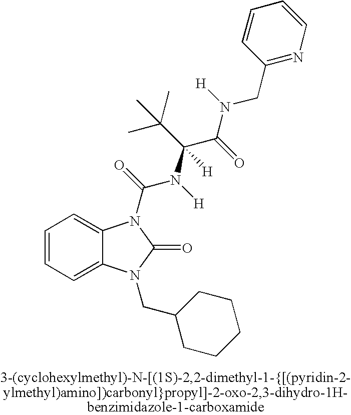 |
| 261 | 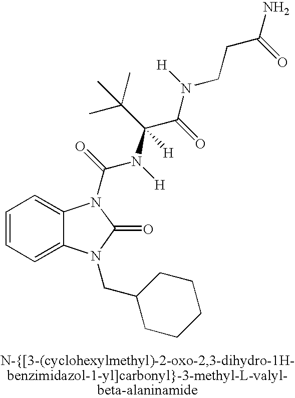 |
| 262 | 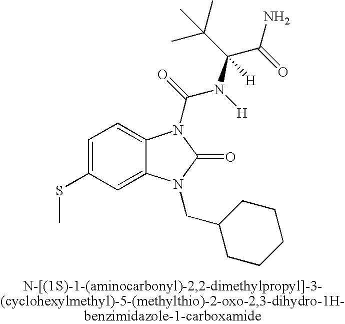 |
| 263 | 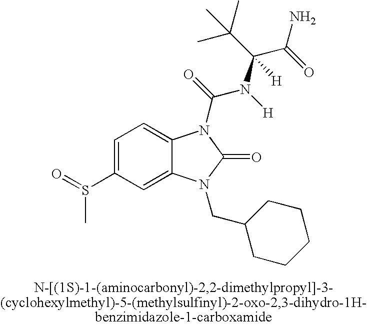 |
| 264 | 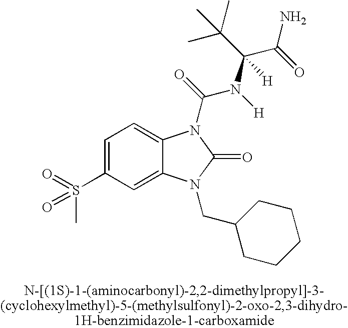 |
| 265 | 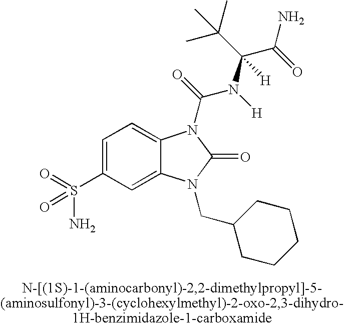 |

| Example | Structure | | Mass |
|---|---|---|---|
| Number | Compound Name | NMR Data | (M) |
| 266 | 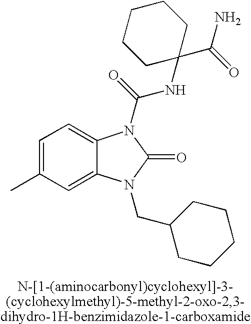 | 1H NMR (400 MHz, DMSO-d6) δ ppm 1.05 (d, J = 10.62 Hz, 2H) 1.17 (d, J = 6.96 Hz, 3H) 1.24 (br. s., 1H) 1.33- 1.44 (m, 1H) 1.39 (d, J = 10.25 Hz, 1H) 1.61 (d, J = 9.88 Hz, 4H) 1.67 (br. s., 3H) 1.83 (br. s., 1H) 1.77 (d, J = 16.11 Hz, 3 H) 2.04 (d, J = 13.18 Hz, 2H) 2.37 (s, 3 H) 3.30 (br. s., 1H) 3.71 (d, J = 7.32 Hz, 2 H) 6.82 (br. s., 1H) 6.95 (d, J = 7.69 Hz, 1 H) 7.15 (s, 1H) 7.36 (br. s., 1H) 7.87 (d, J = 8.05 Hz, 1H) 8.98 (s, 1H) | 412.2 |
| 267 | 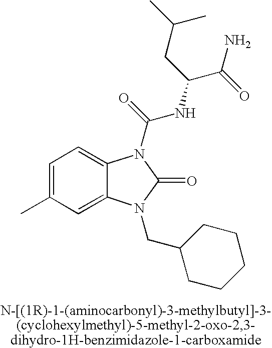 | 1H NMR (400 MHz, DMSO-d6) δ ppm 0.93 (d, J = 4.39 Hz, 6H) 1.07 (br. s., 1H) 1.03 (d, J = 9.88 Hz, 2H) 1.16 (d, J = 6.22 Hz, 3H) 1.24 (d, J = 15.37 Hz, 1H) 1.57- 1.69 (m, 7H) 1.82 (d, J = 19.03 Hz, 1H) 1.81 (d, J = 4.76 Hz, 1H) 2.37 (s, 3H) 3.69 (d, J = 6.95 Hz, 2H) 4.37 (d, J = 5.12 Hz, 1H) 6.95 (d, J = 8.05 Hz, 1H) 7.12 (d, J = 16.47 Hz, 2H) 7.64 (br. s., 1H) 7.89 (d, J = 8.42 Hz, 1H) 8.96 (d, J = 8.05 Hz, 1H) | 400.2 |
| 268 | 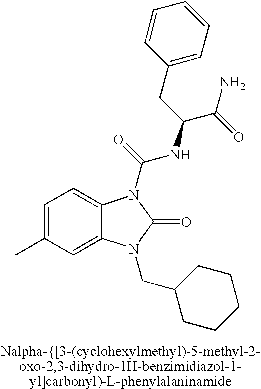 | 1H NMR (400 MHz, DMSO-d6) δ ppm 0.97-1.08 (m, 1H) 0.97-1.08 (m, 1H) 1.12-1.20 (m, 3H) 1.58-1.64 (m, 3H) 1.64-1.69 (m, 2H) 1.81 (br. s., 1H) 2.36 (s, 3H) 2.96 (dd, J = 13.73, 7.87 Hz, 1H) 3.15 (dd, J = 13.91, 5.12 Hz, 1H) 3.67 (d, J = 7.32 Hz, 2H) 4.58-4.64 (m, 1H) 6.93 (d, J = 8.42 Hz, 1H) 7.12 (s, 1 H) 7.17-7.27 (m, 5H) 7.67 (br. s., 1H) 7.83 (d, J = 8.42 Hz, 1H) 9.00 (d, J = 7.69 Hz, 1H) | 434.2 |
| 269 | 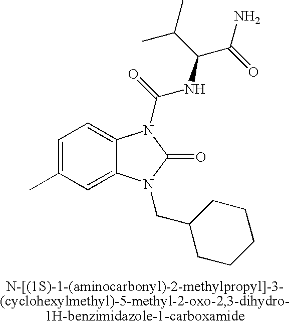 | 1H NMR (400 MHz, DMSO-d6) δ ppm 0.88-0.97 (m, 6H) 1.04 (d, J = 9.52 Hz, 2H) 1.08 (br. s., 1H) 1.16 (d, J = 6.95 Hz, 3H) 1.64 (d, J = 16.84 Hz, 5H) 1.83 (br. s., 1H) 2.14 (dd, J = 12.45, 6.22 Hz, 1H) 2.37 (s, 3H) 3.23 (br. s., 1H) 3.69 (br. s., 1H) 3.71 (d, J = 1.83 Hz, 1H) 4.28 (dd, J = 8.42, 5.12 Hz, 1H) 6.95 (d, J = 8.05 Hz, 1H) 7.15 (s, 2H) 7.60 (br. s., 1H) 7.89 (d, J = 8.05 Hz, 1H) 9.05 (d, J = 8.42 Hz, 1H) | 386.2 |
| 270 | 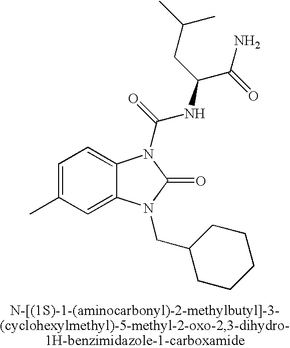 | 1H NMR (400 MHz, DMSO-d6) δ ppm 8.95 (1H, d, J = 8.1 Hz), 7.88 (1H, d, J = 8.1 Hz), 7.84 (1H, br. s.), 7.12 (2H, d, J = 16.8 Hz), 6.96 (1H, d, J = 8.1 Hz), 4.37 (1H, d, J = 5.5 Hz), 3.68 (2H, d, J = 7.0 Hz), 2.36 (3H, s), 1.81 (1H, dd, J = 13.2, 6.2 Hz), 1.57-1.68 (8H, m), 1.15 (3H, d, J = 6.6 Hz), 1.07 (1H, br. s.), 0.93 (4H, d, J = 1.8 Hz), 0.92-1.04 (1H, m), 0.92 (3H, br. s.) | 400.2 |
| 271 | 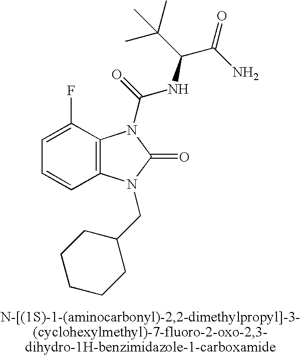 | 1H NMR (400 MHz, DMSO-d6) δ ppm 8.70 (1H, d, J = 9.2 Hz), 7.61 (1H, br. s.), 7.23 (1H, d, J = 3.7 Hz), 7.16 (2H, d, J = 7.3 Hz), 6.98 (1H, dd, J = 11.0, 8.8 Hz), 4.22 (1H, d, J = 9.2 Hz), 3.72 (2H, d, J = 7.0 Hz), 1.76-1.86 (1H, m), 1.63 (5H, d, J = 17.2 Hz), 1.16 (3H, t, J = 7.1 Hz), 1.00 (11H, s) | 404.2 |
| 272 | 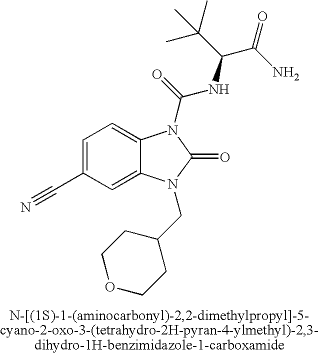 | 1H NMR (400 MHz, DMSO-d6) δ ppm 9.10 (1H, d, J = 8.8 Hz), 8.16 (1H, d, J = 8.4 Hz), 7.98 (1H, s), 7.59-7.65 (2 H, m), 7.16 (1H, br. s.), 4.26 (1H, d, J = 8.8 Hz), 3.81 (3H, d, J = 7.3 Hz), 3.85 (1H, br. s.), 3.21-3.24 (1H, m), 2.09 (1 H, br. s.), 1.55 (2H, d, J = 11.3 Hz), 1.31 (0H, d, J = 3.7 Hz), 1.27-1.38 (2H, m), 1.00 (11H, s) | 413.2 |
| 273 | 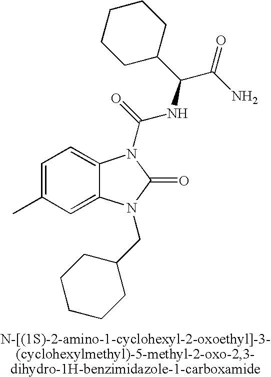 | 1H NMR (400 MHz, DMSO-d6) δ ppm 0.98-1.21 (m, 9H) 1.54-1.75 (m, 4H) 1.54-1.75 (m, 8H) 1.80 (br. s., 0H) 2.35 (s, 3H) 2.48 (br. s., 2H) 3.67 (dd, J = 7.32, 3.66 Hz, 2H) 4.20-4.30 (m, 1 H) 6.93 (d, J = 8.78 Hz, 1H) 7.12 (br. s., 2 H) 7.57 (br. s., 1H) 7.86 (d, J = 8.05 Hz, 1 H) 9.02 (d, J = 8.78 Hz, 1H) | 426.2 |
| 274 | 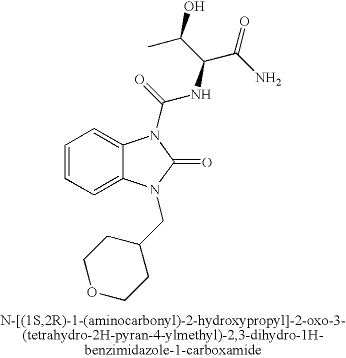 | 1H NMR (400 MHz, DMSO-d6) δ ppm 1.12 (d, J = 6.22 Hz, 3H) 1.35 (br. s., 2H) 1.55 (br. s., 2H) 2.02-2.13 (m, 1H) 3.18-3.27 (m, 2H) 3.79 (d, J = 6.95 Hz, 3H) 3.85 (br. s., 2H) 4.11-4.22 (m, 1 H) 4.14 (dd, J = 7.69, 2.93 Hz, 1H) 7.14 (d, J = 7.69 Hz, 2H) 7.23 (t, J = 7.50 Hz, 1 H) 7.36 (d, J = 8.05 Hz, 1H) 7.44 (br. s., 1 H) 8.04 (d, J = 8.05 Hz, 1H) 9.19 (d, J = 7.69 Hz, 1H) | 376.2 |
| 275 | 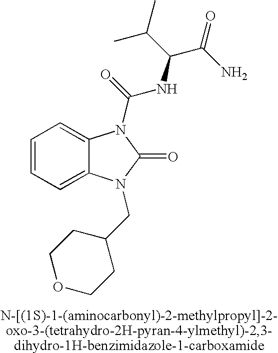 | 1H NMR (400 MHz, DMSO-d6) δ ppm 9.07 (1H, d, J = 8.4 Hz), 8.04 (1H, d, J = 8.1 Hz), 7.61 (1H, br. s.), 7.37 (1H, d, J = 7.3 Hz), 7.24 (1H, t, J = 7.7 Hz), 7.12- 7.19 (2H, m), 6.97 (1H, s), 4.29 (1H, dd, J = 8.4, 4.8 Hz), 3.79 (2H, d, J = 7.0 Hz), 3.83 (2H, d, J = 7.7 Hz), 3.67 (1H, d, J = 7.3 Hz), 3.19-3.25 (2H, m), 2.12- 2.17 (1H, m), 1.49-1.56 (2H, m), 1.32 (0H, d, J = 8.4 Hz), 1.26-1.37 (2H, m), 0.89-0.98 (6H, m), 0.81 (1H, d, J = 6.6 Hz) | 374.2 |
| 276 | 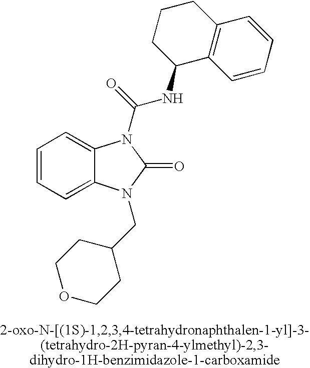 | 1H NMR (400 MHz, DMSO-d6) δ ppm 9.00 (1H, d, J = 8.1 Hz), 8.11 (1H, d, J = 8.1 Hz), 7.36 (2H, t, J = 8.1 Hz), 7.15- 7.25 (4H, m), 5.10 (1H, d, J = 6.2 Hz), 3.82 (1H, d, J = 2.9 Hz), 3.79 (1H, d, J = 2.2 Hz), 3.74 (2H, d, J = 7.3 Hz), 3.21 (2H, t, J = 11.5 Hz), 2.82 (1H, t, J = 5.1 Hz), 2.77 (1H, t, J = 5.5 Hz), 2.05 (1H, d, J = 6.6 Hz), 2.00-2.10 (1H, m), 1.91 (1 H, d, J = 6.2 Hz), 1.82 (1H, br. s.), 1.84 (2 H, d, J = 5.5 Hz), 1.50 (2H, d, J = 13.2 Hz), 1.28 (1H, dd, J = 12.3, 4.2 Hz), 1.23- 1.34 (1H, m) | 406.2 (M + H) |
| 277 | 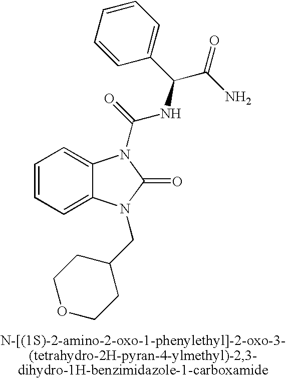 | 1H NMR (400 MHz, DMSO-d6) δ ppm 9.72 (1H, d, J = 6.6 Hz), 7.98 (1H, d, J = 8.1 Hz), 7.84 (1H, br. s.), 7.48 (2H, d, J = 7.3 Hz), 7.36 (4H, dt, J = 12.5, 7.6 Hz), 7.13 (1H, t, J = 7.9 Hz), 5.45 (1H, d, 7.31 (1H, br. s.), 7.22 (1H, t, J = 7.5 Hz), J = 7.0 Hz), 3.79 (3H, d, J = 7.0 Hz), 3.84 (1H, d, J = 11.3 Hz), 3.24 (2H, t, J = 11.0 Hz), 2.02-2.13 (1H, m), 1.54 (2H, d, J = 11.7 Hz), 1.32 (1H, d, J = 12.1 Hz), 1.33 (1H, q, J = 12.2 Hz) | 408.2 |
| 278 | 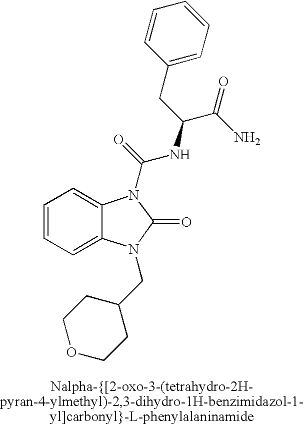 | 1H NMR (400 MHz, DMSO-d6) δ ppm 9.02 (1H, d, J = 7.7 Hz), 7.98 (1H, d, J = 8.1 Hz), 7.67 (1H, br. s.), 7.35 (1H, d, J = 7.7 Hz), 7.19-7.28 (7H, m), 7.13 (2 H, t, J = 7.7 Hz), 4.62 (1H, d, J = 5.1 Hz), 3.83 (2H, d, J = 7.7 Hz), 3.77 (2H, d, J = 7.0 Hz), 3.18 (1H, d, J = 5.5 Hz), 3.17- 3.25 (2H, m), 2.98 (1H, dd, J = 14.1, 7.9 Hz), 2.04 (1H, td, J = 11.3, 3.8 Hz), 1.49 (2H, d, J = 11.0 Hz), 1.45-1.55 (1H, m), 1.26-1.36 (2H, m, J = 12.0, 12.0, 11.7, 3.8 Hz) | 422.2 |
| 279 | 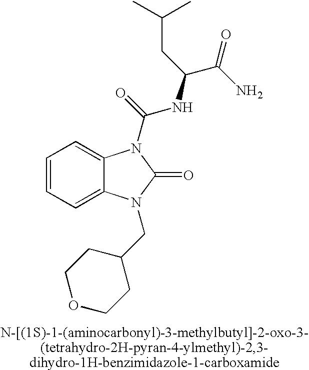 | 1H NMR (400 MHz, DMSO-d6) δ ppm 8.98 (1H, d, J = 7.7 Hz), 8.03 (1H, d, J = 8.1 Hz), 7.64 (1H, br. s.), 7.37 (1H, d, J = 8.1 Hz), 7.23 (1H, t, J = 7.7 Hz), 7.11 (1H, br. s.), 7.15 (1H, t, J = 7.7 Hz), 6.97 (1H, s), 4.39 (1H, d, J = 5.1 Hz), 3.80 (2 H, d, J = 8.1 Hz), 3.83 (1H, d, J = 7.7 Hz), 3.77 (1H, br. s.), 3.24 (1H, br. s.), 3.23 (1H, d, J = 18.3 Hz), 2.06 (1H, td, J = 11.1, 3.8 Hz), 1.67 (1H, dd, J = 11.9, 5.7Hz), 1.59 (1H, d, J = 7.0 Hz), 1.53 (2 H, d, J = 11.7 Hz), 1.27-1.38 (2H, m), 0.88 (2H, t, J = 6.0 Hz), 0.93 (5H, d, J = 59 Hz) | 388.2 |
| 280 | 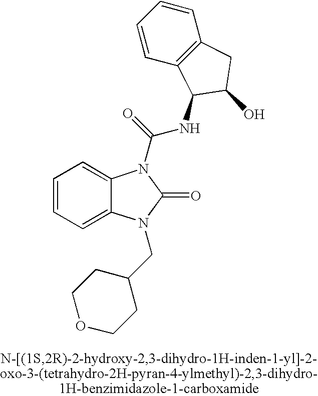 | 1H NMR (400 MHz, DMSO-d6) δ ppm 9.32 (1H, d, J = 8.1 Hz), 8.15 (1H, d, J = 8.1 Hz), 7.38 (1H, d, J = 7.7 Hz), 7.23 (1H, s), 7.28 (2H, dd, J = 16.7, 5.5 Hz), 7.20 (2H, d, J = 11.0 Hz), 5.27-5.37 (1 H, m), 5.35 (1H, d, J = 4.0 Hz), 4.55 (1H, d, J = 4.4 Hz), 3.78 (3H, d, J = 7.3 Hz), 3.82 (2H, d, J = 9.5 Hz), 3.20 (0H, br. s.), 3.24 (3H, t), 3.12 (1H, d, J = 4.8 Hz), 2.85 (1H, d, J = 16.1 Hz), 2.00-2.11 (1 H, m), 1.52 (2H, d, J = 12.1 Hz), 1.32 (2 H, dd), 1.29 (0H, d, J = 4.4 Hz) | 407.2 |
| 281 | 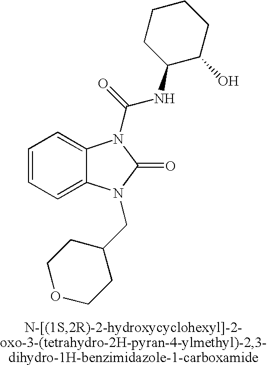 | 1H NMR (400 MHz, DMSO-d6) δ ppm 8.74 (1H, d, J = 7.3 Hz), 8.06 (1H, d, J = 7.7 Hz), 7.36 (1H, d, J = 8,1 Hz), 7.23 (1H, t, J = 7.7 Hz), 7.15 (1H, t, J = 7.7 Hz), 4.82 (1H, d, J = 5.1 Hz), 3.78 (2H, d, J = 7.3 Hz), 3.76-3.85 (2H, m), 3.36 (1H, dd, J = 8.4, 4.4 Hz), 3.23 (1H, br. s.), 3.23 (1H, d, J = 19.4 Hz), 2.04 (2H, d, J = 5.1 Hz), 2.06 (1H, br. s.), 1.88 (1H, dd, J = 9.1, 2.6 Hz), 1.58-1.68 (2H, m), 1.54 (1H, br. s.), 1.50 (1H, d, J = 1.5 Hz), 1.33 (2H, d, J = 4.4 Hz), 1.27 (4H, d, J = 7.3 Hz) | 373.2 |
| 282 | 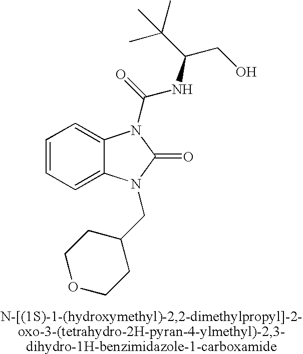 | 1H NMR (400 MHz, DMSO-d6) δ ppm 8.82 (1H, d, J = 9.9 Hz), 8.07 (1H, d, J = 8.1 Hz), 7.37 (1H, d, J = 7.7 Hz), 7.23 (1H, t, J = 7.7 Hz), 7.15 (1H, t, J = 7.7 Hz), 6.97 (1H, s), 4.65 (1H, t, J = 5.3 Hz), 3.78-3.86 (1H, m), 3.81 (3H, dd, J = 13.9, 7.3 Hz), 3.70 (1H, d, J = 8.8 Hz), 3.67 (1H, d, J = 7.0 Hz), 3.48 (1H, dd, J = 10.4, 5.7 Hz), 3.23 (2H, d, J = 10.6 Hz), 2.07 (1H, dd, J = 10.6, 4.0 Hz), 1.53 (2H, d, J = 12.8 Hz), 1.26-1.36 (1H, m), 1.33 (1H, d, J = 13.5 Hz), 0.96 (7H, s), 0.81 (2H, s) | 375.2 |
| 283 | 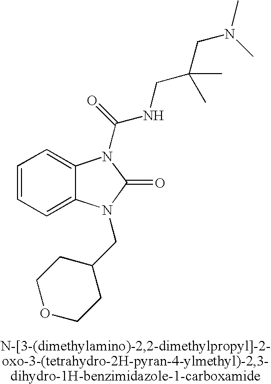 | 1H NMR (400 MHz, DMSO-d6) δ ppm 8.97 (1H, t, J = 5.3 Hz), 8.05 (1H, d, J = 7.7 Hz), 7.36 (1H, d, J = 7.7 Hz), 7.22 (1H, t, J = 7.7 Hz), 7.14 (1H, t, J = 7.7 Hz), 3.79 (3H, d, J = 7.3 Hz), 3.84 (1H, br. s.), 3.24 (3H, d, J = 5.5 Hz), 3.19 3.25 (1H, m), 2.25 (6H, s), 2.16 (2H, s), 2.02-2.11 (1H, m), 1.53 (2H, d), 1.50 (0H, br. s.), 1.33 (0H, d, J = 4.0 Hz), 1.30 (2H, d, J = 3.7 Hz), 0.90 (6H, s) | 388.3 |
| 284 | 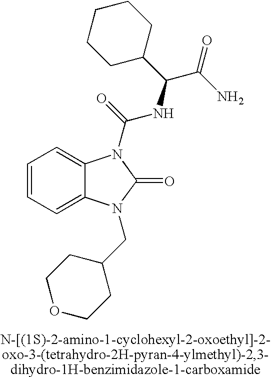 | 1H NMR (400 MHz, DMSO-d6) δ ppm 9.07 (1H, d, J = 8.4 Hz), 8.03 (1H, d, J = 7.7 Hz), 7.61 (1H, br. s.), 7.37 (1H, d, J = 7.7 Hz), 7.23 (1H, t, J = 7.5 Hz), 7.12- 7.17 (2H, m), 4.28 (1H, dd, J = 8.2, 5.7 Hz), 3.85 (2H, br. s.), 3.82 (1H, d, J = 3.3 Hz), 3.79 (2H, d, J = 7.0 Hz), 3.18-3.26 (2H, m), 2.07 (1H, dd, J = 8.8, 6.6 Hz), 1.78 (1H, br. s.), 1.63 (1H, br. s.), 1.70 (4H, t, J = 14.3 Hz), 1.53 (2H, d, J = 13.2 Hz), 1.37 (1H, br. s.), 1.33 (1H, d, J = 8.4 Hz) 1.17 (1H, d, J = 11.3 Hz), 1.20 (1H, br. s.), 1.02 (1H, d, J = 11.0 Hz), 1.05 (1 H, d, J = 10.6 Hz) | 414.2 |
| 285 | 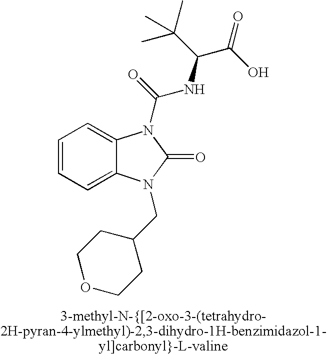 | 1H NMR (400 MHz, DMSQ-d6) δ ppm 0.99 (s, 8H) 1.28 (d, J = 3.76 Hz, 1H) 1.34 (d, J = 4.83 Hz, 1H) 1.50 (d, J = 13.16 Hz, 2H) 1.96-2.11 (m, 1H) 3.20 (t, J = 11.55 Hz, 2H) 3.81 (d, J = 3.76 Hz, 1H) 3.77 (d, J = 7.52 Hz, 3H) 4.16 (d, J = 8.59 Hz, 1H) 7.13 (t, J = 8.32 Hz, 1 H) 7.22 (t, J = 7.12 Hz, 1H) 7.37 (d, J = 7.52 Hz, 1H) 7.99 (d, J = 7.25 Hz, 1H) 9.24 (d, J = 8.59 Hz, 1H) 12.94 (br. s., 2 H) | 389.2 |
| 286 | 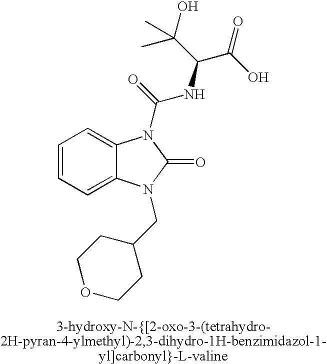 | 1H NMR (400 MHz, DMSO-d6) δ ppm 1.26 (s, 7H) 1.33 (d, J = 9.15 Hz, 2H) 1.51 (br. s., 2H) 2.08 (br. s., 1H) 3.16- 3.26 (m, 2H) 3.80 (d, J = 7.32 Hz, 5H) 4.23 (d, J = 7.69 Hz, 1H) 7.16 (d, J = 7.69 Hz, 1H) 7.24 (t, J = 7.69 Hz, 1H) 7.38 (d, J = 8.05 Hz, 1H) 8.03 (d, J = 8.05 Hz, 1H) 9.30 (d, J = 7.69 Hz, 1H) | 391.2 |
| 287 | 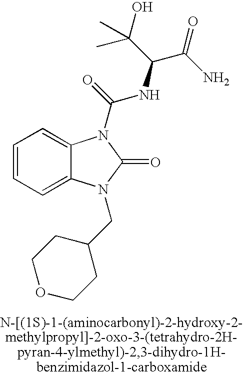 | 1H NMR (400 MHz, DMSO-d6) δ ppm 1.21 (d, J = 12.44 Hz, 6H) 1.32 (br. s., 1 H) 1.34 (d, J = 3.66 Hz, 1H) 1.52 (br. s., 2 H) 2.08 (br. s., 1H) 3.24 (t, J = 11.53 Hz, 3H) 3.79 (d, J = 7.32 Hz, 4H) 4.25 (d, J = 8.05 Hz, 1H) 7.15-7.25 (m, 3H) 7.37 (d, J = 8.05 Hz, 1H) 7.48 (br. s., 1H) 8.04 (d, J = 8.05 Hz, 1H) 9.26 (d, J = 8.05 Hz, 1H) | 390.2 |
| 288 | 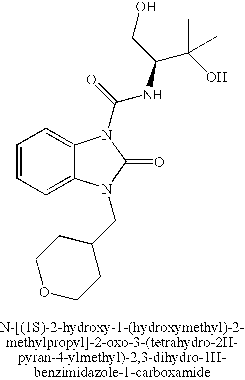 | 1H NMR (400 MHz, DMSO-d6) δ ppm 1.13 (s, 3H) 1.18-1.23 (m, 3H) 1.25- 1.37 (m, 3H) 1.53 (d, J = 12.44 Hz, 2H) 2.07 (td, J = 7.41, 3.48 Hz, 1H) 3.18- 3.26 (m, 2H) 3.57 (dd, J = 10.98, 6.22 Hz, 1H) 3.74-3.85 (m, 1H) 3.79 (q, J = 7.56 Hz, 5H) 4.55 (br. s., 1H) 7.15 (t, J = 7.32 Hz, 1H) 7.23 (t, J = 7.14 Hz, 1H) 7.36 (d, J = 7.69 Hz, 1H) 8.08 (d, J = 7.69 Hz, 1H) 8.91 (d, J = 9.15 Hz, 1H) | 377.2 |
| 289 | 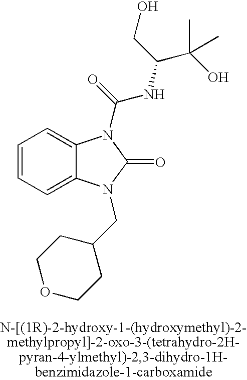 | 1H NMR (400 MHz, DMSO-d6) δ ppm 1.13 (s, 3H) 1.18-1.23 (m, 3H) 1.25- 1.37 (m, 3H) 1.53 (d, J = 12.08 Hz, 2H) 2.06 (dt, J = 7.23, 3.89 Hz, 1H) 3.17- 3.25 (m, 2H) 3.57 (dd, J = 10.80, 6.04 Hz, 1H) 3.74-3.85 (m, 3H) 3.79 (d, J = 6.95 Hz, 3H) 4.57 (br. s., 1H) 7.15 (t, J = 7.32 Hz, 1H) 7.23 (t, J = 7.32 Hz, 1H) 7.36 (d, J = 7.69 Hz, 1H) 8.08 (d, J = 7.69 Hz, 1H) 8.90 (d, J = 9.15 Hz, 1H) | 377.2 |
| 290 | 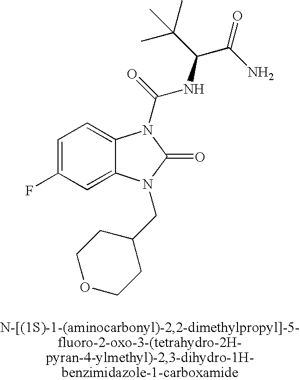 | 1H NMR (400 MHz, DMSO-d6) δ ppm 0.96 (s, 8H) 1.19-1.36 (m, J = 12.35, 12.35, 12.22, 4.16 Hz, 2H) 1.49 (dd, J = 12.76, 2.28 Hz, 2H) 1.96-2.09 (m, 1 H) 3.20 (t, J = 11.68 Hz, 2H) 3.80 (dd, J = 11.01, 3.49 Hz, 1H) 3.75 (dd, J = 7.25, 1.07 Hz, 2H) 4.21 (d, J = 9.13 Hz, 1H) 7.07 (ddd, J = 9.67, 8.86, 2.69 Hz, 1H) 7.18 (s, 1H) 7.37 (dd, J = 8.86, 4.56 Hz, 1H) 7.64 (s, 1H) 7.78 (dd, J = 9.67, 2.69 Hz, 1H) 9.13 (d, J = 8.86 Hz, 1H) | 406.2 |
| 291 | 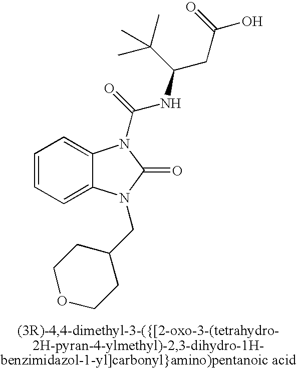 | 1H NMR (400 MHz, DMSO-d6) δ ppm 0.94 (s, 12H) 1.52 (d, J = 12.05 Hz, 3H) 3.24 (t, J = 11.16 Hz, 3H) 3.82 (d, J = 11.35 Hz, 3H) 3.78 (d, J = 7.32 Hz, 3 H) 7.15 (s, 1H) 7.23 (s, 1H) 7.36 (s, 1 H) 8.04 (d, J = 8.05 Hz, 1H) 8.78 (s, 1H) | 403.2 |
| 292 | 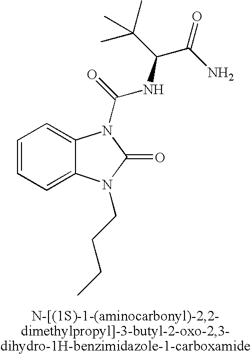 | 1H NMR (400 MHz, DMSO-d6) δ ppm 9.19 (1H, d, J = 8.8 Hz), 8.04 (1H, d, J = 8.1 Hz), 7.62 (1H, br. s.), 7.28-7.35 (1H, m), 7.24 (1H, t, J = 7.5 Hz), 7.14 (2 H, d, J = 5.9 Hz), 4.25 (1H, d, J = 9.1 Hz), 3.89-3.94 (2H, m), 3.88 (0H, br. s.), 1.67 (2H, t), 1.70 (0H, br. s.), 1.34 (1H, d, J = 7.7 Hz), 1.29-1.39 (1H, m), 1.00 (9H, s), 0.93 (3H, d, J = 7.3 Hz), 0.90 (1 H, br. s.) | 346.2 |
| 293 | 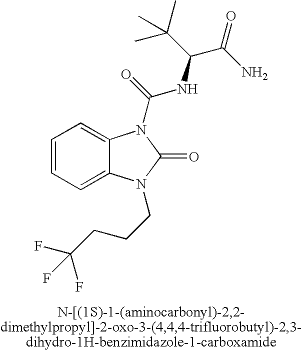 | 1H NMR (400 MHz, DMSO-d6) δ ppm 0.96 (s, 7H) 1.13 (dd, J = 19.77, 6.59 Hz, 2H) 1.88 (qd, J = 7.56, 7.32 Hz, 1H) 2.30-2.43 (m, 1H) 2.37 (d, J = 8.05 Hz, 1H) 2.46 (br. s., 1H) 3.39 (d, J = 7.69 Hz, 1H) 3.94 (t, J = 6.77 Hz, 2H) 4.21 (d, J = 9.15 Hz, 1H) 7.10 (d, J = 13.18 Hz, 1 H) 7.14 (s, 1H) 7.21 (t, J = 7.69 Hz, 1H) 7.32 (d, J = 7.69 Hz, 1H) 7.57 (br. s., 1H) 8.01 (d, J = 7.69 Hz, 1H) 9.12 (d, J = 9.15 Hz, 1H) | 400.2 |
| 294 | 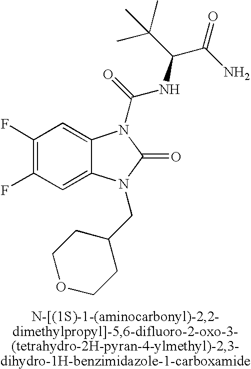 | 1H NMR (400 MHz, DMSO-d6) δ ppm 0.93 (s, 9H) 1.27 (td, J = 11.80, 8.23 Hz, 2H) 1.45 (d, J = 12.81 Hz, 2H) 2.00 (dd, J = 10.98, 5.86 Hz, 1H) 3.22 (t, J = 11.35 Hz, 3H) 3.65 (d, J = 7.32 Hz, 2H) 3.68 (s, 1H) 3.82 (d, J = 8.05 Hz, 2H) 7.03 (t, J = 8.78 Hz, 1H) 7.39 (t, J = 8.97 Hz, 1H) 7.90 (br. s., 1H) | 424.2 |
| 295 | 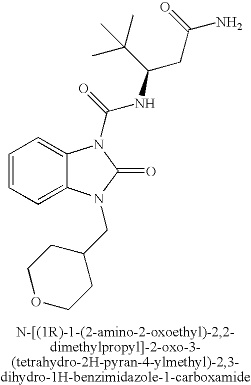 | 1H NMR (400 MHz, DMSO-d6) δ ppm 0.86 (s, 1H) 0.93 (s, 10H) 1.33 (br. s., 2 H) 1.53 (d, J = 12.08 Hz, 2H) 3.15-3.26 (m, 3H) 3.81 (br. s., 3H) 3.79 (d, J = 6.95 Hz, 2H) 3.84 (br. s., 1H) 7.15 (s, 1H) 7.23 (s, 1H) 7.36 (d, J = 7.69 Hz, 2H) 8.03 (s, 1H) 8.70 (s, 1H) | 402.2 |
| 296 | 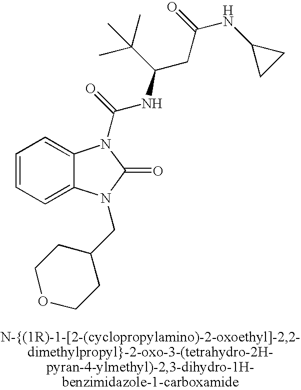 | 1H NMR (400 MHz, DMSO-d6) δ ppm 0.46 (d, J = 4.03 Hz, 1H) 0.44 (d, J = 6.95 Hz, 1H) 0.77 (br. s., 1H) 0.79 (s, 7H) 0.91 (br. s., 1H) 0.88 (s, 5H) 1.25 (br. s., 1H) 1.27 (d, J = 7.32 Hz, 1H) 1.44 (d, J = 15.01 Hz, 3H) 2.39-2.48 (m, 2H) 3.18 (t, J = 10.43 Hz, 2H) 3.20 (br. s., 1 H) 3.63 (6, J = 6.95 Hz, 1H) 3.70-3.79 (m, 1H) 3.76 (d, J = 3.29 Hz, 2H) 3.80 (br. s., 1H) 6.93 (s, 2H) 7.10 (d, J = 8.78 Hz, 1H) | 442.3 |
| 297 | 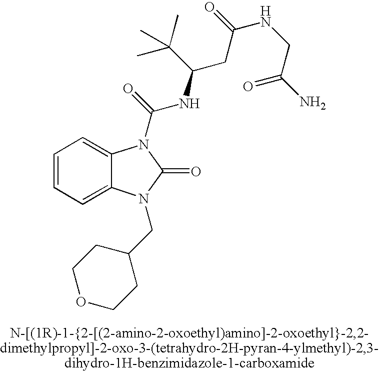 | 1H NMR (400 MHz, DMSO-d6) δ ppm 0.85 (s, 2H) 0.90 (s, 10H) 1.29 (br. s., 2 H) 1.49 (d, J = 12.44 Hz, 2H) 2.46 (6, J = 1.83 Hz, 2H) 3.10-3.20 (m, 2H) 3.49 (d, J = 5.49 Hz, 1H) 3.54 (6, J = 5.86 Hz, 1H) 3.73 (br. s., 1H) 3.78 (dd, J = 11.90, 9.33 Hz, 3H) 7.10 (s, 2H) 7.19 (s, 1H) 7.31 (s, 1H) 7.98 (d, J = 8.05 Hz, 1H) 8.05 (br. s., 1H) 8.72 (d, J = 9.52 Hz, 1H) | 459.2 |
| 298 | 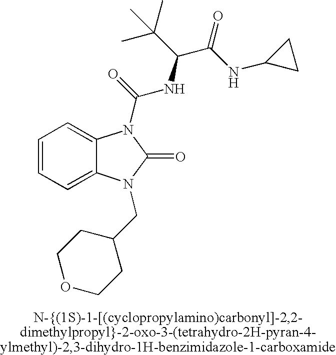 | 1H NMR (400 MHz, DMSO-d6) δ ppm 9.21 (1H, d, J = 8.8 Hz), 8.25 (1H, d, J = 4.0 Hz), 8.03 (1H, d, J = 7.7 Hz), 7.37 (1H, d, J = 7.7 Hz), 7.23 (1H, t, J = 7.7 Hz), 7.15 (1H, t, J = 7.7 Hz), 4.20 (1H, d, J = 9.2 Hz), 3.84 (1H, br. s.), 3.80 (3H, d, J = 7.7 Hz), 3.16-3.25 (2H, m), 2.66 (1 H, dd, J = 7.1, 3.5 Hz), 2.08 (1H, dd, J = 17.4, 3.1 Hz), 1.53 (2H, d, J = 12.1 Hz), 1.33 (2H, d, J = 10.6 Hz), 1.28- 1.39 (1H, m), 0.96 (9H, s), 0.63 (2H, d, J = 7.3 Hz), 0.40 (1H, br. s.), 0.42 (1H, d, J = 4.0 Hz) | 429.3 (M + H) |
| 299 | 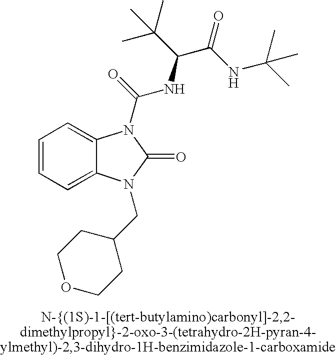 | 1H NMR (400 MHz, DMSO-d6) δ ppm 0.98 (s, 9H) 1.27 (s, 9H) 1.34 (d, J = 12.08 Hz, 2H) 1.53 (d, J = 12.44 Hz, 2 H) 2.02-2.14 (m, 1H) 3.25 (s, 2H) 3.82 (br. s., 1H) 3.79 (d, J = 7.32 Hz, 2H) 3.85 (br. s., 1H) 4.34 (d, J = 9.52 Hz, 1H) 7.14 (t, J = 7.69 Hz, 1H) 7.23 (t, J = 7.50 Hz, 1 H) 7.36 (d, J 8.05 Hz, 1H) 7.78 (s, 1H) 8.05 (d, J = 8.05 Hz, 1H) 9.17 (d, J = 9.15 Hz, 1H) | 445.3 (M + H) |
| 300 | 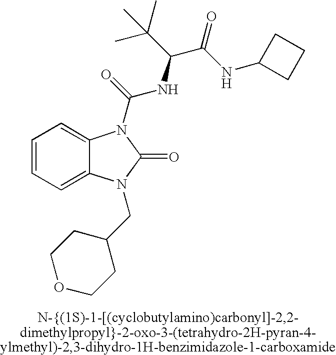 | 1H NMR (400 MHz, DMSO-d6) δ ppm 9.15 (1H, d, J = 9.2 Hz), 8.39 (1H, d, J = 7.3 Hz), 8.00 (1H, d, J = 8.1 Hz), 7.32 (1H, d, J = 8.1 Hz), 719 (1H, t, J = 7.5 Hz), 7.10 (1H, t, J = 7.7 Hz), 4.12-4.22 (1H, m), 4.21 (1H, d, J = 9.2 Hz), 3.80 (1 U, br. s.), 3.75 (2H, d, J = 7.3 Hz), 3.78 (1 H, br. s.), 3.20 (2H, br. s.), 2.11 (1H, d, J = 7.0 Hz), 2.14 (1H, br. s.),2.04 (1H, br. s.), 1.78-1.95 (2H, m), 1.60 (1H, d, J = 7.3 Hz), 1.56-1.65 (1H, m), 1.49 (2 H, d, J = 12.1 Hz), 1.29 (2H, dd, J = 12.1, 3.3 Hz), 0.93 (9H, s) | 442.3 |
| 301 | 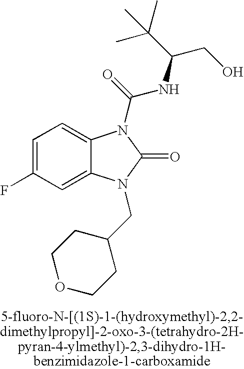 | 1H NMR (400 MHz, DMSO-d6) δ ppm 8.78 (1H, d, J = 9.5 Hz), 7.85 (1H, dd, J = 9.7, 2.4 Hz), 7.39 (1H, dd, J = 8.8, 4.8 Hz), 7.09 (1H, dd, J = 18.3, 2.6 Hz), 4.66 (1H, t, J = 5.1 Hz), 3.84 (1H, br. s.), 3.81 (2H, d, J = 7.0 Hz), 3.79 (2H, d, J = 5.1 Hz), 3.69 (1H, dd, J = 6.6, 2.6 Hz), 3.65 (1H, d, J = 4.4 Hz), 3.48 (1H, t, J = 11.3 Hz), 3.17-3.26 (2H, m), 2.06 (1H, dd, J = 15.4, 5.9 Hz), 1.53 (2H, d, J = 12.1 Hz), 1.23-1.39 (2H, in), 0.95 (9H, s) | 393.2 |
| 302 | 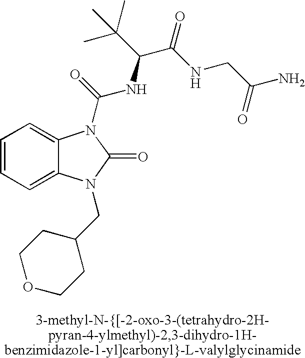 | 1H NMR (400 MHz, DMSO-d6) δ ppm 9.23 (1H, d, J = 8.4 Hz), 8.39 (1H, t, J = 4.8 Hz), 8.03 (1H, d, J = 7.7 Hz), 7.37 (1H, d, J = 7.7 Hz), 7.23 (1H, d, J = 8.4 Hz), 7.15 (1H, t, J = 7.9 Hz), 6.97 (1H, br. s.), 4.32 (1H, d, J = 8.4 Hz), 3.80 (3H, d, J = 7.7 Hz), 3.84 (1H, br. s.), 3.68 (2H, t, J = 5.1 Hz), 3.16-3.25 (2H, m), 2.08 (1 H, dd, J = 8.1, 2.9 Hz), 1.53 (2H, d, J = 11.3 Hz), 1.33 (2H, qd, J = 12.2, 4.0 Hz), 1.01 (9H, s) | 445.2 |
| 303 | 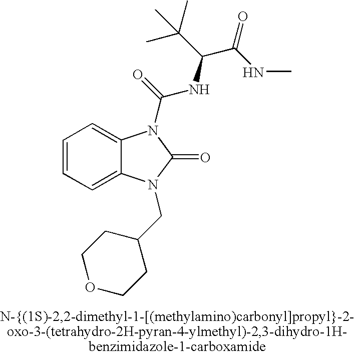 | 1H NMR (400 MHz, DMSO-d6) δ ppm 9.22 (1H, d, J = 8.8 Hz), 8.12 (1H, d, J = 4.4 Hz), 8.03 (1H, d, J = 7.7 Hz), 7.37 (1H, d, J = 7.7 Hz), 7.23 (1H, t, J = 7.5 Hz), 7.15 (1H, t, J = 7.7 Hz), 4.24 (1H, d, J = 8.8 Hz), 3.77-3.86 (3H, m), 3.84 (1 H, br. s.), 3.20-3.25 (1H, m), 2.61 (3H, d, J = 4.4 Hz), 2.00-2.16 (1H, m), 1.53 (2H, d, J = 12.4 Hz), 1.33-1.40 (1H, m), 1.23-1.33 (1H, m), 0.98 (9H, s) | 393.2 |
| 304 | 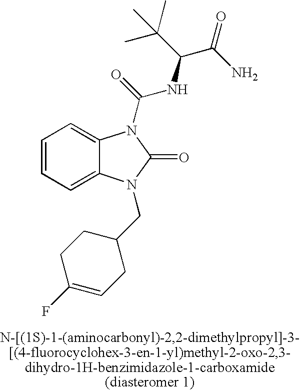 | 1H NMR (400 MHz, DMSO-d6) δ ppm 9.18 (1H, d, J = 8.8 Hz), 8.05 (1H, d, J = 8.1 Hz), 7.62 (1H, br. s.), 7.36 (1H, d, J = 8.1 Hz), 7.23 (1H, t, J = 7.5 Hz), 7.15 (2H, d, J = 8.4 Hz), 5.18 (1H, d, J = 16.5 Hz), 4.25 (1H, d, J = 8.8 Hz), 3.85 (2H, d, J = 2.6 Hz), 2.20 (2H, br. s.), 2.04- 2.14 (2H, m), 1.90 (1H, br. s.), 1.77- 1.87 (1H, m), 1.41-1.56 (1H, m), 1.00 (9H, s) | 402.2 |
| 305 | 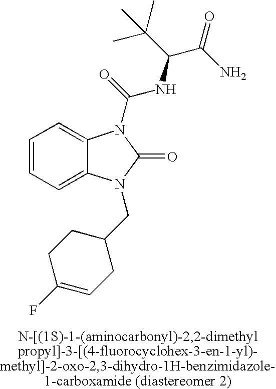 | 1H NMR (400 MHz, DMSO-d6) δ ppm 9.18 (1H, d, J = 9.2 Hz), 8.05 (1H, d, J = 8.1 Hz), 7.62 (1H, br. s.), 7.35 (1H, d, J = 8.1 Hz), 7.23 (1H, t, J = 7.5 Hz), 7.14 (2H, d, J = 8.8 Hz), 5.18 (1H, d, J = 17.6 Hz), 4.25 (1H, d, J = 8.8 Hz), 3.85 (2H, d, J = 5.5 Hz), 2.20 (2H, br. s.), 2.04- 2.14 (2H, m), 1.89 (1H, br. s.), 1.78- 1.87 (1H, m), 1.41-1.56 (1H, m), 1.00 (9H, s) | 402.2 |
| 306 | 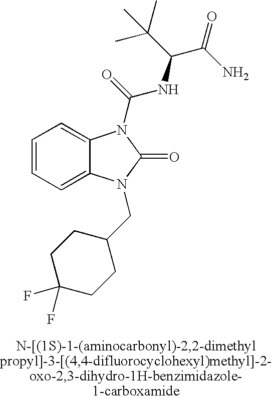 | 1H NMR (400 MHz, DMSO-d6) δ ppm 9.18 (1H, d, J = 8.8 Hz), 8.05 (1H, d, J = 8.1 Hz), 7.62 (1H, br. s.), 7.37 (1H, d, J = 7.7 Hz), 7.24 (1H, t, J = 7.5 Hz), 7.14 (2H, d, J = 8.1 Hz), 4.25 (1H, d, J = 8.8 Hz), 3.82 (2H, d, J = 7.0 Hz), 2.00 (3H, br. s.), 1.70-1.78 (3H, m), 1.30-1.39 (2H, m), 1.00 (9H, s) | 422.2 |
| 307 | 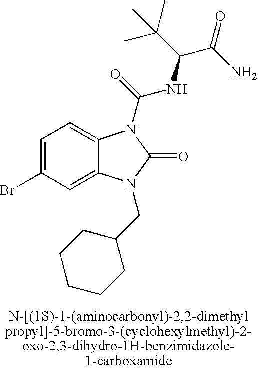 | 1H NMR (400 MHz, DMSO-d6) δ ppm 7.62 (1H, br. s.), 7.32 (1H, d, J = 8.4 Hz), 7.14 (1H, d, J = 1.8 Hz), 4.24 (1H, d, J = 9.2 Hz), 3.72 (2H, dd, J = 7.0, 2.6 Hz), 3.24 (2H, s), 1.80 (1H, td, J = 6.8, 3.7 Hz), 1.66 (3H, dd, J = 5.3, 2.7 Hz), 1.61 (2H, br. s.), 1.14 (1H, br. s.), 1.16 (2H, d, J = 2.9 Hz), 0.99 (10H, s), 0.94 (2H, s) | 464.1 |
| 308 | 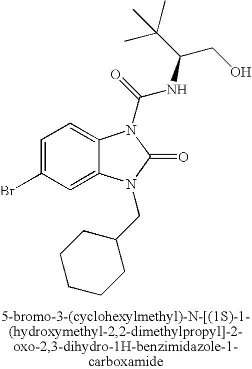 | 1H NMR (400 MHz, DMSO-d6) δ ppm 8.74 (1H, d, J = 9.5 Hz), 7.32 (1H, d, J = 8.4 Hz), 4.65 (1H, t, J = 5.1 Hz), 3.71 (2H, d, J = 4.4 Hz), 3.67-3.74 (1H, m), 3.65 (1H, d, J = 5.5 Hz), 3.49 (1H, d, J = 4.8 Hz), 1.81 (1H, d, J = 3.7 Hz), 1.63 (5H, d, J = 18.3 Hz), 1.15 (3H, d, J = 7.3 Hz), 1.03 (2H, d, J = 11.3 Hz), 1.07 (1H, br. s.), 0.95 (9H, s), 0.82 (1H, s) | 451.2 |
| 309 | 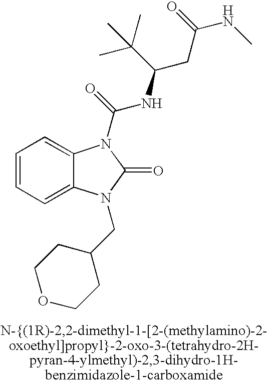 | 1H NMR (400 MHz, DMSO-d6) δ ppm 0.92 (s, 8H) 1.31 (br. s., 2H) 1.51 (br. s., 2H) 2.18 (br. s., 1H) 3.21 (s, 1H) 3.28 (d, J = 10.98 Hz, 6H) 3.76-3.86 (m, 4H) 4.14 (br. s., 1H) 7.15 (s, 1H) 7.23 (s, 1H) 7.35 (s, 1H) 7.78 (br. s., 1H) 8.71 (d, J = 9.52 Hz, 1H) | 416.2 |
| 310 | 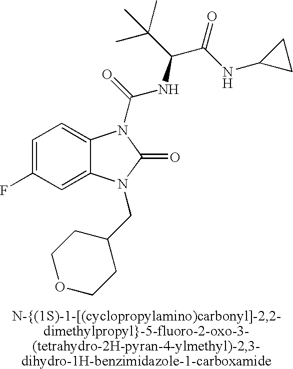 | 1H NMR (400 MHz, DMSO-d6) δ ppm 0.41 (d, J = 9.52 Hz, 1H) 0.63 (d, J = 7.32 Hz, 1H) 0.77 (d, J = 13.91 Hz, 2H) 0.90 (s. 4H) 0.96 (s, 6H) 1.29 (dd, J = 7.32, 4.39 Hz, 2H) 1.33 (br. s., 1H) 1.45 (br. s., 1H) 1.53 (d, J = 11.71 Hz, 1H) 2.04 (br. s., 1H) 3.17-3.28 (m, 1H) 3.23 (d, J = 10.98 Hz, 2H) 3.66 (d, J = 6.95 Hz, 1 H) 3.80 (d, J = 5.86 Hz, 1H) 3.77 (br. s., 1 H) 3.82 (br. s., 1H) 6.82 (d, J = 9.52 Hz, 1 H) 7.11 (t, J = 8.97 Hz, 1H) 8.13 (s, 1H) 8.26 (d, J = 4.03 Hz, 1H) | 446.2 |
| 311 | 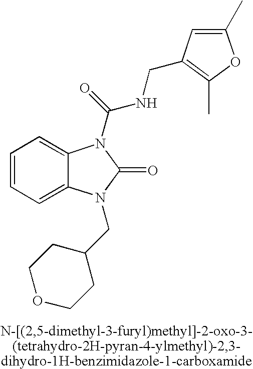 | 1H NMR (400 MHz, DMSO-d6) δ ppm 8.82 (1H, t, J = 5.1 Hz), 8.06 (1H, d, J = 8.1 Hz), 7.35 (1H, d, J = 7.7 Hz), 7.23 (1H, t, J = 7.3 Hz), 7.15 (1H, t, J = 7.7 Hz), 6.01 (1H, s), 4.22 (2H, d, J = 5.5 Hz), 3.81 (2H, d, J = 11.3 Hz), 3.76 (2H, 383.2 d, J = 7.0 Hz), 3.22 (1H, s), 3.22 (1H, d, J = 19.4 Hz), 2.24 (3H, s), 2.17 (3H, s), 2.05 (1H, dd, J = 15.2, 7.5 Hz), 1.49 (2 H, br. s.), 1.30 (1H, dd, J = 12.3, 3.5 Hz), 1.24-1.35 (1H, m) | 383.2 |
| 312 | 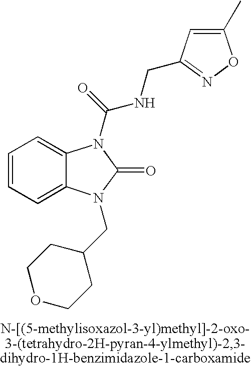 | 1H NMR (400 MHz, DMSO-d6) δ ppm 9.15 (1H, t, J = 5.7 Hz), 8.04 (1H, d, J = 7.7 Hz), 7.37 (1H, d, J = 7.7 Hz), 7.24 (1H, t, J = 7.7 Hz), 7.16 (1H, t, J = 7.5 Hz), 4.55 (2H, d, J = 5.5 Hz), 3.84 (2H, d, J = 2.2 Hz), 3.79 (3H, d, J = 7.0 Hz), 3.19-3.24 (2H, m), 2.38 (3H, s), 2.07 (1H, dd, J = 7.7, 3.7 Hz), 1.53 (2H, d, J = 11.7 Hz), 1.32 (2H, dd, J = 12.1, 4.0 Hz), 1.26-1.37 (1H, m) | 370.2 |
| 313 | 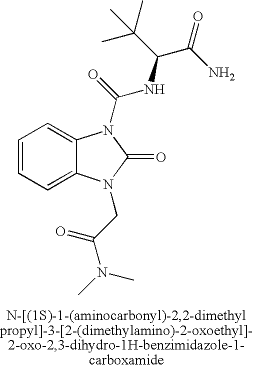 | 1H NMR (400 MHz, DMSO-d6) δ ppm 9.13 (1H, d, J = 8.8 Hz), 8.04 (1H, d, J = 7.7 Hz), 7.62 (1H, br. s.), 7.17-7.23 (2H, m), 7.08-7.17 (2H, m), 4.86 (2H, s), 4.26 (1H, d, J = 8.8 Hz), 3.12 (3H, s), 3.09 (1H, br. s.), 2.86 (3H, s), 2.84 (1 H, br. s.), 1.00 (8H, s), 0.94 (2H, s) | 375.2 |
| 314 | 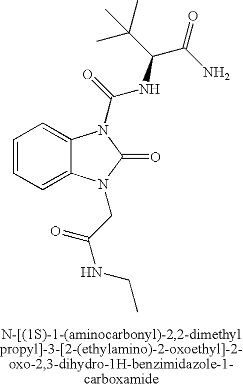 | 1H NMR (400 MHz, DMSO-d6) δ ppm 9.12 (1H, d, J = 8.8 Hz), 8.22 (1H, br. s.), 8.05 (1H, d, J = 7.3 Hz), 7.62 (1H, br. s.), 7.18 (1H, d, J = 8.8 Hz), 7.15 (2H, d, J = 8.1 Hz), 6.92-6.99 (1H, m), 4.51 (2 H, s), 4.36 (1H, s), 4.27 (1H, d, J = 8.8 Hz), 3.13-3.24 (1H, m), 3.11 (1H, d, J = 6.6 Hz), 0.98-1.06 (10H, m), 0.94 (3 H, s) | 375.2 |
| 315 | 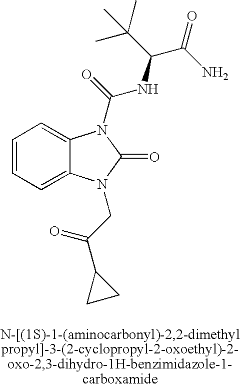 | 1H NMR (400 MHz, DMSO-d6) δ ppm 9.08 (1H, d, J = 8.8 Hz), 7.63 (1H, br. s.), 7.12-7.22 (3H, m), 6.91-7.00 (1H, m), 5.09 (1H, s), 4.87 (1H, s), 4.26 (1 H, d, J = 8.8 Hz), 2.24 (1H, br. s.), 0.99 (8 H, s), 1.03 (2H, d, J = 7.7 Hz), 0.94 (5H, s) | 372.2 |
| 316 | 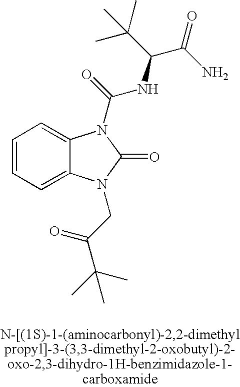 | 1H NMR (400 MHz, DMSO-d6) δ ppm 9.09 (1H, d, J = 9.2 Hz), 8.06 (1H, d, J = 7.7 Hz), 7.83 (1H, br. s.), 7.18 (1H, d, J = 4.4 Hz), 7.15 (2H, br. s.), 5.09 (2H, s), 4.27 (1H, d, J = 8.8 Hz), 1.25 (8H, s), 1.22 (1H, br. s.), 0.99 (9H, s), 0.94 (1 H, s) | 388.2 |
| 317 | 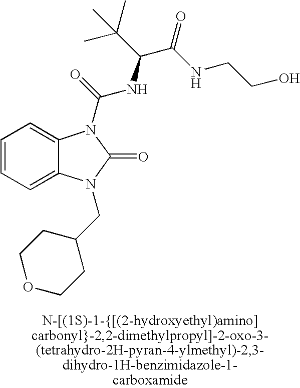 | 1H NMR (400 MHz, DMSO-d6) δ ppm 9.21 (1H, d, J = 8.8 Hz), 8.18 (1H, t, J = 4.9 Hz), 8.04 (1H, d, J = 8.1 Hz), 7.36 (1H, d, J = 7.7 Hz), 7.23 (1H, t, J = 7.5 Hz), 7.14 (1H, t, J = 7.7 Hz), 4.60 (1H, t, J = 5.1 Hz), 4.30 (1H, d, J = 8.8 Hz), 3.84 (1H, br. s.), 3.79 (3H, d, J = 7.7 Hz), 3.42 (2H, q, J = 5.9 Hz), 3.20 (1H, br. s.), 3.23 (2H, d, J = 11.0 Hz), 3.13 (1H, dt, J = 12.7, 6.3 Hz), 2.08 (1H, dd, J = 14.3, 7.0 Hz), 1.53 (2H, d, J = 12.1 Hz), 1.33 (2H, dd, J = 12.1, 3.3 Hz), 0.98 (9H, s) | 432.2 |
| 318 | 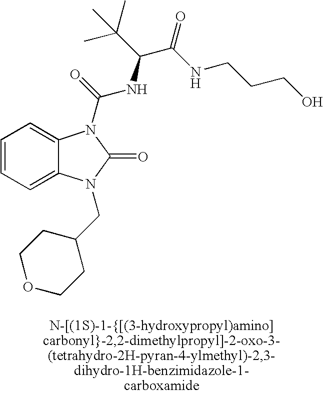 | 1H NMR (400 MHz, DMSO-d6) δ ppm 9.21 (1H, d, J = 8.8 Hz), 8.16 (1H, t, J = 4.8 Hz), 8.04 (1H, d, J = 8.1 Hz), 7.36 (1H, d, J = 7.7 Hz), 7.23 (1H, t, J = 7.5 Hz), 7.15 (1H, t, J = 7.7 Hz), 4.38 (1H, t, J = 4.9 Hz), 4.26 (1H, d, J = 8.8 Hz), 3.79 (3H, d, J = 7.7 Hz), 3.84 (1H, br. s.), 3.42 (2H, d, J = 5.5 Hz), 3.19 (1H, br. s.), 3.23 (2H, d, J = 12.1 Hz), 3.07 (1H, dt, J = 12.7, 6.3 Hz), 2.07 (1H, dt, J = 7.0, 3.5 Hz), 1.56 (3H, dd, J = 10.2, 2.6 Hz), 1.50- 1.60 (1H, m), 1.34 (1H, d, J = 3.3 Hz), 1.32 (1H, br. s.), 0.98 (8H, s), 0.93 (1H, s) | 446.3 |
| 319 | 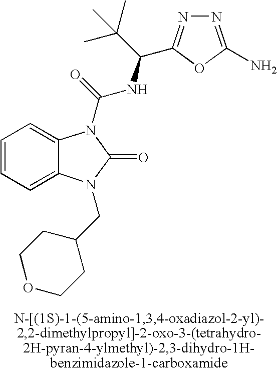 | 1H NMR (400 MHz, DMSO-d6) δ ppm 9.42 (1H, d, J = 8.8 Hz), 8.01 (1H, d, J = 8.1 Hz), 7.39 (1H, d, J = 7.7 Hz), 7.25 (1H, t, J = 7.7 Hz), 7.16 (1H, 1, J = 7.7 Hz), 7.01 (2H, s), 4.87 (1H, d, J = 8.4 Hz), 3.80 (3H, d, J = 7.7 Hz), 3.84 (1H, br. s.), 3.19-3.25 (2H, m), 2.07 (1H, dd, J = 10.4, 3.5 Hz), 1.54 (2H, d, J = 12.8 Hz), 1.34 (1H, d, J = 2.9 Hz), 1.28 (1H, d, J = 6.6 Hz), 1.32 (1H, br. s.), 1.03 (9H, s) | 428.2 |
| 320 | 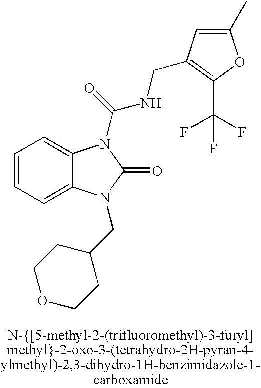 | 1H NMR (400 MHz, DMSO-d6) δ ppm 9.09 (1H, t, J = 5.9 Hz), 803 (1H, d, J = 7.7 Hz), 7.36 (1H, d, J = 7.3 Hz), 7.24 (1H, t, J = 7.7 Hz), 7.15 (1H, t, J = 7.9 Hz), 6.37 (1H, s), 4.47 (2H, d, J = 4.8 Hz), 3.82 (2H, dd, J = 11.5, 2.7 Hz), 3.78 (2H, d, J = 7.3 Hz), 3.19-3.26 (2H, m), 2.30 (3H, s), 2.05 (1H, td, J = 7.4, 3.8 Hz), 1.53 (2H, d, J = 12.8 Hz), 1.26- 1.37 (2H, m, J = 12.4, 12.4, 11.9, 4.4 Hz) | 437.2 |
| 321 | 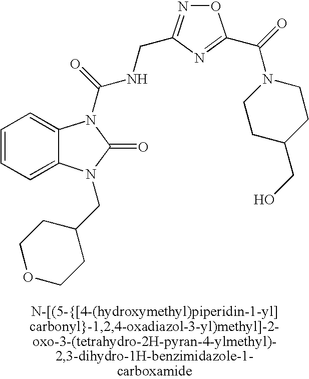 | 1H NMR (400 MHz, DMSO-d6) δ ppm 9.29 (1H, t, J = 5.7 Hz), 8.02 (1H, d, J = 7.7 Hz), 7.38 (1H, d, J = 8.1 Hz), 7.25 (1H, t, J = 7.3 Hz), 7.17 (1H, d, J = 7.0 Hz), 6.95-7.02 (1H, m), 4.78 (1H, d, J = 5.5 Hz), 4.49 (1H, t, J = 4.9 Hz), 4.39 (1H, d, J = 5.9 Hz), 4.43 (1H, t, J = 6.4 Hz), 3.80 (4H, d, J = 7.0 Hz), 3.84 (1H, 3.26 (4H, m), 2.89 (1H, td, J = 12.7, 3.1 Hz), 2.08 (1H, td, J = 7.5, 3.7 Hz), 1.81 (1H, br. s.), 1.70 (1H, t, J = 8.4 Hz), 1.66- 1.77 (1H, m), 1.47 (1H, d, J = 12.4 Hz), 1.54 (2H, d, J = 12.8 Hz), 1.32 (1H, dd, J = 12.6, 4.2 Hz), 1.26-1.37 (1H, m), 1.09-1.19 (1H, m), 1.14 (1H, d, J = 10.2 Hz) | 498.2 |
| 322 | 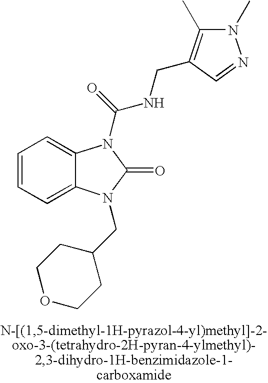 | 1H NMR (400 MHz, DMSO-d6) δ ppm 1.23-1.34 (m, J = 12.21, 12.21, 11.99, 4.21 Hz, 2H) 1.49 (br. s., 1H) 1.52 (d, J = 1.83 Hz, 1H) 2.04 (ddd, J = 11.35, 7.50, 4.21 Hz, 1H) 2.26 (s, 3H) 3.21 (t, J = 10.98 Hz, 2H) 3.70 (s, 3H) 3.74- 3.83 (m, 2H) 3.75 (d, J = 7.32 Hz, 3H) 4.30 (d, J = 5.49 Hz, 2H) 7.15 (t, J = 7.87 Hz, 1H) 7.22 (t, J = 7.32 Hz, 1H) 7.33- 7.37 (m, 2H) 8.06 (d, J = 8.05 Hz, 1H) 8.79 (s, 1H) | 383.2 |
| 323 | 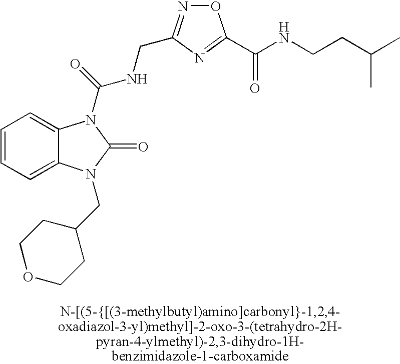 | 1H NMR (400 MHz, DMSO-d6) δ ppm 9.34 (1H, br. s.), 9.29 (1H, t, J = 5.9 Hz), 8.02 (1H, d, J = 8.1 Hz), 7.38 (1H, d, J = 7.7 Hz), 7.25 (1H, t, J = 7.3 Hz), 7.16 (1H, t, J = 7.7 Hz), 4.78 (2H, d, J = 5.5 Hz), 3.80 (3H, d, J = 7.3 Hz), 3.84 (1H, d, J = 2.9 Hz), 3.23 (1H, d, J = 13.9 Hz), 3.25 (2H, d, J = 8.1 Hz), 2.07 (1H, ddd, J = 11.3, 7.2, 3.8 Hz), 1.54 (2H, d, J = 11.7 Hz), 1.51-1.61 (1H, m), 1.42 (3H, q, J = 7.0 Hz), 1.32 (2H, dd, J = 12.8, 4.0 Hz), 0.89 (3H, s), 0.89 (2H, d, J = 6.6 Hz, 0.87 (2H, s) | 470.2 |
| 324 | 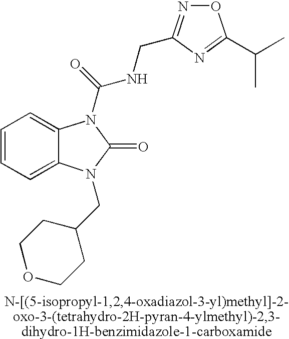 | 1H NMR (400 MHz, DMSO-d6) δ ppm 9.22 (1H, t, J = 5.9 Hz), 8.03 (1H, d, J = 8.1 Hz), 7.38 (1H, d, J = 8.1 Hz), 7.25 (1H, t, J = 7.1 Hz), 7.17 (1H, d, J = 8.1 Hz), 4.67 (2H, d, J = 5.9 Hz), 4.33-4.44 (1H, m), 3.80 (3H, d, J = 7.3 Hz), 3.75- 3.85 (2H, m), 3.20-3.26 (1H, m), 3.25 (1H, d, J = 8.8 Hz), 2.06 (1H, ddd, J = 11.3, 7.7, 4.0 Hz), 1.47-1.56 (2H, m), 1.32 (5H, d, J = 7.0 Hz), 1.29-1.36 (2H, m) | 399.2 |
| 325 | 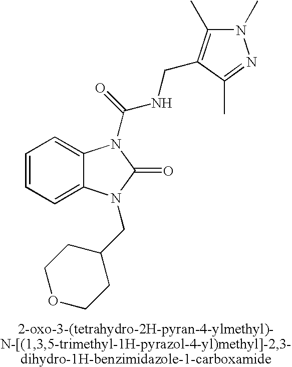 | 1H NMR (400 MHz, DMSO-d6) δ ppm 10.77 (1H, br. s.), 9.92-9.98 (1H, m), 8.55 (1H, s), 8.47 (1H, d, J = 3.3 Hz), 7.74 (1H, d, J = 8.1 Hz), 7.36 (1H, q, J = 7.8 Hz), 7.14 (1H, d, J = 5.9 Hz), 6.96- 7.01 (2H, m), 4.48 (2H, d, J = 4.8 Hz), 4.39 (1H, d, J = 5.9 Hz), 4.44 (1H, d, J = 6.2 Hz), 3.80-3.84 (1H, m), 3.82 (1 H, d, J = 13.9 Hz), 3.67 (2H, d, J = 7.3 Hz), 3.25 (1H, br. s.), 3.22 (1H, s), 3.22 H, t, J = 10.6 Hz), 2.02 (1H, ddd, J = 11.3, 7.3, 3.7 Hz), 1.46 (2H, t, J = 13.0 Hz), 1.42-1.52 (1H, m), 1.29 (2H, td, J = 12.1, 4.0 Hz) | 398.3 (M + H) |
| 326 | 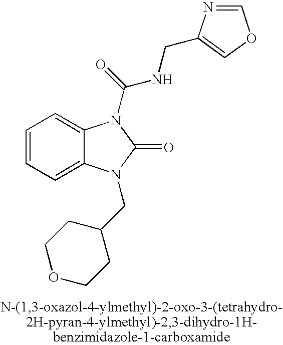 | 1H NMR (400 MHz, DMSO-d6) δ ppm 9.05 (1H, t, J = 5.7 Hz), 8.05 (1H, d, J = 7.0 Hz), 7.36 (1H, d, J = 7.7 Hz), 7.24 (1H, t, J = 7.7 Hz), 7.16 (1H, t, J = 7.9 Hz), 4.46 (2H, d, J = 5.5 Hz), 3.82 (2H, dd, J = 11.3, 2.6 Hz), 3.78 (2H, d, J = 7.0 Hz), 3.18-3.26 (3H, m), 1.99-2.11 (1 H, m, J = 11.3, 7.5, 3.8, 3.8 Hz), 1.54 (1 H, d, J = 1.5 Hz), 1.51 (2H, br. s.), 1.31 (2 H, qd, J = 12.2, 4.4 Hz) | 356.2 |
| 327 | 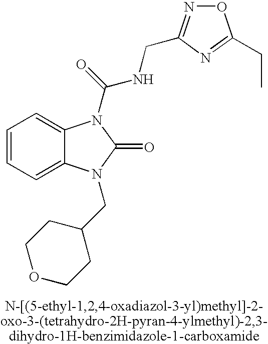 | 1H NMR (400 MHz, DMSO-d6) δ ppm 9.22 (1H, t, J = 5.7 Hz), 8.03 (1H, d, J = 7.7 Hz), 7.38 (1H, d, J = 7.7 Hz), 7.25 (1H, t, J = 7.7 Hz), 7.16 (1H, t, J = 7.9 Hz), 4.67 (2H, d, J = 5.9 Hz), 3.80 (3H, d, J = 7.3 Hz), 3.84 (1H, d, J = 2.9 Hz), 3.19-3.26 (2H, m), 2.94 (2H, q, J = 7.4 Hz), 2.07 (1H, td, J = 7.5, 3.7 Hz), 1.53 (2H, d, J = 12.4 Hz), 1.28 (4H, t, J = 7.5 Hz), 1.25-1.37 (1H, m) | 385.2 |
| 328 | 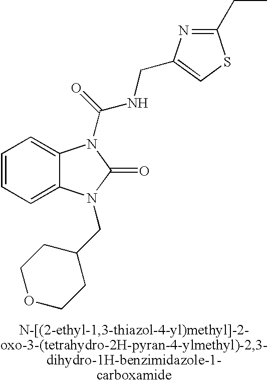 | 1H NMR (400 MHz, DMSO-d6) δ ppm 9.13 (1H, t, J = 5.5 Hz), 8.05 (1H, d, J = 8.1 Hz), 7.34-7.39 (2H, m), 7.24 (1 H, t, J = 7.7 Hz), 7.15 (1H, t, J = 7.9 Hz), 4.58 (2H, d, J = 5.5 Hz), 3.82 (2H, dd, J = 11.5, 2.7 Hz), 3.78 (2H, d, J = 7.3 Hz), 3.19-3.26 (2H, m), 2.98 (2H, q, J = 7.7 Hz), 2.06 (1H, td, J = 7.5, 3.7 Hz), 1.53 (2H, d, J = 11.3 Hz), 1.30 (4H, 1, J = 7.5 Hz), 1.26-1.37 (1H, m) | 400.2 |
| 329 | 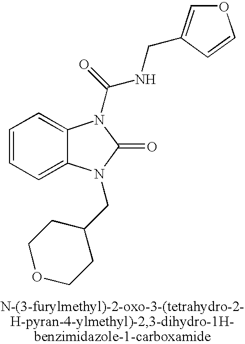 | 1H NMR (400 MHz, DMSO-d6) δ ppm 8.93 (1H, t, J = 5.5 Hz), 8.06 (1H, d, J = 8.1 Hz), 7.63 (1H, d, J = 14.6 Hz), 7.36 (1H, d, J = 7.7 Hz), 7.23 (1H, t, J = 7.1 Hz), 7.16 (1H, t, J = 7.9 Hz), 6.52 (1H, s), 4.37 (2H, d, J = 5.5 Hz), 3.77 (2H, d, J = 7.3 Hz), 3.74-3.84 (3H, m), 3.22 (1 H, t, J = 10.8 Hz), 3.24 (1H, d, J = 15.7 Hz), 2.05 (1H, ddd, J = 11.3, 7.6, 3.8 Hz), 1.53 (1H, d, J = 1.8 Hz), 1.50 (1H, br. s.), 1.25-1.36 (2H, m, J = 12.3, 12.3, 12.0, 4.4 Hz) | 555.2 |
| 330 | 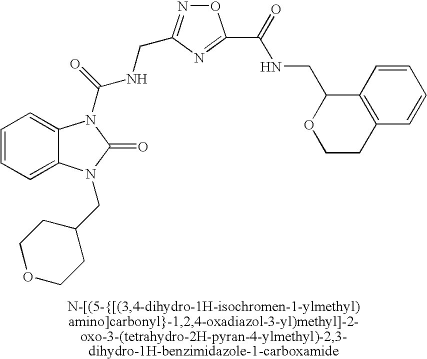 | 1H NMR (400 MHz, DMSO-d6) δ ppm 9.30 (1H, t, J = 5.7 Hz), 9.38 (1H, t, J = 5.7 Hz), 8.03 (1H, d, J = 8.1 Hz), 7.39 (1H, d, J = 7.7 Hz), 7.25 (1H, t, J = 7.9 Hz), 7.13-7.20 (1H, m), 7.19 (4H, d, J = 2.9 Hz), 4.91 (1H, dd, J = 8.4, 3.3 Hz), 4.79 (2H, d, J = 5.5 Hz), 4.03 (1H, dt, J = 11.3, 5.5 Hz), 3.81 (3H, d, J = 7.3 Hz), 3.84 (1H, br. s.), 3.62-3.74 (4H, m), 3.19-3.26 (2H, m), 2.71-2.81 (2H, m), 2.07 (1H, dd, J = 11.3, 7.3 Hz), 1.53 (1H, d, J = 1.5 Hz), 1.46-1.57 (1H, m), 1.27-1.38 (2H, m, J = 12.4, 12.4, 12.1, 4.6 Hz) | 546.2 |
| 331 | 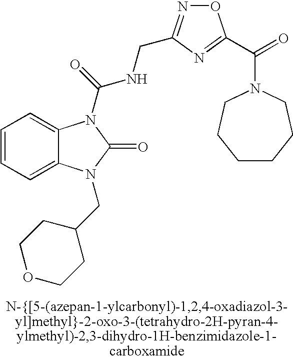 | 1H NMR (400 MHz, DMSO-d6) δ ppm 9.29 (1H, t, J = 5.7 Hz), 8.02 (1H, d, J = 8.1 Hz), 7.38 (1H, d, J = 8.1 Hz), 7.25 (1H, t, J = 7.7 Hz), 7.16 (1H, t, J = 7.9 Hz), 4.78 (2H, d, J = 5.9 Hz), 3.80 (3H, d, J = 7.0 Hz), 3.84 (1H, d, J = 2.6 Hz), 3.61 (1H, d, J = 6.2 Hz), 3.58-3.68 (1H, m), 3.49 (2H, t, J = 6.0 Hz), 3.19-3.26 (2 H, m), 2.07 (1H, td, J = 7.3, 3.3 Hz), 1.64- 1.75 (2H, m), 1.64-1.75 (2H, m, J = 5.8, 5.8, 5.8, 5.8 Hz), 1.50 (1H, br. s.), 1.53 (5H, d, J = 10.2 Hz), 1.32 (1H, dd, J = 13.2, 4.4 Hz), 1.26-1.37 (1H, m) | 482.2 |
| 332 | 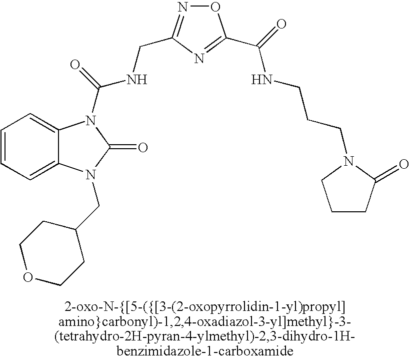 | 1H NMR (400 MHz, DMSO-d6) δ ppm 9.29 (1H, t, J = 5.9 Hz), 9.26-9.35 (1H, m), 8.02 (1H, d, J = 8.1 Hz), 7.38 (1H, d, J = 7.7 Hz), 7.25 (1H, t, J = 7.3 Hz), 7.16 (1H, t, J = 8.4 Hz), 6.94-7.05 (1H, m), 4.78 (1H, d, J = 5.9 Hz), 3.80 (3H, d, J = 7.3 Hz), 3.84 (1H, d, J = 2.9 Hz), 3.31- 3.36 (1H, m), 3.17-3.26 (6H, m), 2.19 (2H, t, J = 7.9 Hz), 2.08 (1H, dt, J = 7.5, 3.9 Hz), 1.91 (1H, d, J = 7.3 Hz), 1.86- 1.96 (1H, m), 1.67-1.76 (2H, m, J = 7.0, 7,0, 7.0, 7.0 Hz), 1.54 (2H, d, J = 11.0 Hz), 1.32 (2H, dd, J = 12.6, 4.2 Hz), 1.26- 1.37 (1H, m) | 525.2 |
| 333 | 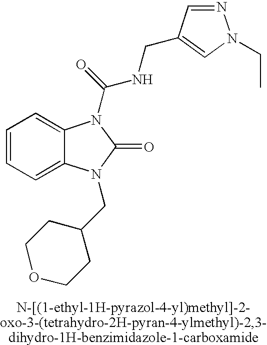 | 1H NMR (400 MHz, DMSO-d6) δ ppm 8.88 (1H, t, J = 5.5 Hz), 8.07 (1H, d, J = 8.1 Hz), 7.71 (1H, s), 7.42 (1H, s), 7.35 (1H, d, J = 7.7 Hz), 7.23 (1H, t, J = 7.3 Hz), 7.15 (1H, t, J = 7.7 Hz), 4.36 (2H, d, J = 5.9 Hz), 4.09 (2H, q, J = 7.3 Hz), 3.81 (2H, dd, J = 11.5, 2.7 Hz), 3.76 (2H, d, J = 7.3 Hz), 3.22 (2H, t, J = 10.8 Hz), 2.04 (1H, td, J = 7.6, 3.8 Hz), 1.51 (2H, d, J = 11.0 Hz), 1.29 (2H, dd, J = 12.6, 4.2 Hz), 1.34 (3H, t, J = 7.1 Hz) | 383.2 |
| 334 | 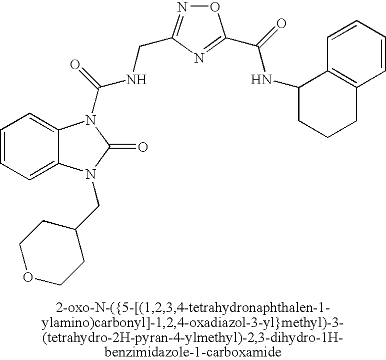 | 1H NMR (400 MHz, DMSO-d6) δ ppm 9.72 (1H, d, J = 8.4 Hz), 9.29 (1H, t, J = 5.9 Hz), 7.38 (1H, d, J = 7.7 Hz), 7.25 (1H, t, J = 7.7 Hz), 7.10-7.21 (5H, m), 6.94-7.05 (1H, m), 5.19 (1H, br. s.), 4.78 (1H, d, J = 5.9 Hz), 3.79 (4H, d, J = 7.3 Hz), 3.19-3.26 (2H, m), 2.74 (2 H, d, J = 4.8 Hz), 2.05 (1H, d, J = 8.1 Hz), 1.97 (2H, br. s.), 1.89 (1H, d, J = 8.8 Hz), 1.74 (1H, br. s.), 1.52 (2H, br. s.), 1.32 (2H, d, J = 7.0 Hz), 1.26-1.37 (1H, m) | 530.2 |
| 335 | 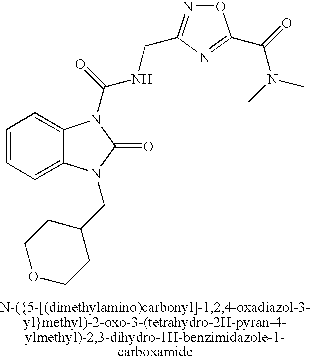 | 1H NMR (400 MHz, DMSO-d6) δ ppm 9.29 (1H, t, J = 5.7 Hz), 8.02 (1H, d, J = 7.7 Hz), 7.38 (1H, d, J = 7.7 Hz), 7.25 (1H, t, J = 7.9 Hz), 7.16 (1H, t, J = 8.2 Hz), 6.97 (1H, s), 4.79 (2H, d, J = 5.5 Hz), 3.80 (4H, d, J = 7.3 Hz), 3.84 (1H, br. s.), 3.24 (1H, d, J = 1.5 Hz), 3.22 (1H, d, J = 6.2 Hz), 3.11 (3H, s), 3.04 (3H, s), 2.07 (1H, ddd, J = 11.3, 7.2, 3.8 Hz), 1.54 (2H, d, J = 1 2.4 Hz), 1.32 (2H, dd, J = 12.6, 4.2 Hz), 1.26-1.37 (1H, m) | 428.2 |
| 336 | 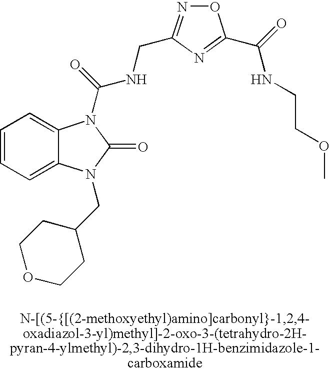 | 1H NMR (400 MHz, DMSO-d6) δ ppm 9.29 (1H, t, J = 5.9 Hz), 8.02 (1H, d, J = 7.7 Hz), 7.35 (1H, d, J = 8.1 Hz), 7.25 (1H, t, J = 7.7 Hz), 7.16 (1H, t, J = 8.2 Hz), 6.97 (1H, s), 4.78 (2H, d, J = 5.9 Hz), 3.80 (3H, d, J = 7.3 Hz), 3.84 (1H, br. s.), 3.67 (1H, d, J = 7.3 Hz), 3.40- 3.48 (4H, m), 3.20-3.26 (5H, m), 2.06 (1H, ddd, J = 18.1, 10.8, 3.7 Hz), 1.47 (1 H, d, J = 14.3 Hz), 1.52 (1H, br. s.), 1.32 (2H, dd, J = 12.6, 4.6 Hz), 1.26-1.37 (1 H, m) | 458.2 |
| 337 | 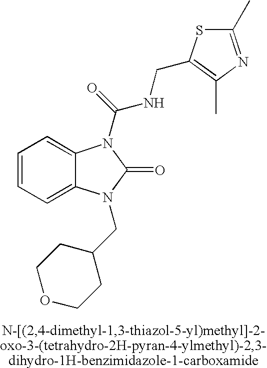 | 1H NMR (400 MHz, DMSO-d6) δ ppm 9.09 (1H, t, J = 5.7 Hz), 8.04 (1H, d, J = 7.7 Hz), 7.35 (1H, d, J = 7.7 Hz), 7.23 (1H, t, J = 7.3 Hz), 7.15 (1H, t, J = 7.7 Hz), 4.60 (2H, d, J = 5.9 Hz), 3.81 (3H, dd, J = 11.5, 3.1 Hz), 3.76 (2H, d, J = 7.0 Hz), 3.22 (2H, t, J = 11.0 Hz), 3.26 (1H, br. s.), 2.34 (3H, s), 1.99-2.09 (1H, m, J = 11.1, 7.4, 3.8, 3.7 Hz), 1.51 (2H, d, Hz), 1.30 (2H, dd, J = 12.6, 4.2 Hz) | 400.2 |
| 338 | 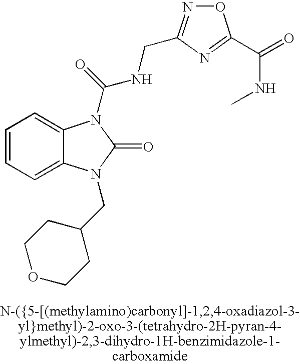 | 1H NMR (400 MHz, DMSO-d6) δ ppm 9.29 (2H, t, J = 5.7 Hz), 8.02 (1H, d, J = 7.7 Hz), 7.38 (1H, d, J = 7.7 Hz), 7.25 (1H, t, J = 7.3 Hz), 7.16 (1H, t, J = 8.2 Hz), 4.78 (1H, d, J = 5.9 Hz), 3.80 (3H, d, J = 7.3 Hz), 3.84 (1H, d, J = 3.7 Hz), 3.19-3.26 (1H, m), 3.24 (1H, d, J = 1.5 Hz), 2.79 (3H, d, J = 4.8 Hz), 2.07 (1H, td, J = 7.5, 3.7 Hz), 1.54 (2H, d, J = 12.4 Hz), 1.42 (1H, s), 1.32 (1H, dd, J = 12.8, 4.4 Hz), 1.26-1.37 (1H, m) | 414.2 |
| 339 | 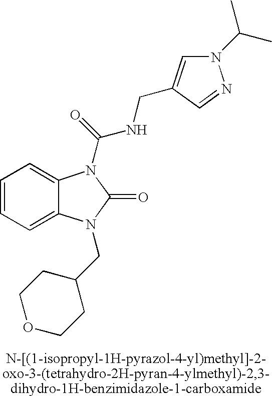 | 1H NMR (400 MHz, DMSO-d6) δ ppm 8.87 (1H, t, J = 5.5 Hz), 8.07 (1H, d, J = 7.7 Hz), 7.73 (1H, s), 7.35 (1H, d, J = 7.7 Hz), 7.23 (1H, t, J = 7.3 Hz), 7.15 (1H, t, J = 8.2 Hz), 4.45 (1H, dt, J = 13.3, 6.7 Hz), 4.36 (2H, d, J = 5.5 Hz), 3.81 (2 H, dd, J = 11.5, 2.7 Hz), 3.76 (2H, d, J = 7.3 Hz), 3.22 (2H, t, J = 10.6 Hz), 2.04 (1H, ddd, J = 11.3, 7.4, 3.7 Hz), 1.51 (2 H, d, J = 11.3 Hz), 1.39 (6H, d, J = 7.0 Hz), 1.30 (1H, t, J = 12.3 Hz), 1.30 (1H, q, J = 12.1 Hz) | 397.2 |
| 340 | 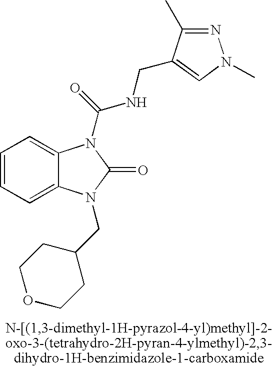 | 1H NMR (400 MHz, DMSO-d6) δ ppm 9.32 (1H, t, J = 5.9 Hz), 8.04 (1H, dd, J = 7.9, 3.5 Hz), 7.39 (1H, d, J = 8.1 Hz), 7.15-7.23 (3H, m), 6.96-7.04 (1H, m), 4.85 (1H, s), 4.82 (2H, d, J = 5.1 Hz), 4.42 (1H, d, J = 5.5 Hz), 3.77-3.89 (6H, m), 3.67 (1H, d, J = 7.0 Hz), 3.19- 3.26 (3H, m), 2.89 (1H, d, J = 5.9 Hz), 2.92 (1H, t, J = 6.0 Hz), 2.34 (1H, s), 2.06 (1H, td, J = 11.1, 7.1 Hz), 1.52 (2H, t, J = 11.7 Hz), 1.32 (1H, d, J = 14.3 Hz), 1.25-1.36 (1H, m) | 383.2 |
| 341 | 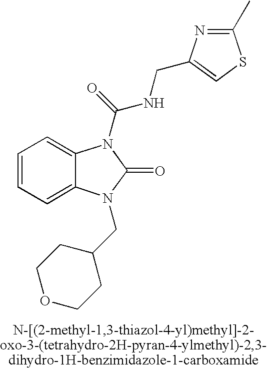 | 1H NMR (400 MHz, DMSO-d6) δ ppm 9.12 (1H, t, J = 5.5 Hz), 8.05 (1H, d, J = 7.7 Hz), 7.34-7.41 (2H, m), 7.24 (1 H, t, J = 7.3 Hz), 7.16 (1H, t, J = 7.9 Hz), 4.57 (2H, d, J = 5.5 Hz), 3.82 (2H, dd, J = 11.7, 2.6 Hz), 3.78 (2H, d, J = 7.3 Hz), 3.23 (1H, s), 3.23 (1H, t, J = 10.8 Hz), 2.65 (3H, s), 2.06 (1H, ddd, J = 11.2, 7.3, 3.8 Hz), 1.53 (2H, d, J = 11.0 Hz), 1.31 (2H, dd, J = 12.8, 4.0 Hz), 1.26- 1.37 (1H, m) | 387.2 (M + H) |
| 342 | 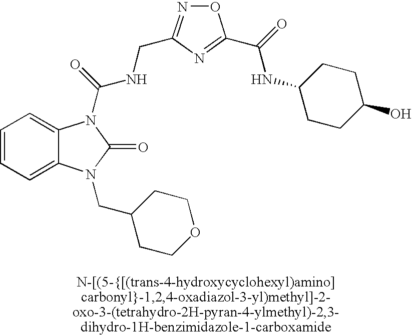 | 1H NMR (400 MHz, DMSO-d6) δ ppm 9.28 (1H, t, J = 5.7 Hz), 9.21 (1H, d, J = 8.1 Hz), 8.23 (1H, none), 8.02 (1H, d, J = 7.7 Hz), 7.38 (1H, d, J = 7.7 Hz), 7.25 (1H, t, J = 7.9 Hz), 7.16 (1H, t, J = 8.2 Hz), 6.95-7.01 (1H, m), 4.77 (1 H, d, J = 5.9 Hz), 4.52 (1H, d, J = 4.4 Hz), 3.80 (3H, d, J = 7.3 Hz), 3.83 (1H, d, J = 7.0 Hz), 3.67 (1H, d, J = 7.3 Hz), 3.65- 3.74 (1H, m), 3.36 (1H, td, J = 11.0, 4.0 Hz), 3.19-3.26 (2H, m), 2.07 (1H, ddd, J = 8.4, 4.8, 4.4 Hz), 1.74-1.86 (4H, m), 1.55 (2H, br. s.), 1.45 (3H, t, J = 12.8 Hz), 1.32 (1H, dd, J = 12.3, 4.2 Hz), 1.22 (2H, d, J = 12.8 Hz), 1.27 (1H, br..s.) | 498.2 |
| 343 | 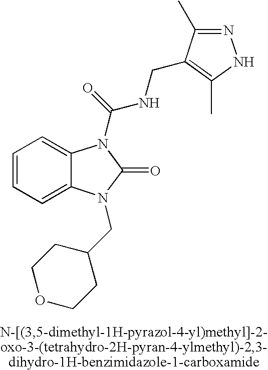 | 1H NMR (400 MHz, DMSO-d6) δ ppm 8.70 (1H, t, J = 5.3 Hz), 8.06 (1H, d, J = 7.7 Hz), 7.35 (1H, d, J = 7.7 Hz), 7.22 (1H, t, J = 7.1 Hz), 7.15 (1H, t, J = 7.7 Hz), 4.28 (2H, d, J = 5.1 Hz), 3.75 (3H, d, J = 7.3 Hz), 3.73-3.84 (3H, m), 3.21 (2H, t, J = 10.8 Hz), 2.18 (5H, br. s.), 2.03 (1H, ddd, J = 11.1, 7.4, 3.8 Hz), 1.51 (1H, d, J = 1.5 Hz), 1.48 (1H, br. s.), 1.28 (2H, qd, J = 12.2, 4.4 Hz) | 384.2 (M + H) |
| 344 | 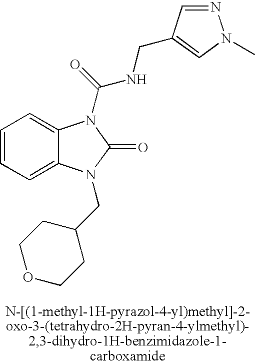 | 1H NMR (400 MHz, DMSO-d6) δ ppm 8.88 (1H, t, J = 5.5 Hz), 8.06 (1H, d, J = 7.7 Hz), 7.67 (1H, s), 7.35 (1H, d, J = 7.7 Hz), 7.41 (1H, s), 7.23 (1H, t, J = 7.1 Hz), 7.15 (1H, t, J = 8.2 Hz), 4.35 (2H, d, J = 5.5 Hz), 3.75-3.83 (7H, m), 3.22 (2H, t, J = 10.6 Hz), 2.04 (1H, ddd, J = 11.1, 7.4, 3.8 Hz), 1.53 (1H, br. s.), 1.49 (1H, d, J = 1.8 Hz), 1.24-1.34 (2H, m, J = 1 2.3, 12.3, 12.0, 4.4 Hz) | 369.2 |
| 345 | 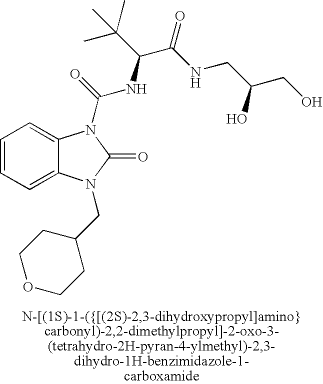 | 1H NMR (400 MHz, DMSO-d6) δ ppm 9.21 (1H, d, J = 9.2 Hz), 8.14 (1H, br. s.), 8.04 (1H, d, J = 8.1 Hz), 7.37 (1H, d, J = 7.7 Hz), 7.23 (1H, t, J = 7.7 Hz), 7.15 (1H, t, J = 7.7 Hz), 4.63 (1H, d, J = 4.8 Hz), 4.47 (1H, t, J = 5.5 Hz), 4.34 (1H, d, J = 8.8 Hz), 3.79 (3H, d, J = 7.7 Hz), 3.84 (1H, br. s.), 3.52 (1H, d, J = 5.1 Hz), 3.30- 3.36 (2H, m), 3.15-3.25 (2H, m), 2.97 (1H, ddd, J = 12.8, 6.2, 5.9 Hz), 2.08 (1H, dd, J = 11.2, 5.3 Hz), 1.53 (2H, d, J = 12.4 Hz), 1.33 (1H, d, J = 14.6 Hz), 1.28-1.38 (1H, m), 0.99 (9H, s) | 462.3 |
| 346 | 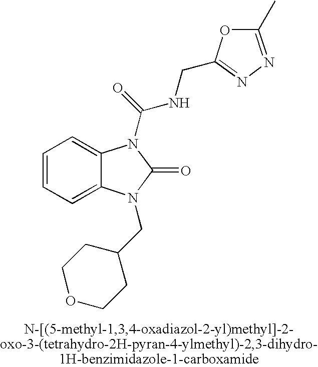 | 1H NMR (400 MHz, DMSO-d6) δ ppm 9.28 (1H, t, J = 5.9 Hz), 8.04 (1H, d, J = 7.7 Hz), 7.40 (1H, d, J = 7.7 Hz), 7.27 (1H, t, J = 7.1 Hz), 7.18 (1H, t, J = 7.3 Hz), 4.78 (2H, d, J = 5.9 Hz), 3.82 (3H, d, J = 7.3 Hz), 3.86 (2H, d, J = 2.9 Hz), 3.25 (1H, s), 3.25 (2H, t, J = 11.0 Hz), 2.52 (1H, br. s.), 2.09 (1H, ddd, J = 11.2, 7.5, 4.0 Hz), 1.54 (2H, br. s.), 1.34 (2H, qd, J = 12.2, 4.4 Hz) | 371.2 |
| 347 | 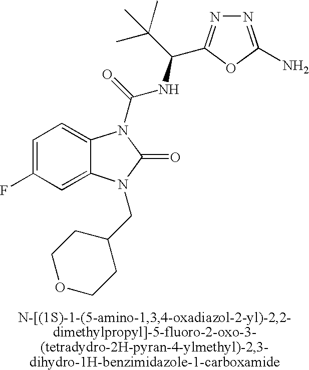 | 1H NMR (400 MHz, DMSO-d6) δ ppm 9.38 (1H, d, J = 8.8 Hz), 7.79 (1H, dd, J = 9.5, 2.6 Hz), 7.41 (1H, dd, J = 8.8, 4.8 Hz), 7.11 (1H, td, J = 9.1, 2.6 Hz), 7.03 (2 H, s), 4.87 (1H, d, J = 8.8 Hz), 3.81 (3H, br. s.), 3.79 (2H, d, J = 7.3 Hz), 3.74 (1H, d, J = 7.0 Hz), 3.19-3.26 (3H, m), 2.05 (1H, td, J = 11.8, 4.6 Hz), 1.54 (2H, d, J = 12.4 Hz), 1.32 (2H, dd, J = 12.4, 4.0 Hz), 1.03 (8H, s) | 446.2 |
| 348 | 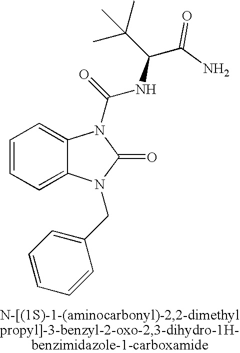 | 1H NMR (400 MHz, DMSO-d6) δ ppm 9.19 (1H, d, J = 8.8 Hz), 8.06 (1H, d, J = 7.3 Hz), 7.63 (1H, br. s.), 7.32-7.39 (4H, m), 7.30 (1H, d, J = 6.6 Hz), 7.20- 7.27 (1H, m), 7.15 (2H, d, J = 5.5 Hz), 7.17 (1H, d, J = 7.0 Hz), 5.13 (2H, d, J = 4.8 Hz), 4.27 (1H, d, J = 8.8 Hz), 1.01 (9H, s), 0.93 (1H, s) | 380.2 |
| 349 | 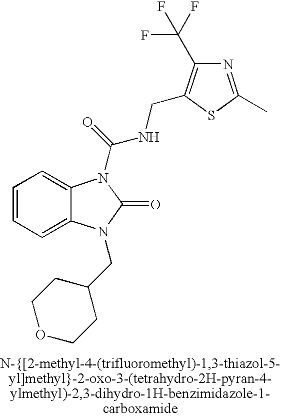 | 1H NMR (400 MHz, DMSO-d6) δ ppm 9.36 (1H, t, J = 5.9 Hz), 8.01 (1H, d, J = 7.7 Hz), 7.37 (1H, d, J = 7.7 Hz), 7.25 (1H, t, J = 7.7 Hz), 7.15 (1H, t, J = 8.2 Hz), 6.97 (1H, s), 4.82 (2H, d, J = 5.5 Hz), 3.78 (2H, d, J = 7.0 Hz), 3.82 (1H, d, J = 11.7 Hz), 3.67 (1H, d, J = 7.3 Hz), 3.23 (2H, t, J = 10.8 Hz), 2.57-2.65 (3 H, m), 2.05 (1H, dd, J = 11.7, 7.7 Hz), 1.47 (1H, d, J = 12.8 Hz), 1.53 (1H, d, J = 11.7 Hz), 1.32 (1H, d, J = 12.8 Hz), 1.31 (1H, q, J = 12.3 Hz) | 454.1 |
| 350 | 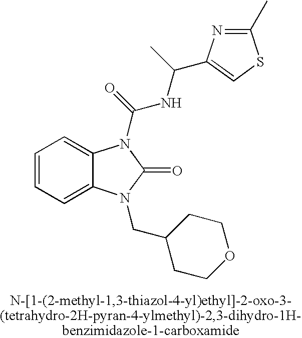 | 1H NMR (400 MHz, DMSO-d6) δ ppm 9.15 (1H, d, J = 7.3 Hz), 8.05 (1H, d, J = 7.7 Hz), 7.38 (1H, s), 7.36 (1H, d, J = 8.4 Hz), 7.23 (1H, t, J = 7.3 Hz), 7.15 (1H, t, J = 7.9 Hz), 5.15 (1H, t, J = 7.1 Hz), 3.82 (2H, d, J = 11.0 Hz), 3.77 (2H, d, J = 7.3 Hz), 3.23 (2H, t, J = 11.3 Hz), 2.66 (3H, s), 2.05 (1H, ddd, J = 7.5, 4.0, 3.8 Hz), 1.52 (5H, d, J = 7.0 Hz), 1.31 (2 H, dd, J = 11.9, 3.5 Hz), 1.26-1.36 (1H, m) | 401.2 (M + H) |
| 351 | 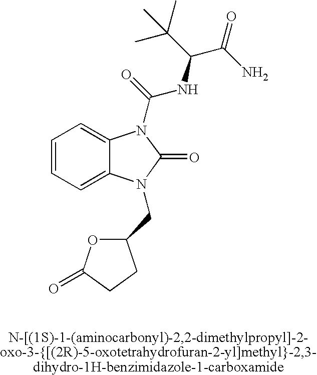 | 1H NMR (400 MHz, DMSQ-d6) δ ppm 9.15 (1H, d, J = 8.8 Hz), 8.05 (1H, d, J = 7.7 Hz), 7.63 (1H, br. s.), 7.38 (1H, d, J = 7.7 Hz), 7.25 (1H, t, J = 7.3 Hz), 7.16 (2H, d, J = 8.8 Hz), 7.19 (1H, s), 4.89 (1 H, dd, J = 6.4, 3.5 Hz), 4.26 (1H, d, J = 9.1 Hz), 4.16 (1H, d, J = 4.0 Hz), 4.12-4.21 (1H, m), 2.34 (1H, dd, J = 13.2, 7.0 Hz), 2.03 (1H, dd, J = 11.0, 9.1 Hz), 1.00 (10 H, s) | 388.2 |
| 352 | 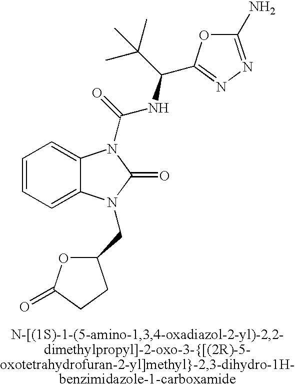 | 1H NMR (400 MHz, DMSO-d6) δ ppm 9.38 (1H, d, J = 8.4 Hz), 8.02 (1H, d, J = 8.1 Hz), 7.40 (1H, d, J = 7.3 Hz), 7.26 (1H, t, J = 7.7 Hz), 7.18 (1H, t, J = 7.7 Hz), 7.03 (2H, s), 4.88 (2H, d, J = 8.8 Hz), 4.11-4.26 (2H, m), 2.56 (1H, s), 2.35 (1H, dd, J = 12.8, 7.0 Hz), 1.97- 2.08 (1H, m), 1.03 (10H, s) | 428.2 |
| 353 | 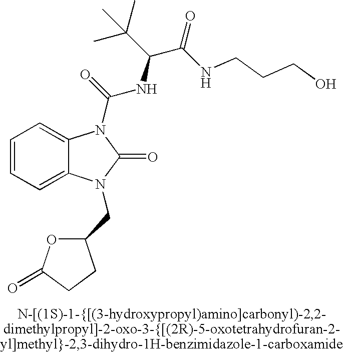 | 1H NMR (400 MHz, DMSO-dB) δ ppm 9.17 (1H, d, J = 8.8 Hz), 8.18 (1H, t, J = 5.3 Hz), 8.05 (1H, d, J = 7.7 Hz), 7.38 (1H, d, J = 7.3 Hz), 7.24 (1H, t, J = 7.7 Hz), 7.17 (1H, t, J = 7.9 Hz), 4.89 (1H, dd, J = 7.3, 4.0 Hz), 4.38 (1H, t, J = 4.9 Hz), 4.27 (1H, d, J = 8.8 Hz), 4.17 (1H, d, J = 9.9 Hz), 4.11-4.21 (1H, m), 3.42 (1H, d, J = 5.1 Hz), 3.42 (1H, d, J = 17.2 Hz), 3.20 (1H, dt, J = 13.4, 6.6 Hz), 3.07 (1H, dt, J = 12.8, 6.4 Hz), 2.34 (1H, d, J = 5.9 Hz), 2.29-2.41 (1H, m), 1.97- 2.09 (1H, m), 1.58 (2H, td, J = 6.7, 2.0 Hz), 0.98 (10H, s) | 446.2 |
| 354 | 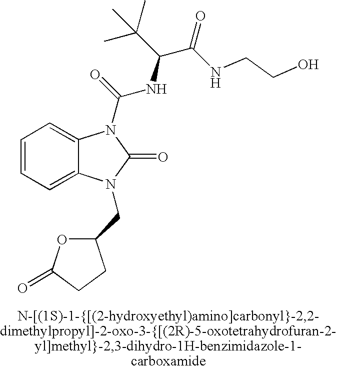 | 1H NMR (400 MHz, DMSO-d6) δ ppm 9.18 (1H, d, J = 8.8 Hz), 8.20 (1H, t, J = 5.3 Hz), 8.05 (1H, d, J = 7.7 Hz), 7.38 (1H, d, J = 7.7 Hz), 7.24 (1H, t, J = 7.3 Hz), 7.17 (1H, t, J = 7.7 Hz), 4.89 (1H, dd, J = 7.1, 3.8 Hz), 4.61 (1H, t, J = 4.8 Hz), 4.31 (1H, d, J = 9.1 Hz), 4.11-4.22 (2H, m), 3.42 (2H, d, J = 5.1 Hz), 3.31 (1 H, s), 3.21 (2H, dd, J = 13.0, 6.4 Hz), 3.13 (1H, ddd, J = 12.3, 6.4, 6.2 Hz), 2.34 (1H, dd, J = 12.8, 7.0 Hz), 1.97- 2.07 (1H, m), 0.98 (9H, s) | 433.2 |
| 355 | 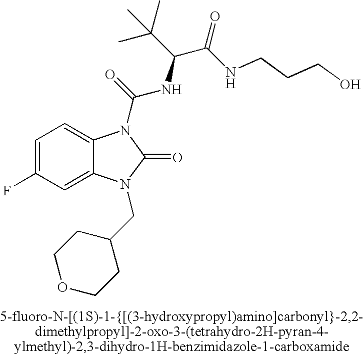 | 1H NMR (400 MHz, DMSO-d6) δ ppm 9.18 (1H, d, J = 9.1 Hz), 8.18 (1H, t, J = 5.3 Hz), 7.81 (1H, dd, J = 9.5, 2.6 Hz), 7.39 (1H, dd, J = 8.8, 4.4 Hz), 7.09 (1H, td, J = 9.1, 2.6 Hz), 4.39 (1H, t, J = 5.1 Hz), 4.25 (1H, d, J = 9.1 Hz), 3.81 (3H, dd, J = 11.2, 7.1 Hz), 3.77 (1H, br. s.), 3.42 (1H, d, J = 5.5 Hz), 3.39 (1H, d, J = 4.8 Hz), 3.19 (1H, br. s.), 3.22 (2H, d, J = 11.0 Hz), 3.01-3.14 (1H, m), 2.06 (1 H, dd, J = 7.3, 4.0 Hz), 1.58 (1H, dd, J = 6.6, 2.6 Hz), 1.53 (2H, d, J = 15.4 Hz), 1.32H, d, J = 12.1 Hz), 1.31 (1H, q, J = 11.5 Hz), 0.92-1.00 (9H, m) | 464.2 |
| 356 | 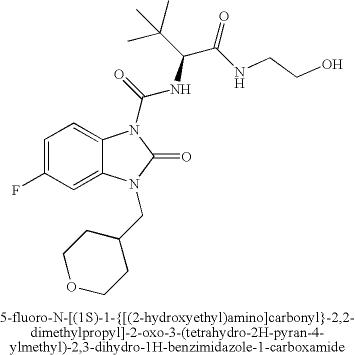 | 1H NMR (400 MHz, DMSO-d6) δ ppm 9.18 (1H, d, J = 8.8 Hz), 8.20 (1H, t, J = 5.9 Hz), 7.81 (1H, dd, J = 9.7, 2.4 Hz), 7.39 (1H, dd, J = 8.6, 4.6 Hz), 4.61 (1H, t, J = 5.3 Hz), 4.29 (1H, d, J = 8.8 Hz), 3.81 (3H, dd, J = 11.2, 7.1 Hz), 3.77 (1H, br. s.), 3.42 (1H, d, J = 5.9 Hz), 3.38- 3.46 (1H, m), 3.19 (1H, d, J = 5.9 Hz), 3.23 (2H, d, J = 9.5 Hz), 3.12 (1H, t, J = 12.6 Hz), 2.06 (1H, ddd, J = 7.2, 4.0, 3.8 Hz), 1.46 (1H, d, J = 13.5 Hz), 1.53 (2H, d, J = 11.7 Hz), 1.32 (1H, d, J = 8.1 Hz), 1.26-1.37 (1H, m), 0.92-1.00 (10 H, m) | 450.2 |
| 357 | 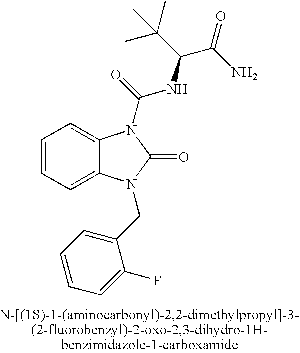 | 1H NMR (400 MHz, DMSO-d6) δ ppm 9.15 (1H, d, J = 9.1 Hz), 8.06 (1H, d, J = 7.3 Hz), 7.63 (1H, br. s.), 7.33 (1H, t, J = 7.1 Hz), 7.37 (1H, d, J = 7.3 Hz), 7.19 (2H, s), 7.24 (1H, d, J = 9.5 Hz), 7.16 (3 H, d, J = 10.6 Hz), 5.18 (2H, s), 4.27 (1 H, d, J = 8.8 Hz), 1.00 (9H, s) | 399.0 (M + H) |
| 358 | 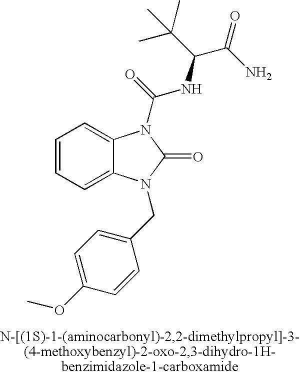 | 1H NMR (400 MHz, DMSO-d6) δ ppm 9.17 (2H, d, J = 8.8 Hz), 8.02 (1H, d, J = 7.3 Hz), 7.61 (1H, br. s.), 7.31 (2H, d, J = 8.1 Hz), 7.20-7.27 (1H, m), 7.13 (2 H, d, J = 6.6 Hz), 7.06-7.20 (1H, m), 6.88 (2H, d, J = 8.1 Hz), 5.03 (2H, d, J = 5.1 Hz), 4.24 (1H, d, J = 8.8 Hz), 3.69 (3H, s), 0.99 (8H, s) | 411.0 (M + H) |
| 359 | 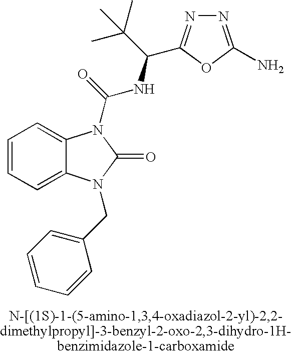 | 1H NMR (400 MHz, DMSO-d6) δ ppm 9.42 (1H, d, J = 8.8 Hz), 8.03 (1H, d, J = 7.0 Hz), 7.29-7.39 (4H, m), 7.14- 7.24 (3H, m), 7.02 (2H, s), 5.14 (2H, s), 4.89 (1H, d, J = 8.8 Hz), 1.04 (9H, s) | 420.2 |
| 360 | 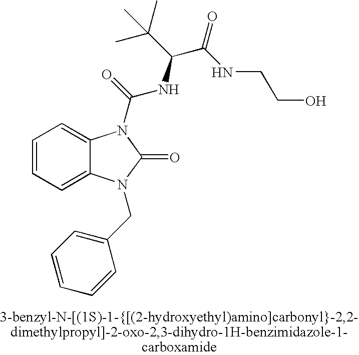 | 1H NMR (400 MHz, DMSO-d6) δ ppm 10.90 (1H, br. s.), 8.18 (1H, br. s.), 7.24- 7.34 (4H, m), 6.99 (1H, d, J = 2.9 Hz), 6.94 (1H, d, J = 4.8 Hz), 6.96 (1H, d, J = 5.9 Hz), 4.99 (2H, s), 4.69 (1H, t, J = 4.8 Hz), 3.71 (1H, s), 3.36-3.47 (4 H, m), 0.99 (1H, s), 0.93 (10H, s) | 424.2 |
| 361 | 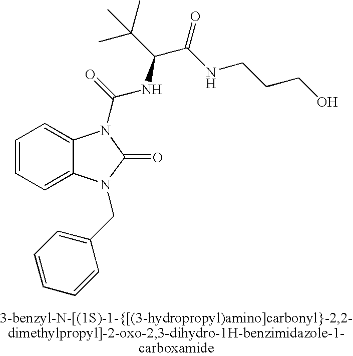 | 1H NMR (400 MHz, DMSO-d6) δ ppm 9.21 (1H, d, J = 8.8 Hz), 8.18 (1H, t, J = 5.5 Hz), 8.05 (1H, d, J = 7.0 Hz), 7.28- 7.39 (4H, m), 7.21-7.25 (1H, m), 7.17 (2H, dd, J = 14.8, 7.5 Hz), 5.13 (2H, d, J = 2.6 Hz), 4.41 (1H, d, J = 8.8 Hz), 4.36- 4.44 (1H, m), 4.27 (1H, d, J = 8.8 Hz), 3.42 (1H, d, J = 5.5 Hz), 3.32-3.41 (1H, m), 3.17-3.27 (2H, m), 3.08 (1H, dt, J = 12.8, 6.4 Hz), 1.54-1.64 (2H, m), 0.99 (8H, s) | 438.2 |
| 362 | 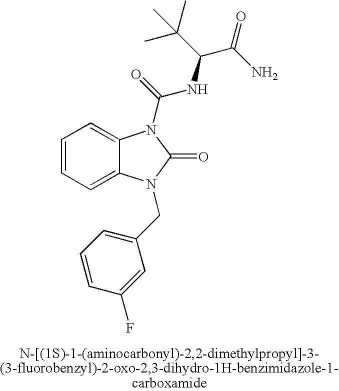 | 1H NMR (400 MHz, DMSO-d6) δ ppm 9.18 (1H, d, J = 9.2 Hz), 8.06 (1H, d, J = 8.8 Hz), 7.61-7.75 (1H, m), 7.35- 7.48 (1H, m), 7.05-7.30 (5H, m), 5.08- 5.22 (2H, in), 4.26 (1H, d, J = 9.2 Hz), 1.00 (8H, s) | 399.3 (M + H) |
| 363 | 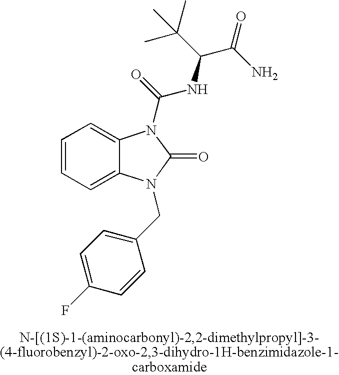 | 1H NMR (400 MHz, DMSO-d6) δ ppm 9.18 (1H, d, J = 9.2 Hz), 8.05 (1H, d, J = 7.7 Hz), 7.65-7.71 (1H, m), 7.44 (2 H, dd, J = 8.8, 5.5 Hz), 7.09-7.29 (5H, m), 5.04-5.19 (2H, m), 4.26 (1H, d, J = 8.8 Hz), 1.00 (9H, s) | 399.3 (M + H) |
| 364 | 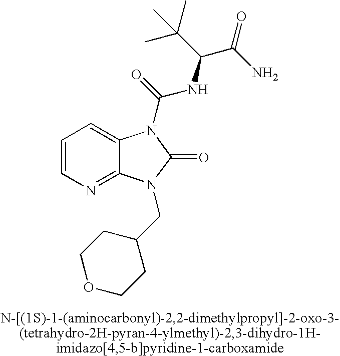 | 1H NMR (400 MHz, DMSO-d6) δ ppm 1.00 (s, 9H) 1.24-1.35 (m, 2H) 1.52- 1.59 (m, 2H) 2.10-2.20 (m, 1H) 3.18- 3.26 (m, 3H) 3.84 (br. s., 1H) 3.81 (d, J = 6.22 Hz, 3H) 4.25 (d, J = 8.78 Hz, 1H) 7.13-7.21 (m, 1H) 7.63 (br. s., 1H) 8.15 (d, J = 5.12 Hz, 1H) 8.19 (d, J = 8.05 Hz, 1H) 8.99 (d, J = 8.78 Hz, 1H) | 389.2 |
| 365 | 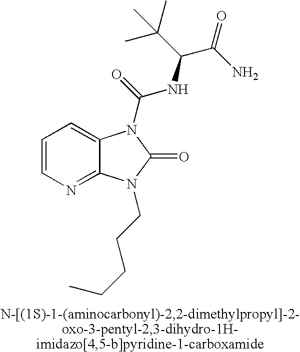 | 1H NMR (400 MHz, DMSQ-d6) δ ppm 9.00 (1H, d, J = 9.1 Hz), 8.26 (1H, d, J = 9.1 Hz), 8.18 (1H, d, J = 8.1 Hz), 8.15 (1H, d, J = 4.4 Hz), 7.63 (1H, br. s.), 7.43 (1H, d, J = 9.1 Hz), 7.18 (1H, d, J = 2.2 Hz), 7.16 (1H, br. s.), 4.25 (1H, d, J = 8.8 Hz), 3.91 (1H, t, J = 7.1 Hz), 3.34-3.45 (1H, m), 1.76 (1H, t), 1.27-1.37 (3H, m), 1.18-1.23 (2H, m), 1.12-1.18 (2 H, m), 1.00 (7H, s), 0.86 (2H, t, J = 6.8 Hz) | 361.2 |
| 366 | 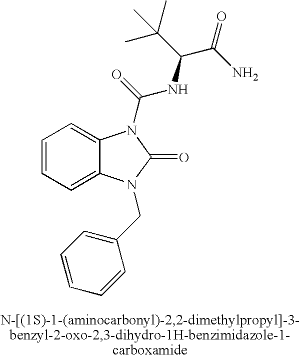 | 1H NMR (400 MHz, DMSO-d6) δ ppm 0.93 (s, 1H) 1.01 (s, 9H) 4.27 (d, J = 8.78 Hz, 1H) 5.13 (d, J = 4.76 Hz, 2H) 7.17 (d, J = 6.95 Hz, 1H) 7.15 (d, J = 5.49 Hz, 2H) 7.21 (s, 1H) 7.29-7.39 (m, 4 H) 7.63 (br. s., 1H) 8.06 (d, J = 7.32 Hz, 1 H) 9.19 (d, J = 8.78 Hz, 1H) | 380.2 |
| 367 | 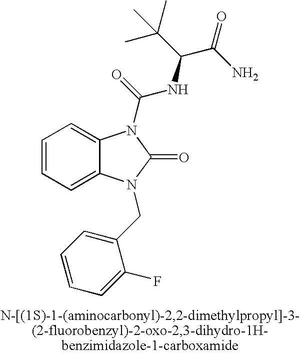 | 1H NMR (400 MHz, DMSQ-d6) δ ppm 9.15 (1H, d, J = 9.1 Hz), 8.06 (1H, d, J = 7.3 Hz), 7.63 (1H, br. s.), 7.33 (1H, t, J = 7.1 Hz), 7.37 (1H, d, J = 7.3 Hz), 7.19 (2H, s), 7.24 (1H, d, J = 9.5 Hz), 7.16 (3 H, d, J = 10.6 Hz), 5.18 (2H, s), 4.27 (1 H, d, J = 8.8 Hz), 1.00 (9H, s) | 399 (M + H) |
| 368 | 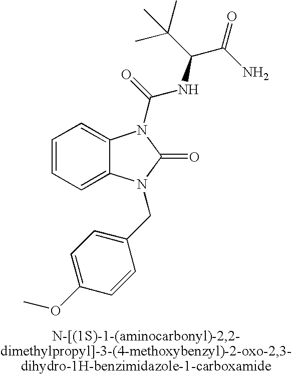 | 1H NMR (400 MHz, DMSO-d6) δ ppm 9.17 (2H, d, J = 8.8 Hz), 8.02 (1H, d, J = 7.3 Hz), 7.61 (1H, br. s.), 7.31 (2H, d, J = 8.1 Hz), 7.20-7.27 (1H, m), 7.13 (2 H, d, J = 6.6 Hz), 7.06-7.20 (1H, m), 6.88 (2H, d, J = 8.1 Hz), 5.03 (2H, d, J = 5.1 Hz), 4.24 (1H, d, J = 8.8 Hz), 3.69 (3H, s), 0.99 (8H, s) | 411 (M + H) |
| 369 | 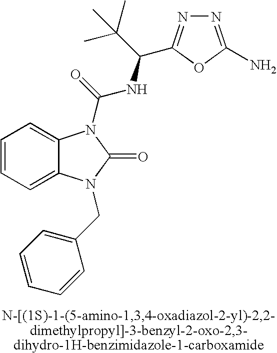 | 1H NMR (400 MHz, DMSO-d6) δ ppm 1.04 (s, 9H) 4.89 (d, J = 8.78 Hz, 1H) 5.14 (s, 2H) 7.02 (s, 2H) 7.14-7.24 (m, 3H) 7.29-7.39 (m, 4H) 8.03 (d, J = 6.95 Hz, 1H) 9.42 (d, J = 8.78 Hz, 1H) | 420.2 |
| 370 | 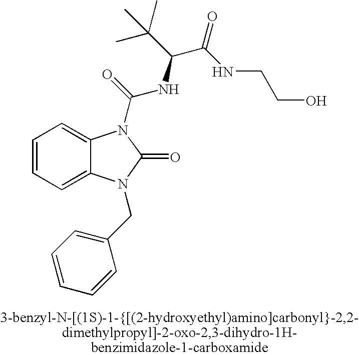 | 1H NMR (400 MHz, DMSO-d6) δ ppm 0.93 (s, 10H) 0.99 (s, 1H) 3.36-3.47 (m, 4H) 3.71 (s, 1H) 4.69 (t, J = 4.76 Hz, 1H) 4.99 (s, 2H) 6.96 (d, J = 5.86 Hz, 1 H) 6.94 (d, J = 4.76 Hz, 1H) 6.99 (d, J = 2.93 Hz, 1H) 7.24-7.34 (m, 4H) 8.18 (br. s., 1H) 10.90 (br. s., 1H) | 424.2 |
| 371 | 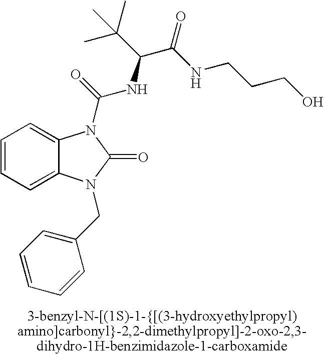 | 1H NMR (400 MHz, DMSO-d6) δ ppm 0.93 (s, 2H) 0.99 (s, 8H) 1.53-1.65 (m, 2H) 3.08 (dt, J = 12.81, 6.40 Hz, 1H) 3.16-3.27 (m, 2H) 3.32-3.41 (m, 1H) 3.42 (d, J = 5.49 Hz, 1H) 4.27 (d, J = 8.78 Hz, 1H) 4.36-4.44 (m, 1H) 5.13 (d, J = 2.56 Hz, 2H) 7.17 (dd, J = 14.82, 7.50 Hz, 2H) 7.21-7.25 (m, 1H) 7.28-7.39 (m, 4H) 8.05 (d, J = 6.95 Hz, 1H) 8,18 (t, J = 5.49 Hz, 1H) 9.21 (d, J = 8.78 Hz, 1H) | 438.2 |
| 372 | 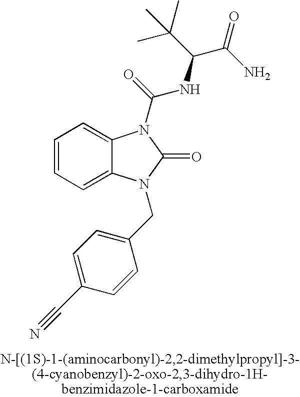 | 1H NMR (400 MHz, DMSO-d6) δ ppm 1.00 (s, 8H) 4.26 (d, J = 8.79 Hz, 1H) 5.24 (s, 2H) 7.13-7.25 (m, 3H) 7.55 (d, J = 8.42 Hz, 2H) 7.64-7.71 (m, 1H) 7.83 (d, J = 8.05 Hz, 1H) 9.15 (d, J = 8.79 Hz, 1H) | 406 (M + H) |
| 373 | 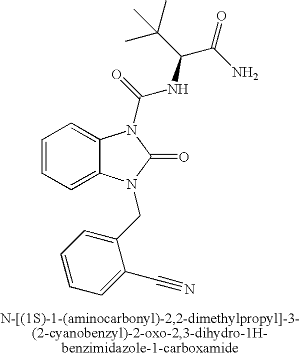 | 1H NMR (400 MHz, DMSO-d6) δ ppm 0.99 (s, 8H) 4.27 (d, J = 9.15 Hz, 1H) 5.34 (d, J = 3.29 Hz, 2H) 7.10-7.21 (m, 3H) 7.23 (d, J = 1.83 Hz, 1H) 7.34 (d, J = 8.05 Hz, 1H) 7.52 (t, J = 7.50 Hz, 1H) 7.62-7.70 (m, 2H) 9.13 (d, J = 9.15 Hz, 1H) | 406 (M + H) |
| 374 | 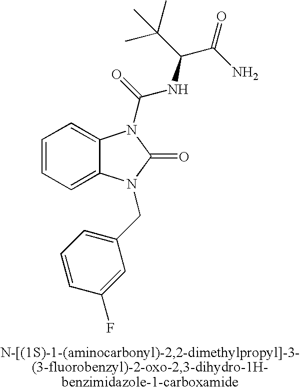 | 1H NMR (400 MHz, DMSO-d6) δ ppm 9.18 (1H, d, J = 9.2 Hz), 8.06 (1H, d, J = 8.8 Hz), 7.61-7.75 (1H, m), 7.35- 7.48 (1H, m), 7.05-7.30 (5H, m), 5.08- 5.22 (2H, m), 4.26 (1H, d, J = 9.2 Hz), 1.00 (8H, s) | 399 (M + H) |
| 375 | 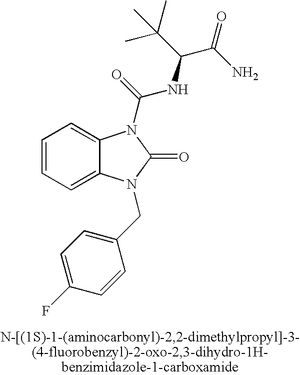 | 1H NMR (400 MHz, DMSO-d6) δ ppm 9.18 (1H, d, J = 9.2 Hz), 8.05 (1H, d, J = 7.7 Hz), 7.65-7.71 (1H, m), 7.44 (2 H, dd, J = 8.8, 5.5 Hz), 7.09-7.29 (5H, m), 5.04-5.19 (2H, m), 4.26 (1H, d, J = 8.8 Hz), 1.00 (9H, s) | 399 (M + H) |
| 376 | 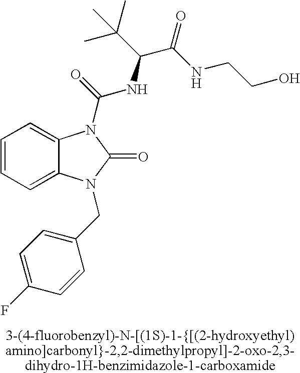 | 1H NMR (400 MHz, DMSO-d6) δ ppm 0.98 (s, 9H) 3.07-3.16 (m, 1H) 3.07- 3.26 (m, 2H) 3.41 (d, J = 5.86 Hz, 2H) 3.41 (d, J = 17.57 Hz, 2H) 4.31 (d, J = 9.15 Hz, 1H) 4.67 (d, J = 10.62 Hz, 1 H) 4.67 (s, 1H) 5.06-5.18 (m, 2H) 7.12- 7.22 (m, 4H) 7.23-7.28 (m, 1H) 7.25 (s, 1H) 7.44 (dd, J = 8.79, 5.49 Hz, 2H) 8.05 (s, 1H) 8.03 (s, 1H) 8.26 (s, 1H) 8.26 (d, J = 11.35 Hz, 1H) 9.21 (d, J = 9.15 Hz, 1H) | 443 (M + H) |
| 377 | 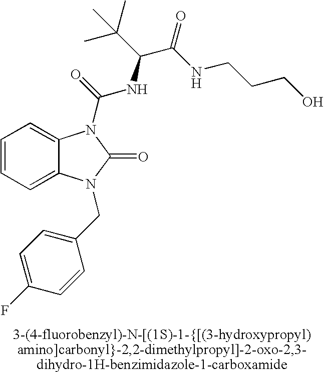 | 1H NMR (400 DMSO-d6) δ ppm 0.98 (s, 8H) 1.51-1.62 (m, J = 9.65, 6.77, 6.61, 6.61 Hz, 2H) 3.00-3.10 (m, 1H) 3.19 (dt, J = 13.18, 6.59 Hz, 1H) 3.38-3.45 (m, 2H) 4.27 (d, J = 8.79 Hz, 1H) 4.44 (t, J = 5.12 Hz, 1H) 5.07-5.17 (m, 2H) 7.10-7.28 (m, 4H) 7.44 (dd, J = 8.79, 5.49 Hz, 2H) 8.23 (t, J = 5.49 Hz, 1H) 9.21 (d, J = 9.15 Hz, 1H) | 457 (M + H) |
| 378 | 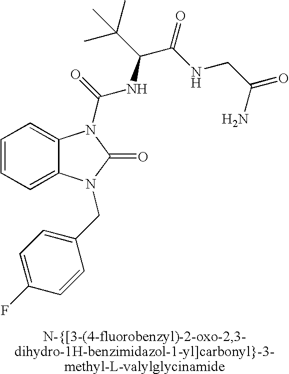 | 1H NMR (400 MHz, DMSO-d6) δ ppm 1.01 (s, 9H) 3.68 (dd, J = 5.67, 4.21 Hz, 2H) 4.34 (d, J = 8.42 Hz, 1H) 5.12 (dd, J = 2.93, 0.73 Hz, 2H) 7.03 (d, J = 0.73 Hz, 1H) 7.12-7.22 (m, 4H) 7.24-7.30 (m, 2H) 7.44 (dd, J = 8.79, 5.49 Hz, 2H) 8.01-8.06 (m, 1H) 8.46 (d, J = 11.71 Hz, 1H) 8.46 (s, 1H) 9.23 (d, J = 8.42 Hz, 1 H) | 456 (M + H) |
| 379 | 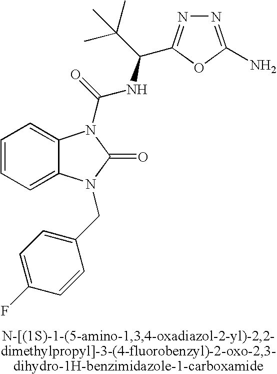 | 1H NMR (400 MHz, DMSO-d6) δ ppm 1.03 (s, 9H) 4.89 (d, J = 8.79 Hz, 1H) 5.13 (s, 2H) 7.09 (s, 2H) 7.18 (t, J = 8.97 Hz, 4H) 7.24 (s, 1H) 7.45 (dd, J = 8.79, 5.49 Hz, 2H) 8.01 (s, 1H) 9.42 (d J = 8.79 Hz, 1H) | 439 (M + H) |

Although the invention has been described above with reference to the disclosed embodiments, those skilled in the art will readily appreciate that the specific experiments detailed are only illustrative of the invention. It should be understood that various modifications can be made without departing from the spirit of the invention.

1. A method for the treatment of a condition mediated by CB1 receptor activity in a mammalian subject including a human, which comprises administering to a mammal in need of such treatment a therapeutically effective amount of the compound of formula (I): 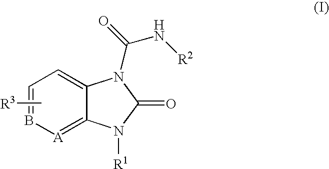
or a pharmaceutically acceptable salt thereof, wherein:
A is a carbon atom or a nitrogen atom;
B is a carbon atom or a nitrogen atom;
R¹ is a C₁-C₄ alkyl group substituted with 1 to 3 substituents independently selected from the group consisting of a halo group, a C₁-C₄ alkyl group; a hydroxy group; a C₁-C₄ alkoxy group; a mercapt group; a C₁-C₄ alkylthio group; a C₁-C₄ alkylsulfinyl group; a C₁-C₄ alkylsulfonyl group; an amino group; a C₁-C₄ alkylamino group; a di(C₁-C₄ alkyl)amino group; a (C₁-C₄ alkyl)(C₁-C₄ alkylsulfonyl)amino group; a cycloalkyl group; a cycloalkyl group substituted with 1 to 3 substituents selected from the group consisting of a hydroxy group, a C₁-C₄ alkoxy group and a C₁-C₄ alkyl group; a heterocyclyl group; a heterocyclyl group substituted with 1 to 3 substituents selected from the group consisting of a hydroxy group, a C₁-C₄ alkoxy group, a C₁-C₄ alkyl group and an oxo group; a cyano group; a heteroaryl group and a C₁-C₄ alkyl heteroaryl group;
R² is a cycloalkyl group; a cycloalkyl group substituted with 1 to 4 substituents selected from the group consisting of a hydroxy group, a C₁-C₄ hydroxyalkyl group, a C₁-C₄ alkoxy group, a C₆-C₁₀ aryloxy group, a mercapt group, a C₁-C₄ alkylthio group, a C₆-C₁₀ arylthio group, a carboxy group, a C₁-C₄ alkoxy-carbonyl group, a C₁-C₄ alkyl group, a C₂-C₄ alkenyl group, a C₂-C₄ alkynyl group and an amino-carbonyl group; a C₆-C₁₀ aryl group; a C₆-C₁₀ aryl group substituted with 1 to 3 substituents selected from the group consisting of a hydroxy group and a C₁-C₄ alkyl group; a heterocyclyl group; a heterocyclyl group substituted with 1 to 3 substituents selected from the group consisting of a hydroxy group and a C₁-C₄ alkyl group; a C₁-C₁₀ alkyl group; or a C₁-C₁₀ alkyl group substituted with 1 to 3 substituents independently selected from the group consisting of a cyano group, a hydroxy group, a trifluoromethyl group, a C₂-C₄ alkenyl group, a C₂-C₄ alkynyl group, a C₁-C₄ alkoxy group, a C₆-C₁₀ aryloxy group, a mercapt group, a C₁-C₄ alkylthio group, a C₁-C₄ alkylsulfinyl group, a C₁-C₄ alkylsulfonyl group, a C₁-C₄ alkylsulfonylamino group, a C₆-C₁₀ arylthio group, a carboxy group, a C₁-C₄alkyl-carbonyl group, a trifluoromethyl-carbonyl group, a C₁-C₄ alkoxy-carbonyl group, an amino carbonyl group, a C₁-C₄ alkylamino-carbonyl group, a C₁-C₄ hydroxyalkylamino-carbonyl group, a di(C₁-C₄ alkyl)amino-carbonyl group, a (C₁-C₄ hydroxyalkyl)(C₁-C₄ alkyl)amino-carbonyl group, a heterocyclyl-carbonyl group, a cycloalkyl group, a heterocyclyl group, a C₁-C₄ alkyl-substituted heterocyclyl group, a C₆-C₁₀ aryl group, a di(C₁-C₄ alkyl)amino group, a C₁-C₄ alkoxy C₁-C₄ alkylamino-carbonyl group, an aryl C₁-C₄alkylamino-carbonyl group, and a heteroaryl C₁-C₄ alkylamino-carbonyl group and
R³ is a hydrogen atom, a halogen atom, a hydroxy group, a C₁-C₄ alkyl group, a C₁-C₄ haloalkyl group, a C₁-C₄ alkoxy group, a C₁-C₄ alkylthio group, a C₁-C₄ alkylsulfonyl group, a C₁-C₄ alkylsulfinyl group or an aminosulfonyl group.

2. The method of claim 1, wherein:
A is a carbon atom or a nitrogen atom;
B is a carbon atom or a nitrogen atom;
R¹ is a C₁-C₄ alkyl group substituted with 1 to 3 substituents independently selected from the group consisting of a C₁-C₄ alkyl group; a hydroxy group; a C₁-C₄ alkoxy group; a mercapt group; a C₁-C₄ alkylthio group; a C₁-C₄ alkylsulfinyl group; a C₁-C₄ alkylsulfonyl group; an amino group; a C₁-C₄ alkylamino group; a di(C₁-C₄ alkyl)amino group; a (C₁-C₄ alkyl)(C₁-C₄ alkylsulfonyl)amino group; a cycloalkyl group; a cycloalkyl group substituted with 1 to 3 substituents selected from the group consisting of a hydroxy group, a C₁-C₄ alkoxy group and a C₁-C₄ alkyl group; a heterocyclyl group; and a heterocyclyl group substituted with 1 to 3 substituents selected from the group consisting of a hydroxy group, a C₁-C₄ alkoxy group and a C₁-C₄ alkyl group;
R² is a cycloalkyl group; a cycloalkyl group substituted with 1 to 4 substituents selected from the group consisting of a hydroxy group, a C₁-C₄ hydroxyalkyl group, a C₁-C₄ alkoxy group, a C₆-C₁₀ aryloxy group, a mercapt group, a C₁-C₄ alkylthio group, a C₆-C₁₀ arylthio group, a carboxy group, a C₁-C₄ alkoxy-carbonyl group, a C₁-C₄ alkyl group, a C₂-C₄ alkenyl group and a C₂-C₄ alkynyl group; a C₆-C₁₀ aryl group; a C₆-C₁₀ aryl group substituted with 1 to 3 substituents selected from the group consisting of a hydroxy group and a C₁-C₄ alkyl group; a heterocyclyl group; a heterocyclyl group substituted with 1 to 3 substituents selected from the group consisting of a hydroxy group and a C₁-C₄ alkyl group; a C₁-C₁₀ alkyl group; or a C₁-C₁₀ alkyl group substituted with 1 to 3 substituents independently selected from the group consisting of a cyano group, a hydroxy group, a trifluoromethyl group, a C₂-C₄ alkenyl group, a C₂-C₄ alkynyl group, a C₁-C₄ alkoxy group, a C₆-C₁₀ aryloxy group, a mercapt group, a C₁-C₄ alkylthio group, a C₁-C₄ alkylsulfinyl group, a C₁-C₄ alkylsulfonyl group, a C₁-C₄ alkylsulfonylamino group, a C₆-C₁₀ arylthio group, a carboxy group, a C₁-C₄ alkyl-carbonyl group, a trifluoromethyl-carbonyl group, a C₁-C₄ alkoxy-carbonyl group, an amino carbonyl group, a C₁-C₄ alkylamino-carbonyl group, a C₁-C₄ hydroxyalkylamino-carbonyl group, a di(C₁-C₄ alkyl)amino-carbonyl group, a (C₁-C₄ hydroxyalkyl)(C₁-C₄ alkyl)amino-carbonyl group, a heterocyclyl-carbonyl group, a cycloalkyl group, a heterocyclyl group, a C₁-C₄ alkyl-substituted heterocyclyl group and a C₆-C₁₀ aryl group; and
R³ is a hydrogen atom, a halogen atom, a hydroxy group, a C₁-C₄ alkyl group, C₁-C₄ haloalkyl group or a C₁-C₄ alkoxy group.

3. The method of claim 1, wherein:
A is a carbon atom or a nitrogen atom;
B is a carbon atom or a nitrogen atom;
R¹ is a C₁-C₄ alkyl group substituted with 1 to 3 substituents independently selected from the group consisting of a halo group, a C₁-C₄ alkyl group; a C₁-C₄ alkylthio group; a cyano group; a heteroaryl group, a C₁-C₄ alkyl heteroaryl group; a cycloalkyl group; a cycloalkyl group substituted with hydroxy group, a heterocyclyl group; and a heterocyclyl group substituted with an oxo group;
R² is a cycloalkyl group substituted with a hydroxy group or an amino carbonyl group; a C₆-C₁₀ aryl group substituted with a hydroxy group; or a C₁-C₁₀ alkyl group substituted with 1 to 3 substituents independently selected from the group consisting of a hydroxy group, an amino carbonyl group, a di(C₁-C₄ alkyl)amino group, a cycloalkyl group, a heterocyclyl group, a C₁-C₄ alkyl-substituted heterocyclyl group, a C₆-C₁₀ aryl group, a C₁-C₄ alkoxy C₁-C₄ alkylamino-carbonyl group, an aryl C₁-C₄alkylamino-carbonyl group, and a heteroaryl C₁-C₄alkylamino-carbonyl group;
R³ is a hydrogen atom, a halogen atom, a hydroxy group, a C₁-C₄ alkyl group, a C₁-C₄ haloalkyl group, a C₁-C₄ alkoxy group, a C₁-C₄ alkylthio group, a C₁-C₄ alkylsulfonyl group, a C₁-C₄ alkylsulfinyl group or an aminosulfonyl group.

4. The method of claim 3 wherein A is a carbon atom and B is a carbon atom.

5. The method of claim 1, wherein said condition is selected from the group consisting of inflammatory pain, nociceptive pain, neuropathic pain, fibromyalgia, chronic low back pain, visceral pain, acute cerebral ischemia, pain, chronic pain, acute pain, post herpetic neuralgia, neuropathies, neuralgia, diabetic neuropathy, HIV-related neuropathy, nerve injury, rheumatoid arthritic pain, osteoarthritic pain, back pain, cancer pain, dental pain, fibromyalgia, neuritis, sciatica, inflammation, neurodegenerative disease, spasticity, epilepsy, Tourette's syndrome, Parkinson's disease, neuroprotection, anxiety, cough, broncho constriction, irritable bowel syndrome (IBS), Inflammatory bowel disease (IBD), colitis, cerebrovascular ischemia, cachexia, nausea, emesis, chemotherapy-induced emesis, rheumatoid arthritis, asthma, Crohn's disease, ulcerative colitis, asthma, dermatitis, seasonal allergic rhinitis, gastroesophageal reflux disease (GERD), constipation, diarrhea, functional gastrointestinal disorder, cutaneous T cell lymphoma, multiple sclerosis, osteoarthritis, psoriasis, systemic lupus erythematosus, diabetes, glaucoma, osteoporosis, glomerulonephritis, renal ischemia, nephritis, hepatitis, cerebral stroke, vasodilation, hypertension, vasculitis, myocardial infarction, cerebral ischemia, reversible airway obstruction, adult respiratory disease syndrome, chronic obstructive pulmonary disease (COPD), cryptogenic fibrosing alveolitis and bronchitis.

6. A compound of formula (I): 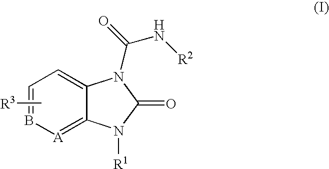
or a pharmaceutically acceptable salt thereof, wherein:
A is a carbon atom or a nitrogen atom;
B is a carbon atom or a nitrogen atom;
R¹ is selected from the group consisting of H, CH₃—(CH₂)₄—, cyano-(CH₂)₃—, cyano-(CH₂)₄—, CF₃—(CH₂)₂—, cyclobutyl-CH₂—, cyclobutyl-(CH₂)₂—, cyclopropyl-(CH₂)₃—, cyclohexyl-CH₂—, OH-cyclohexyl-CH₂—, tetrahydrofuranyl-CH₂—, tetrahydropyranyl-CH₂—, oxo-tetrahydrofuranyl-CH₂—, oxo-pyrrolidinyl-CH₂—, pyridinyl-CH₂—, pyrazinyl-CH₂—, pyrimidinyl-CH₂—, CH₃-pyrazolyl-CH₂—, CH₃-oxazolyl-CH₂—, CH₃-isoxazolyl-CH₂—, CH₃-oxadiazolyl-CH₂—, CH₃-thiazolyl-CH₂—, and CH₃-thiadiazolyl-CH₂—;
R² is selected from the group consisting of H, NR⁴R⁵—C(O)—CR⁶R⁷—, CR⁸R⁹R¹⁰—, (CH₃)₂—N—CH₂—C(CH₃)₂—CH₂—, tetrahydronaphthalenyl, OH-dihydroindenyl, OH-cyclohexyl, and CH₃—CH₂-pyrrolidinyl-CH₂—;
R³ is selected from the group consisting of H, F, Cl, methyl, cyano, methoxy, trifluoromethyl, methylthio, methylsulfinyl, methylsulfonyl and aminosulfonyl;
R⁴ and R⁵ are H, OH—(CH₂)₂—, NH₂—C(O)—(CH₂)₂—, CH₃—O—(CH₂)₂—, benzyl, or pyridinyl;
R⁶ and R⁷ are H, (CH₃)₃—C—, CH₃, benzyl, phenyl, tetrahydropyranyl, or cyclohexyl;
R⁸, R⁹ and R¹⁰ are H, (CH₃)₃—C—, NH₂—C(O), OH—CH₂—, (CH₃CH₂)₂—N—CH₂—, or CH₃; or two of R⁸, R⁹, or R¹⁰ form a cyclohexyl.

7. The compound of claim 6 or a pharmaceutically acceptable salt thereof, wherein:
A is a carbon atom;
B is a carbon atom;
R¹ is selected from the group consisting of H, CH₃—(CH₂)₄—, cyano-(CH₂)₃—, cyano-(CH₂)₄—, CF₃—(CH₂)₂—, cyclobutyl-CH₂—, cyclobutyl-(CH₂)₂—, cyclopropyl-(CH₂)₃—, cyclohexyl-CH₂—, OH-cyclohexyl-CH₂—, tetrahydrofuranyl-CH₂—, tetrahydropyranyl-CH₂—, oxo-tetrahydrofuranyl-CH₂—, oxo-pyrrolidinyl-CH₂, pyridinyl-CH₂—, pyrazinyl-CH₂—, pyrimidinyl-CH₂—, CH₃-pyrazolyl-CH₂—, CH₃-oxazolyl-CH₂—, CH₃-isoxazolyl-CH₂—, CH₃-oxadiazolyl-CH₂—, CH₃-thiazolyl-CH₂—, and CH₃-thiadiazolyl-CH₂—;
R² is selected from the group consisting of H, NR⁴R⁵—C(O)—CR⁶R⁷—, CR⁸R⁹R¹⁰—, (CH₃)₂—N—CH₂—C(CH₃)₂—CH₂—, tetrahydronaphthalenyl, OH-dihydroindenyl, OH-cyclohexyl, and CH₃—CH₂-pyrrolidinyl-CH₂—;
R³ is selected from the group consisting of H, F, Cl, methyl, cyano, methoxy, trifluoromethyl, methylthio, methylsulfinyl, methylsulfonyl and aminosulfonyl;
R⁴ and R⁵ are H, OH—(CH₂)₂—, NH₂—C(O)—(CH₂)₂—, CH₃—O—(CH₂)₂—, benzyl, or pyridinyl;
R⁶ and R⁷ are H, (CH₃)₃—C—, CH₃, benzyl, phenyl, tetrahydropyranyl, or cyclohexyl;
R⁸, R⁹ and R¹⁰ are H, (CH₃)₃—C—, NH₂—C(O)—, OH—CH₂—, (CH₃CH₂)₂—N—CH₂—, or CH₃; or two of R⁸, R⁹, or R¹⁰ form a cyclohexyl.

8. The compound of claim 7 or a pharmaceutically acceptable salt thereof, wherein R¹ is selected from the group consisting of

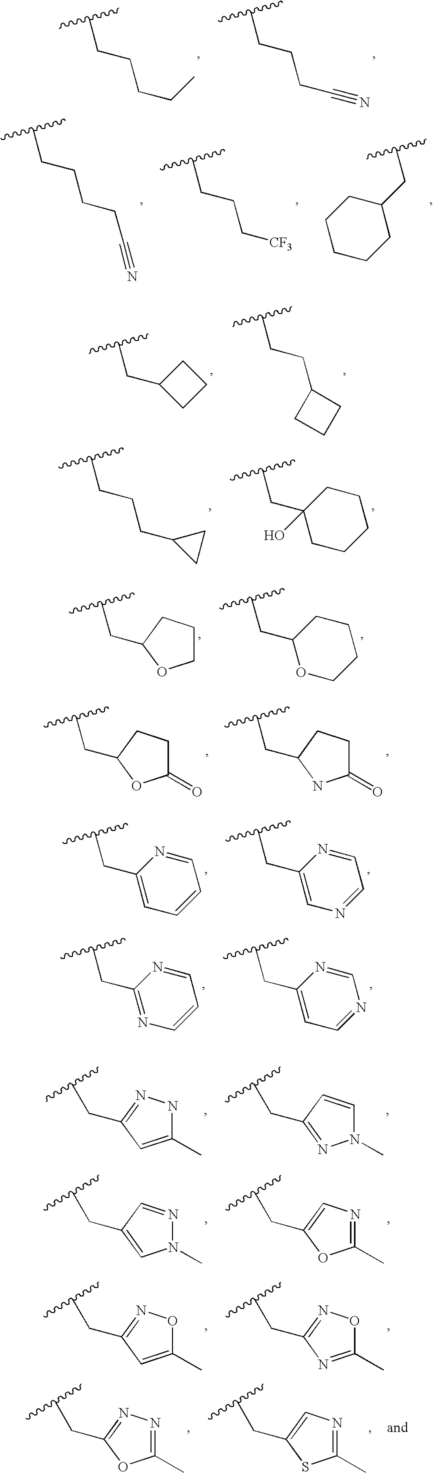

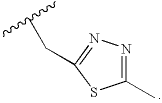

9. The compound of claim 7 or a pharmaceutically acceptable salt thereof, wherein R² is selected from the group consisting of

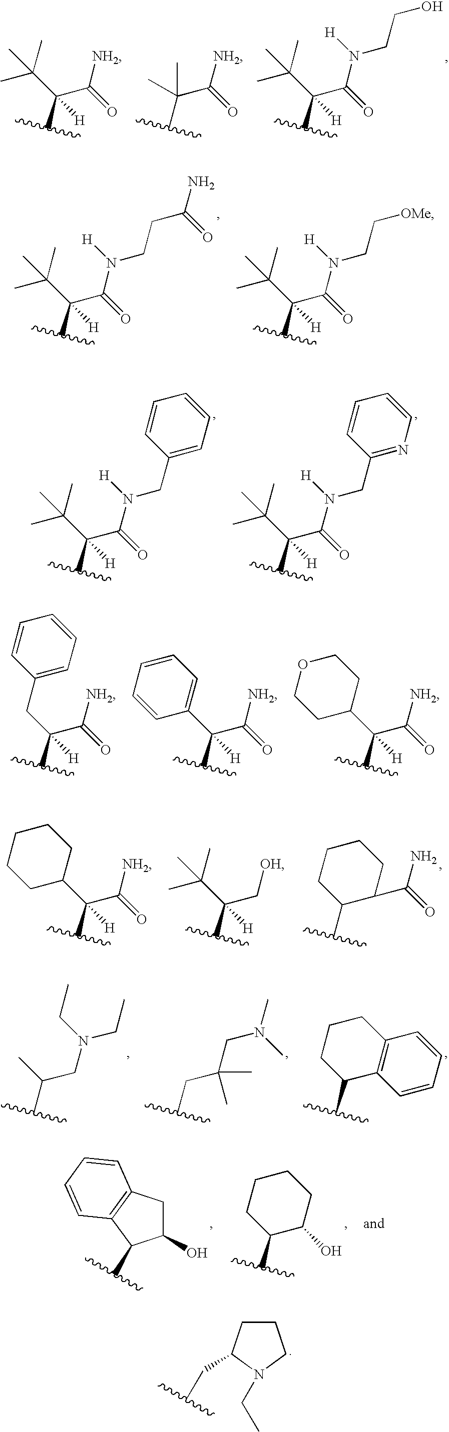

10. A method of treatment comprises administering to a mammal in need of such treatment a therapeutically effective amount of the compound N-[(1S)-1-(aminocarbonyl)-2,2-dimethylpropyl]-2-oxo-3-(tetrahydro-2H-pyran-4-ylmethyl)-2,3-dihydro-1H-benzimidazole-1-carboxamide or N-[(1S)-1-(aminocarbonyl)-2,2-dimethylpropyl]-4-methyl-3-[2-(methylthio)ethyl]-2-oxo-2,3-dihydro-1H-benzimidazole-1-carboxamide or a pharmaceutically acceptable salt thereof.

11. The compound of claim 7 selected from the group consisting of
N-[(1S)-1-(aminocarbonyl)-2,2-dimethylpropyl]-2-oxo-3-(tetrahydro-2H-pyran-4-ylmethyl)-2,3-dihydro-1H-benzimidazole-1-carboxamide;
N-[(1S)-1-(aminocarbonyl)-2,2-dimethylpropyl]-3-(cyclohexylmethyl)fluoro-2-oxo-2,3-dihydro-1H-benzimidazole-1-carboxamide;
N-[(1S)-1-(aminocarbonyl)-2,2-dimethylpropyl]-6-chloro-3-cyclohexylmethyl)-2-oxo-2,3-dihydro-1H-benzimidazole-1-carboxamide;
N-[(1S)-1-(aminocarbonyl)-2,2-dimethylpropyl]-3-(cyclohexylmethyl)-6-methyl-2-oxo-2,3-dihydro-1H-benzimidazole-1-carboxamide;
N-[(1S)-1-(aminocarbonyl)-2,2-dimethylpropyl]-6-cyano-3-(cyclohexylmethyl)-2-oxo-2,3-dihydro-1H-benzimidazole-1-carboxamide;
N-[(1S)-1-(aminocarbonyl)-2,2-dimethylpropyl]-3-cyclohexylmethyl)-5-fluoro-2-oxo-2,3-dihydro-1H-benzimidazole-1-carboxamide;
N-[(1S)-1-(aminocarbonyl)-2,2-dimethylpropyl]-5-chloro-3-(cyclohexylmethyl)-2-oxo-2,3-dihydro-1H-benzimidazole-1-carboxamide;
N-[(1S)-1-(aminocarbonyl)-2,2-dimethylpropyl]-5-cyano-3-(cyclohexylmethyl)-2-oxo-2,3-dihydro-1H-benzimidazole-1-carboxamide;
N-[(1S)-1-(aminocarbonyl)-2,2-dimethylpropyl]-3-(cyclohexylmethyl)-4-fluoro-2-oxo-2,3-dihydro-1H-benzimidazole-1-carboxamide;
N-[(1S)-1-(aminocarbonyl)-2,2-dimethylpropyl]-4-chloro-3-(cyclohexylmethyl)-2-oxo-2,3-dihydro-1H-benzimidazole-1-carboxamide;
N-[(1S)-1-(aminocarbonyl)-2,2-dimethylpropyl]-3-(cyclohexylmethyl)-4-methyl-2-oxo-2,3-dihydro-1H-benzimidazole-1-carboxamide;
N-[(1S)-1-(aminocarbonyl)-2,2-dimethylpropyl]-4-cyano-3-(cyclohexylmethyl)-2-oxo-2,3-dihydro-1H-benzimidazole-1-carboxamide;
3-(cyclohexylmethyl)-N-[(1S)-1-(hydroxymethyl)-2,2-dimethylpropyl]-2-oxo-2,3-dihydro-1H-benzimidazole-1 carboxamide;
3-(cyclobutylmethyl)-N-[(1S)-1-(hydroxymethyl)-2,2-dimethylpropyl]-2-oxo-2,3-dihydro-1H-benzimidazole-1-carboxamide;
N-[(1S)-1-(hydroxymethyl)-2,2-dimethylpropyl]-2-oxo-3-pentyl-2,3-dihydro-1H-benzimidazole-1-carboxamide;
3-(cyclohexylmethyl)-N-{[(2S)-1-ethylpyrrolidin-2-yl]methyl}-2-oxo-2,3-dihydro-1H-benzimidazole-1-carboxamide;
3-(cyclobutylmethyl)-N-{[(2S)-1-ethylpyrrolidin-2-yl]methyl}-2-oxo-2,3-dihydro-1H-benzimidazole-1-carboxamide;
N-{[(2S)-1-ethylpyrrolidin-2-yl]methyl}-2-oxo-3-pentyl-2,3-dihydro-1H-benzimidazole-1-carboxamide;
3-(cyclohexylmethyl)-N-[3-(dimethylamino)-2,2-dimethylpropyl]-2-oxo-2,3-dihydro-1H-benzimidazole-1-carboxamide;
3-(cyclobutylmethyl)-N-[3-(dimethylamino)-2,2-dimethylpropyl]-2-oxo-2,3-dihydro-1H-benzimidazole-1-carboxamide;
N-[3-(dimethylamino)-2,2-dimethylpropyl]-2-oxo-3-pentyl-2,3-dihydro-1H-benzimidazole-1-carboxamide;
3-(cyclohexylmethyl)-N-[2-(diethylamino)-1-methylethyl]-2-oxo-2,3-dihydro-1H-benzimidazole-1-carboxamide;
3-(cyclobutylmethyl)-N-[2-diethylamino)-1-methylethyl]-2-oxo-2,3-dihydro-1H-benzimidazole-1-carboxamide;
N-[2-(diethylamino)-1-methylethyl]-2-oxo-3-pentyl-2,3-dihydro-1H-benzimidazole-1-carboxamide;
3-(cyclohexylmethyl)-N-[(1S,2R)-2-hydroxy-2,3-dihydro-1H-inden-1-yl]-2-oxo-2,3-dihydro-1H-benzimidazole-1-carboxamide;
3-(cyclobutylmethyl)-N-[(1S,2R)-2-hydroxy-2,3-dihydro-1H-inden-1-yl]-2-oxo-2,3-dihydro-1H-benzimidazole-1-carboxamide;
N-[(1S,2R)-2-hydroxy-2,3-dihydro-1H-inden-1-yl]-2-oxo-3-pentyl-2,3-dihydro-1H-benzimidazole-1-carboxamide;
3-(cyclohexylmethyl)-N-[(1S,2S-2-hydroxycyclohexyl]-2-oxo-2,3-dihydro-1H-benzimidazole-1-carboxamide;
3-(cyclobutylmethyl)-N-[(1S,2S)-2-hydroxycyclohexyl]-2-oxo-2,3-dihydro-1H-benzimidazole-1-carboxamide;
N-[(1S,2S)-2-hydroxycyclohexyl]-2-oxo-3-pentyl-2,3-dihydro-1H-benzimidazole-1-carboxamide;
3-(cyclohexylmethyl)-N-(trans-4-hydroxycyclohexyl)-2-oxo-2,3-dihydro-1H-benzimidazole-1-carboxamide;
3-(cyclobutylmethyl)-N-(trans-4-hydroxycyclohexyl)-2-oxo-2,3-dihydro-1H-benzimidazole-1-carboxamide;
N-(trans-4-hydroxycyclohexyl)-2-oxo-3-pentyl-2,3-dihydro-1H-benzimidazole-1-carboxamide;
3-(cyclohexylmethyl)-2-oxo-N-[(1S)-1,2,3,4-tetrahydronaphthalen-1-yl]-2,3-dihydro-1H-benzimidazole-1-carboxamide;
3-(cyclobutylmethyl)-2-oxo-N-[(1S)-1,2,3,4-tetrahydronaphthalen-1-yl]-2,3-dihydro-1H-benzimidazole-1-carboxamide;
2-oxo-3-pentyl-N-[(1S)-1,2,3,4-tetrahydronaphthalen-1-yl]-2,3-dihydro-1H-benzimidazole-1-carboxamide;
N-[(1S)-1-(aminocarbonyl)-2,2-dimethylpropyl]-3-(cyclohexylmethyl)-2-oxo-2,3-dihydro-1H-benzimidazole-1-carboxamide;
N-[(1S)-1-(aminocarbonyl)-2,2-dimethylpropyl]-3-(cyclobutylmethyl)-2-oxo-2,3-dihydro-1H-benzimidazole-1-carboxamide;
N-[(1S)-1-(aminocarbonyl)-2,2-dimethylpropyl]-2-oxo-3-pentyl-2,3-dihydro-1H-benzimidazole-1-carboxamide;
N-[(1S)-1-(aminocarbonyl)-2-methylpropyl]-3-(cyclohexylmethyl)-2-oxo-2,3-dihydro-1H-benzimidazole-1-carboxamide;
N-[(1S)-1-(aminocarbonyl)-2-methylpropyl]-3-(cyclobutylmethyl)-2-oxo-2,3-dihydro-1H-benzimidazole-1-carboxamide;
N-[(1S)-1-(aminocarbonyl)-2-methylpropyl]-2-oxo-3-pentyl-2,3-dihydro-1H-benzimidazole-1-carboxamide;
N-(2-amino-1,1-dimethyl-2-oxoethyl)-3-(cyclohexylmethyl)-2-oxo-2,3-dihydro-1H-benzimidazole-1-carboxamide;
N-(2-amino-1,1-dimethyl-2-oxoethyl)-3-(cyclobutylmethyl)-2-oxo-2,3-dihydro-1H-benzimidazole-1-carboxamide;
N-(2-amino-1,1-dimethyl-2-oxoethyl)-2-oxo-3-pentyl-2,3-dihydro-1H-benzimidazole-1-carboxamide;
N-[1-(aminocarbonyl)cyclohexyl]-3-cyclohexylmethyl)-2-oxo-2,3-dihydro-1H-benzimidazole-1-carboxamide;
N-[1-(aminocarbonyl)cyclohexyl]-3-(cyclobutylmethyl)-2-oxo-2,3-dihydro-1H-benzimidazole-1-carboxamide;
N-[1-(aminocarbonyl)cyclohexyl]-2-oxo-3-pentyl-2,3-dihydro-1H-benzimidazole-1-carboxamide;
N-[(1S)-2-amino-2-oxo-1-phenylethyl]-3-(cyclohexylmethyl)-2-oxo-2,3-dihydro-1H-benzimidazole-1-carboxamide;
N-[(1S)-2-amino-2-oxo-1-phenylethyl]-3-(cyclobutylmethyl)-2-oxo-2,3-dihydro-1H-benzimidazole-1-carboxamide;
N-[(1S)-2-amino-2-oxo-1-phenylethyl]-2-oxo-3-pentyl-2,3-dihydro-1H-benzimidazole-1-carboxamide;
N-alpha-{[3-(cyclohexylmethyl)-2-oxo-2,3-dihydro-1H-benzimidazol-1-yl]carbonyl}-L-phenylalaninamide;
N-alpha-{[3-(cyclobutylmethyl)-2-oxo-2,3-dihydro-1H-benzimidazol-1-yl]carbonyl}-L-phenylalaninamide;
N-alpha-[(2-oxo-3-pentyl-2,3-dihydro-1H-benzimidazol-1-yl)carbonyl]-L-phenylalaninamide;
N-[(1S)-1-(aminocarbonyl)-2,2-dimethylpropyl]-3-(2-cyclobutylethyl)-2-oxo-2,3-dihydro-1H-benzimidazole-1-carboxamide;
N-[(1S)-1-(aminocarbonyl)-2,2-dimethylpropyl]-3-(3-cyclopropylpropyl)-2-oxo-2,3-dihydro-1H-benzimidazole-1-carboxamide;
N-[(1S)-1-(aminocarbonyl)-2,2-dimethylpropyl]-2-oxo-3-(pyridin-2-ylmethyl)-2,3-dihydro-1H-benzimidazole-1-carboxamide;
N-[(1S)-1-(aminocarbonyl)-2,2-dimethylpropyl]-2-oxo-3-(pyridin-2-ylmethyl)-2,3-dihydro-1H-benzimidazole-1-carboxamide;
N-[(1S)-1-(aminocarbonyl)-2,2-dimethylpropyl]-2-oxo-3-(pyrimidin-2-ylmethyl)-2,3-dihydro-1H-benzimidazole-1-carboxamide;
N-[(1S)-(aminocarbonyl-2,2-dimethylpropyl]-2-oxo-3-(pyridazin-3-ylmethyl)-2,3-dihydro-1H-benzimidazole-1-carboxamide;
N-[(1S)-1-(aminocarbonyl)-2,2-dimethylpropyl]-2-oxo-3-(pyrimidin-4-ylmethyl)-2,3-dihydro-1H-benzimidazole-1-carboxamide;
N-[(1S)-1-(aminocarbonyl)-2,2-dimethylpropyl]-3-[(5-methylisoxazol-3-yl)methyl]-2-oxo-2,3-dihydro-1H-benzimidazole-1-carboxamide;
N-[(1S)-1-(aminocarbonyl)-2,2-dimethylpropyl]-3-[(5-methyl-1H-pyrazol-3-yl)methyl]-2-oxo-2,3-dihydro-1H-benzimidazole-1-carboxamide;
N-[(1S)-1-(aminocarbonyl)-2,2-dimethylpropyl]-3-[(5-methyl-1,2,4-oxadiazol-3-yl)methyl]-2-oxo-2,3-dihydro-1H-benzimidazole-1-carboxamide;
N-[(1S)-1-(aminocarbonyl)-2,2-dimethylpropyl]-3-[(5-methyl-1,3,4-oxadiazol-2-yl)methyl]-2-oxo-2,3-dihydro-1H-benzimidazole-1-carboxamide;
N-[(1S)-1-(aminocarbonyl)-2,2-dimethylpropyl]-3-[(1-methyl-1H-pyrazol-4-yl)methyl]-2-oxo-2,3-dihydro-1H-benzimidazole-1-carboxamide;
N-[(1S)-1-(aminocarbonyl)-2,2-dimethylpropyl]-3-[(1-methyl-1H-pyrazol-3-yl)methyl]-2-oxo-2,3-dihydro-1H-benzimidazole-1-carboxamide;
N-[(1S)-1-aminocarbonyl)-2,2-dimethylpropyl]-3-[(2-methyl-1,3-oxazol-5-yl)methyl]-2-oxo-2,3-dihydro-1H-benzimidazole-1-carboxamide;
N-[(1S)-1-(aminocarbonyl)-2,2-dimethylpropyl]-3-[(2-methyl-1,3-thiazol-5-yl)methyl]-2-oxo-2,3-dihydro-1H-benzimidazole-1-carboxamide;
N-[(1S)-1-(aminocarbonyl)-2,2-dimethylpropyl]-3-[(5-methyl-1,3,4-thiadiazol-2-yl)methyl]-2-oxo-2,3-dihydro-1H-benzimidazole-1-carboxamide;
N-[(1S)-1-(aminocarbonyl)-2,2-dimethylpropyl]-3-(3-cyanopropyl)-2-oxo-2,3-dihydro-1H-benzimidazole-1-carboxamide;
N-[(1S)-1-(aminocarbonyl)-2,2-dimethylpropyl]-3-(4-cyanobutyl)-2-oxo-2,3-dihydro-1H-benzimidazole-1-carboxamide;
N-[(1S)-1-(aminocarbonyl)-2,2-dimethylpropyl]-2-oxo-3-(tetrahydro-2H-pyran-2-ylmethyl)-2,3-dihydro-1H-benzimidazole-1-carboxamide;
N-[(1S)-1-(aminocarbonyl)-2,2-dimethylpropyl]-2-oxo-3-(tetrahydrofuran-2-ylmethyl)-2,3-dihydro-1H-benzimidazole-1-carboxamide;
N-(2-amino-1,1-dimethyl-2-oxoethyl)-3-(2-cyclobutylethyl)-2-oxo-2,3-dihydro-1H-benzimidazole-1-carboxamide;
N-(2-amino-1,1-dimethyl-2-oxoethyl)-3-[(5-methyl-1,3,4-oxadiazol-2-yl)methyl]-2-oxo-2,3-dihydro-1H-benzimidazole-1-carboxamide;
N-(2-amino-1,1-dimethyl-2-oxoethyl)-3-(3-cyanopropyl)-2-oxo-2,3-dihydro-1H-benzimidazole-1-carboxamide;
N-[(1S)-2-amino-2-oxo-1-phenylethyl]-2-oxo-3-(pyridin-2-ylmethyl)-2,3-dihydro-1H-benzimidazole-1-carboxamide;
N-[(1S)-2-amino-2-oxo-1-phenylethyl]-2-oxo-3-(pyrazin-2-ylmethyl)-2,3-dihydro-1H-benzimidazole-1-carboxamide;
N-[(1S)-2-amino-2-oxo-1-phenylethyl]-3-[(5-methylisoxazol-3-yl)methyl]-2-oxo-2,3-dihydro-1H-benzimidazole-1-carboxamide;
N-alpha-({3-[(1-methyl-1H-pyrazol-3-yl)methyl]-2-oxo-2,3-dihydro-1H-benzimidazol-1-yl}-carbonyl)-L-phenylalaninamide;
N-alpha-({3-[(5-methyl-1,3,4-thiadiazol-2-yl)methyl]-2-oxo-2,3-dihydro-1H-benzimidazol-1-yl}-carbonyl)-L-phenylalaninamide;
N-alpha-({2-oxo-3-[(5-oxotetrahydrofuran-2-yl)methyl]-2,3-dihydro-1H-benzimidazol-1-yl}carbonyl)-L-phenylalaninamide;
N-alpha-({2-oxo-3-[(5-oxopyrrolidin-2-yl)methyl]-2,3-dihydro-1H-benzimidazol-1-yl}carbonyl)-L-phenylalaninamide;
N-[(1S)-1-(aminocarbonyl)-2,2-dimethylpropyl]-3-(cyclohexylmethyl)-4-methoxy-2-oxo-2,3-dihydro-1H-benzimidazole-1-carboxamide;
N-[(1S)-1-(aminocarbonyl)-2,2-dimethylpropyl]-3(cyclohexylmethyl)-5-methoxy-2-oxo-2,3-dihydro-1H-benzimidazole-1-carboxamide;
N-[(1S)-1-(aminocarbonyl)-2,2-dimethylpropyl]-3-(cyclohexylmethyl)-2-oxo-5-(trifluoromethyl)-2,3-dihydro-1H-benzimidazole-1-carboxamide;
N-[(1S)-1-(aminocarbonyl)-2,2-dimethylpropyl]-3-(cyclohexylmethyl)-2-oxo-2,3-dihydro-1H-imidazo[4,5-c]pyridine-1-carboxamide;
N-[(1S)-1-(aminocarbonyl)-2,2-dimethylpropyl]-3-(cyclohexylmethyl)-2-oxo-2,3-dihydro-1H-imidazo[4,5-b]pyridine-1-carboxamide;
N-[(1S)-2-amino-1 cyclohexyl-2-oxoethyl]-3-(cyclohexylmethyl)-2-oxo-2,3-dihydro-1H-benzimidazole-1-carboxamide;
N-[(1S)-2-amino-2-oxo-1-(tetrahydro-2H-pyran-4-yl)ethyl]-3-(cyclohexylmethyl)-2-oxo-2,3-dihydro-1H-benzimidazole-1-carboxamide;
N-[(1S)-1-(aminocarbonyl)-2,2-dimethylpropyl]-3-[(1-hydroxycyclohexyl)methyl]-2-oxo-2,3-dihydro-1H-benzimidazole-1-carboxamide;
3-(cyclohexylmethyl)-N-[(1S)-1-{[(2-hydroxyethyl)amino]carbonyl}-2,2-dimethylpropyl]-2-oxo-2,3-dihydro-1H-benzimidazole-1-carboxamide;
3-(cyclohexylmethyl)-N-[(1S)-1-{[(2-methoxyethyl)amino]carbonyl}-2,2-dimethylpropyl]-2-oxo-2,3-dihydro-1H-benzimidazole-1-carboxamide;
N-{(1S)-1-[(benzylamino)carbonyl]-2,2-dimethylpropyl}-3(cyclohexylmethyl)-2-oxo-2,3-dihydro-1H-benzimidazole-1-carboxamide;
3-(cyclohexylmethyl)-N-[(1S)-2,2-dimethyl-1-{[(pyridin-2-ylmethyl)amino]carbonyl}propyl]-2-oxo-2,3-dihydro-1H-benzimidazole-1-carboxamide;
N-{[3(cyclohexylmethyl)-2-oxo-2,3-dihydro-1H-benzimidazol-1-yl]carbonyl}-3-methyl-L-valyl-beta-alaninamide;
N-[(1S)-1-(aminocarbonyl)-2,2-dimethylpropyl]-3-(cyclohexylmethyl)-5-(methylthio-2-oxo-2,3-dihydro-1H-benzimidazole-1-carboxamide;
N-[(1S)-1-(aminocarbonyl)-2,2-dimethylpropyl]-3(cyclohexylmethyl)-5(methylsulfinyl)-2-oxo-2,3-dihydro-1H-benzimidazole-1-carboxamide;
N-[(1S)-1-(aminocarbonyl)-2,2-dimethylpropyl]-3(cyclohexylmethyl)-5-(methylsulfonyl)-2-oxo-2,3-dihydro-1H-benzimidazole-1-carboxamide;
N-[(1S)-1-(aminocarbonyl)-2,2-dimethylpropyl]-5-(aminosulfonyl)-3-(cyclohexylmethyl)-2-oxo-2,3-dihydro-1H-benzimidazole-1-carboxamide; and
N-[(1S)-1-(aminocarbonyl)-2,2-dimethylpropyl]-2-oxo-3-(3,3,3-trifluoropropyl)-2,3-dihydro-1H-benzimidazole-1-carboxamide.

12. A compound of formula (I): 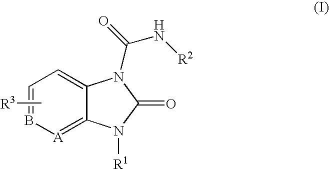
or a pharmaceutically acceptable salt thereof, wherein:
A is a carbon atom or a nitrogen atom;
B is a carbon atom or a nitrogen atom;
R¹ is selected from the group consisting of H, CH₃—(CH₂)₄—, CH₃—(CH₂)₃—, cyano-(CH₂)₃—, cyano-(CH₂)₄—, CF₃—(CH₂)₂—, cyclobutyl-CH₂—, cyclobutyl-(CH₂)₂—, cyclopropyl-(CH₂)₃—, cyclopropyl-C(O)CH₂—, CH₃—CH₂—NH—C(O)—CH₂—, (CH₃)₃—C—C(O)—CH₂—, cyclohexyl-CH₂—, OH-cyclohexyl-CH₂—, F₂-cyclohexyl-CH₂—, F-cyclohexenyl-CH₂—, tetrahydrofuranyl-CH₂—, tetrahydropyranyl-CH₂—, fluoro-benzyl, CH₃—O-benzyl, cyano-benzyl, methyl-benzyl, chloro-benzyl, oxo-tetrahydrofuranyl-CH₂—, oxo-pyrrolidinyl-CH₂—, pyridinyl-CH₂—, pyrazinyl-CH₂—, pyrimidinyl-CH₂—, CH₃-pyrazolyl-CH₂—, CH₃-oxazolyl-CH₂—, CH₃-isoxazolyl-CH₂—, CH₃-oxadiazolyl-CH₂—, CH₃-thiazolyl-CH₂—, and CH₃-thiadiazolyl-CH₂—;
R² is selected from the group consisting of H, NR⁴R⁵—C(O)—CR⁶R⁷—, CR⁸R⁹R¹⁰—, (CH₃)₂—N—CH₂—C(CH₃)₂—CH₂—, tetrahydronaphthalenyl, OH-dihydroindenyl, OH-cyclohexyl, CH₃CH₂-pyrrolidinyl-CH₂—, oxadiazolyl-CR¹¹R¹²— optionally substituted with CH₃, NH₂, (CH₃)₂—N—C(O)—, CH₃—NH—C(O)—, tetrahydronaphthalenyl-NH—C(O)—, azepanyl-C(O)—, oxopyrrolidinyl-(CH₂)₃—NH—C(O)—, CH₃—O—(CH₂)₂—NH—C(O)—, OH-cyclohexyl-NH—C(O)—, OH—CH₂-piperidinyl-C(O)—, CH₃—CH₂—, (CH₃)₂—CH—(CH₂)₂—NH—C(O)—, or (CH₃)₂—CH—; isoxazolyl-CR¹¹R¹²— optionally substituted with CH₃; furyl-CR¹¹R¹²— optionally substituted with CH₃ or CF₃, pyrazolyl-CR¹¹R¹²— optionally substituted with CH₃, (CH₃)₂—CH—, or CH₃—CH₂—; thiazolyl-CR¹¹R¹²— optionally substituted with CH₃, CH₃—CH₂—, or CF₃; and dihydroisochromenyl-CR¹¹R¹²—;
R³ is selected from the group consisting of H, F, Cl, bromo, difluoro, methyl, cyano, methoxy, trifluoromethyl, methylthio, methylsulfinyl, methylsulfonyl and aminosulfonyl;
R⁴ and R⁵ are H, OH—(CH₂)₂—, NH₂—C(O)—(CH₂)₂—, CH₃—O—(CH₂)₂—, benzyl, pyridinyl, cyclobutyl, (CH₃)₃—C—, cyclopropyl, CH₃, OH—(CH₂)₃—, or (OH)₂—CH₂—CH—CH₂—;
R⁶ and R⁷ are H, (CH₃)₃—C—, CH₃, benzyl, phenyl, tetrahydropyranyl, cyclohexyl, (CH₃)₂—CH—CH₂—, (CH₃)₂—CH—, or (OH)(CH₃)—CH—;
R⁸, R⁹ and R¹⁰ are H, (CH₃)₃—C—, NH₂—C(O)—, OH—CH₂—, (CH₃CH₂)₂—N—CH₂—, CH₃, (OH) (CH₃)₂—CH—, CH₃—NH—C(O)—CH₂—, cyclopropyl-NH—C(O)—CH₂—, NH₂—C(O)—CH₂—NH—C(O)—CH₂—, or COOH—CH₂—; or two of R⁸, R⁹, or R¹⁰ form a cyclohexyl, and
R¹¹ and R¹² are H, CH₃, or (CH₃)₃—C—.

13. The compound of claim 12 or a pharmaceutically acceptable salt thereof, wherein:
A is a carbon atom;
B is a carbon atom;
R¹ is selected from the group consisting of H, CH₃—(CH₂)₄—, CH₃—(CH₂)₃—, cyano-(CH₂)₃—, cyano-(CH₂)₄—, CF₃—(CH₂)₂—, cyclobutyl-CH₂—, cyclobutyl-(CH₂)₂—, cyclopropyl-(CH₂)₃—, cyclopropyl-C(O)CH₂—, CH₃—CH₂—NH—C(O)—CH₂—, (CH₃)₃—C—C(O)—CH₂—, cyclohexyl-CH₂—, OH-cyclohexyl-CH₂—, F₂-cyclohexyl-CH₂—, F-cyclohexenyl-CH₂—, tetrahydrofuranyl-CH₂—, tetrahydropyranyl-CH₂—, fluoro-benzyl, CH₃—O-benzyl, cyano-benzyl, methyl-benzyl, chloro-benzyl, oxo-tetrahydrofuranyl-CH₂—, oxo-pyrrolidinyl-CH₂—, pyridinyl-CH₂—, pyrazinyl-CH₂—, pyrimidinyl-CH₂—, CH₃-pyrazolyl-CH₂—, CH₃-oxazolyl-CH₂—, CH₃-isoxazolyl-CH₂—, CH₃-oxadiazolyl-CH₂—, CH₃-thiazolyl-CH₂—, and CH₃-thiadiazolyl-CH₂—;
R² is selected from the group consisting of H, NR⁴R⁵—C(O)—CR⁶R⁷—, CR⁸R⁹R¹⁰—, (CH₃)₂—N—CH₂—C(CH₃)₂—CH₂—, tetrahydronaphthalenyl, OH-dihydroindenyl, OH-cyclohexyl, CH₃—CH₂-pyrrolidinyl-CH₂—, oxadiazolyl-CR¹¹R¹²— optionally substituted with CH₃, NH₂, (CH₃)₂—N—C(O)—, CH₃—NH—C(O)—, tetrahydronaphthalenyl-NH—C(O)—, azepanyl-C(O)—, oxopyrrolidinyl(CH₂)₃—NH—C(O)—, CH₃—O—(CH₂)₂—NH—C(O)—, OH-cyclohexyl-NH—C(O)—, OH—CH₂-piperidinyl-C(O)—, CH₃—CH₂—, (CH₃)₂—CH—(CH₂)₂—NH—C(O)—, or (CH₃)₂—CH—; isoxazolyl-CR¹¹R¹²— optionally substituted with CH₃; furyl-CR¹¹R¹²— optionally substituted with CH₃ or CF₃: pyrazolyl-CR¹¹R¹²— optionally substituted with CH₃, (CH₃—CH—, or CH₃—CH₂—; thiazolyl-CR¹¹R¹²— optionally substituted with CH₃, CH₃—CH₂—, or CF₃; and dihydroisochromenyl-CR¹¹R¹²—;
R³ is selected from the group consisting of H, F, Cl, bromo, difluoro, methyl, cyano, methoxy, trifluoromethyl, methylthio, methylsulfinyl, methylsulfonyl and aminosulfonyl;
R⁴ and R⁵ are H, OH—(CH₂)₂—, NH₂—C(O)—(CH₂)₂—, CH₃—O—(CH₂)₂—, benzyl, pyridinyl, cyclobutyl, (CH₃)₃—C—, cyclopropyl, CH₃, OH—(CH₂)₃—, or (OH)₂—CH₂-CH—CH₂—;
R⁶ and R⁷ are H, (CH₃)—C—, CH₃, benzyl, phenyl, tetrahydropyranyl, cyclohexyl, (CH₃)₂—CH—CH₂—, (CH₃)₂—CH—, or (OH)(CH₃)CH—;
R⁸, R⁹ and R¹⁰ are H, (CH₃)₃—C—, NH₂—C(O)—, OH—CH₂, (CH₃CH₂)₂—N—CH₂—, CH₃, (OH) (CH₃)₂—CH—, CH₃—NH—C(O)—CH₂—, cyclopropyl-NH—C(O)—CH₂—, NH₂—C(O)—CH₂—NH—C(O)—CH₂—, or COOH—CH₂—; or two of R⁸, R⁹, or R¹⁰ form a cyclohexyl, and
R¹¹ and R¹² are H, CH₃, or (CH₃)₃—C—.

14. The compound of claim 13 or a pharmaceutically acceptable salt thereof, wherein R¹ is selected from the group consisting of

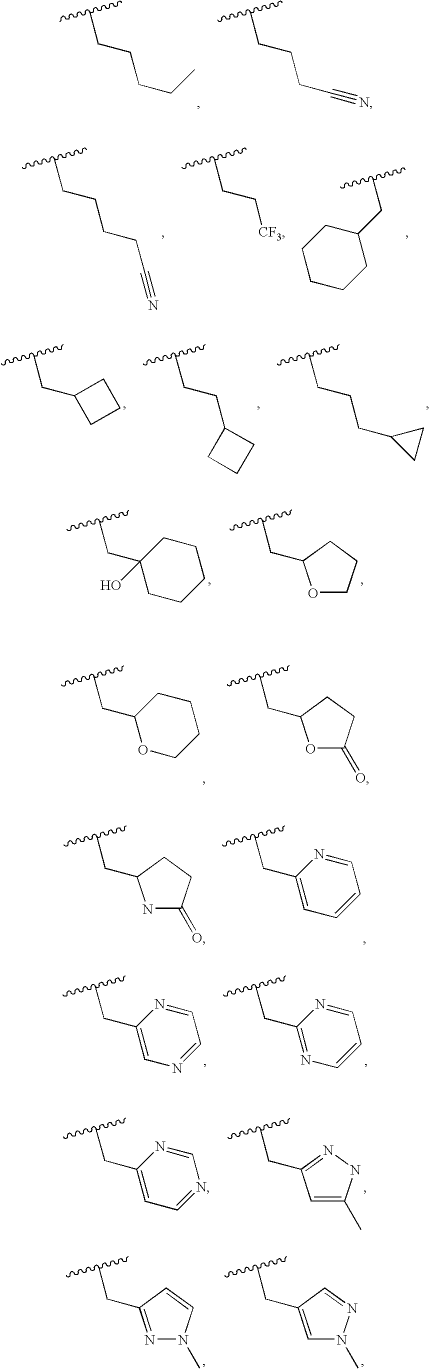

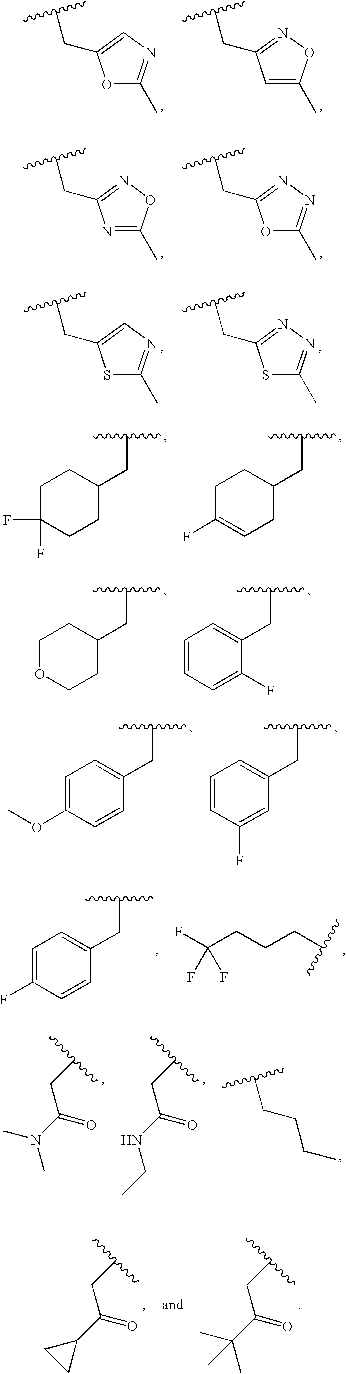

15. The compound of claim 13 or a pharmaceutically acceptable salt thereof, wherein R¹ is selected from the group consisting of

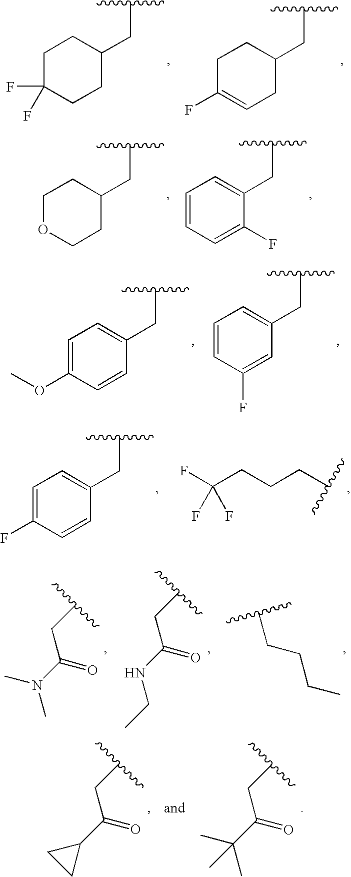

16. The compound of claim 13 or a pharmaceutically acceptable salt thereof, wherein R² is selected from the group consisting of

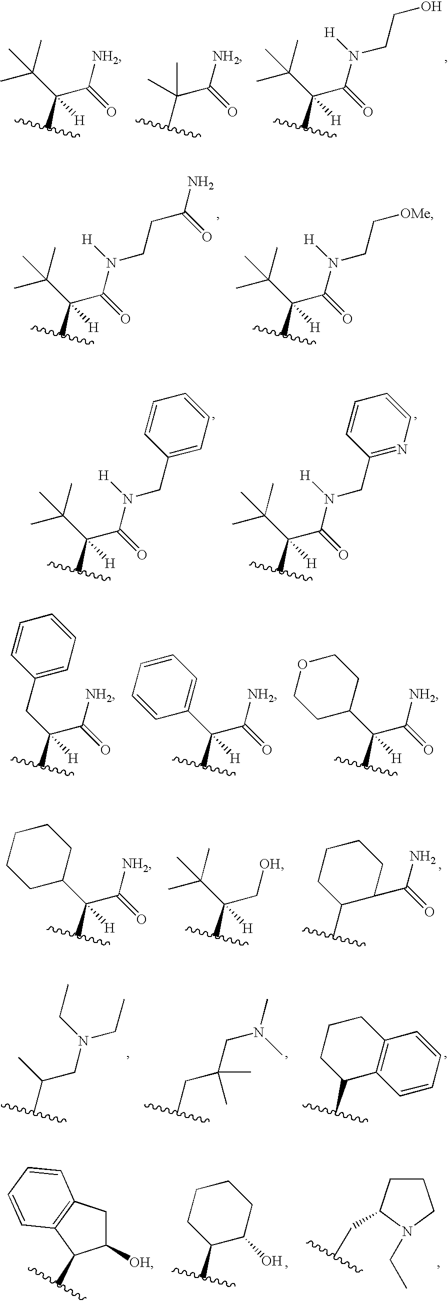

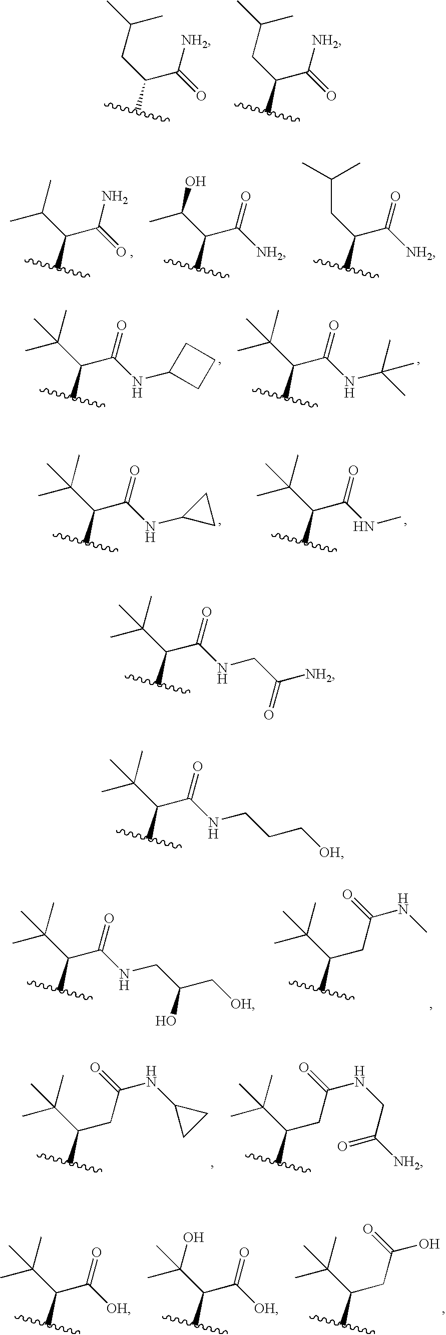

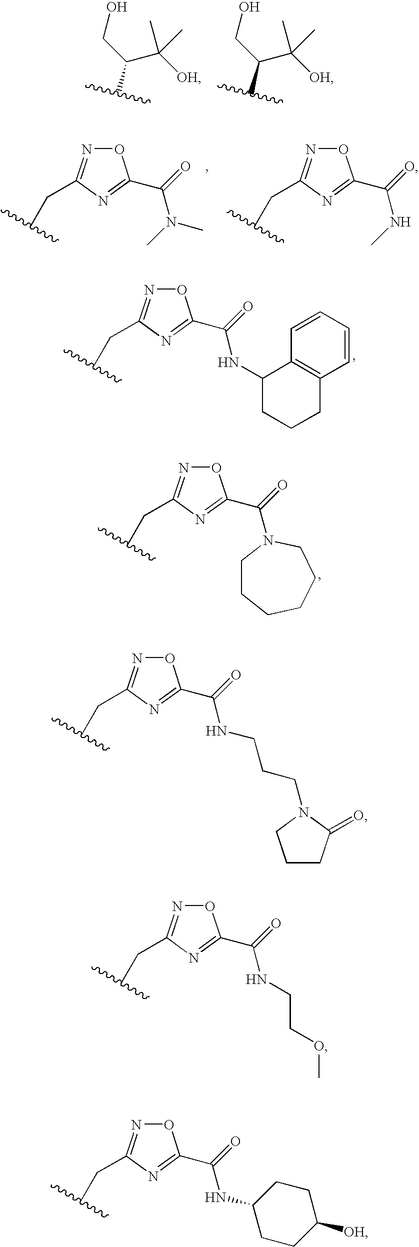

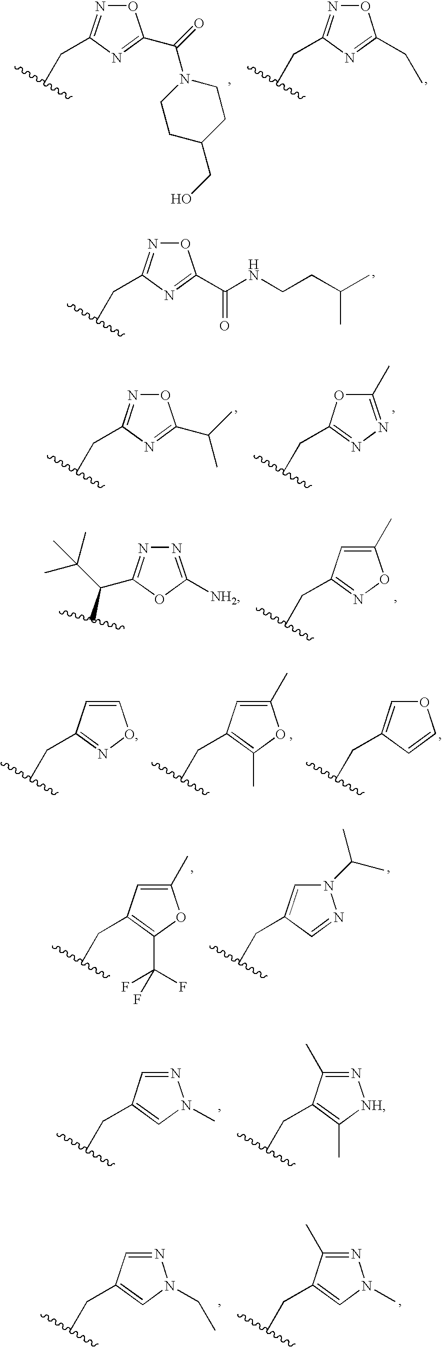

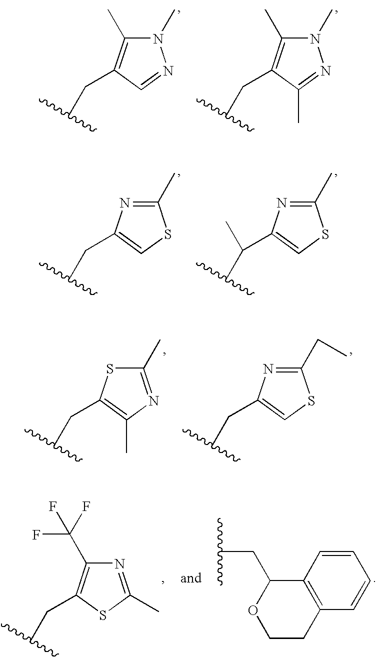

17. The compound of claim 13 or a pharmaceutically acceptable salt thereof, wherein R² is selected from the group consisting of

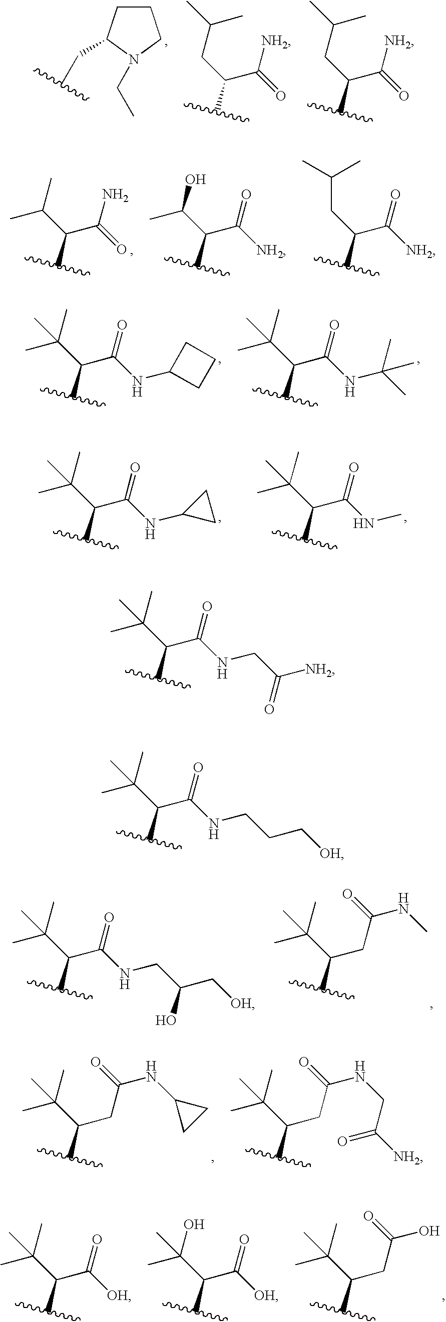

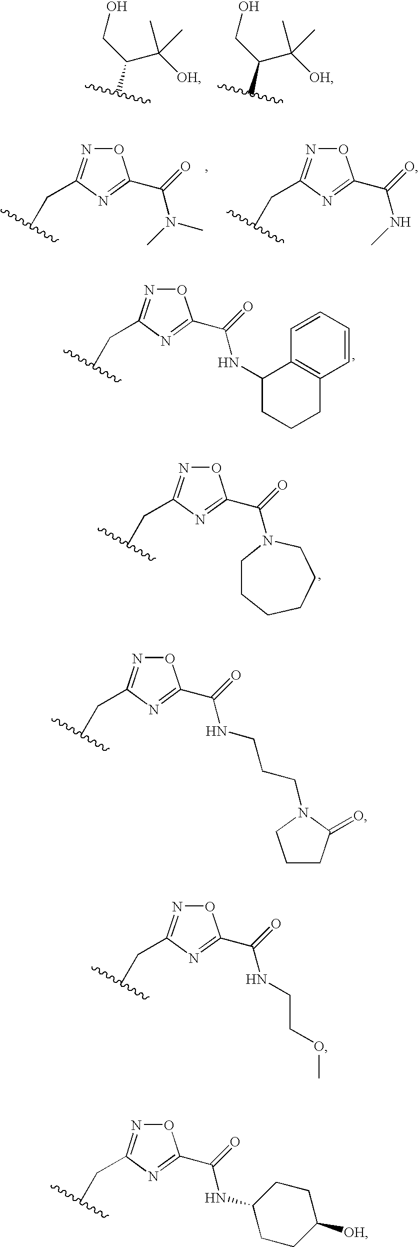

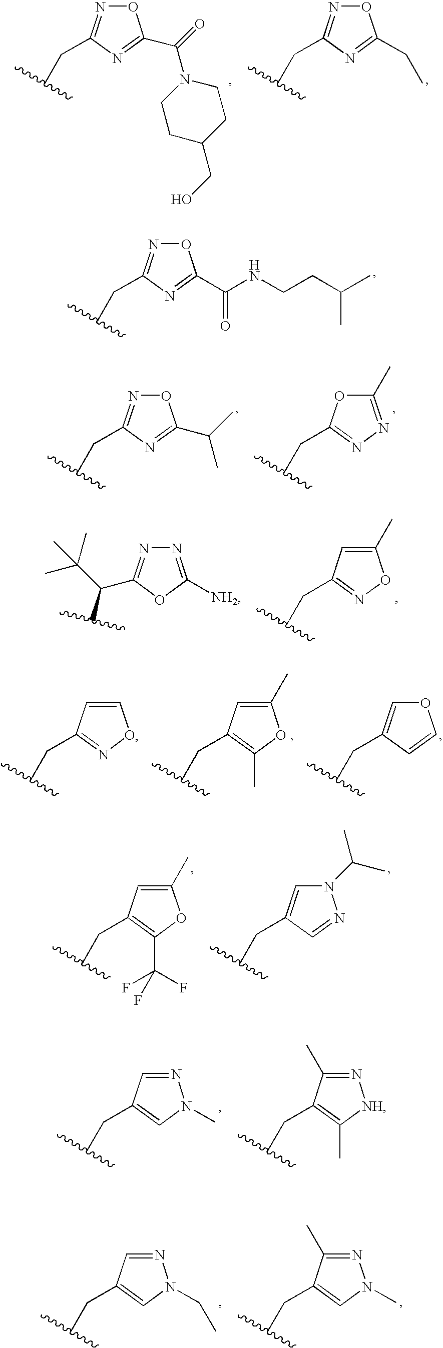

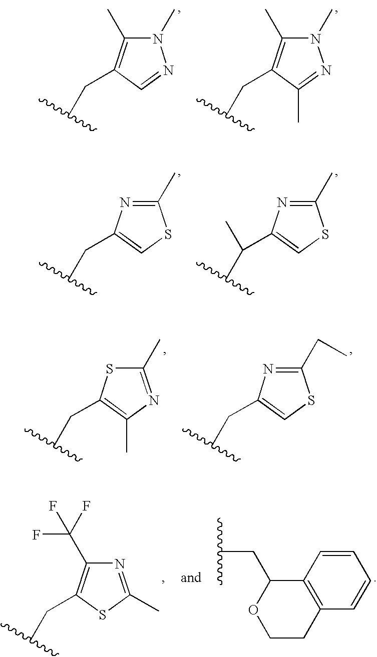

18. The compound selected from the group consisting of
N-[1-aminocarbonyl)cyclohexyl]-8-(cyclohexylmethyl)-5-methyl-2-oxo-2,3-dihydro-1H-benzimidazole-1-carboxamide;
N-[(1R)-1-(aminocarbonyl)-3-methylbutyl]-3-(cyclohexylmethyl)-5-methyl-2-oxo-2,3-dihydro-1H-benzimidazole-1-carboxamide;
Nalpha-{[3-(cyclohexylmethyl)-5-methyl-2-oxo-2,3-dihydro-1H-benzimidazol-1-yl]carbonyl}-L-phenylalaninamide;
N-[(1S)-1-(aminocarbonyl)-2-methylpropyl]-3-(cyclohexylmethyl)-5-methyl-2-oxo-2,3-dihydro-1H-benzimidazole-1-carboxamide;
N-[(1S)-1-(aminocarbonyl)-3-methylbutyl]-3-(cyclohexylmethyl)-(methyl-2-oxo-2,3-dihydro-1H-benzimidazole-1-carboxamide;
N-[(1S)-1-(aminocarbonyl)-2,2-dimethylpropyl]-3-(cyclohexylmethyl)-7-fluoro-2-oxo-2,3-dihydro-1H-benzimidazole-1-carboxamide;
N-[(1S)-1-(aminocarbonyl)-2,2-dimethylpropyl]-5-cyano-2-oxo-3-(tetrahydro-2H-pyran-4-ylmethyl)-2,3-dihydro-1H-benzimidazole-1-carboxamide
N-[(1S)-2-amino-1-cyclohexyl-2-oxoethyl]-3-cyclohexyl methyl)-5-methyl-2-oxo-2,3-dihydro-1H-benzimidazole-1-carboxamide;
N-[(1S,2R)-1-(aminocarbonyl)-2-hydroxypropyl]-2-oxo-3-(tetrahydro-2H-pyran-4-ylmethyl)-2,3-dihydro-1H-benzimidazole-1-carboxamide;
N-[(1S)-1-(aminocarbonyl)-2-methylpropyl]-2-oxo-3-(tetrahydro-2H-pyran-4-ylmethyl)-2,3-dihydro-1H-benzimidazole-1-carboxamide;
2-oxo-N-[(1S)-1,2,3,4-tetrahydronaphthalen-1-yl]-3-(tetrahydro-2H-pyran-4-ylmethyl)-2,3-dihydro-1H-benzimidazole-1-carboxamide;
N-[(1S)-2-amino-2-oxo-1-phenylethyl]-2-oxo-3-(tetrahydro-2H-pyran-4-ylmethyl)-2,3-dihydro-1H-benzimidazole-1-carboxamide;
Nalpha-{[2-oxo-3-(tetrahydro-2H-pyran-4-ylmethyl)-2,3-dihydro-1H-benzimidazol-1-yl]carbonyl}-L-phenylalaninamide;
N-[(1S)-1-(aminocarbonyl)-3-methylbutyl]-2-oxo-3-(tetrahydro-2H-pyran-4-ylmethyl-2,3-dihydro-1H-benzimidazole-1-carboxamide;
N-[(1S,2R)-2-hydroxy-2,3-dihydro-1H-inden-1-yl]-2-oxo-3-(tetrahydro-2H-pyran-4-ylmethyl)-2,3-dihydro-1H-benzimidazole-1-carboxamide;
N-[(1S,2S-2-hydroxycyclohexyl]-2-oxo-3-(tetrahydro-2H-pyran-4-ylmethyl)-2,3-dihydro-1H-benzimidazole-1-carboxamide,
N-[(1S)-1-(hydroxymethyl) 2,2-dimethylpropyl]-2-oxo-3-(tetrahydro-2H-pyran-4-ylmethyl)-2,3-dihydro-1H-benzimidazole-1-carboxamide;
N-[3-(dimethylamino)-2,2-dimethylpropyl]-2-oxo-3-(tetrahydro-2H-pyran-4-ylmethyl)-2,3-dihydro-1H-benzimidazole-1-carboxamide;
N-[(1S)-2-amino-1-cyclohexyl-2-oxoethyl]-2-oxo-3-(tetrahydro-2H-pyran-4-ylmethyl)-2,3-dihydro-1H-benzimidazole-t-carboxamide;
3-methyl-N-{[2-oxo-3-(tetrahydro-2H-pyranyl-4-methyl)-2,3-dihydro-1H-benzimidazol-1-yl]carbonyl}-L-valine;
3-hydroxy-N-{[2-oxo-3-(tetrahydro-2H-pyran-4-ylmethyl)-2,3-dihydro-1H-benzimidazol-1-yl]carbonyl}-L-valine;
N-[(1S)-1-(aminocarbonyl)-2-hydroxy-2-methylpropyl]-2-oxo-3-(tetrahydro-2H-pyranyl-4-methyl)-2,3-dihydro-1H-benzimidazole-1-carboxamide;
N-[(1S)-2-hydroxy-1-(hydroxymethyl)-2-methylpropyl]-2-oxo-3-(tetrahydro-2H-pyran-4-ylmethyl)-2,3-dihydro-1H-benzimidazole-1-carboxamide;
N-[(1R)-2-hydroxy-1-(hydroxymethyl)-2-methylpropyl]-2-oxo-3-tetrahydro-2H-pyran-4-ylmethyl)-2,3-dihydro-1H-benzimidazole-1 carboxamide;
N-[(1S)-1-(aminocarbonyl)-2,2-dimethylpropyl]-fluoro-2-oxo-3-(tetrahydro-2H-pyranyl-4-methyl)-2,3-dihydro-1H-benzimidazole-1-carboxamide;
(3R)-4,4-dimethyl-3-({[2-oxo-3-(tetrahydro-2H-pyran-4-ylmethyl)-2,3-dihydro-1H-benzimidazol-1-yl]carbonyl}amino)pentanoic acid;
N-[(1S)-1-(aminocarbonyl)-2,2-dimethylpropyl]-3-butyl-2-oxo-2,3-dihydro-1H-benzimidazole-1-carboxamide;
N-[(S)-1-(aminocarbonyl)-2,2-dimethylpropyl]-2-oxo-3-(4,4,4-trifluorobutyl)-2,3-dihydro-1H-benzimidazole-1-carboxamide;
N-[(1S)-1-(aminocarbonyl)-2,2-dimethylpropyl]-5,6-difluoro-2-oxo-3-(tetrahydro-2H-pyran-4-ylmethyl)-2,3-dihydro-1H-benzimidazole-1-carboxamide;
N-[(1R)-1-(2-amino-2-oxoethyl)-2,2-dimethylpropyl]-2-oxo-3-(tetrahydro-2H-pyran-4-ylmethyl)-2,3-dihydro-1H-benzimidazole-1-carboxamide;
N-{(1R)-1-[2-(cyclopropylamino)-2-oxoethyl]-2,2-dimethylpropyl}-2-oxo-3-(tetrahydro-2H-pyran-4-ylmethyl)-2,3-dihydro-1H-benzimidazole-1-carboxamide;
N-[(1R)-1-{2-[2-amino-2-oxoethyl}amino]-2-oxoethyl)-2,2-dimethylpropyl]-2-oxo-3-(tetrahydro-2H-pyran-4-ylmethyl)-2,3-dihydro-1H-benzimidazole-1-carboxamide;
N-{(1S)-1-[(cyclopropylamino)carbonyl]-2,2-dimethylpropyl}-2-oxo-3(tetrahydro-2H-pyran-4-ylmethyl)-2,3-dihydro-1H-benzimidazole-1-carboxamide;
N-{(1S)-1-[(tert-butylamino)carbonyl]-2,2-dimethylpropyl}-2-oxo-3-(tetrahydro-2H-pyran-4-ylmethyl)-2,3-dihydro-1H-benzimidazole-1-carboxamide;
N-{(1S)-1-[(cyclobutylamino)carbonyl]-2,2-dimethylpropyl}-2-oxo-3-(tetrahydro-2H-pyran-4-ylmethyl)-2,3-dihydro-1H-benzimidazole-1-carboxamide;
5-fluoro-N-[(1S)-1-(hydroxymethyl)-2,2-dimethylpropyl]-2-oxo-3-(tetrahydro-2H-pyran-4-ylmethyl)-2,3-dihydro-1H-benzimidazole-1-carboxamide;
3-methyl-N-{[2-oxo-3-(tetrahydro-2H-pyran-4-ylmethyl-2,3-dihydro-1H-benzimidazol-1-yl]carbonyl}L-valylglycinamide;
N-{(1S)-2,2-dimethyl-1-[(methylamino)carbonyl]propyl}-2-oxo-3-(tetrahydro-2H-pyran-4-ylmethyl)-2,3-dihydro-1H-benzimidazole-1-carboxamide;
N-[(1S)-1-(aminocarbonyl)-2,2-dimethylpropyl]-3-[(4-fluorocyclohex-3-en-1-yl)methyl]-2-oxo-2,3-dihydro-1H-benzimidazole-1-carboxamide (diastereomer 1);
N-[(1S)-1-(aminocarbonyl)-2,2-dimethylpropyl]-3-[(4-fluorocyclohex-3-en-1-yl)methyl]-2-oxo-2,3-dihydro-1H-benzimidazole-1-carboxamide (diastereomer 2);
N-[(1S)-1-(aminocarbonyl)-2,2-dimethylpropyl]-3-[(4,4-difluorocyclohexyl)methyl]-2-oxo-2,3-dihydro-1H-benzimidazole-1-carboxamide;
N-[(1S)-1-(aminocarbonyl)-2,2-dimethylpropyl]-5-bromo-3-(cyclohexylmethyl)-2-oxo-2,3-dihydro-1H-benzimidazole-1-carboxamide;
5-bromo-3-(cyclohexylmethyl)-N-[(1S)-1-(hydroxymethyl)-2,2-dimethylpropyl]-2-oxo-2,3-dihydro-1H-benzimidazole-1-carboxamide;
N-{(1R)-2,2-dimethyl-1-[2-(methylamino)-2-oxoethyl]propyl}-2-oxo-3-tetrahydro-2H-pyran-4-ylmethyl)-2,3-dihydro-1H-benzimidazole-1-carboxamide;
N-{(1S)-1-[(cyclopropylamino)carbonyl]-2,2-dimethylpropyl}-5-fluoro-2-oxo-3-(tetrahydro-2H-pyran-4-ylmethyl)-2,3-dihydro-1H-benzimidazole-1-carboxamide;
N-[(2,5-dimethyl-3-furyl)methyl]-2-oxo-3-(tetrahydro-2H-pyran-4-ylmethyl)-2,3-dihydro-1H-benzimidazole-1-carboxamide;
N-[(5-methylisoxazol-3-yl)methyl]-2-oxo-3-(tetrahydro-2H-pyran-4-ylmethyl)-2,3-dihydro-1H-benzimidazole-1-carboxamide;
N-[(1S)-1-(aminocarbonyl)-2,2-dimethylpropyl]-3-[2-(dimethylamino)-2-oxoethyl]-2-oxo-2,3-dihydro-1H-benzimidazole-1-carboxamide;
N-[(1S)-1-(aminocarbonyl)-2,2-dimethylpropyl]-3-[2-(ethylamino)-2-oxoethyl]-2-oxo-2,3-dihydro-1H-benzimidazole-1-carboxamide;
N-[(1S)-1-(aminocarbonyl)-2,2-dimethylpropyl]-3-(2-cyclopropyl-2-oxoethyl)-2-oxo-2,3-dihydro-1H-benzimidazole-1-carboxamide;
N-[(1S)-1-(aminocarbonyl)-2,2-dimethylpropyl]-3-(3,3-dimethyl-2-oxobutyl)-2-oxo-2,3-dihydro-1H-benzimidazole-1-carboxamide;
N-[(1S)-1-{[(2-hydroxyethyl)amino]carbonyl}-2,2-dimethylpropyl]-2-oxo-3-(tetrahydro-2H-pyran-4-ylmethyl)-2,3-dihydro-1H-benzimidazole-1-carboxamide;
N-[(1S)-1-{[(3-hydroxypropyl)amino]carbonyl}-2,2-dimethylpropyl]-2-oxo-3-(tetrahydro-2H-pyran-4-ylmethyl)-2,3-dihydro-1H-benzimidazole-1-carboxamide;
N-[(1S)-1-(5-amino-1,3,4-oxadiazol-2-yl)-2,2-dimethylpropyl]-2-oxo-3-(tetrahydro-2H-pyran-4-ylmethyl)-2,3-dihydro-1H-benzimidazole-1-carboxamide;
N-{[5-methyl-2-(trifluoromethyl)-3-furyl]methyl}-2-oxo-3-(tetrahydro-2H-pyran-4-ylmethyl)-2,3-dihydro-1H-benzimidazole-1-carboxamide;
N-[(5-{[4-(hydroxymethyl)piperidin-1-yl]carbonyl}-1,2,4-oxadiazol-3-yl)methyl]-2-oxo-3-(tetrahydro-2H-pyran-4-ylmethyl)-2,3-dihydro-1H-benzimidazole-1-carboxamide;
N-[(1,5-dimethyl-1H-pyrazol-4-yl)methyl]-2-oxo-3-(tetrahydro-2H-pyran-4-ylmethyl)-2,3-dihydro-1H-benzimidazole-1-carboxamide;
N-[(5-{[(3-methylbutyl)amino]carbonyl}-1,2,4-oxadiazol-3-yl)methyl]-2-oxo-3-(tetrahydro-2H-pyran-4-ylmethyl)-2,3-dihydro-1H-benzimidazole-1-carboxamide;
N-[(5-isopropyl-1,2,4-oxadiazol-3-yl)methyl]-2-oxo-3-(tetrahydro-2H-pyran-4-ylmethyl)-2,3-dihydro-1H-benzimidazole-1-carboxamide;
2-oxo-3-(tetrahydro-2H-pyran-4-ylmethyl)-N-[(1,3,5-trimethyl-1H-pyrazolyl)methyl]-2,3-dihydro-1H-benzimidazole-1-carboxamide;
N-(1,3-oxazol-4-ylmethyl)-2-oxo-3-(tetrahydro-2H-pyran ylmethyl)-2,3-dihydro-1H-benzimidazole-1-carboxamide;
N-[(5-ethyl-1,2,4-oxadiazol-3-yl)methyl]-2-oxo-3-(tetrahydro-2H-pyran-4-ylmethyl)-2,3-dihydro-1H-benzimidazole-1-carboxamide;
N-[(2-ethyl-1,3-thiazol-4-yl)methyl]-2-oxo-3-(tetrahydro-2H-pyran-4-ylmethyl)-2,3-dihydro-1H-benzimidazole-1-carboxamide;
N-(3-furylmethyl)-2-oxo-3-(tetrahydro-2H-pyran-4-ylmethyl)-2,3-dihydro-1H-benzimidazole-1-carboxamide;
N-[(5-{[(3,4-dihydro-1H-isochromen-1-ylmethyl)amino]carbonyl}-1,2,4-oxadiazol-3-yl)methyl]-2-oxo-3-(tetrahydro-2H-pyran-4-ylmethyl)-2,3-dihydro-1H-benzimidazole-1-carboxamide;
N-{[5-(azepan-1-ylcarbonyl)-1,2,4-oxadiazol-3-yl]methyl}-2-oxo-3-(tetrahydro-2H-pyran-4-ylmethyl)-2,3-dihydro-1H-benzimidazole-1-carboxamide;
2-oxo-N-{[5-({[3-(2-oxopyrrolidin-1-yl)propyl]amino}carbonyl)-1,2,4-oxadiazol-3-yl]methyl}-3-(tetrahydro-2H-pyran-4-ylmethyl)-2,3-dihydro-1H-benzimidazole-1-carboxamide;
N-[(1-ethyl-1H-pyrazol-4-yl)methyl]-2-oxo-3-(tetrahydro-2H-pyran-4-ylmethyl)-2,3-dihydro-1H-benzimidazole-1-carboxamide;
2-oxo-N-({5-[(1,2,3,4-tetrahydronaphthalen-1-ylamino)carbonyl]-1,2,4-oxadiazol-3-yl}methyl)-3-(tetrahydro-2H-pyran-4-ylmethyl)-2,3-dihydro-1H-benzimidazole-1-carboxamide;
N-({5-[(dimethylamino)carbonyl]-1,2,4-oxadiazol-3-yl}methyl)-2-oxo-3-(tetrahydro-2H-pyran-4-ylmethyl)-2,3-dihydro-1H-benzimidazole-1-carboxamide;
N-[(5-{[(2-methoxyethyl)amino]carbonyl}-1,2,4-oxadiazol-3-yl)methyl]-2-oxo-3-(tetrahydro-2H-pyran-4-ylmethyl)-2,3-dihydro-1H-benzimidazole-1-carboxamide;
N-[(2,4-dimethyl-1,3-thiazol-5-yl)methyl]-2-oxo-3-(tetrahydro-2H-pyran-4-ylmethyl)-2,3-dihydro-1H-benzimidazole-1-carboxamide;
N-({5-[(methylamino)carbonyl]-1,2,4-oxadiazol-3-yl}methyl)-2-oxo-3-(tetrahydro-2H-pyran-4-ylmethyl)-2,3-dihydro-1H-benzimidazole-1-carboxamide;
N-[(1-isopropyl-1H-pyrazolyl)methyl]-2-oxo-3-(tetrahydro-2H-pyran-4-ylmethyl)-2,3-dihydro-1H-benzimidazole-1-carboxamide;
N-[(1,3-dimethyl-1H-pyrazol-4-yl)methyl]-2-oxo-3-(tetrahydro-2H-pyran-4-ylmethyl)-2,3-dihydro-1H-benzimidazole-1-carboxamide;
N-[(2-methyl-1,3-thiazolyl)methyl]-2-oxo-3-(tetrahydro-2H-pyran-4-ylmethyl)-2,3-dihydro-1H-benzimidazole-1-carboxamide;
N-[(5-{[(trans-4-hydroxycyclohexyl)amino]carbonyl}-1,2,4-oxadiazol-3-yl)methyl]-2-oxo-3-(tetrahydro-2H-pyran-4-ylmethyl)-2,3-dihydro-1H-benzimidazole-1-carboxamide;
N-[(3,5-dimethyl-1H-pyrazol-4-yl)methyl]-2-oxo-3-(tetrahydro-2H-pyran-4-ylmethyl)-2,3-dihydro-1H-benzimidazole-1-carboxamide;
N-[(1-methyl-1H-pyrazol-4-yl)methyl]-2-oxo-3-(tetrahydro-2H-pyran-4-ylmethyl)-2,3-dihydro-1H-benzimidazole-1-carboxamide;
N-[(1S)-1-({[(2S)-2,3-dihydroxypropyl]amino}carbonyl)-2,2-dimethylpropyl]-2-oxo-3-(tetrahydro-2H-pyran-4-ylmethyl)-2,3-dihydro-1H-benzimidazole-1-carboxamide;
N-[(5-methyl-1,3,4-oxadiazol-2-yl)methyl]-2-oxo-3-(tetrahydro-2H-pyran-4-ylmethyl)-2,3-dihydro-1H-benzimidazole-1-carboxamide;
N-[(1S)-1-(5-amino-1,3,4-oxadiazol-2-yl)-2,2-dimethylpropyl]-5-fluoro-2-oxo-3(tetrahydro-2H-pyran-4-ylmethyl)-2,3-dihydro-1H-benzimidazole-1-carboxamide;
N-[(1S)-1-(aminocarbonyl)-2,2-dimethylpropyl]-3-benzyl-2-oxo-2,3-dihydro-1H-benzimidazole-1-carboxamide;
N-[2-methyl-4-(trifluoromethyl)-1,3-thiazol-5-yl]methyl)-2-oxo-3-tetrahydro-2H-pyran-4-ylmethyl)-2,3-dihydro-1H-benzimidazole-1-carboxamide;
N-[1-(2-methyl-1,3-thiazol-4-yl)ethyl]-2-oxo-3-(tetrahydro-2H-pyran-4-ylmethyl)-2,3-dihydro-1H-benzimidazole-1-carboxamide;
N-[(1S)-1-(aminocarbonyl)-2,2-dimethylpropyl]-2-oxo-3-{[(2R)-5-oxotetrahydrofuran-2-yl]methyl}-2,3-dihydro-1H-benzimidazole-1-carboxamide;
N-[(1S)-1-(5-amino-1,3,4-oxadiazol-2-yl)-2,2-dimethylpropyl]-2-oxo-3-{[(2R)-5-oxotetrahydrofuran-2-yl]methyl}-2,3-dihydro-1H-benzimidazole-1-carboxamide;
N-[(1S)-1-{[(3-hydroxypropyl)amino]carbonyl}-2,2-dimethylpropyl]-2-oxo-3-{[(2R)-5-oxotetrahydrofuran-2-yl]methyl}-2,3-dihydro-1H-benzimidazole-1-carboxamide;
N-[(1S)-1-{[(2-hydroxyethyl)amino]carbonyl}-2,2-dimethylpropyl]-2-oxo-3-{[(2R)-5-oxotetrahydrofuran-2-yl]methyl}-2,3-dihydro-1H-benzimidazole-1-carboxamide;
5-fluoro-N-[(1S)-1-{[(3-hydroxypropyl)amino]carbonyl}-2,2-dimethylpropyl]-2-oxo-3-(tetrahydro-2H-pyran-4-ylmethyl)-2,3-dihydro-1H-benzimidazole-1-carboxamide;
5-fluoro-N-[(1S)-1-{[(2-hydroxyethyl)amino]carbonyl}-2,2-dimethylpropyl]-2-oxo-3-(tetrahydro-2H-pyran-4-ylmethyl)-2,3-dihydro-1H-benzimidazole-1-carboxamide;
N-[(1S)-1-(aminocarbonyl)-2,2-dimethylpropyl]-3-(2-fluorobenzyl)-2-oxo-2,3-dihydro-1H-benzimidazole-1-carboxamide;
N-[(1S)-1-(aminocarbonyl)-2,2-dimethylpropyl]-3-(4-methoxybenzyl)-2-oxo-2,3-dihydro-1H-benzimidazole-1-carboxamide;
N-[(1S)-1-(5-amino-1,3,4-oxadiazol-2-yl)-2,2-dimethylpropyl]-3-benzyl-2-oxo-2,3-dihydro-1H-benzimidazole-1-carboxamide;
3-benzyl-N-[(1S)-1-{[(2-hydroxyethyl)amino]carbonyl}-2,2-dimethylpropyl]-2-oxo-2,3-dihydro-1H-benzimidazole-1-carboxamide;
3-benzyl-N-[(1S)-1-{[(3-hydroxypropyl)amino]carbonyl}-2,2-dimethylpropyl]-2-oxo-2,3-dihydro-1H-benzimidazole-1-carboxamide;
N-[(1S)-1-aminocarbonyl)-2-(2-dimethylpropyl]-3-(3-fluorobenzyl)-2-oxo-2,3-dihydro-1H-benzimidazole-1-carboxamide;
N-[(1S)-1-(aminocarbonyl)-2,2-dimethylpropyl]-3-(4-fluorobenzyl)-2-oxo-2,3-dihydro-1H-benzimidazole-1-carboxamide;
N-[(1S)-1-aminocarbonyl)-2,2-dimethylpropyl]-2-oxo-3-(tetrahydro-2H-pyran-4-ylmethyl)-2,3-dihydro-1H-imidazo[4,5-b]pyridine-1-carboxamide;
N-[(1S)-1-(aminocarbonyl)-2,2-dimethylpropyl]-2-oxo-3-pentyl-2,3-dihydro-1H-imidazo[4,5-b]pyridine-1-carboxamide;
N-[(1S)-1-(aminocarbonyl)-2,2-dimethylpropyl]-3-benzyl-2-oxo-2,3-dihydro-1H-benzimidazole-1-carboxamide;
N-[(1S)-(aminocarbonyl)-2,2-dimethylpropyl]-3-(2-fluorobenzyl)-2-oxo-2,3-dihydro-1H-benzimidazole-1-carboxamide;
N-[(1S)-1-(aminocarbonyl)-2,2-dimethylpropyl]-3-(4-methoxybenzyl)-2-oxo-2,3-dihydro-1H-benzimidazole-1-carboxamide;
N-[(1S)-1-(5-amino-1,3,4-oxadiazol-2-yl)-2,2-dimethylpropyl]-3-benzyl-2-oxo-2,3-dihydro-1H-benzimidazole-1-carboxamide;
3-benzyl-N-[(1S)-1-{[(2-hydroxyethyl)amino]carbonyl}-2,2-dimethylpropyl]-2-oxo-2,3-dihydro-1H-benzimidazole-1-carboxamide;
3-benzyl-N-[(1S)-{[3-hydroxypropyl)amino]carbonyl}-2,2-dimethylpropyl]-2-oxo-2,3-dihydro-1H-benzimidazole-1-carboxamide;
N-[(1S)-1-(aminocarbonyl)-2,2-dimethylpropyl]-3-(4-cyanobenzyl)-2-oxo-2,3-dihydro-1H-benzimidazole-1-carboxamide;
N-[(1S)-1-(aminocarbonyl)-2,2-dimethylpropyl]-3-(2-cyanobenzyl)-2-oxo-2,3-dihydro-1H-benzimidazole-1-carboxamide;
N-[(1S)-1-(aminocarbonyl)-2,2-dimethylpropyl]-3-(3-fluorobenzyl)-2-oxo-2,3-dihydro-1H-benzimidazole-1-carboxamide;
N-[(1S)-1-(aminocarbonyl)-2,2-dimethylpropyl]-3-(4-fluorobenzyl)-2-oxo-2,3-dihydro-1H-benzimidazole-1-carboxamide;
3-(4-fluorobenzyl)-N-[(1S)-1-{[(2-hydroxyethyl)amino]carbonyl}-2,2-dimethylpropyl]-2-oxo-2,3-dihydro-1H-benzimidazole-1-carboxamide;
3-(4-fluorobenzyl)-N-[(1S)-1-{[(3-hydroxypropyl)amino]carbonyl}-2,2-dimethylpropyl]-2-oxo-2,3-dihydro-1H-benzimidazole-1-carboxamide;
N-{[3-(4-fluorobenzyl)-2-oxo-2,3-dihydro-1H-benzimidazol-1-yl]carbonyl}-3-methyl-L-valylglycinamide; and
N-[(1S)-1-(5-amino-1,3,4-oxadiazol-2-yl)-2,2-dimethylpropyl]-3-(4-fluorobenzyl)-2-oxo-2,3-dihydro-1H-benzimidazole-1-carboxamide;
or a pharmaceutically acceptable salt thereof.

19. A method for the treatment of a condition mediated by CB1 receptor activity in a mammalian subject including a human, which comprises administering to a mammal in need of such treatment a therapeutically effective amount of a compound or pharmaceutically acceptable salt thereof of Formula I.

20. The method of claim 19, wherein said condition is selected from the group consisting of inflammatory pain, nociceptive pain, neuropathic pain, fibromyalgia, chronic low back pain, visceral pain, acute cerebral ischemia, pain, chronic pain, acute pain, post herpetic neuralgia, neuropathies, neuralgia, diabetic neuropathy, HIV-related neuropathy, nerve injury, rheumatoid arthritic pain, osteoarthritic pain, back pain, cancer pain, dental pain, fibromyalgia, neuritis, sciatica, inflammation, neurodegenerative disease, spasticity, epilepsy, Tourette's syndrome, Parkinson's disease, neuroprotection, anxiety, cough, broncho constriction, irritable bowel syndrome (IBS), Inflammatory bowel disease (IBD), colitis, cerebrovascular ischemia, cachexia, nausea, emesis, chemotherapy-induced emesis, rheumatoid arthritis, asthma, Crohn's disease, ulcerative colitis, asthma, dermatitis, seasonal allergic rhinitis, gastroesophageal reflux disease (GERD), constipation, diarrhea, functional gastrointestinal disorder, cutaneous T cell lymphoma, multiple sclerosis, osteoarthritis, psoriasis, systemic lupus erythematosus, diabetes, glaucoma, osteoporosis, glomerulonephritis, renal ischemia, nephritis, hepatitis, cerebral stroke, vasodilation, hypertension, vasculitis, myocardial infarction, cerebral ischemia, reversible airway obstruction, adult respiratory disease syndrome, chronic obstructive pulmonary disease (COPD), cryptogenic fibrosing alveolitis and bronchitis.
